(19) United States
(12) Patent Application Publication
(10) Pub. No.: US 2025/0304919 A1
(43) Pub. Date: Oct. 2, 2025

(54) PRODUCTION OF BIOLOGICAL SCALABLE NANORODS

(71) Applicant: MASSEY VENTURES LIMITED, Palmerston North (NZ)

(72) Inventors: Jasna Rakonjac, Palmerston North (NZ); Rayen Leon Quezada, Palmerston North (NZ); Catherine Davenport, Palmerston North (NZ); Vuong Van Hung Le, Da Nang City (VN); Sofia Khanum, Palmerston North (NZ); Majela Gonzalez-Miro, Lund (SE); Jun Zhou, Hamilton (NZ); Sean Bisset, Auckland (NZ); Marina Rajic, Novi Sad (RS)

(21) Appl. No.: 18/839,589

(22) PCT Filed: Feb. 20, 2023
(86) PCT No.: PCT/IB2023/051546

§ 371 Date: Aug. 19, 2024

(30) Foreign Application Priority Data
Feb. 21, 2022 (AU) 2022900380
Oct. 17, 2022 (AU) 2022903032

(51) Int. Cl. C12N 7/00; C07K 14/01; C12N 15/70; C12N 15/86; C12R 1/19

(52) U.S. Cl. CPC C12N 7/00; C07K 14/01; C12N 15/70; C12N 15/86; C12N 2795/14031; C12N 2795/14051; C12N 2795/14052; C12R 2001/19

(57) ABSTRACT

Disclosed herein are nanorod productions systems (NPS) useful for the production of biological scalable functionalization-ready nanorods (BSFnano). The nanorods produced are derived from filamentous phage Ff (f1, M13 or fd). The NPS disclosed herein permits efficient biological production of non-infectious, heat-stable isomorphic proteinaceous nanorods comprising modifications allowing site-specific recombinant, chemical and enzymatic attachment of peptide and non-peptide functionalities in an orthogonal manner. Also disclosed are methods of making and using these nanorods, such as in methods of detecting target molecules.

### FIELD OF INVENTION

The invention relates generally to systems for producing biological scalable functionalization-ready nanorods (BSFnano) derived from filamentous phage Ff (f1, M13 or fd). The system permits efficient biological production of non-infectious, heat-stable isomorphic proteinaceous nanorods comprising modifications allowing site-specific recombinant, chemical and enzymatic attachment of peptide and non-peptide functionalities in an orthogonal manner.

### BACKGROUND

Multitude of medical and nanotechnology applications require the use of particles that can be functionalized orthogonally by peptide (protein) or non-protein functionalities (Sarikaya et al., 2003). While non-biological nanoparticles have been used for a range of diagnostic and nanotechnology applications, they create several problems, such as toxicity of the particles themselves and sustainability issues due to the use of toxic chemicals in production of the particles (Wang and Tang, 2020). The toxicity precludes medical therapeutic applications that require direct introduction into the patients. Furthermore, production of non-biological nanorods that are isomorphic and orthogonally modifiable is very difficult (Corrigan et al., 2021).

A limited number of biological nanoparticles (including nanorods) have been used to date in nanotechnological and biomedical applications. The most prominent of these biological nanoparticles are filamentous bacteriophages Ff, bacterial viruses of Escherichia coli K12.

Ff bacteriophages are central to phage display technology and have been used as biological particles that are suitable for attachment of functional groups. A number of medical and nanotechnology applications using the whole phage or long, phage-derived filaments containing complete plasmids, called phagemids, are known (Barbas III et al., 2001).

Ff filamentous bacteriophage (encompassing f1, fd and M13 species) carry the DNA sequences required for replication and packaging in their intergenic (IG) sequence (Model and Russel, 1988; Rakonjac et al., 2017). Ff phage replicate using a rolling circle mode, one strand at a time. The genome of the Ff phage is single-stranded circular (positive; +) strand ssDNA. The second (negative; −) strand is synthetized from the (−) on by host enzymes, resulting in a double-stranded circular DNA replicative form of the genome (RF). The RF serves as the template for transcription and translation of phage proteins required for replication and assembly of the progeny phage. Rolling circle replication from the positive (+) strand origin of replication (ori) that uses the RF as the template requires the phage-encoded replication protein, pII, and results in single-stranded circular DNA (ssDNA) that is the filamentous phage genome.

A long hairpin structure in this ssDNA genome serves as the packaging signal required for assembly of the filamentous virion. Early in the infection cycle, the ssDNA undergoes replication from the (−) on to increase the RF copy number (up to 50 copies per cell). This is in contrast to later stages of infection where the ssDNA is coated by protein pV forming the “packaging substrate” required for assembly of the virion. The ssDNA in the packaging substrate forms a Watson-Crick-like helix, each strand interacting with one subunit of the pV dimers. The exception is the packaging signal, a true DNA helix that is not covered by pV. This complex, called the “packaging substrate”, is targeted to the trans-envelope assembly-secretion machinery that assembles the virion.

The (+) on has a site at which the replication protein pII makes a cut in the (+) strand, allowing initiation of replication from the 3′OH end serving as the primer. As the new (+) strand is synthesized, the “old” (+) strand is displaced. Once the (+) strand replication completes the full circle, a cut is made by pII at the same site as at the start, and both the “old” ssDNA (+) strand and the new strand are sealed. The “old” strand either serves as a template for the (−) strand replication, to allow production of more dsDNA that in turn becomes a template for a new round of (+) strand replication or is coated by pV to form the packaging substrate for assembly of the progeny virion.

Ff-derived phagemid particles are similar to Ff phages, however their genomes correspond to plasmids (called phagemids) that include a plasmid origin of replication, an antibiotic resistance gene as a selectable marker, an Ff origin of replication and typically one of the virion-coat-protein-encoding Ff genes (Barbas et al., 1991). An issue that arises with the use of Ff filamentous phages and derived phagemid particles in medical and diagnostic applications is that these phages and phage derived particles are generally available under most conditions as long filaments only. In particular, the high length-to-diameter ratio of Ff phage or phagemid particles interferes with applications that rely on diffusion, such as lateral flow diagnostic or analyte-detection devices.

It has been reported that duplication of a minor portion of the phage genome including the IG sequence that occurs at low frequency in phage population results in production of two types of virus-like particles, short (short interfering particles) and long (the original phage genome), by virtue of replicating the (+) strand ssDNA from the first (+) on until the second (duplicated) (+) on (Enea et al., 1977; Ravetch et al., 1979).

The (+) on is composed of an essential portion (named A or I) and a non-essential portion (named B or II). The complete origin is required for 100% activity with the wild-type replication protein pII, whereas the essential portion replicates at 1% efficiency relative to the full origin, unless specific mutants of replication protein pII are used, that have increased affinity for the (+) on A (Dotto et al., 1984b).

Extensive research on mapping of the (+) origin function showed that a truncated (+) on A domain, from which 29 residues (Δ29) at the 3′ end have been deleted, allows cutting by pII (replication protein) if, at a minimum, a complete on (+) A domain is present in the same plasmid, upstream of the mutated on (+) (Dotto et al., 1982, 1984a). In this arrangement, the complete on (+) functions as an initiator of (+) strand replication, whereas the (+) oriΔ29 functions as a terminator. When placed next to each other, these two (+) on sequences allow production of short circular ssDNA between the initiator and terminator cut sites, and assembly of very short Ff-derived nanorods (50 nm in length), provided that all required Ff proteins are supplied from a helper phage.

In this system both the short ssDNA and the full-length helper phage DNA were replicated and packaged into two types of particles, short (50 nm) nanorods and full-length (900 nm) filamentous viruses (Specthrie et al., 1992). The produced short nanorods were further functionalized through construction of protein fusion in the helper phage between pIII, a minor coat protein and a high-affinity fibronectin-binding domain (Fibronectin-Binding repeats; FnB) of Streptococcus pyogenes protein Serum opacity factor serotype 22 (Sof22), to allow display of FnB on the surface of the nanorods. Purified 50 nm particles displaying FnB were used in a lateral-flow (dip-stick) assay to detect fibronectin, and shown to demonstrate a cleaner signal than the FnB-displaying 900 nm long full-length phage particles of identical coat protein composition (Sattar et al., 2015).

However, the nanorods produced as outlined above are difficult to purify from the full-length helper phage also produced, resulting in nanorod preparations comprising nanorods of variable sizes, including high levels of contamination with full length virions. Additionally, the steps required to remove the full-length helper phage (the majority of the produced particles) result in a low final yield of nanorods, adding significant cost to production and purification. Further, in the above system, the total number of circular ssDNA copies produced per cell is limited, as is the replication efficiency.

Another issue that arises with the use of Ff phage and phagemid vectors for the production of filaments, rods and/or particles used in diagnostic and/or medical applications relates to the retention, in the filaments, rods and/or particles, of the antibiotic resistance genes used as selectable markers of transformed cells comprising these expression vectors.

Specifically, template plasmid recombination can result in the replication and packaging of the complete template plasmid. In a typical purified nanorod sample, this can result in contamination with longer particles at that carry antibiotic resistance genes (at 1/10⁶ frequency). Given that the number of particles used in a typical vaccination procedure (e.g., 10¹² per mouse), this level of contamination with antibiotics resistance encoding gene sequences is not tolerable as it would potentially result in 10⁶ infectious particles containing Amp^{R} gene per injection.

Based on what is known about the filamentous phage infection process, antibiotic resistance genes contained within the Ff phages and phagemid particles can be transferred to other bacteria within the gut or in the environment, spreading the antibiotic resistance genes (Russel et al., 1988). Furthermore, DNA from the phage or phagemid filaments has been shown to be internalized into the mammalian cells (Burg et al., 2002; Larocca and Baird, 2001), resulting in expression of genes that are encoded by its DNA, which includes antibiotic resistance.

Accordingly, it is an object of the invention to go at least some way towards addressing the deficiencies in the prior art as highlighted above by providing a system for producing scalable biological nanorods for use in various medical and diagnostic methods, including medical applications requiring direct introduction of nanorods into a subject, wherein the scalable nanorods can be produced from Ff phage particles and/or Ff phage derived particles with relatively high yields and/or relatively low contamination from longer Ff phage or Ff phage derived filaments and/or where the nanorods produced are free or substantially free of antibiotic resistance genes, and/or that will at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF INVENTION

Disclosed herein is a virus-free nanorod production system (NPS). The disclosed NPS is either a single plasmid or two plasmid system that directs the expression and assembly of Ff-bacteriophage-derived short scalable DNA-protein nanorods. Nanorods produced by an NPS as disclosed herein are not phage. Nanorods produced by an NPS as described herein have a 40 nm minimum length (FIG. 1), are not infectious, do not carry antibiotic resistance genes and cannot replicate in susceptible hosts because they do not encode phage proteins required for replication and virion assembly. Furthermore, the NPS disclosed herein is designed to control the amount and the length of produced nanorods as well as allowing the skilled worker to produce a range of nanorod variants for specific and orthogonal recombinant, enzymatic and chemical modifications.

Accordingly, in a first aspect, the present invention relates to a nanorod production system (NPS) comprising a single nucleic acid expression construct, the construct comprising
a BSFnano replication-assembly cassette
at least one auxotrophic marker,
at least one inducible promoter operably linked to a nucleic acid sequence encoding
at least one Ff phage protein, and
at least one plasmid origin of replication not located in the BSFnano
replication-assembly cassette.

In a second aspect, the invention relates to a nanorod production system (NPS) comprising
i) a nucleic acid replication construct comprising
   a BSFnano replication-assembly cassette,
   at least one auxotrophic marker, and
at least one plasmid origin of replication not located in the BSFnano replication-assembly cassette, and
ii) a helper nucleic acid expression construct comprising
   at least one selective marker
at least one inducible promoter operably linked to a nucleic acid sequence encoding
at least one modified Ff phage-encoded protein.

Various embodiments of the different aspects of the invention as discussed above are also set out below in the detailed description of the invention, but the invention is not limited thereto. Other aspects of the invention may become apparent from the following description that is given by way of example only and with reference to the accompanying drawings.

### BRIEF DESCRIPTION OF DRAWINGS

The invention will now be described by way of example only and with reference to the drawings in which:

FIG. 1. Transmission Electron Micrographs (TEMs) of negatively stained BSFnano (Biological Scalable functionalization-ready Ff-derived nanorods).

FIG. 2. Schematic drawings of the BSFnano Nanorod Production Systems (NPS).

FIG. 3. Ff (f1, fd or M13) phage-encoded proteins and their functions.

FIG. 4. Schematic depiction of the BSFp (A) and BSFpn (B) replication-assembly cassettes and their replication. Minimal size of circular ssDNA obtained from each of the replication-assembly cassettes and approximate production of corresponding nanorods are indicated.

FIG. 5. Schematic representation of the secondary structures of the Ff on and BSFnano replication-assembly cassettes. Wild-type Ff ori, also known as “intergenic sequence” (A); BSFpn (B) and BSFp (C), BSFnano replication-assembly cassettes, depicting secondary structures and coordinates corresponding to the f1 genome sequence. “Scaffold” indicates the sequence that is replicated to generate the (+) strand circular ssDNA forming the backbone of the nanorods. “Filler” indicates the positions where DNA can be inserted to gauge the size of the nanorods or to express a functionality of interest (i.e., pVII and pIX).

FIG. 6. Maps of the BSFnano replication-assembly cassettes. A. BSFpn replication-assembly cassette, containing (+) and (−) Ff origins of replication and packaging signal (PS). B. BSFpn cassette containing gVII and gIX expressed from within the BSF replication-assembly cassette. C. BSFp replication cassette containing only the Ff (+) ori's and PS. Replication-assembly cassette, scaffold, filler sequences and sequences required for the nanorod ssDNA replication and assembly are indicated on the maps.

FIG. 7. Map of the pPop-up plasmid series. pPop-up plasmids contain variations of six functional blocks: i) BSFnano replication-assembly cassette, ii) promoter upstream of gII; iii), genes encoding Ff replication functions [gII (gX)] and packaging-substrate-forming function (gV); iv), genes encoding virion proteins that allow display of peptides or attachment handles (gVII, gIX, gVIII, gIII, gVI); v), the remaining portion of the plasmid containing the assembly-function-encoding genes (gIV and gI) and plasmid origin of replication; vi) marker. BSFnano replication-assembly cassette (i) examples: BSFp (152 nt; 221 nt); BSFpn (289 nt, 313 nt, 395 nt, 529 nt). Promoter (ii) examples: Ff pA; lacUV5. Replication and ssDNA-binding proteins (iii) variant examples: gII IR1B (Thr182Ile); virion proteins (iv) variant examples; encoded by engineered alleles: gVIII-20am; gVIII 2am; gVIII-20am nAAGG, ΔP6 S17L A27S; gVIII-20am nGGGG A1G; gVIII-20am Y21M. gIII wild-type; gIII::MCS; gIIIC::MCS 3Cys; FnB-gIII; C121-FnB (C121 is a SARS-CoV-2 Spike-specific scFv); N3-gIII (N3 is a SARS-CoV-2 nucleocapsid (NC) protein-specific camelid antibody VHH). Marker (vi) examples; Km^{R}; nadC. Arrow and the small ellipse in the top left corner of the image represent the excised BSFnano replication cassette. T, transcriptional terminator; Pz, phage promoter driving expression of gIII-gVI-gI(gXI)-gIV operon. The circular ssDNA product of the BSFnano cassette is the backbone of the nanorods. AhdI, XhoI, PstI, SaIlI, HpaI, SnaBI, BamHI, AfeI, PacI, restriction sites used in construction of plasmid variants; MCS, multiple cloning site for inserting sequences encoding fusion peptides displayed using minor coat protein pIII as the platform.

FIG. 8. Map of the pHP helper plasmid series. The pHP plasmids contain variations of five functional blocks: i) promoter upstream of gII; ii), genes encoding Ff replication [gII (gX)] and packaging-substrate-forming function (gV); iii), genes encoding virion proteins that allow display of peptides or attachment handles (gVII, gIX, gVIII, gIII, gVI); iv), the remaining portion of the plasmid containing the assembly-function-encoding genes (gIV and gI) and plasmid origin of replication; v) marker. Promoter (i) examples: Ff pA; lacUV5. Replication and ssDNA-binding proteins (ii) variant examples: gII IR1B (Thr182Ile); virion proteins (iii) variant examples; encoded by engineered alleles: gVIII-20am; gVIII 2am; gVIII-20am A9M S17L M28L; gVIII 2am nGGGG S17L; gVIII-20am nAAGG, ΔP6 S17L A27S; gVIII-20am nGGGG A1G; gVIII-20am Y21M. gIII wild-type; gIII::MCS; gIIIC::MCS 3Cys; FnB-gIII; C121-gIII (C121 is a SARS-CoV-2 Spike-specific scFv); N3-gIII (N3 is a SARS-CoV-2 nucleocapsid protein-specific heavy-chain-only antibody VHH); Marker (v) examples; Km^{R}. T, transcriptional terminator; Pz, phage promoter driving expression of gIII-gVI-gI(gXI)-gIV operon. AhdI, XhoI, PstI, SaIlI, HpaI, SnaBI, BamHI, AfeI, PacI, restriction sites used in construction of plasmid variants. MCS, multiple cloning site for inserting sequences encoding fusion peptides displayed using minor coat protein pIII as the platform.

FIG. 9. Map of a pBSFnano plasmid series containing the nanorod replication-assembly cassettes. pBSFnano plasmids contain three functional blocks, i) BSFnano replication cassette; ii) marker; iii) origin of replication. The BSFnano replication cassette (i) examples, BSFp (152, 221); BSFpn (289, 313, 395, 529, 711, 728, 79a, 791ac, 1400). Small circle denotes the circular ssDNA replicated from the BSF replication-assembly cassette (BSFnano). This circular ssDNA is assembled into the BSF nanorods in the presence of the assembly/secretion and virion proteins. Marker (ii) examples: Amp^{R}, Cm^{R} or nadC.

FIG. 10. Nanorods produced by the single-plasmid production systems pBSFpn, positive-and-negative-origin replication-assembly cassette (pPop-up529YM) or pBSFp, a positive-strand-origin-only replication-assembly cassette (pPop-up221YM). DNA from SDS-disassembled nanorods was separated by agarose gel electrophoresis and visualised by EtBr staining. Lanes: Ladder, 1 kb plus ladder (a double-stranded linear DNA standard used as a signpost for migration due to the lack of appropriate circular ssDNA standards; numbers indicate sizes of the standard bands in base-pairs); Lanes: 1, nanorods derived from the cells transformed with plasmids pPop-up529Y; 2, pPop-up221YM. Nanorods were concentrated 1000-fold by PEG precipitation from the supernatant of the cultures derived from pooled cells transformed with pPop-up529YM or pPop-up221YM (containing BSFpn529 or BSFp221 replication-assembly cassette, respectively). Nanorods were further purified by CsCl gradient and ion-exchange chromatography.

FIG. 11: Comparison of the BSF nanorods made using the BSF plasmids with or without genes gVII and gIX. CsCl-purified nanorods were analysed by agarose gel electrophoresis. A. DNA from SDS-disassembled nanorods visualised by EtBr staining. B. Native nanorods visualised by EtBr after in situ NaOH-mediated removal of virion proteins. Lanes: Helper plasmid pHP2 was combined with: 1, pBSFnano711; 2, pBSFnano79a (707 nt); 3, pBSFnano791ac (748 nt), IPTG-induced; 4, pBSFnano79Lac, uninduced. L, 1 kb plus ladder (a double-stranded linear DNA standard used as a signpost for migration due to the lack of appropriate circular ssDNA standards; numbers indicate sizes of the standard bands in base-pairs). Nanorods were PEG-precipitated from the supernatant of double-transformed cell pool containing a helper and a pBSF plasmid, and further purified by CsCl gradient centrifugation. Single, double, triple, and quadruple asterisks correspond to single-, double-, triple- and quadruple-length nanorods.

FIG. 12. Inducible expression of protein pII increases BSFnano nanorod production. DNA from SDS-disassembled nanorods (A); or native nanorods treated in situ by NaOH to expose their DNA and stained with ethidium bromide (B); produced in the single-plasmid (pPop-up) system containing a 529-nt replication-assembly cassette (BSFpn529). Expression of the replication protein pII was driven by the constitutive f1 phage promoter pA (plasmid pPop-up529YM), or by inducible promoter placUV5 (plasmid pPop-up529LacYM). Lanes: L, Ladder, 1 kb plus ladder (a double-stranded linear DNA standard used as a signpost for migration due to the lack of appropriate circular ssDNA standards; numbers indicate sizes of the standard bands in base-pairs); 1, particles derived from the plasmid pPop-up529YM; 2, pPop-up529LacYM without IPTG induction; 3, pPop-up529LacYM plasmid in the presence of the placUV5 promoter inducer (IPTG; 0.1 mM). Nanorods were concentrated 1000-fold by PEG precipitation from the supernatant of a 1 L culture of pooled cells transformed with pPop-up529YM or pPop-up529LacYM. Where applicable, IPTG was added to the culture of the pooled transformed cells at OD₆₀₀ of 0.1.

FIG. 13. Comparison of the BSFnano529 nanorods produced by one- and two-plasmid inducible nanorod production systems. A. purified nanorod circular ssDNA; B. whole native particles of PEG-precipitated nanorods. Lanes: L, 1 kb Plus (a double-stranded linear DNA standard used as a signpost for migration due to the lack of appropriate circular ssDNA standards; numbers indicate sizes of the standard bands in base-pairs).; 1, pPop-up529YM; 2, pHP1AevLac+pBSF529, no IPTG; 3, pHP1AevLac+pBSF529, 0.1 mM IPTG; 4, pHP1AevLac+pBSF529, 1 mM IPTG; 5, pPop-up529LacAev no IPTG; 6, pPop-up529LacAev 0.1 mM IPTG. Nanorods were concentrated 1000-fold by PEG precipitation from the supernatant of a 1 L culture of the culture derived from the cells transformed with pPop-up or double-transformed with a pHP1 helper plasmid variant and pBSFpn529 nanorod replication-assembly plasmid.

FIG. 14. 395-nt (70-nm) nanorods produced from the inducible single-plasmid system. BSFnano replication-assembly cassettes contain both positive and negative ori's (pPop-up529LacYM and pPop-up395LacYM). A; DNA from SDS-disassembled nanorods was separated by agarose gel electrophoresis and visualised by EtBr staining. B; native nanorods separated by agarose gel electrophoresis. Bands corresponding to the native/intact particles were visualized after soaking the gel in 0.2 M NaOH to strip off the virion proteins in situ and staining the gel with EtBr. Lanes: L, Ladder 1 Kb Plus (a double-stranded linear DNA standard used as a signpost for migration due to the lack of appropriate circular ssDNA standards; numbers indicate sizes of the standard bands in base-pairs); 1, BSFpn529YM particles produced by the pPop-up529LacYM plasmid; 2, BSFpn395 particles produced by the pPop-up395LacYM plasmid

FIG. 15. 152-nt (40 nm) nanorods produced from the inducible single-plasmid system. The BSF-nano replication-assembly cassettes contain only the positive on (pPop-up221LacYM and pPop-up152LacYM) A; DNA from SDS-disassembled nanorods was separated by agarose gel electrophoresis and visualised by EtBr staining. B; native nanorods separated by agarose gel electrophoresis. Bands corresponding to the native/intact particles were visualized after soaking the gel in 0.2 M NaOH to strip off the virion proteins in situ and staining the gel with EtBr. Lanes: L, Ladder 1 Kb Plus (a double-stranded linear DNA standard used as a signpost for migration due to the lack of appropriate circular ssDNA standards; numbers indicate sizes of the standard bands in base-pairs).; 1, BSFp221YM particles produced by the pPop-up221LacYM plasmid; 2, BSFp152 particles produced by the pPop-up152LacYM plasmid. The upper band in A is not DNA. It is eliminated after purification of nanorod DNA (not shown). It likely represents EtBr-stained detergent-associated membrane or peptidoglycan fragments.

FIG. 16. BSF nanorods made using a template plasmid containing auxotrophic marker NadC. Lanes: L, 1 kb plus ladder (a double-stranded linear DNA standard used as a signpost for migration due to the lack of appropriate circular ssDNA standards; numbers indicate sizes of the standard bands in base-pairs). 1, BSF529 nanorods derived from the pBSFpn529N plasmid encoding auxotrophic marker NadC. Nanorods were concentrated by pressure-mediated ultrafiltration from the supernatant of the pooled double-transformed cells and further purified by CsCl gradient centrifugation.

FIG. 17. TEM analysis of purified BSFnano221YM2 221-nt nanorods. A. Electron micrographs of negatively stained 221-nt nanorods. B. Histogram of the nanorod length distribution plotted from the length measurements of 100 well-separated particles using the ImageJ software.

FIG. 18. TEM of spontaneously formed BSFnano221.2 liquid crystals.

FIG. 19. TEM analysis of purified BSFnano529YM2 529-nt nanorods. A; B. Electron micrographs of negatively stained 529-nt nanorods. C. Histogram of the nanorod length distribution plotted from the length measurements of 300 well-separated particles using the ImageJ software.

FIG. 20. Nanorod-based lateral flow assay for detection of fibronectin. A. Detection of FnB-pIII fusions in the BSF nanorods by SDS-PAGE and western blotting. Left panel: All proteins in the gel were detected by Coomassie blue staining; Right panel: FnB-pIII fusion was detected by western blotting using a pIII-specific antibody (Rakonjac and Model, 1998). Lanes: M, molecular weight standard (kDa); 1, control particles BSFnano711G82 without the fusion protein; 2, BSFnano711G8FnB2, particles displaying FnB-pIII fusion. B. Series of fibronectin dilutions analysed using BSFnano-based dipstick assay. Each assay contained 10¹¹ nanorods in the total volume of 100 μL; Fn concentrations are indicated below each stick. C. Fibronectin dipstick assay using fluorescently labelled BSFnano711G8FnB2 nanorods. Signal was detected using a fluoroimager (Azure c600). A total of 10¹¹ nanorods were mixed with 1 μg of fibronectin diluted in PBS (100 μL final volume), or PBS alone, and incubated for 30 min at room temperature. Test strips were dipped into the mixture, allowing lateral flow for 15 min. Particles that carry Fn bound to collagen were visualised on the bottom (Test) line. Control line captured all the BSFnano particles (BSFnano711G82 and BSFnano711G8FnB2). Stick 1, assay in the presence of analyte (Fn); stick 2, assay in the absence of Fn.

FIG. 21. Enzymatic modification of Ff phage-derived nanorods by S. pyogenes Sortase A (SrtA Sp). Mechanism of the sortase reaction between a functionality (grey circle) and pVIII subunits within the nanorod. A functionality (a small molecule or a protein) contains a C-terminal LPETA sortase recognition motif, whereas pVIII subunits in the nanorod contain an N-terminal double alanine. The active site of Streptococcal sortase A cleaves the LPETA motif between the threonine and alanine to form an acyl intermediate. The Ala-Ala nucleophile attacks the acyl intermediate bond, releasing the Sortase and forming an amide bond between the two target proteins.

FIG. 22. Agarose gel electrophoresis of enzymatically FITC-labelled nanorods. A, image without staining, detecting only the FITC-labelled nanorods. B, Image after the protein coat was removed by NaOH treatment and exposed DNA stained by ethidium bromide. Lanes: L, Ladder, 1 kb plus ladder NEB (a double-stranded liner DNA standard used as a signpost for migration due to the lack of appropriate circular ssDNA standards; numbers indicate sizes of the standard bands in base-pairs); 1, FITC labelled nanorods displaying SARS-CoV-2 Spike-specific scFv (BSFnano728Aev1C121-FITC); 2, Unlabelled nanorods control with no antibody displayed (BSFnano728Aev1); 3, Unlabelled nanorods displaying Spike-specific scFv (BSFnano728Aev1C121).

FIG. 23. TEM-immunogold analysis of enzymatic biotin conjugation to BSFnano728Aev1. nanorods. Nanorods were produced using pHP1AevLac helper plasmid and pBSFpn728 nanorod replication-assembly plasmid and enzymatically conjugated to LPETA-biotin using Sortase A from S. pyogenes (SrtA Sp). Images are of BSFnano728LacAev1 nanorods reacted with 20 μM biotin-KLPETAA and 50 μM Sortase A (A-F) and 20 μM biotin-KLPETAA without Sortase A (G-I). Black dots indicate presence of the Streptavidin-gold nanoparticles. Scale bars represent 200 nm.

FIG. 24. Enzymatic modification of BSF nanorods. A. biotinylated and non-biotinylated nanorods were analysed by native particle agarose gel electrophoresis and western blotting. 1, biotinylated BSFnano728Aev1C121; 2, non-biotinylated BSFnano728Aev1C121. In each lance, 10¹⁰ particles were loaded. Signal was developed by BCIP/NBT substrate after incubation with Streptavidin-Alkaline phosphatase. Nanorods (5 nM or 3×10¹²) were biotinylated by reaction containing 200 μM biotin-KLPETAA and 50 μM S. pyogenes Sortase A (Lane 1). B. Enzymatic attachment of a reporter (β-glucuronidase of E. coli; UidA or GUS) tagged with LPETG peptide using the S. aureus Sortase to GGGGG-tagged nanorods. 1, GUS-modified BSFnano728Gly5; 2, Unmodified BSFnano728G8. Modified and non-modified nanorods were analysed by native particle agarose gel electrophoresis. The gel was directly stained via the GUS reporter reaction in the presence of a chromogenic substrate (100 mM NaPO4 pH 7.0, 1 mM X-GLUC:Na, 200 μM NBT). 10¹⁰ particles were loaded in each lane.

FIG. 25. Dot-blot SARS-CoV-2 nanorod-based antigen (Spike) detection. A. Schematic representation of the experiment: dilutions of the Spike protein were spotted on a nitrocellulose filter. The Spike protein was detected by enzymatically biotinylated nanorods (10¹¹ nanorods per assay), followed by Avidin-Alkaline Phosphatase conjugate and reaction with a chromogenic substrate. B. Experimental result, showing detection of 1 ng of Spike protein. Positive control (+) are directly immobilised nanorods. Negative (−) control is buffer.

FIG. 26. Sandwich ELISA assays using SARS-CoV-2-specific detector particles. Assays were performed as detailed in Material and Methods. Antibody CR3022 specific to the SARS-CoV Spike protein extracellular domain (ECD) was used for capture serially diluted ECD (A, B). A. Captured ECD was detected by nanorods displaying SARS-CoV-2 ECD-specific antibody C121 fused to pIII (BSFnano728Aev1C121). Bound nanorods were visualised by nanorod-specific antibodies and secondary HRP-conjugated antibodies. B. Enzymatically biotinylated BSFnano728Aev1C121 nanorods were used to detect the ECD bound to the plates via the capture antibody. Streptavidin-HRP conjugate was used for visualisation of the bound nanorods. C. SARS-CoV-2 nucleocapsid (NC) protein sandwich ELISA. Aminated aptamer (ssDNA molecule) specific for SARS-CoV nucleocapsid (NC) protein immobilised to ELISA plates was used for capture of the SARS-CoV-2 nucleocapsid (NC) protein. Enzymatically biotinylated nanorods displaying a VHH specific for NC (BSFnano728Aev1N3) were used to detect the SARS-CoV-2 nucleocapsid (NC) protein bound to the aptamer. Streptavidin-HRP conjugate was used for visualisation of the bound nanorods.

FIG. 27. Lateral flow assay using SARS-CoV-2-specific nanorods. SARS-CoV antigen-specific capture molecule (antibody or aptamer) was immobilised in the test line (B) or test dot (C), whereas a nanorod-specific capture molecule was immobilised on the control line (B) or control dot (C). + and − denote presence or absence of antigen. B. Extracellular domain of the Spike protein (ECD) was detected by biotinylated nanorods displaying SARS-CoV-2 ECD-specific antibody C121 (binding to a different epitope from the capture antibody) fused to pIII (BSFnano728Aev1C121). C. Enzymatically biotinylated nanorods displaying a VHH specific for NC (BSFnano728Aev1N3) were used to detect the SARS-CoV-2 nucleocapsid (NC) protein bound to the aptamer. Streptavidin-Alkaline Phosphatase conjugate was used for visualisation of the bound nanorods as described in Material and Methods.

FIG. 28. A list of nucleic acid and amino acid sequences disclosed herein.

FIG. 29. Wild-type pII amino acid sequence and corresponding gII CDS nucleic acid sequence.

FIG. 30. Amino acid and corresponding nucleotide sequence of the IR1B mutant of gII used in the nanorod production system (pHP and pPop-up plasmids).

FIG. 31. pV, pVII and pIX wild type used in the nanorod production system.

FIG. 32. Sequence of the wild-type and modified pVIII/gVIII. Vertical arrow denotes the signal sequence cleavage site. Bold underlined residues indicate mutations and amino acid changes.

FIG. 33. Sequences of the modified pVIII/gVIII variants. Vertical arrow denotes the signal sequence cleavage site. Bold underlined sequence indicates mutations or amino acid changes.

FIG. 34. Wild type pIII and gIII. Vertical arrow denotes the signal sequence cleavage site; underlined sequence, BamHI site.

FIG. 35. Modified pIII and gIII, full length. pIII::MCS, pIII displaying an inserted peptide encoded by the multiple cloning site (corresponding to the highlighted sequence in the gIII::MCS nucleotide sequence). Vertical arrow, the signal sequence cleavage site. Underlined, BamHI recognition site.

FIG. 36. Modified pIII and gIII, C-terminal domain; f1 wild-type pVI and gVI. pIIIC::MCS, pIII displaying an inserted peptide encoded by the multiple cloning site (corresponding to the highlighted sequence in the gIIIC::MCS nucleotide sequence). Vertical arrow, the signal sequence cleavage site. Underlined, BamHI recognition site.

FIG. 37. Amino acid and nucleic acid sequences of the pIII-displayed FnB-pIII fusion. The shaded sequence corresponds to FnB, fibronectin-binding repeats from Streptococcus pyogenes M22 serum opacity factor, strain D734 (Rakonjac et al., 1995).

FIG. 38. Amino acid and nucleic acid sequences of the scFvC121-pIII fusion. Top, amino acid sequence; bottom, nucleic acid sequence. The shaded sequence corresponds to scFvC121, single-chain variable domain of the antibody against the SARS-CoV-2 Spike protein (Robbiani et al., 2020).

FIG. 39. Sequences of the BSFpn728 and BSFpn711 replication-assembly cassettes and scaffolds. [symbol] indicates the pII cut sites. The circular ssDNA within the nanorods corresponds to the sequence between the two pII cut sites.

FIG. 40. Sequences of components within the BSFpn728 and BSFpn711 replication-assembly cassettes. [symbol] indicates the pII cut sites. The circular ssDNA within the nanorods corresponds to the sequence between the two pII cut sites.

FIG. 41. Sequences of the BSFpn79a and BSFpn79lac replication-assembly cassettes. [symbol] indicates the pII cut sites. The circular ssDNA within the nanorods corresponds to the sequence between the two pII cut sites.

FIG. 42. Sequences of components within the BSFpn79a and BSFpn79lac replication-assembly cassettes. [symbol] indicates the pII cut sites. The circular ssDNA within the nanorods (the scaffold) corresponds to the sequence between the two pII cut sites.

FIG. 43. Sequences of the BSFpn529 and BSFpn395 replication-assembly cassettes and scaffolds. [symbol] indicates the pII cut sites. The circular ssDNA within the nanorods corresponds to the sequence between the two pII cut sites.

FIG. 44. Sequences of components within the BSFpn529 and BSFpn395 replication-assembly cassettes. [symbol] indicates the pII cut sites. The circular ssDNA within the nanorods corresponds to the sequence between the two pII cut sites.

FIG. 45. Sequences of the BSFpn313 and BSFpn289 replication-assembly cassettes and scaffolds. [symbol] indicates the pII cut sites. The circular ssDNA within the nanorods corresponds to the sequence between the two pII cut sites.

FIG. 46. Sequences of components within the BSFpn313 and BSFpn289 replication-assembly cassettes. [symbol] indicates the pII cut sites. The circular ssDNA within the nanorods corresponds to the sequence between the two pII cut sites.

FIG. 47. Sequences of the BSFpn221 and BSFpn152 replication-assembly cassettes and scaffolds. [symbol] indicates the pII cut sites. The circular ssDNA within the nanorods corresponds to the sequence between the two pII cut sites.

FIG. 48. Sequences of components within the BSFp221 and BSFp152 replication-assembly cassettes. [symbol] indicates the pII cut sites. The circular ssDNA within the nanorods corresponds to the sequence between the two pII cut sites.

FIG. 49. The pII recognition sequence and sequences of the promoters driving gII(gX)-gV-gVII-gIX-gVIII expression in the pHP1 and pPop-up plasmid series. Top row, pII recognition sequence; vertical arrow denotes the nick site. Middle row, wild-type Ff promoter pA; bottom row, placUV5 in the pPop-upLac and pHP1Lac plasmid series. Highlighted ATG in the middle and bottom row indicates the first codon of the gII orf.

FIG. 50. Auxotrophic marker NadC. Top row, amino acid sequence of NadC. Middle and bottom rows, nucleic acid sequence of the marker block. Underlined, pairs of the restriction sites flanking the marker block used for cloning: BamHI and SnaBI in the pBSFnano; AhdI and XhoI in the pPop-up plasmids.

FIG. 51. Sequence of a representative pPop-up plasmid, pPop-upBSFpnLac529YM (FASTA format).

FIG. 52. Sequence of a representative pHP helper plasmid series, pHP1Lac (FASTA format).

FIG. 53. Sequence of a representative pBSFnano replication-assembly plasmid series, pBSFnano529N (FASTA format).

FIG. 54. Sequences of the modified pVIII/gVIII variant for display of a GGGGG tag. Vertical arrow denotes the signal sequence cleavage site. Bold underlined sequence indicates mutations or amino acid changes.

FIG. 55. Amino acid and nucleic acid sequences of the VHH N3-pIII fusion. Top, amino acid sequence; bottom, nucleic acid sequence. The shaded sequence corresponds to VHH N3, a single-chain variable domain of the antibody against the SARS-CoV-2 nucleocapsid (NC) protein (Sherwood and Hayhurst, 2021).

FIG. 56. Sequences of the BSFpn1400 replication-assembly cassette and scaffold. [symbol] indicates the pII cut sites. The circular ssDNA within the nanorods corresponds to the sequence between the two pII cut sites.

FIG. 57. Sequences of components within the BSFpn1400 replication-assembly cassette. [symbol] indicates the pII cut sites. The ssDNA within the nanorods corresponds to the sequence between the two pII cut sites.

### DETAILED DESCRIPTION OF INVENTION (DESCRIPTION OF EMBODIMENTS)

### Definitions

The following definitions are presented to better define the present invention and as a guide for those of ordinary skill in the art in the practice of the present invention. Unless otherwise specified, all technical and scientific terms used herein are to be understood as having the same meanings as is understood by one of ordinary skill in the relevant art to which this disclosure pertains. Examples of definitions of common terms in microbiology, molecular biology, pharmacology, and biochemistry can be found in (Lederberg, 2000; Lewin et al., 2011; Madigan et al., 2009; Meyers, 1995; Reddy, 2007; Singleton and Sainsbury, 2006).

It is also believed that practice of the present invention can be performed using standard microbiological, molecular biology, pharmacology and biochemistry protocols and procedures as known in the art, and as described, for example in (Burtis et al., 2015; Lewin et al., 2011; Reddy, 2007; Sambrook and Russell, 2001; Whitby and Whitby, 1993) and other commonly available reference materials relevant in the art to which this disclosure pertains, and which are all incorporated by reference herein in their entireties.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

The term “BSFnano replication-assembly cassette” as used herein refers a nucleic acid sequence comprising at least one positive-strand origin of replication, (+) ori.

The term “(+) ori” as used herein means the nucleic acid sequence functioning as a positive DNA strand origin of replication.

The term “(−) ori” as used herein means the nucleic acid sequence functioning as a negative DNA strand origin of replication.

In one embodiment the BSFnano replication-assembly cassette comprises at least one (+) ori and at least one (−) ori. In one embodiment the BSFnano replication-assembly cassette comprises at least two (+) ori. In one embodiment at least one (+) ori is an initiator of replication. In one embodiment at least one (+) ori is a terminator of replication.

In one embodiment the BSFnano replication-assembly cassette comprises at least one (−) ori.

The term “fusion gene” as used herein refers to a gene coding for a translational fusion between a peptide and a filamentous bacteriophage major (pVIII) and minor (pIII, pVI, pVII and pIX) coat proteins or part thereof, preferably an Ff phage coat protein, or a part thereof. A fusion protein as described herein is encoded by a fusion gene.

The term “polynucleotide(s),” as used herein, refers in its broadest sense to a single or double-stranded deoxyribonucleotide or ribonucleotide polymer of any length, and includes as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to nucleic acids, nucleic acid molecules, nucleotide sequences and polynucleotide sequences is to be similarly understood.

In some embodiments the polynucleotides described herein are isolated.

Nucleic acids as contemplated herein may be, or include (but not limited thereto), deoxyribonucleic acids (DNAs), ribonucleic acids (RNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), threose nucleic acids (TNAs), ethylene nucleic acids (ENA), cyclohexenyl nucleic acids (CeNA), glycol nucleic acids (GNAs), or chimeras or combinations thereof.

In some embodiments, a nucleic acid or polynucleotide as described herein is a messenger RNA (mRNA). The term “messenger RNA” (mRNA) as used herein refers to any polynucleotide that encodes a polypeptide of interest, such as one described herein, and that can be translated in vitro, in vivo, ex vivo or in situ to produce the polypeptide.

The encoded polypeptide may be a naturally occurring, non-naturally occurring, or modified polymer of amino acids. In a preferred embodiment, the encoded polypeptide is a non-naturally occurring polypeptide. As used herein unless specifically indicated otherwise, DNA polynucleotide sequences described herein will recite thymine (T) whereas RNA polynucleotide sequences the thymine is replaced with uracil (U).

Accordingly, the skilled person recognizes that any of the polynucleotides encoded by a specifically identified DNA (i.e., by a SEQ ID NO: 2), is considered to comprise the corresponding RNA (e.g., mRNA) sequence where each thymine the DNA sequence is substituted with uracil (i.e., T>U substitution).

The person skilled in the art also appreciates that an mRNA that can be translated into a polypeptide of interest will also include some or all of the following features: a 5′ cap, a 5′ untranslated region (UTR), at least one coding region, a 3′ UTR, and a poly-A tail.

The term “open reading frame” means a continuous stretch of DNA beginning with a start codon (e.g., methionine (ATG)), and ending with a stop codon (e.g., TAA, TAG or TGA). An open reading frame encodes a polypeptide.

The term “amber mutation” refers to a mutation in which a polypeptide chain is terminated prematurely. Amber mutations are the result of a base substitution that converts a codon specifying an amino acid into a stop codon, e.g., UAG, which signals chain termination. Other mutations that convert an amino-acid codon to a stop codon are known as ochre (UAA) and opal (UGA).

The term “3′ untranslated region” (3′UTR) is used herein as understood by the skilled person and refers to a region of an mRNA that is directly downstream (i.e., 3′) from the stop codon (i.e., the codon of an mRNA transcript that signals a termination of translation). The 3′UTR does not comprise an open reading frame and/or is not translated into a polypeptide.

The term “5′ untranslated region” (5′UTR) is used herein as understood by the skilled person and refers to a region of an mRNA that is directly upstream (i.e., 5′) from the start codon (i.e., the first codon of an mRNA transcript translated by a ribosome). The 5′UTR does not comprise an open reading frame and/or is not translated into a polypeptide.

As used herein, the term “polyA tail” means a region of mRNA that is downstream (i.e., 3′) from the 3′ UTR and that contains multiple, consecutive adenosine monophosphates (A residues). As is appreciated in the art, the function of the poly(A) tail is to protect an mRNA from enzymatic degradation as well as to facilitate both transcription termination and mRNA export from the nucleus. The number of consecutive A residues in a “poly A tail” may vary, e.g., from 10 to 300. By way of example only, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 A residues.

The term “vector” as used herein refers to any type of polynucleotide molecule that may be used to manipulate genetic material so that it can be amplified, replicated, manipulated, partially replicated, modified and/or expressed, but not limited thereto. In some embodiments a vector may be used to transport a polynucleotide comprised in that vector into a cell or organism. In some embodiments a vector is selected from the group consisting of plasmids, bacterial artificial chromosomes (BACs), P1-derived artificial chromosomes (PACs), yeast artificial chromosomes (YACs), bacteriophage, phagemids, and cosmids. In a preferred embodiment, a vector is a plasmid.

In some embodiments a nucleic acid expression construct, nucleic acid replication construct and/or a helper nucleic acid expression construct as described herein is, or is comprised in, a vector. In some embodiments a nucleic acid expression construct, nucleic acid replication construct and/or a helper nucleic acid expression construct as described herein is or is comprised in, a plasmid. In some embodiments, a vector or plasmid may consist essentially of a nucleic acid expression construct, nucleic acid replication construct and/or a helper nucleic acid expression construct as described herein. In some embodiments a vector or plasmid may consist of a nucleic acid expression construct, nucleic acid replication construct and/or a helper nucleic acid expression construct as described herein.

The term “coding region” or “open reading frame” (ORF) refers to the sense strand of a genomic DNA sequence or a cDNA sequence that is capable of producing a transcription product and/or a polypeptide under the control of appropriate regulatory sequences.

The coding sequence is identified by the presence of a 5′ translation start codon and a 3′ translation stop codon. When inserted into a genetic construct or an expression cassette, a “coding sequence” is capable of being expressed when it is operably linked to promoter and terminator sequences and/or other regulatory elements.

“Operably-linked” means that the sequence to be expressed is placed under the control of regulatory elements.

“Regulatory elements” as used herein refers to any nucleic acid sequence element that controls or influences the expression of a polynucleotide insert from a vector, genetic construct or expression cassette and includes promoters, transcription control sequences, translation control sequences, origins of replication, tissue-specific regulatory elements, temporal regulatory elements, enhancers, polyadenylation signals, repressors, and terminators. Regulatory elements can be “homologous” or “heterologous” to the polynucleotide insert to be expressed from a genetic construct, expression cassette or vector as described herein. When a nucleic acid expression construct, expression cassette or vector as described herein is present in a cell, a regulatory element can be “endogenous”, “exogenous”, “naturally occurring” and/or “non-naturally occurring” with respect to cell.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to non-transcribed cis-regulatory elements upstream of the coding region that regulate the transcription of a polynucleotide sequence. Promoters comprise cis-initiator elements which specify the transcription initiation site and conserved boxes. In one non-limiting example, bacterial promoters may comprise a “Pribnow box” (also known as the −10 region), and other motifs that are bound by transcription factors and promote transcription. Promoters can be homologous or heterologous with respect to polynucleotide sequence to be expressed. When the polynucleotide sequence is to be expressed in a cell, a promoter may be an endogenous or exogenous promoter. Promoters can be constitutive promoters, inducible promoters or regulatable promoters as known in the art. In a preferred embodiment contemplated herein a promoter is an inducible promoter.

The term “polypeptide(s),” as used herein, is used in a broad sense to include naturally occurring polypeptides, artificial polypeptides, synthetic polypeptides, gene products, homologs, orthologs, paralogs, variants, fragments, and other equivalents, as well as analogs of such as would be appreciated by a skilled person in the art. A polypeptide may be a single molecule or may part of a molecular complex. Such complexes include, but are not limited to, dimers, trimers, tetramers, hexamers, and the like. A polypeptide can comprise a single chain of amino acids (i.e., a single polypeptide), or, in the case of a molecular complex, multiple chains of amino acids (multiple polypeptides). Frequently, molecular complexes comprising multiple polypeptides comprise disulfide bridges or linkages between certain amino acid residues. As used herein, the term “polypeptide” also refers to polymers of amino acid residues comprising at least one modified amino acid residue, including as a non-limiting example, an artificial chemical analogue of a corresponding naturally occurring amino acid.

“Naturally occurring” as used herein with reference to a polypeptide or polynucleotide refers to a polynucleotide or polypeptide sequence having a primary nucleic acid or amino acid sequence that is found in nature. A synthetic polynucleotide or polypeptide sequence that is identical to a wild-type polynucleotide sequence is, for the purposes of this disclosure, considered a naturally occurring sequence. What is important for a naturally occurring polynucleotide or polypeptide sequence is that the actual sequence of nucleotide bases or amino acid residues that make up the polynucleotide or polypeptide respectively, is as found or as known from nature.

The term “wild-type” is used here as generally understood in the art. For example, a wild-type polynucleotide sequence is a naturally occurring polynucleotide sequence. A naturally occurring polynucleotide sequence also refers to variant polynucleotide sequences as found in nature that differ from wild-type. For example, allelic variants and naturally occurring recombinant polynucleotide sequences due to hybridization or horizontal gene transfer, but not limited thereto.

“Non-naturally occurring” as used herein with reference to a polypeptide or polynucleotide refers to a polynucleotide or polypeptide having a primary nucleic acid or amino acid sequence that is not found in nature. Such peptides are also called “artificial polypeptides” (and grammatical variations thereof) herein.

Examples of non-naturally occurring polynucleotide and polypeptide sequences include artificially produced mutant and variant polynucleotide and polypeptide sequences, made for example by point mutation, insertion, or deletion, domain rearrangement, but not limited thereto. Non-naturally occurring polynucleotide and polypeptide sequences also include chemically evolved sequences. What is important for a non-naturally occurring polynucleotide or polypeptide sequence as described herein is that the actual sequence of nucleotide bases or amino acid residues that makes up the polynucleotide or polypeptide respectively, are not found in or known from nature.

The term “fused” as used herein with reference to polypeptides and portions of polypeptides that are “fused” together (including other grammatical variations) means that the amino acid sequences are covalently joined to each other by peptide bonds.

The “fusion polypeptides” disclosed in the present application are artificial polypeptides, i.e., the fusion polypeptides disclosed herein are non-naturally occurring. As described herein, a fusion polypeptide or fusion protein (these terms are used interchangeably and mean the same thing), is expressed from a fusion gene.

“Homologous” as used herein with reference to a polynucleotide or polypeptide or part thereof means a polynucleotide or polypeptide or part thereof that is a naturally occurring polynucleotide or polypeptide or part thereof.

“Heterologous” as used herein with reference to a polynucleotide or polypeptide or part thereof means a polynucleotide or polypeptide or part thereof that is a non-naturally occurring polynucleotide or polypeptide or part thereof.

A homologous polynucleotide or part thereof may be operably linked to one or more different polynucleotides or parts thereof to form a single polynucleotide that can be expressed or translated in a cell to form a polypeptide of interest, preferably an antigenic polypeptide. In some embodiments the different polynucleotides or parts thereof are homologous polynucleotides or parts thereof. In some embodiments the different polynucleotides or parts thereof are heterologous polynucleotides or parts thereof.

Likewise, a heterologous polypeptide or part thereof may be fused to one or more different polypeptides or parts thereof to form a single polypeptide of interest, preferably an antigenic polypeptide. In some embodiments the different polypeptides or parts thereof are homologous polypeptides or parts thereof. In some embodiments the different polypeptides or parts thereof are heterologous polypeptides or parts thereof.

The term “functional variant or fragment thereof” of a polypeptide refers to a subsequence of the polypeptide that performs a function that is required for the biological activity or binding of that polypeptide and/or provides the three-dimensional structure of the polypeptide. The term may refer to a polypeptide, an aggregate of a polypeptide such as a dimer or other multimer, a fusion polypeptide, a polypeptide fragment, a polypeptide variant, or functional polypeptide derivative thereof performs the polypeptide activity.

“Isolated” as used herein with reference to polynucleotide or polypeptide sequences describes a sequence that has been removed from its natural cellular environment or from a cellular environment in which it was synthesized or expressed. An isolated molecule may be obtained by any method or combination of methods as known and used in the art, including biochemical, recombinant, and synthetic techniques. The polynucleotide or polypeptide sequences may be prepared by at least one purification step.

In some embodiments a fusion polypeptide as described herein is isolated. In some embodiments a polynucleotide as described herein is isolated.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues, and orthologues. In certain embodiments, variants of the polynucleotides and polypeptides described herein have biological activities that are the same, similar, or substantially similar to those of a corresponding wild-type molecule, i.e., the naturally occurring polypeptides or polynucleotides. In certain embodiments the similarities are similar activity and/or binding specificity.

In certain embodiments, variants of the polynucleotides and polypeptides described herein have biological activities that differ from their corresponding wild-type molecules.

In certain embodiments the differences are altered activity and/or binding specificity.

The term “variant” with reference to polynucleotides and polypeptides encompasses all forms of polynucleotides and polypeptides as defined herein.

### Polynucleotide Variants

Variant polynucleotide sequences preferably exhibit at least 50%, at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 8 nucleotide positions, preferably at least 10 nucleotide positions, preferably at least 15 nucleotide positions, preferably at least 20 nucleotide positions, preferably at least 27 nucleotide positions, preferably at least 40 nucleotide positions, preferably at least 50 nucleotide positions, preferably at least 60 nucleotide positions, preferably at least 70 nucleotide positions, preferably at least 80 nucleotide positions, preferably over the entire length of a polynucleotide as described herein.

Polynucleotide variants also encompass those which exhibit a similarity to one or more of the specifically identified sequences that is likely to preserve the functional equivalence of those sequences, and which could not reasonably be expected to have occurred by random chance.

Polynucleotide sequence identity and similarity can be determined readily by those of skill in the art.

Variant polynucleotides also encompass polynucleotides that differ from the polynucleotide sequences described herein but that, due to the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

In the context of the present description, a “functional variant or fragment thereof” of a polynucleotide is one that comprises additions, substitutions and/or deletions in the nucleotide residues that code for non-essential amino acid residues, and/or of non-essential amino acid sequences (e.g., of SEQ ID NO: 1), where “non-essential” means amino acid residues or sequences that do not affect the functionality of the protein expressed.

In some embodiments, a functional variant of a fusion polypeptide as described herein is a fusion polypeptide comprising a specific peptide or polypeptide inserted between the signal sequence and the mature portion of the variant fusion polypeptide.

In some embodiments a functional variant of a polynucleotide as described herein is a polynucleotide comprising short nucleotide sequence or single residue replacement that allow site-specific (targeted) chemical or enzymatic modifications of a displayed polypeptide expressed from the polynucleotide variant.

### Polypeptide Variants

The term “variant” with reference to polypeptides also encompasses naturally occurring, recombinantly and synthetically produced polypeptides. Variant polypeptide sequences preferably exhibit at least 35%, preferably at least 40%, preferably at least 50%, preferably at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 2 amino acid positions, preferably at least 3 amino acid positions, preferably at least 4 amino acid positions, preferably at least 5 amino acid positions, preferably at least 7 amino acid positions, preferably at least 10 amino acid positions, preferably at least 15 amino acid positions, preferably at least 20 amino acid positions, preferably over the entire length of a polypeptide as described herein.

The terms “variant polypeptide”, “polypeptide variant” and “modified polypeptide” (including grammatical variations thereof) are used interchangeably herein and mean the same thing.

Polypeptide variants also encompass those which exhibit a similarity to one or more of the specifically identified sequences that is likely to preserve the functional equivalence of those sequences, and which could not reasonably be expected to have occurred by random chance.

Polypeptide sequence identity and similarity can be determined readily by those of skill in the art.

A variant or modified polypeptide includes a polypeptide wherein the amino acid sequence differs from a polypeptide herein by one or more conservative amino acid or non-conservative substitutions, deletions, additions, or insertions which do not affect the biological activity of the peptide.

Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine.

Analysis of evolved biological sequences has shown that not all sequence changes are equally likely, reflecting at least in part the differences in conservative versus non-conservative substitutions at a biological level. For example, certain amino acid substitutions may occur frequently, whereas others are very rare. Evolutionary changes or substitutions in amino acid residues can be modelled by a scoring matrix also referred to as a substitution matrix. Such matrices are used in bioinformatics analysis to identify relationships between sequences and are known to the skilled worker.

Other variants include peptides with modifications which influence peptide stability.

Such analogs may contain, for example, one or more non-peptide bonds (which replace the peptide bonds) in the peptide sequence. Also included are analogs that include residues other than naturally occurring L-amino acids, e.g., D-amino acids or non-naturally occurring synthetic amino acids, e.g., beta or gamma amino acids and cyclic analogs.

Substitutions, deletions, additions, or insertions may be made by mutagenesis methods known in the art. A skilled worker will be aware of methods for making phenotypically silent amino acid substitutions. See for example (Bowie et al., 1990).

A polypeptide as used herein can also refer to a polypeptide that has been modified during or after synthesis, for example, by biotinylation, benzylation, glycosylation, phosphorylation, amidation, by derivatization using blocking/protecting groups and the like. Such modifications may increase stability or activity of the polypeptide.

In the context of the present description, a “functional variant or fragment thereof” of a polypeptide, including a fusion polypeptide, is one that comprises additions, substitutions and/or deletions of non-essential amino acid residues, and/or of non-essential amino acid sequences where “non-essential” means amino acid residues or sequences that do not affect the functionality of the expressed polypeptide.

Antibiotic resistance selective marker is used here as known in the art, and comprises, in a polynucleotide as described herein, antibiotic resistance genes that are expressed from a nucleic acid expression construct to produce polypeptides that provide a host cell into which they have been transformed and expressed, resistance to at least one antibiotic used in a culture medium to select for cells transformed with the polynucleotide.

The term “origin of replication” and grammatical variations thereof as used herein means a nucleic acid origin of replication as known and used in the art.

The term “Ff phage genes” and grammatical variations thereof as used herein refers to the polynucleotide or nucleic acid sequences that encode the replication and coat proteins of an Ff phage as described herein. Ff phage genes may be organized into operons as known in the art and as described herein.

The term “scaffold nucleic acid sequence” and grammatical variations thereof as used herein refers to the DNA sequence corresponding to the (+) strand circular ssDNA that is replicated from a BSFnano replication-assembly cassette and subsequently packaged into a nanorod.

The term “functionalization ready” and grammatical variations thereof as used herein with reference to a nanorod as described herein refers to at least one polypeptide comprised in the nanorod that comprises a modifiable amino acid sequence in an appropriate position and/or context within the nanorod and the polypeptide per se, such that the modifiable amino acid sequence is available to be modified to allow attachment, to the nanorod, of a chemical moiety.

In some embodiments the chemical moiety is a small molecule, antibody, polypeptide, polynucleotide, small organic molecules such as biotin, fluorescent dyes such as FITC, various affinity tags, or immune adjuvant molecules such as alpha-galactoceramide (α-GalCer).

The term “producing” (and grammatical variations thereof) as used herein with reference to nanorods made using an NPS as described herein refers to the expression, replication, and assembly of nanorods from an NPS as described herein.

The term “at least one” as used herein with reference to described features, including but not limited to “at least one inducible promoter”, “at least one selective marker”, “at least one auxotrophic marker” and other such usages, means that at least one of the stated features is present. However, this term as used herein also specifically contemplates as an embodiment, the singular “the”, “a”, “an”, and/or “one” (including other such grammatical variations).

The term “(+) strand DNA” and grammatical variations thereof as used herein means a (+) strand circular single-stranded DNA (ssDNA).

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION OF INVENTION

The inventors have found that the problems outlined herein, related to the efficient production of biological scalable functionalization-ready nanorods (BSFnano), can be overcome by providing a biological system that is capable of efficiently providing relatively high yields of substantially pure short nanorods that do not comprise antibiotic resistance genes in their DNA backbone.

Accordingly, described herein is a platform for production of biological scalable functionalization-ready nanorods (BSFnano) of the following dimensions 6 nm×≥40 nm. The platform is disclosed as two systems. A first system comprises a single plasmid termed a “Pop-up” plasmid and includes single plasmid variants as described herein (the Pop-up series). A second system described herein comprises two plasmids, a helper plasmid (pHP) and a nanorod replication-assembly plasmid (pBSFnano). Included in the second system are variants of the pHP (the pHP series) and the pBSFnano (pBSFnano series) helper and nanorod replication-assembly plasmids, respectively.

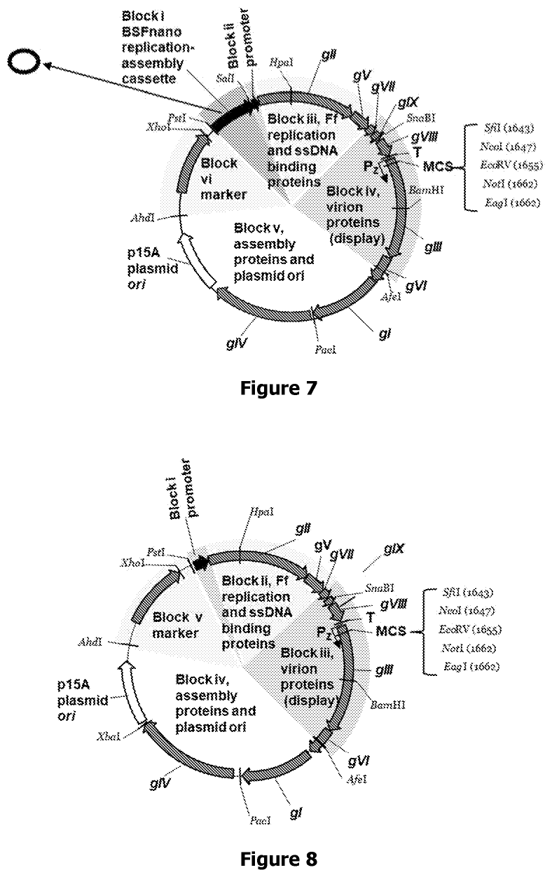

As noted above, each of these two systems includes variants, these variants being suitable for specific applications. Variants of the system are, in turn, constructed by combining a series of exchangeable sequence units within each of the plasmids (Tables 2, 3, 6 and 7; FIGS. 7-9; 29-57).

Bacterial cells containing the plasmids described above are used for the nanorod production. These cells belong to strains containing specific mutations that are required for various aspects of the coat production and vary depending on the characteristics of specific functional units suitable for a particular application (bacterial genotypes are listed in Table 1).

As outlined in the current disclosure, the inventors identified a surprising and unexpected technical solution that allows the skilled worker to overcome the problems outlined herein, particularly by allowing the production of Ff phage-derived biological scalable nanorods without the concurrent production of longer filamentous Ff phage particles.

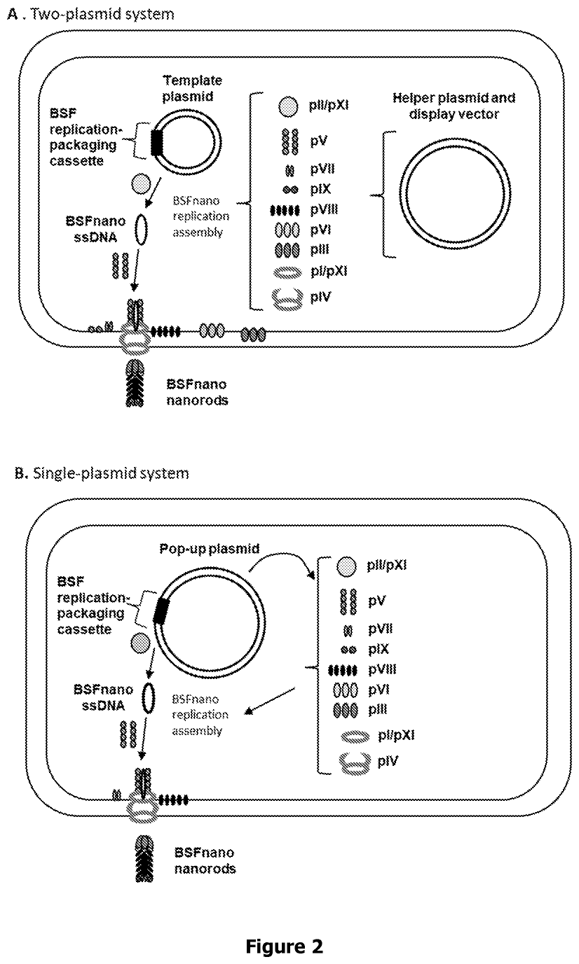

As disclosed herein, the inventors have replaced helper phage with a helper plasmid which does not assemble into phage particles, but nevertheless provides all Ff phage proteins required for replication of short nanorods from a nanorod replication-assembly cassette. In this manner, the inventors have eliminated the use of helper phage per se, including all associated disadvantages (FIG. 2A). Further, the inventors have identified that the same advantages related to eliminating the use of helper phage or helper plasmids per se can be achieved, using a single plasmid system, the single plasmid system comprising a single nucleic acid expression construct comprising all the functions of a nucleic acid expression construct comprising a replication assembly cassette and a helper construct as described above, for the production of short nanorods. The single plasmid system is termed herein pPop-up (FIG. 2B).

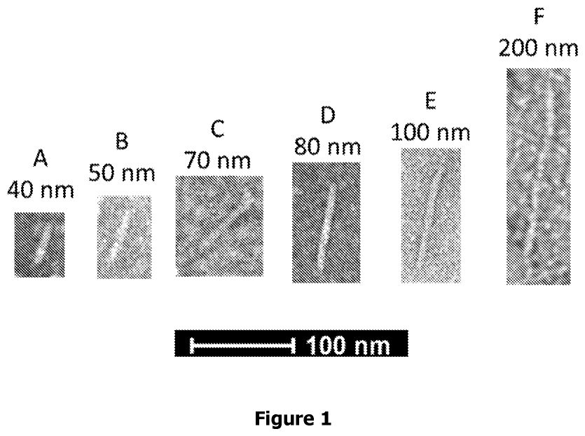

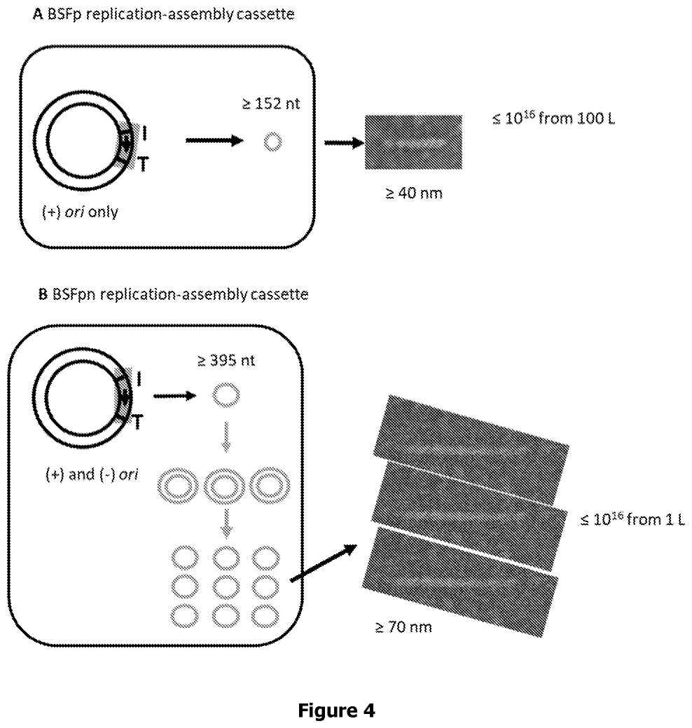

In a further technical advantage described herein, the inventors have found that by extending the replication-assembly cassette for production of the short nanorod backbone, by including the (−) strand origin of replication (“(−) on”) and a complete (+) on as the initiator (FIGS. 4B, 5B, 6A and B), the pPop-up and dual plasmid systems described herein allow for a higher production efficiency of nanorods having a longer minimal length (70 nm; FIG. 1C) and is termed BSFpn (for Biological Scalable Ff replication-assembly cassette, positive and negative origin). In contrast, a pPop-up system comprising only a BSFnano replication-assembly cassette as described herein containing a positive origin only is named BSFp (p standing for positive origin; FIGS. 1A, B, 4A, 5C, 6C), In a further technical advantage described herein, the inventors have found that biological scalable nanorods can be produced without the use of an antibiotic-resistance marker in the BSF nano replication-assembly cassette (single plasmid system) or nanorod replication-assembly plasmid (two-plasmid system). In the present disclosure, selection for positive transformants was carried out using an auxotrophic marker, nadC, encoding enzyme in the biosynthesis pathway of NAD (nicotine amid dinucleotide), an essential metabolite. In this manner, using a nanorod production system (NPS) as described herein, biological scalable nanorods that are entirely free of antibiotic resistance gene sequences are produced.

Although the inventors have identified that the use of helper plasmids can eliminate the production of the helper phage, the introduction into E. coli, of a nanorod replication-assembly plasmid, can introduce a bottleneck due to the limitation in the absolute number of transformed cells to ˜10⁷ per transformation. To expand the number of cells that produce nanorods, and therefore the total yield of the nanorods, the transformation reaction needs to be inoculated into the fresh medium (e.g., 1 L) and incubated over at least 13 generations to reach the exponential phase of growth (10¹¹ cells per L).

Due to a regulatory circuit that controls production and function of the replication protein pII, replication of Ff (and BSFnano by derivation) and the number of produced particles per cell decreases progressively over the 13 generations required to reach the cell density of 10¹¹ per L (Lerner and Model, 1981; Merriam, 1977).

To overcome these shortcomings, the inventors have introduced yet another technical advantage of their system as described herein. Specifically, the inventors enable the inducible expression of genes involved in replication of Ff phage by replacing the constitutive promoter P_{A} upstream of gII (FIG. 7, 8, 49; SEQ NO: 89) with an inducible lacUV5 promoter (FIG. 7, 8, 49; SEQ NO: 90). This replacement is effective in both the single and two plasmid nanorod production systems described herein (e.g., in the helper plasmid (pHP) of a two-plasmid system, and the in the pPop-up single plasmid system (Table 7, 8). This modification allows the inventors exquisite control of the timing of nanorod production as described herein, in which the initiation of replication, and hence the production of nanorods, is delayed until the density of the pBSF template-plasmid-containing cells in the transformed culture reaches a desired value (Table 8). In one embodiment the desired value of cells/mL that is equivalent to an exponentially growing culture (e.g., about 10⁸/mL or 10¹¹/L).

Based on their overall concept, the inventors have designed a series of embodiments comprising elements, within the plasmids of a two-plasmid system, or within a single plasmid pPop-up system, that can be used to adjust the production of nanorods depending on the desired functionalization(s): recombinant, enzymatic or chemical, and the marker (antibiotic or auxotrophic) (FIGS. 7-9; Examples 1 and 2).

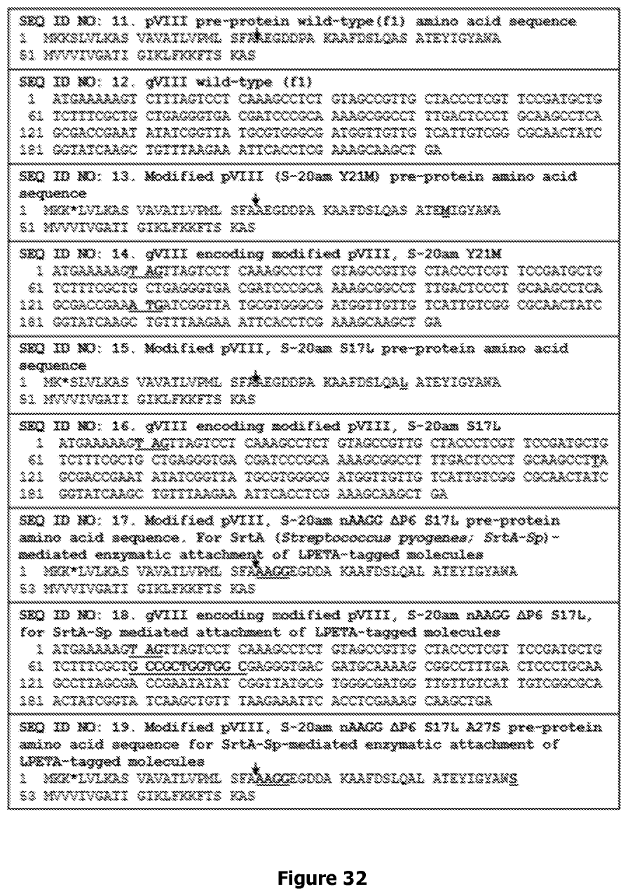

In some embodiments, at least one variant as described herein is a variant of the major coat protein pVIII that has been modified to comprise functional groups that are suitable for chemical or enzymatic modification (SEQ ID: 13; SEQ ID: 15, SEQ ID: 17, SEQ ID: 19, SEQ ID: 21, SEQ ID: 23, SEQ ID: 25, SEQ ID: 27; SEQ ID: 97, FIGS. 32, 33, 54).

In some embodiments, at least one variant as described herein is a variant of a minor coat protein (for example of pIII, pVI, pVII or pIX but not limited thereto) that has been modified to comprise functional groups that are suitable for chemical or enzymatic modification.

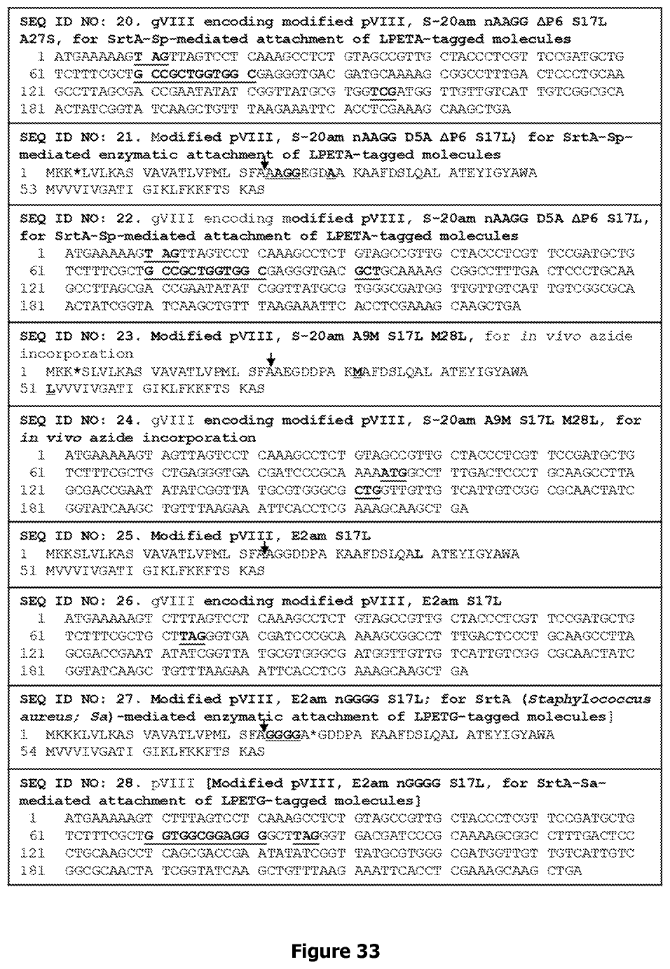

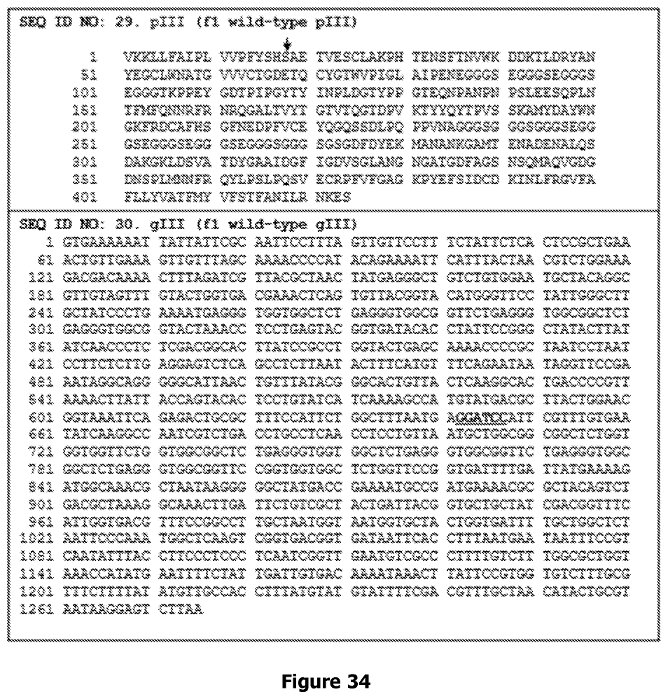

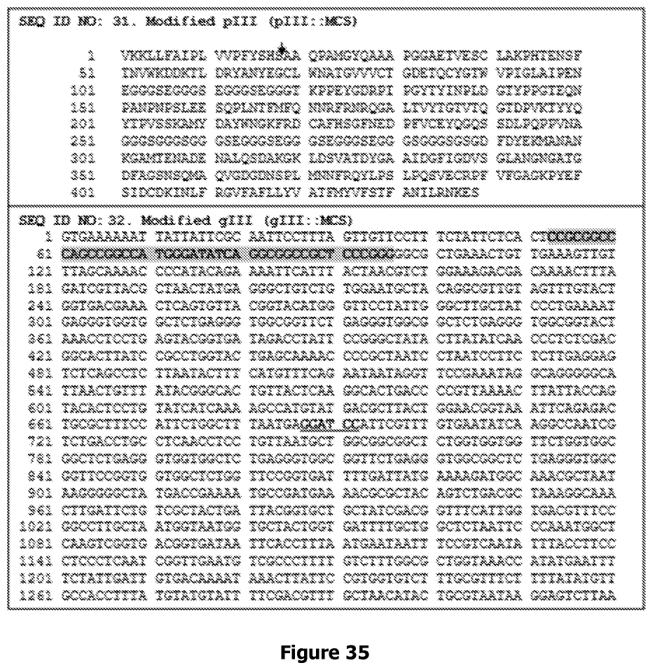

In one embodiment of a modification to comprise functional groups that are suitable for enzymatic modification, an AlaGlyGly is inserted at position 2 of mature pVIII coupled with deletion of Pro at position 6). This modification resulted in an N-terminal AlaAla motif, but very low nanorod production (FIG. 32, SEQ ID NO: 17, SEQ ID NO: 18). To overcome this problem, this gVIII variant was introduced into the Ff recombinant bacteriophage, causing poor replication and pinpoint plaques, and produced stocks of low titres. The virus was then “evolved” through three rounds of growth, resulting in mutants that recovered production, as evidenced by wild-type-like plaque size and titre (FIG. 32, SEQ ID NO: 19; FIG. 33, SEQ ID NO: 20; SEQ ID NO: 21 and SEQ ID NO: 22).

In a particular embodiment, described herein are two evolved variants that gave the highest titres of phage. Both variants had missense mutations in gVIII that resulted in amino acid changes in the mature portion of pVIII. One evolved mutant had Ala replaced by Ser at position 27 (FIG. 32, SEQ ID NO: 19 and FIG. 33, SEQ ID NO: 20) and another mutant had Asp replaced by Ala at position 5 (FIG. 33, SEQ ID NO: 21, SEQ ID NO: 22) as counted in the wild-type mature pVIII. The mutated gVIII sequence was then introduced back into the helper plasmid pHP1 or pPop-up and shown to have restored production of BSF nanorods (FIGS. 32-33; SEQ ID NO: 19, SEQ ID NO: 20, SEQ ID NO: 21, SEQ ID NO: 22).

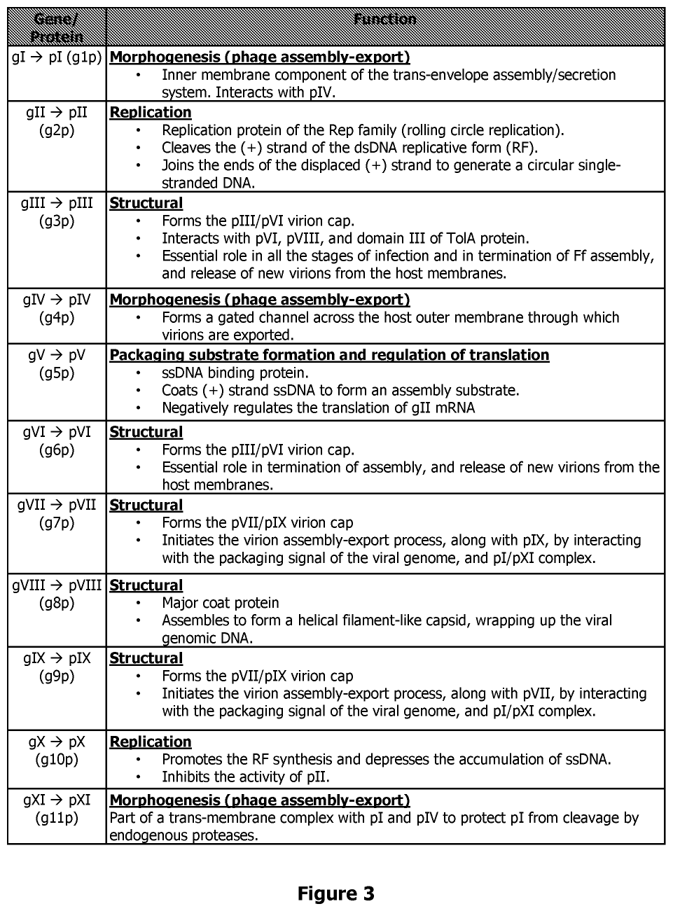

The Pop-up single-plasmid system One type of the BSF nanorod production system (NPS) is composed of a single plasmid that is expressed in the appropriate host cells (FIG. 7). The Pop-up plasmid is composed generally of three major components (A, B and C):
A) The BSFnano replication-assembly cassette;
B) Ff phage genes (listed in FIG. 3);
C) Plasmid origin of replication and selection marker(s) for maintenance of the plasmid in E. coli cells.

Each of these components is assembled from smaller exchangeable units or blocks that can be combined to attribute specific properties to the BSF nanorods (FIG. 7).

### A) the BSFnano Replication-Assembly Cassette (Block i):

The BSFnano replication-assembly cassette serves as a template for Ff rolling-circle replication and gives rise to a plurality of (+) strand circular ssDNAs which serve as backbones for assembly of the short nanorods termed BSFnano herein (FIGS. 4-6; 39-48; 56-57; SEQ ID NOs: 41-52; 101-104). The skilled person appreciates that these backbone ssDNAs are also termed herein “scaffolds” that mediate the assembly of the Ff phage proteins into nanorods as described herein.

In one example, a BSFnano replication-assembly cassette in the pPop-up plasmid series is a combination of the following units:
Initiator (+) ori1, a functional positive-strand origin of replication (+) on that allows binding of the replication protein pII (a DNA-strand-transferase) and cutting of the (+) strand to form a primer (FIGS. 4-6; 39-48; 56-57; SEQ ID NO: 45, SEQ ID NO: 65, SEQ ID NO: 81; SEQ ID NO: 103).
Packaging signal (PS) that is required for targeting of the (+) strand circular ssDNA replicated from a BSFnano replication-assembly cassette to the trans-envelope assembly machinery for assembly of the nanorods (FIGS. 4-6; 40, 42, 44, 46, 48; 57; SEQ ID NO: 48, SEQ ID NO: 68, SEQ ID NO: 76, SEQ ID NO: 83, SEQ ID NO: 84).
The (−) on allows replication of the negative strand using the short BSFnano (+) strand ssDNA as a template, to increase the copy number of the (+) stand circular ssDNAs produced from a BSFnano replication-assembly cassette (FIGS. 4-6; 40, 42, 44, 46, 57; SEQ ID NO: 50).
Terminator ((+) ori2) is a truncated (+) on mutant (Δ29) that allows cutting of the template (+) strand whose replication started at (+) ori1, and ligation of the two ends of the (+) strand to produce a (+) strand circular ssDNA that serves as a backbone for nanorod assembly as described herein (FIGS. 4-6; 40, 42, 44, 46, 48, 57; SEQ ID NO: 51, SEQ ID NO: 69, SEQ ID NO: 87).
Properties and variants of the BSFnano replication-assembly cassettes:

The initiator, (+) ori1, can be either the minimal or core domain of (+) on (A or I) only (FIGS. 4-6, 46, 48; SEQ ID NO: 74, SEQ ID NO: 81), or the complete (+) on (both A and B domains; (FIGS. 4-6, 40, 42, 44, 57; Seq ID NO: 45, SEQ ID NO: 65, SEQ ID NO: 103), with the latter being more efficient at initiation than the former, due to the presence of the complete pII binding sequence.

The lengths of produced nanorods are determined by the sizes of scaffold nucleic acid sequences comprised in the BSFnano replication-assembly cassettes as described herein. The scaffold nucleic acid sequences are positioned between a first pII nick site in (+) ori1 and a second pII nick site in (+) ori2 (GTTCTT [symbol] 1rAATA)(SEQ ID NO: 88) in the BSFnano replication-assembly cassettes (FIGS. 4-6; 39-48; 56-57; SEQ ID NO: 41, SEQ ID NO: 43, SEQ ID NO: 52, SEQ ID NO: 54, SEQ ID NO: 61, SEQ ID NO: 63. SEQ ID NO: 70, SEQ ID NO: 72, SEQ ID NO: 77, SEQ ID NO: 79, SEQ ID NO: 101)

### BSFp Replication-Assembly Cassette

For example, a BSFnano replication-assembly cassette composed of the initiator (+) ori1 comprising only (+) on core (or domain A), packaging signal and terminator (+) ori2 corresponding to (+) on Δ29, we named here BSFp, results in production of the circular (+) ssDNA of 152 or 221 nt and assembly, respectively, nanorods of 40 or 50 nm in length (FIGS. 1, 4-6, 47-48, SEQ ID NO: 79, SEQ ID NO: 80, SEQ ID NO: 77, SEQ ID NO: 78). The 40 nm nanorods are the shortest Ff-derived nanorods produced to date.

### BSFpn Replication-Assembly Cassettes

In another example, replication-assembly cassette we named BSFpn contains a combination of initiator ((+) ori1) corresponding to the complete (+) on (domains AB), a packaging signal, a (−) on and (+) ori2 (a terminator, (+) on Δ29). In the presence of pII this replication-assembly cassette results in replication of the (+) strand ssDNA of 395, 529, 707, 711, 728, 748 nt, and nanorods that are 70, 80, 100 or 110 nm in length (FIGS. 1, 4-6, 39-44, SEQ ID NO: 41, SEQ ID NO: 43, SEQ ID NO: 52, SEQ ID NO: 54, SEQ ID NO: 61, SEQ ID NO: 63). Longer BSFnano nanorods can be produced if DNA sequence be inserted between the (+) ori1 and the PS.

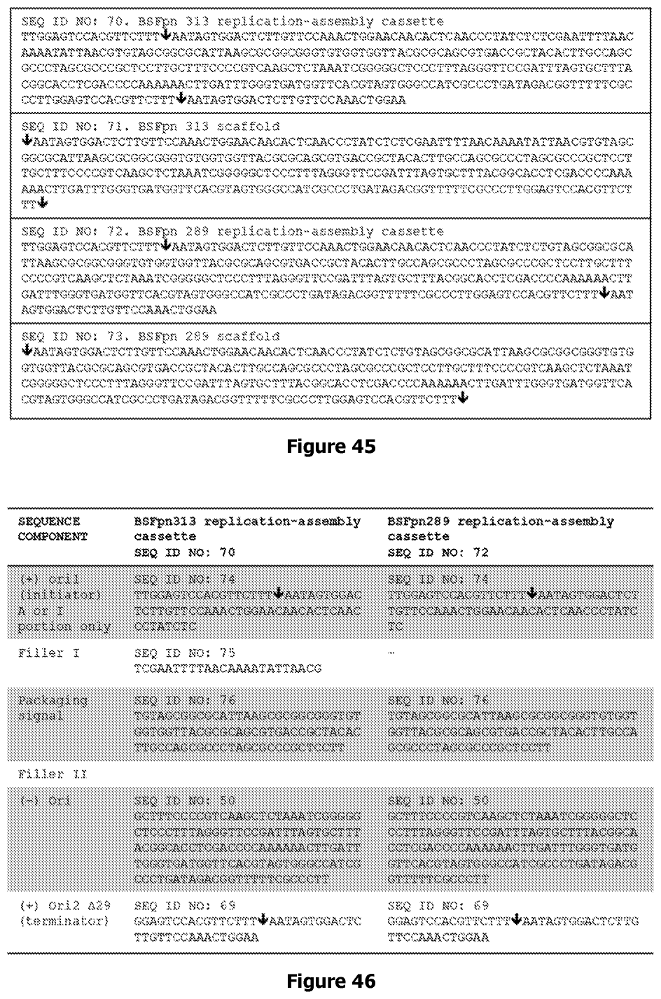

Another variation of the BSFpn replication-assembly cassette is possible where the (+) ori1 would contain only the core (+) on (domain A) as the initiator and would still include the (−) on (FIGS. 45, 46; SEQ ID NO: 70, SEQ ID NO: 72; SEQ ID NO: 74). The ssDNA produced from such a BSFpn cassette would be 313 or 289 nt, resulting in nanorod of a calculated length of, respectively, 57 or 54 nm (˜55-60 nm).

### Scalability of BSF Nanorods

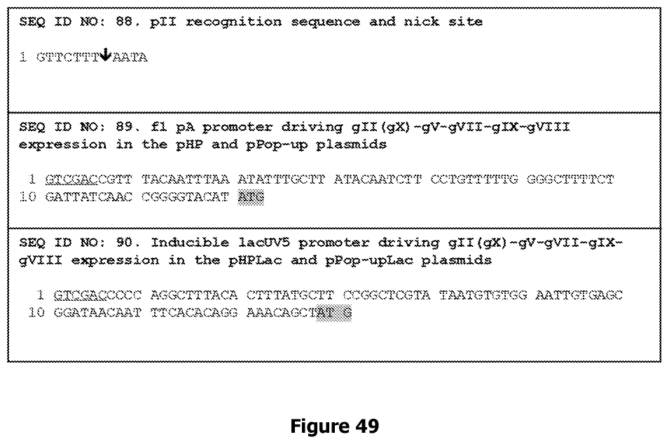

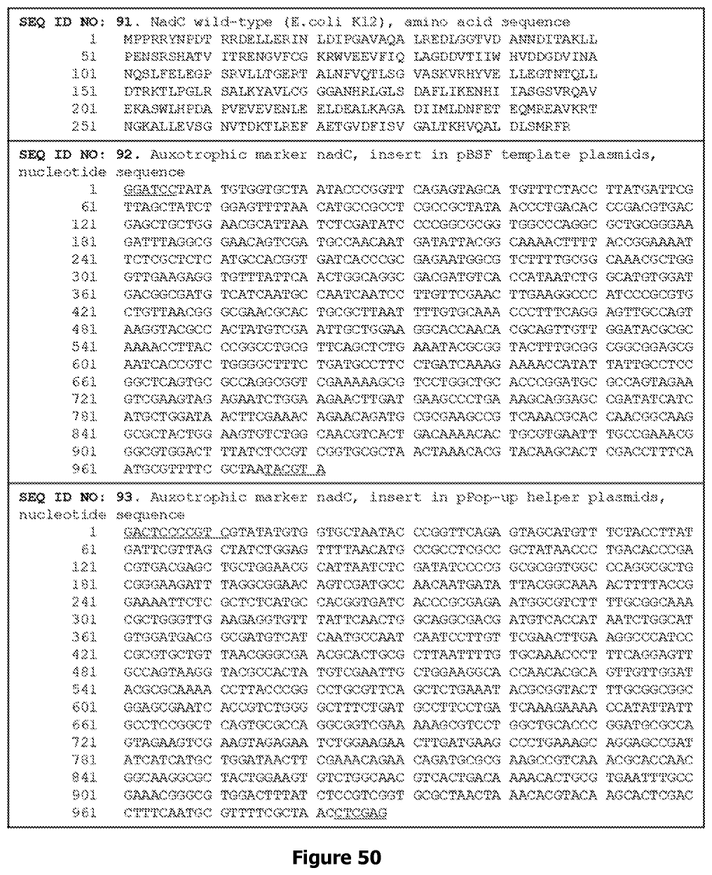

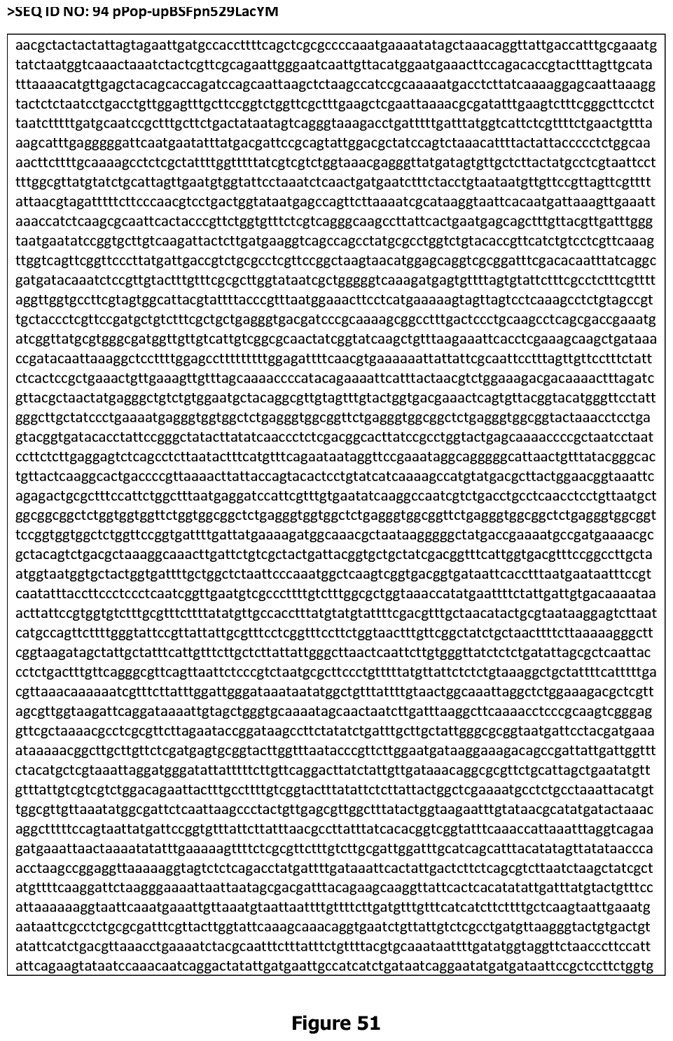

In both the single and dual plasmid NPSs described herein, a scaffold nucleic acid sequence is comprised in the BSFnano replication-assembly cassette between the pII cut sites ((GTTCTT [symbol] AATA) (SEQ ID NO:88, FIG. 49) in (+) ori1 (initiator) and in (+) ori2 (terminator; FIGS. 1,4-6, 39-48). A person of skill in the art recognizes that a scaffold nucleic acid sequence of the appropriate size to produce a nanorod and/or plurality of nanorods of a desired size can be readily selected for use in an NPS as described herein based on the disclosure of the present specification and as known in the art.

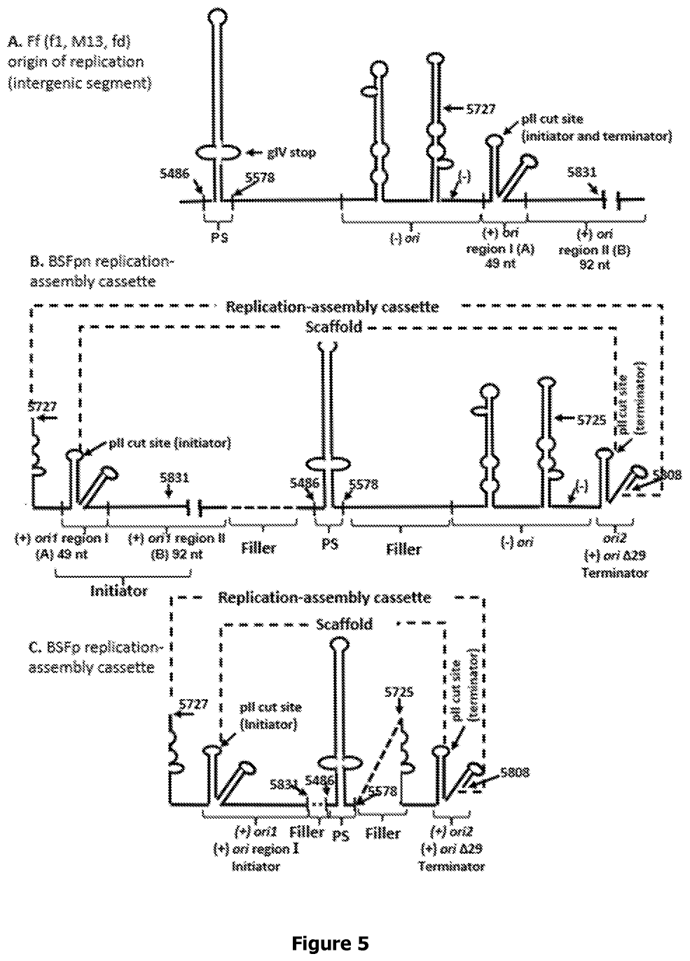

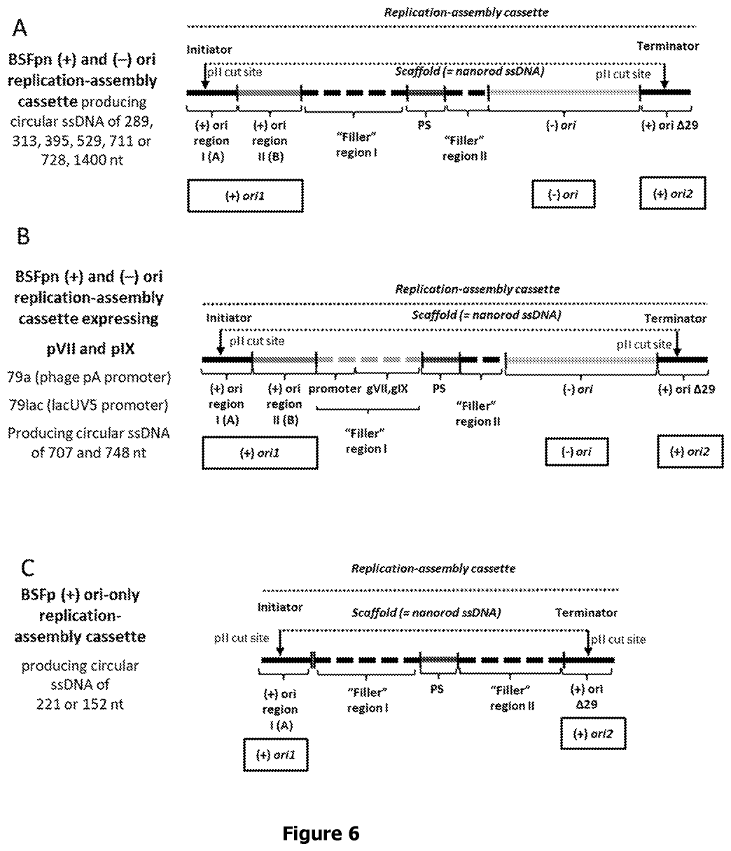

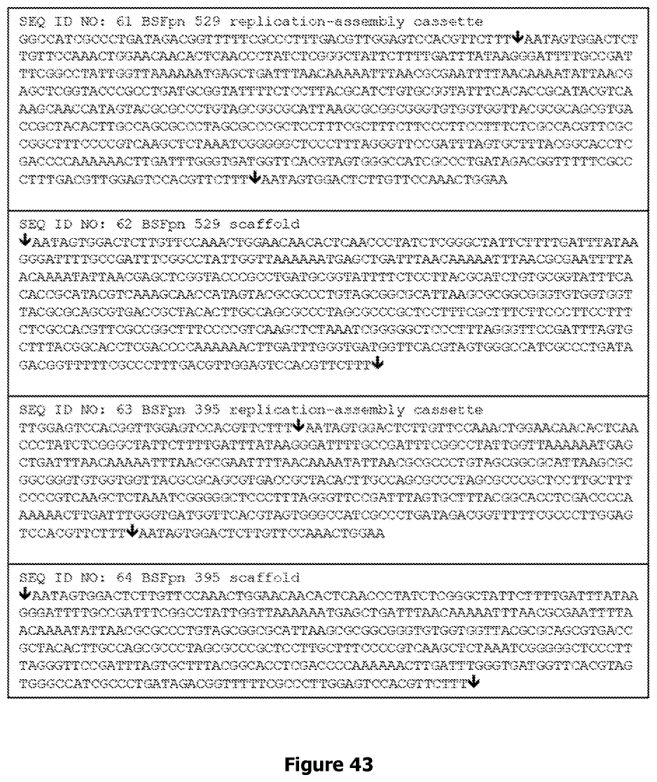

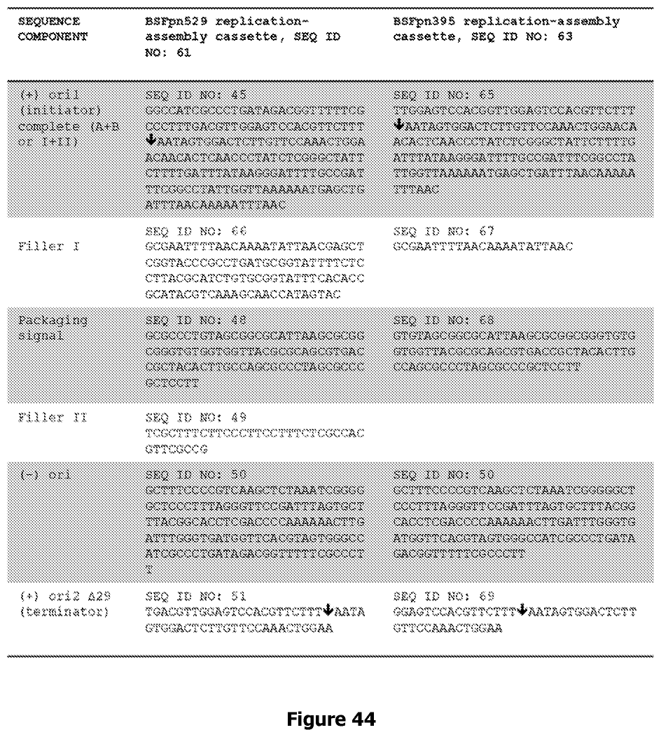

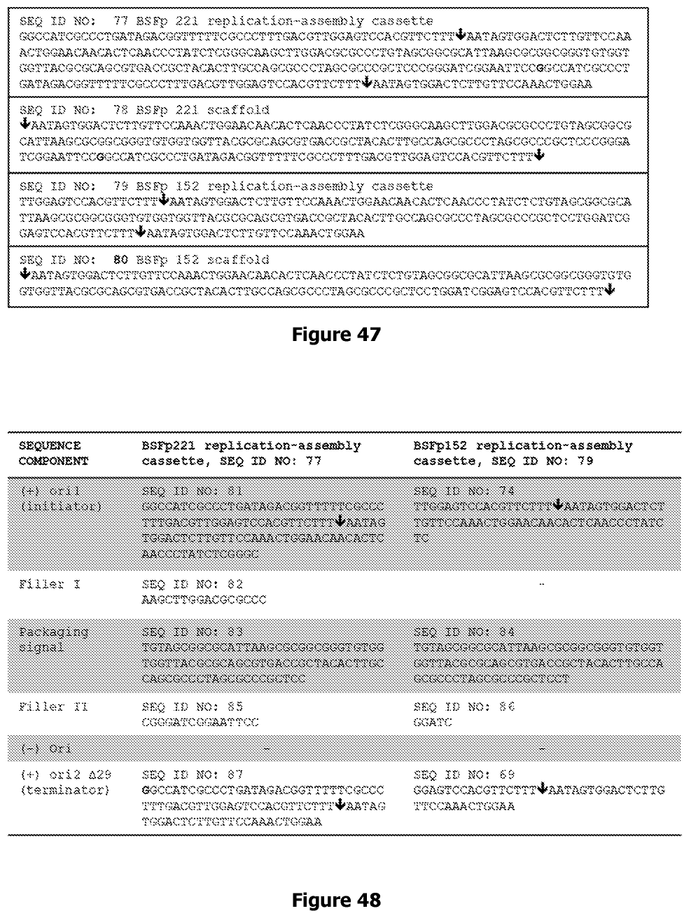

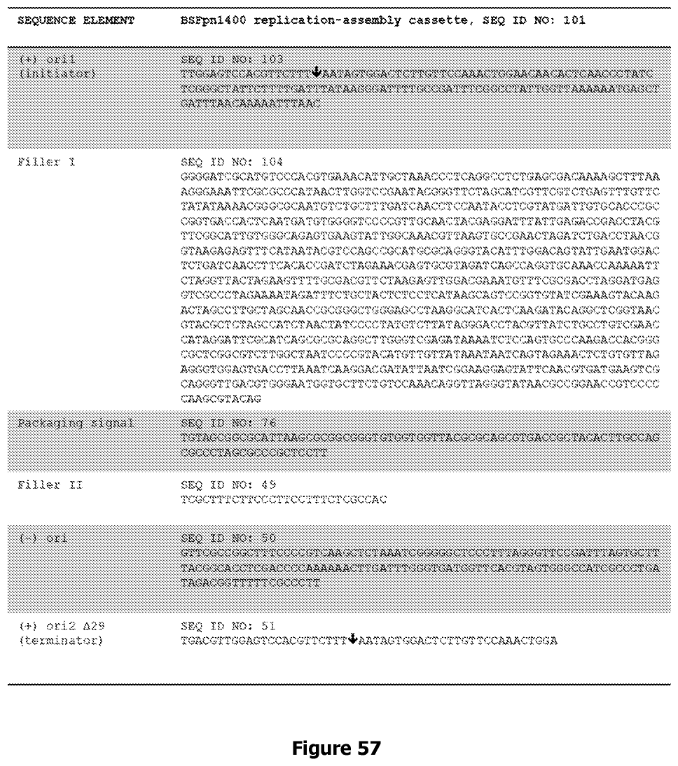

As noted previously, the length of the (+) strand circular ssDNA backbone (scaffold) produced by rolling circle replication of the BSFnano replication-assembly cassettes is determined by the number of nucleotides between the pII cut sites in the (+) ori1 (initiator) and (+) ori2 (terminator). The length of the scaffold nucleic acid sequence can be decreased in order to reduce the size of the nanorods by removing the (−) on (as done in the BSFp replication-assembly cassettes, completely removing the filler sequences and by reducing the size of the (+)ori1 and (+)ori2 in BSFpn replication-assembly cassette (Table 9; e.g. FIGS. 43 and 44, SEQ ID NO: 63, SEQ ID NO: 64, SEQ ID NO: 66 vs SEQ ID NO: 67; FIGS. 45 and 46, SEQ ID NO: 70, SEQ ID NO: SEQ ID NO: 71, SEQ ID NO:72, SEQ ID NO: 73; SEQ ID NO: 74 vs SEQ ID NO: 65; FIGS. 47 and 48, SEQ ID NO: 77, SEQ ID NO: 78, SEQ ID NO: 79, SEQ ID NO: 80, SEQ ID NO: 74). Conversely, the length of the nanorods can be extended by inserting “filler” nucleic acid sequences between the initiator ((+) ori1) and the PS, and between the PS and the (−) on in BSFpn or PS and the (+) ori2 in BSFp replication-assembly cassette (FIG. 5B and C; FIG. 6; FIGS. 56-57, SEQ ID 104). Consequently, the length of the nanorod is extended to a desired length by designing filler nucleic acid sequences of a suitable length. Based on structural analyses of the Ff phage shaft, it can be calculated precisely that addition of every nucleotide to the ssDNA genome increases the length of the nanorod by 0.133 nm (Newman et al., 1977).

Protein-Encoding Genes within the Replication-Assembly Cassette

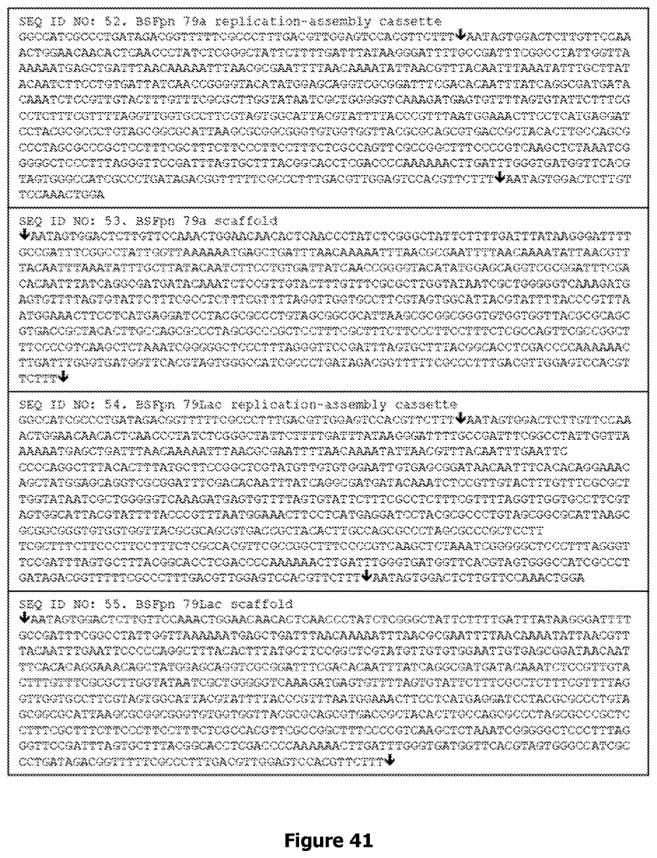

Also contemplated herein, the filler nucleic acid sequences can encode a second copy of gVIII that will be used as a platform for expression of pVIII fusion to long peptides or proteins (FIG. 7, Block i). Alternatively, a second copy of gVIII can be encoded on a compatible plasmid, supplying this Ff phage protein to be incorporated into the nanorods produced as it is usually done in phage display art. An example of expression of Ff phage proteins from a filler nucleic acid sequence comprised in a BSFnano replication-assembly cassette is provided by expression of pVII and pIX from a BSFnano replication assembly cassette as shown in FIG. 6B and FIG. 41 (SEQ NOs: 52-55). In addition to Ff phage proteins expressed in E. coli, filler sequences could be used to accommodate a eukaryotic gene expression cassette for expression in eukaryotic cells.

### B) Ff Phage Genes

Ff phage genes are organized into two operons, gII(gX)-gV-gVII-gIX-gVIII and gIII-gVI-gI-gIV, encoding all functions required for replication of the BSFnano replication-assembly cassettes and assembly of the BSF nanorods. Ff genes are functionally categorized into those encoding replication functions, gII(gX) and gV; (Block iii in the pPop-up plasmids, FIG. 7); coat proteins, gVII, gIX, gVIII, gIII and gVI (Block iv in the pPop-up plasmids, FIG. 7), and assembly, gI(gXI) and gIV (Block v in the pPop-up plasmids, FIG. 7). In some embodiments, a nucleic acid construct comprising these operons further comprises multiple elements whose modification serves to increase the nanorod production or introduce functional groups in an orthogonal fashion, in specific positions and copy number in the nanorods, depending on the application for which the system is engineered.
   Promoter of the gII(gX)-gV-gVII-gIX-gVIII Operon
A surprising technical advance provided by the present disclosure is the ability to regulate production of replication functions, encoded by gII(gX) and packaging-substrate-forming function encoded by gV, in order to induce replication of the BSF replication-assembly cassette at sufficiently high cell density. The nucleic acid constructs of the NPS as described herein (e.g., pBSFp, pBSFpn or pPop-up plasmids) are introduced into E. coli by transformation at a transformation efficiency of about 10⁷ transformed cells per litre of culture (in contrast to a full culture that has a total of about 3×10¹² cells per litre). Therefore, the number of generations (cell divisions) between the transformation and harvesting of the nanorods is ˜20. Based on the quantitative monitoring and derived mathematical modelling (Smeal et al., 2017a, b), phage production falls to a baseline after 107 E. coli cell division times. Applied to the culture of transformed cells (starting number 10⁷ per L), 7 generations correspond to only 10⁹ cells per litre, an equivalent to only 1 mL of full overnight culture. Given that each cell produces a finite number of nanorods, this small number of cells producing the nanorods decreases the overall yield of the nanorods that can be produced from a litre of transformed cells.

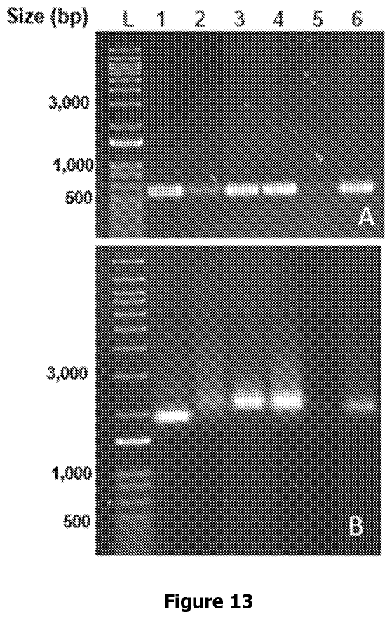

What the inventors have surprisingly determined is that, if pII expression is induced only after the transformed cell culture reaches a higher cell density but while the culture is still in the exponential growth phase (˜10¹¹ cells per L; OD₆₀₀˜0.1), the production of nanorods will peak when the culture contains the highest cell numbers (10¹¹-6×10¹² per L). In this way, a drop in the nanorod production by the time that the culture reaches higher density is avoided. To achieve delayed pII production, gII(gX)-gV-gVII-gIX-gVIII operon expression was placed under an inducible promoter by replacement of the native (constitutive) Ff promoter P_{A} with an inducible promoter (lacUV5; Block ii, SEQ ID NO: 90, FIG. 49). The new family of constructs were engineered that contained lacUV5 promoter instead of the P_{A} promoter in the pPop-up or helper plasmids, resulting, respectively, in the pPop-upLac and pHP1Lac series (FIGS. 7 and 8). Analyses of the nanorod production showed that synchronization of the optimal cell density with the efficient BSF nanorod production by inducible expression of pII increased the nanorod numbers by 10-fold, from 4.6×10¹⁴ to 4.8×10¹⁵ (Table 8; FIGS. 12 and 13, Example 6).

### The gII Allele

The phage-encoded pII used in this disclosure contains a mutation IR1-B (Enea and Zinder, 1982) that allows efficient replication from the core (+) on (domain A).

### Coat Proteins

Ff phage (and the BSF nanorods) are composed of five different coat proteins. Of those, pVIII (50 aa in length) is the major coat protein forming the shaft of the nanorod, present in large number of copies. The exact copy number of pVIII per nanorod depends on the length of the packaged ssDNA (1 pVIII subunit per 2.3 nt (Newman et al., 1977)). The remaining two pairs of “minor” coat proteins are present in small, fixed numbers (5 each per virion), forming two distinct ends of the virion (pIII and pVI at the proximal end and pVII and pIX on the distal end). The nanorod itself has a fivefold axial symmetry (Newman et al., 1977).

As shown in the art of phage display technology, Ff coat proteins each represent a platform for display of functionalities of interest, guided by specific applications (O'Neil and Hoess, 1995; Petrenko, 2008; Rakonjac et al., 2011). Protein fusions are constructed between the coat proteins and heterologous protein sequences, resulting in display of heterologous sequence on the surface of the virions. Alternatively, specific mutations or additional codons are introduced into the coding sequences of the coat proteins to serve as handles for site-specific modification (by “tag and modify” strategy; (Chalker et al., 2011)).
Insertion of heterologous peptide sequences or modification of the coat proteins has to be done in such a way that they do not interfere with the nanorod assembly. Each coat protein has a specific optimal site of insertion and/or segments that can be modified. Heterologous sequences can be inserted between the signal sequence and the mature portion of pIII and pVIII, at the C-terminus of pIII or pVI, or at the N-terminus of pVII and pIX (Fuh and Sidhu, 2000; Gao et al., 1999; Haaparanta and Huse, 1995; Jespers et al., 1996). Some of these fusions do not prevent assembly into the virion if the wild-type copies are also present but cannot mediate phage assembly on their own. In the latter case co-expression of a wild-type copy with a mutated counterpart (fusion) in the same cell allows assembly of the nanorods. As described herein an additional copy of the wild-type or fusion coat protein (i.e., the mutated counterpart) can be either inserted into a single plasmid containing the rest of the phage genes, or expressed from a second plasmid in the same cell (Barbas III et al., 2001). Fusions that can be incorporated into the Ff phage but cannot drive assembly on their own include insertions at the C-termini of pIII and pVI (Fuh and Sidhu, 2000; Jespers et al., 1996) and inserts longer than 6 amino acid residues between the signal sequence of pVIII and its mature portion. The 6-residue insertion effect is sequence-specific, with some sequences tolerated better than others (Iannolo et al., 1995). In some embodiments contemplated herein, a second copy of pVIII (a pVIII fusion to long peptides or proteins) can be expressed from a BSF nano replication-assembly cassette as described herein (FIG. 7, Block i). In one non-limiting example, expression of a second copy of an Ff phage protein from a BSF replication-assembly cassette filler nucleic acid sequence is shown by expression of pVII and pIX from such a cassette as shown in FIG. 6B and FIG. 41 (SEQ NOs: 52-55).

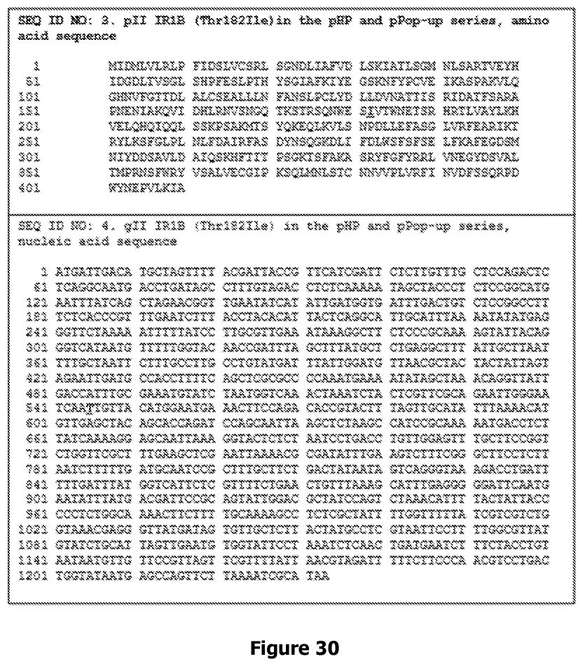

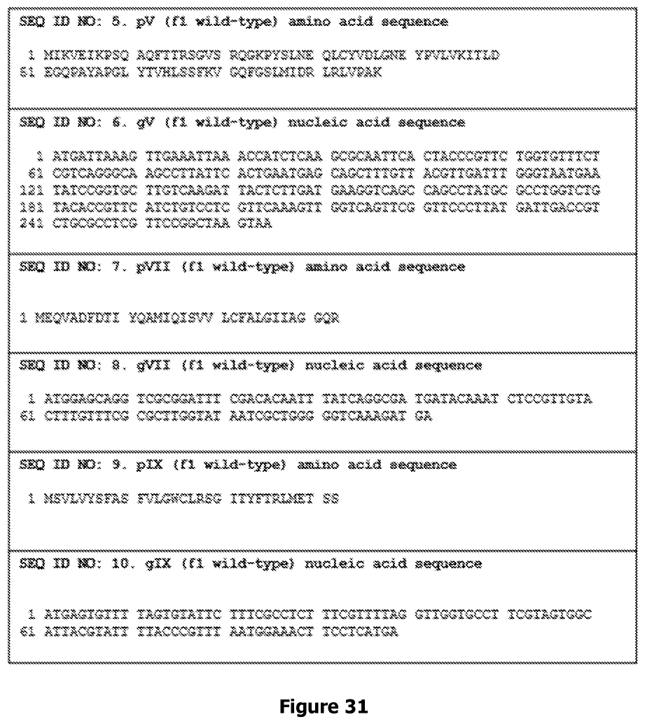

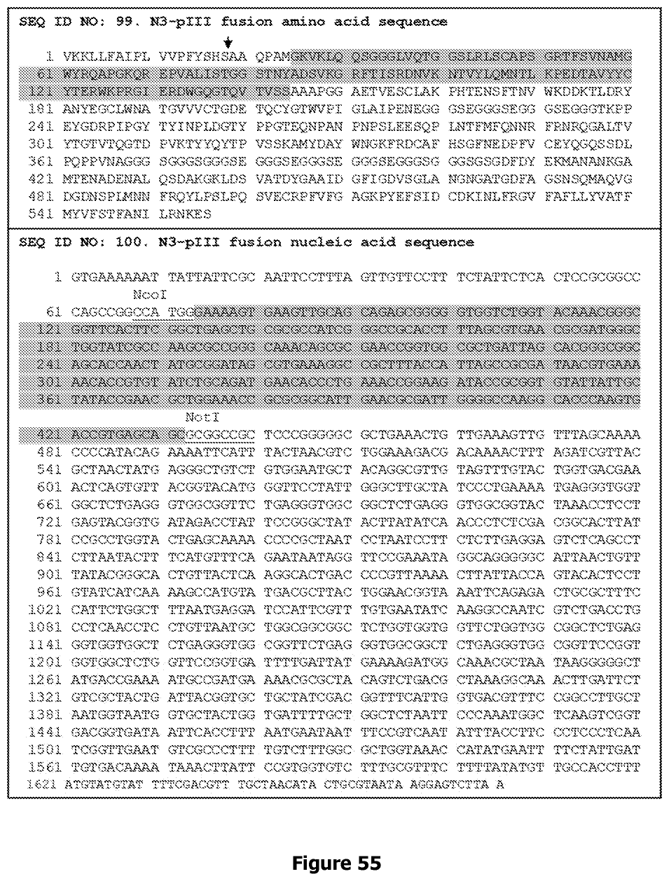

Further contemplated herein, additional expression constructs, including plasmids can be used to supply secondary copies of pVIII coat proteins when the inserted heterologous sequences interfere with assembly of the nanorods in the absence of the wild-type counterpart. These additional plasmids have to have an origin of replication compatible to the Pop-up plasmid, e.g., chloramphenicol resistance (cat; Cm^{R}) marker and ColD origin of replication.
Further modifications to the coat-protein-encoding genes to create functionalization handles, known as “tag and modify” strategy, are made to allow targeted chemical or enzymatic modification. For example, engineering pVIII containing extra ≥3 Glycines or ≥2 Alanines at the N-terminus of the mature coat protein pVIII or pIII or pVII and pIX (addition of a heterologous signal sequence may be required for the two latter proteins) creates a motif that can be used for enzymatic attachment of protein or non-protein molecules conjugated to C-terminal LPXTA or LPXTG motifs, where the attachment of a molecule of interest is catalyzed by the enzyme sortase A (SrtA) of Streptococcus pyogenes (SrtA Sp) or Staphylococcus aureus (SrtA Sa), respectively (Hess et al., 2012). Exchangeable blocks (FIG. 7, Block iv) have been generated for our NPS that produce nanorods with pVIII displaying, at the N-terminus, 4 Gly residues, or 2 Ala residues (FIGS. 30-32 and SEQ NOs: 19-23; 27-28) or 5 residues (FIG. 54, SEQ NOs: 97-98).

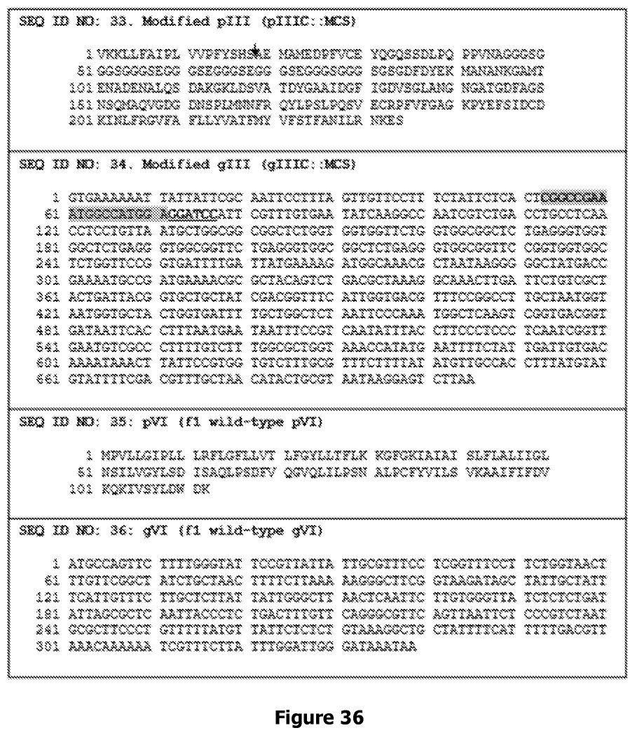

Reactive groups of amino acids, such as the amine groups of the N-terminal residues, lysines, cysteines, tyrosines, aspartic acids, and glutamic acids can be used for chemical modification (Bernard and Francis, 2014). Alternatively, other motifs that are subject to enzymatic or chemical covalent attachment to non-protein molecules, such as SNAP-tag, be directly or indirectly inserted into the nanorods, to allow attachment of a diverse array of molecules. Also described herein are exchangeable blocks that display unpaired Cys residues on pIII, to allow modifications by maleimide-conjugated proteins and small molecules or other chemistries targeting —SH groups (FIG. 7, Block iv; FIG. 36, SEQ NOs: 33-34).

Also described herein is insertion of ATG codons into the coding sequences corresponding to exposed residues of pVIII allows in vivo labelling with unnatural amino acid azidohomoalanine (structurally similar to ATG-encoded residue Met) during translation. Azide groups on the surface of the nanorod provide reactive groups for attachment of molecules using “click” chemistry (Petrie, 2015). This was achieved by synthetizing exchangeable blocks (FIG. 7; Block iv) containing a pVIII variant comprising exposed Met residues (Ala9 mutated to Met) and buried Met residue 28 mutated to Leu. The latter mutation serves to prevent Azide-mediated destabilization of the nanorod structure (FIG. 33; SEQ ID NOs: 23, 24).

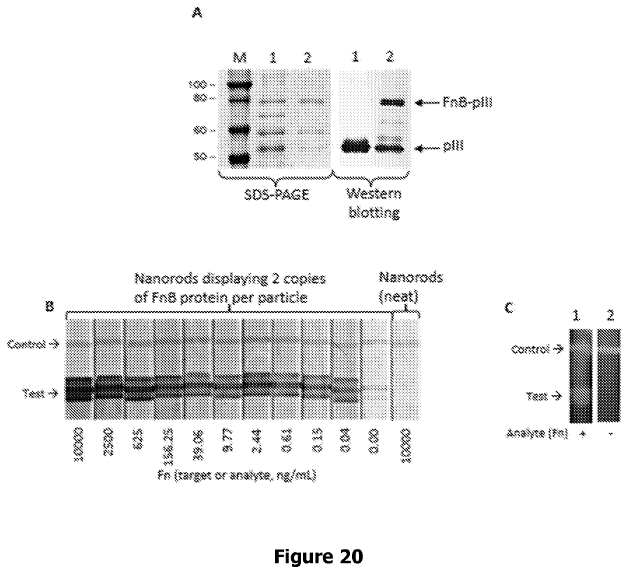

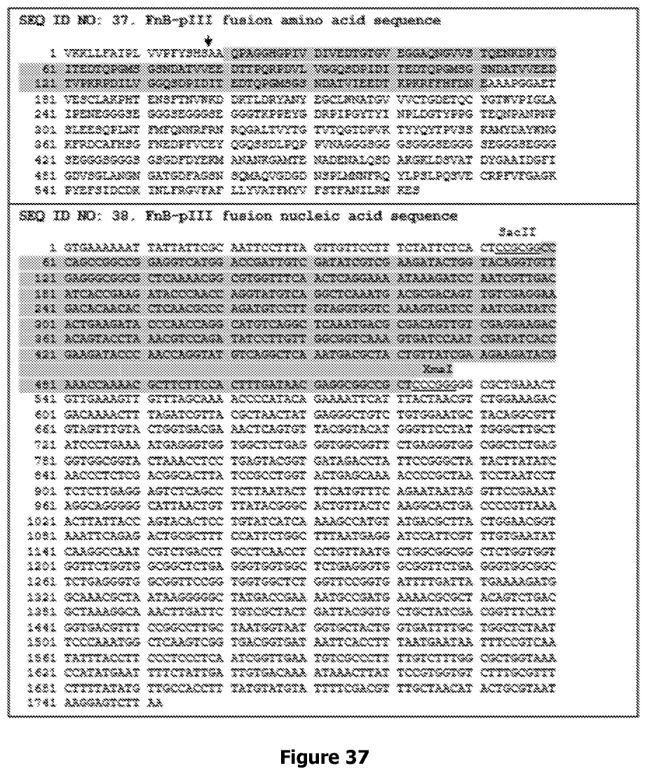

In one non-limiting example, described herein is the fluorescent labelling of the BSF nanorods with amine-reactive fluorescent dye DyLight 550 (FIG. 20, Example 11). These nanorods display a binding molecule (the fibronectin-binding domain of S. pyogenes protein SOF22 (Rakonjac et al., 1995) as the fusion to pIII; SEQ NOs: 37, 38, FIG. 37) and have been used in a lateral flow assay for detection of an analyte, fibronectin (FIG. 20C, Example 11). As will be appreciated, by the skilled worker using this approach any amine-reactive fluorescent or any other dye or other small molecule or biological or chemical polymer that is designed to be amine-reactive is expected to be suitable for attachment to nanorods. Each pVIII subunit has three acidic amino acid residues containing each a side-chain carboxyl groups (Glu2, Asp4 and Asp5) exposed on the surface of the nanorod. Accordingly, carboxyl-reactive molecules can also be chemically conjugated to the nanorods. Other reactive groups, such as the Tyr residue aromatic hydroxyl group can also been used to attach suitable reactive groups as is known in the art (Bernard and Francis, 2014). As will be appreciated, by the skilled worker using this approach any carboxyl-reactive fluorescent or any other dye or other small molecule or biological or chemical polymer that is designed to be amine-reactive is expected to be suitable for attachment to nanorods.

The molecules attached to the nanorods as described herein may be organic molecules of any kind, including, but not limited to biotin, which serves to bind commercially available or in-house made fusions of biotin-binding proteins such as avidin. In this fashion, the nanorods described herein may be modified to display a broad array of avidin fusions to antibodies, dyes or other functional molecules, providing a skilled worker with multiple methods of indirectly visualizing nanorods. As will also be appreciated by the skilled person, nanorods displaying a detector molecule as described herein can bind an analyte and be visualized either indirectly via a phage-specific antibody or directly, such as by chemically attached fluorescent molecules (FIG. 20).

In some embodiments, the nanorods described herein may labelled with two or more different chemically attached detector molecules, e.g., different fluorescent molecules, allowing such multiply labelled nanorods to be used in methods of differential labelling, such as, but not limited to, multiplex detection.

The skilled worker will appreciate that all of the known modifications applied in the Ff-based phage display and material science applications can also be applied for functionalization of nanorods as described herein. In one non-limiting example, the insertion of 4 Gly residues at the N-terminus of mature pVIII that we constructed (FIG. 33, SEQ ID NO 27, SEQ ID NO 28), results in a minor drop in the nanorod production. In contrast, insertion of Ala followed by Gly residue between Ala1 and Gly2 and deletion of Pro6 of the wild-type mature pVIII at the N-terminus of the mature pVIII that we constructed (FIG. 32, SEQ ID NO 17, SEQ ID NO 18) results in the interference of nanorod production. To overcome this latter problem, we “evolved” the gVIII sequence to increase efficiency of this functionalized pVIII variant. This was achieved by transferring the coding sequence into the backbone of an Ff phage (VCSM13). The resulting modified phage gave very small plaques and low titres, however three rounds of phage growth where the host cells were infected at a low multiplicity of infection (1 phage to 1000 E. coli cells) resulted in the appearance of “large-plaque” mutants. Sequencing of gVIII from the evolved phage identified two gVIII variants containing each a different compensatory mutation (D5A and L27S as described herein above; FIGS. 32, 33; SEQ ID NO 19, SEQ ID NO 20, SEQ ID NO 21, SEQ ID NO 22). These two alleles were each transferred back into the inducible pPop-up plasmid backbone and showed to give rise to the BSF nanorods. As will be appreciated by the skilled person, the inventors believe that it is possible to evolve the coding sequences of various Ff phage proteins to allow other modifications that may interfere with the BSF nanorod assembly.

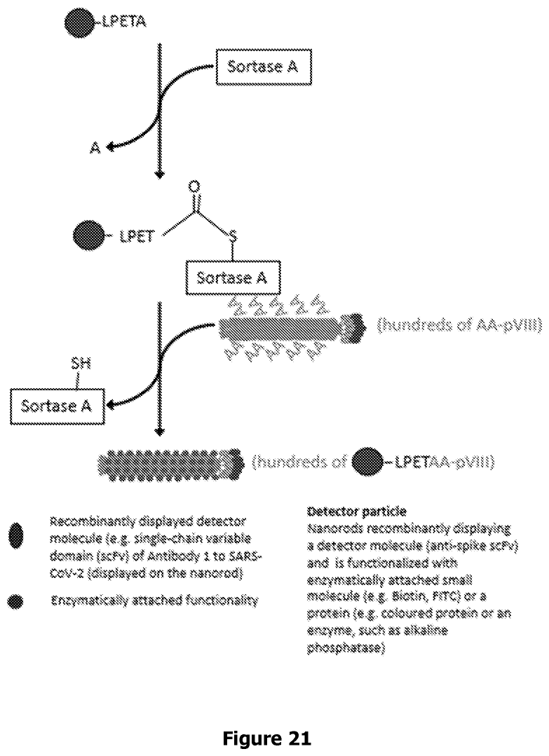

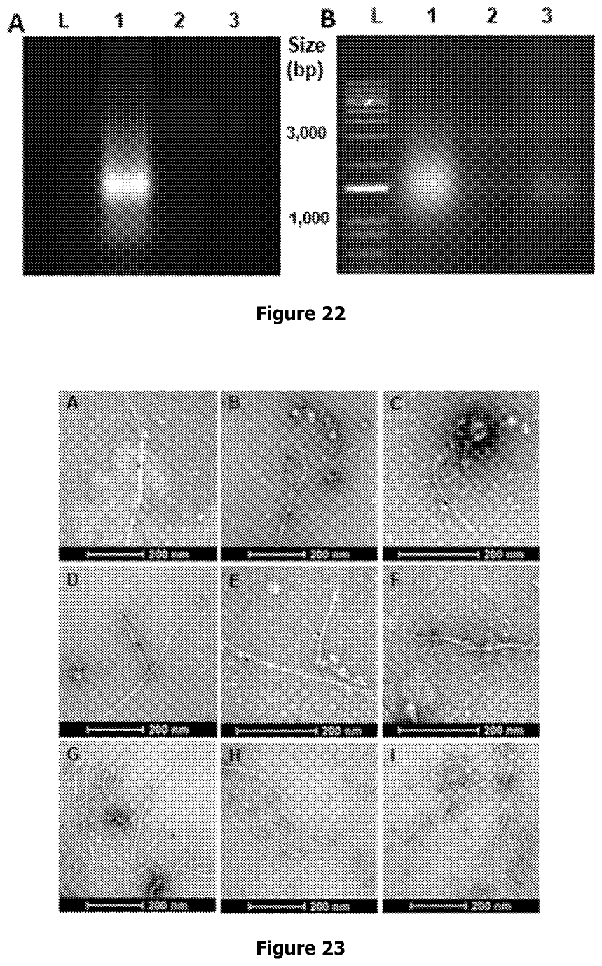

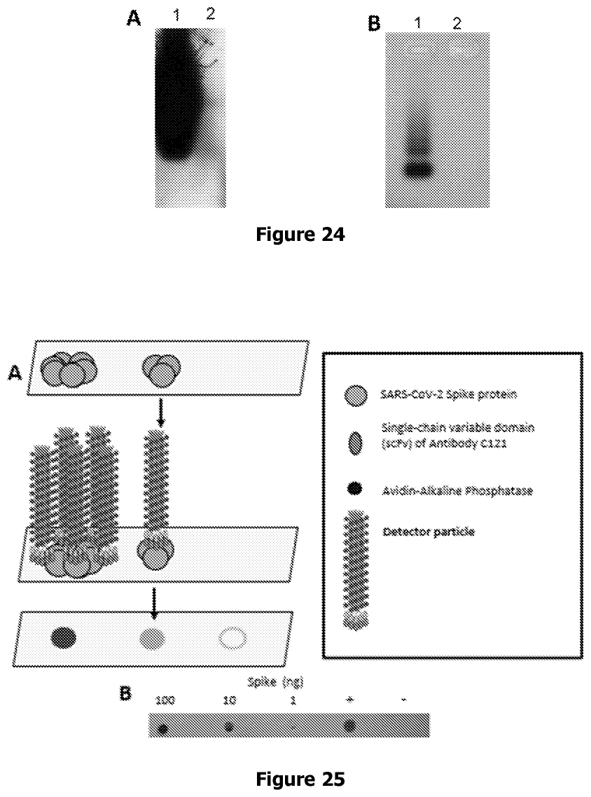

In one non-limiting example of enzymatic modification, BSF nanorods were produced that contain the evolved pVIII (SEQ NOs: 19, 20) displaying AlaAlaGlyGly motif on each pVIII copy along the nanorod. They were further enzymatically modified with LPETA-(Leu Pro Glu Thr Ala)-tagged fluorescent dye FITC or the small molecule biotin via enzymatic attachment using S. pyogenes Sortase (SrtA Sp; FIG. 21). Analysis by native virion electrophoresis showed high intensity fluorescence corresponding to the nanorod band after the LPTA-FITC enzymatic conjugation (FIG. 22). Analysis of enzymatically biotinylated nanorods by transmission electron microscopy using avidin-coated gold beads shows Sortase-dependent binding along the length of nanorods (FIG. 23). For immunodetection assays avidin-alkaline phosphatase may be attached to nanorods (FIG. 24A; 25-27). Enzymatic visualization of such avidin-alkaline phosphatase labeled nanorods was carried out by native agarose gel electrophoresis, blotted onto a membrane and detected using a chromogenic substrate (FIG. 24A).

In another non-limiting example LPETG-β-glucosidase (GUS) was enzymatically attached directly to nanorods displaying an N-terminal 5-Gly peptide. Attachment of GUS to the nanorods was analysed by agarose gel electrophoresis followed by in-gel assay using a chromogenic substrate (FIG. 24B).

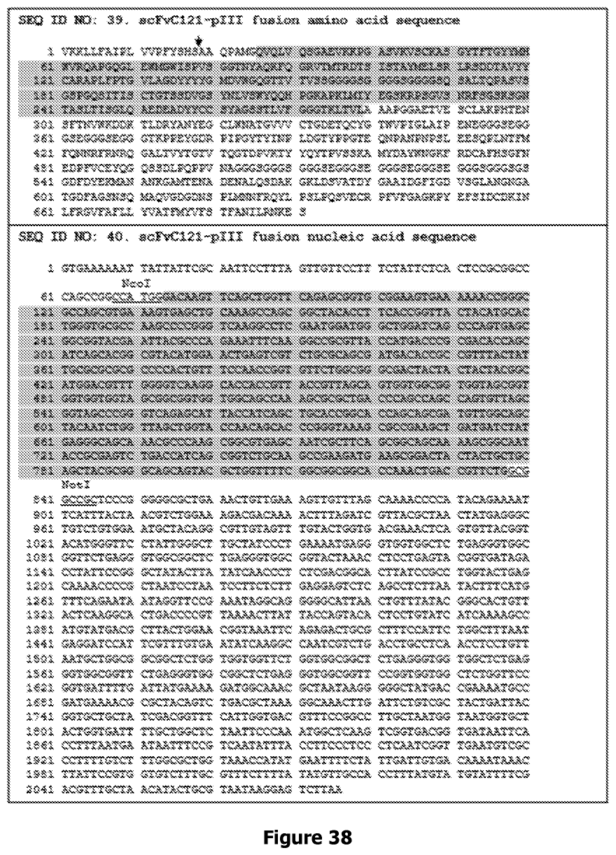

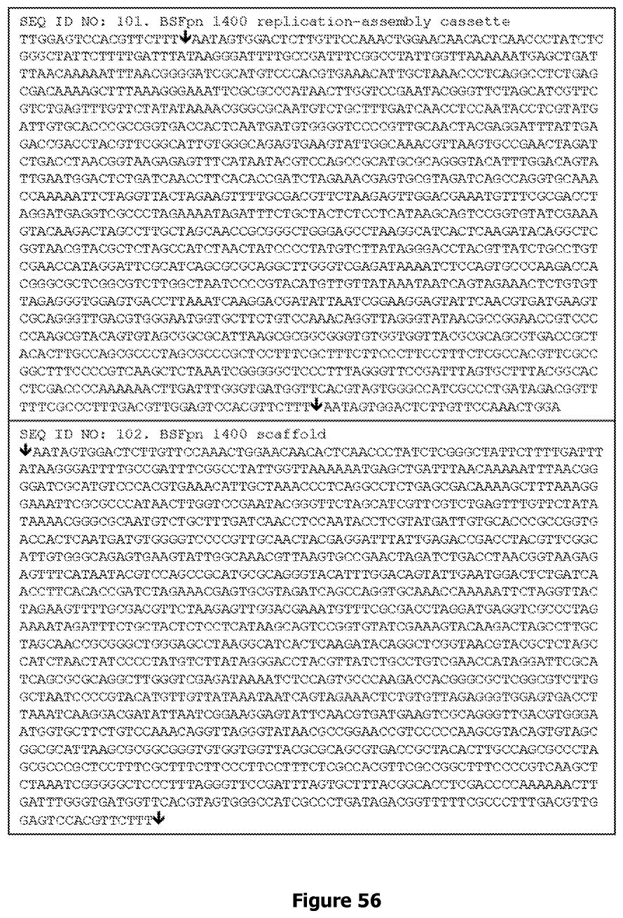

Labelled nanorods displaying analyte-specific molecules such as antibodies can also be used in immunoassays. In one non-limiting example, nanorods were produced that display pIII fusion proteins that specifically bind a SARS-CoV-2 spike-specific single-chain antibody (FIG. 38, SEQ NOs: 39, 40) or a SARS-CoV-2 nucleoprotein-specific camelid single-domain antibody VHH (FIG. 55, SEQ NOs: 99, 100). These pIII fusions were combined with pVIII displaying N-terminal Ala-Ala-Gly-Gly (AAGG) evolved to assemble nanorods efficiently (FIG. 32, SEQ NO: 18; FIG. 33, SEQ NO: 20). LPETA-biotin has been enzymatically attached to the nanorods using S. pyogenes Sortase A as described in the Methods section. Thus, modified nanorods were used in dot-blot, ELISA and lateral flow assays (FIGS. 25-27) as described in methods. Avidin-alkaline phosphatase or avidin-horseradish-peroxidase fusions were used as secondary or indirect detection reagents of the biotin-modified nanorods, to allow alkaline-phosphatase- or horseradish-peroxidase-mediated enzymatic visualization using chromogenic or chemiluminescent substrates of these two enzymes (FIGS. 25-27).
The copy number and position of displayed functionalities, be it heterologous proteins or “handles” for modifications, depends on the coat protein that is used as the platform. In one non-limiting example, use of pVIII as a platform for display allows a high-copy-number of displayed peptides along the shaft of the nanorod. The copy number of displayed peptides (or other functionalities) depends on the number of pVIII subunits per nanorod, which in turn depends on the length of the ssDNA scaffold. The copy number of functionalities (fluorescent dyes, small molecules, polymers and/or enzymes) depends on the length of the nanorod. For example, for a phage that is 1,000 nm in length (containing ˜3,000 copies of pVIII per virion) it is expected that ˜400 copies of a fluorophore or biotin per phage nanorod may be attached (˜1 fluorophore per 7 pVIII subunits), whether chemically or enzymatically. More than one different fluorescent dye can be mixed for the purpose of labelling to allow bar-coding or other more complex methods or detection.

Use of the minor coat proteins as platforms allows display of up to 5 copies per nanorod (for each pIII, pVII and pIX; reviewed in (Rakonjac et al., 2017). Furthermore, display on both pVII and pIX allows up to 10 copies per nanorod. Using different fusions or attached molecules to different minor Ff phage coat proteins, a number of different functionalities can be displayed on a single nanorod, such as with two functionalities being displayed at one end of the nanorod (the pVII-pIX end) and one functionality being displayed at the other (at the pIII end). Such modifications have been demonstrated in various methods of phage display using the full-length Ff phage.

As described herein, the toxicity of the major coat protein pVIII has been overcome by introduction of amber mutations. Major coat protein pVIII is toxic to E. coli when expressed in the absence of phage assembly. This toxicity leads to mutations that remove the gVIII promoter in the course of cloning, or in poor growth of transformed E. coli cells expressing pVIII, even when expression is controlled by an inducible promoter. To overcome this problem, gVIII suppressible (nonsense) mutants were used to construct helper plasmids. Construction was carried out in an E. coli host that does not contain a suppressor mutation, thereby preventing translation of most of the pVIII protein. Two different amber (TAG) mutants were used, one containing a G to T mutation that converted the GAG codon 25 encoding Glutamic acid at position two of the mature protein to TAG (SEQ NOs: 13-24, FIGS. 32-33), and one where TCT codon 4 for Serine within the signal sequence was replaced with TAG (SEQ NOs: 25-28, FIG. 33). A suppressor D mutation (supD) of the serine tRNA was used to suppress these two amber mutations, with an E. coli strain containing this mutation used for nanorod production (Table 1).

An additional advantage of the gVIII suppressed amber mutants described herein as compared to E. coli cell expressing wild-type gVIII is seen in a decrease of pVIII produced in the cells. This decrease is due to the lower translation efficiency of the suppressor tRNA in comparison to the cognate tRNA reading the sense codons, favoring assembly of short over long nanorods by decreasing the ratio of the shaft protein pVIII vs. the end-cap proteins pIII, pVI, pVII and pIX.

### C) Plasmid Origin of Replication and Selective Marker

### Plasmid Origin of Replication

In one embodiment, the plasmid origin of replication p15A is used for the pPop-up and the helper plasmids to allow replication in E. coli. The skilled worker will appreciate that, based on the disclosure of the present specification, other suitable plasmid origins of replication may be used in an NPS as described herein.

### Selective Marker

a marker for selection of transformed E. coli cells, either an antibiotic selective marker, for example [Kanamycin resistance marker aph (3′)-Ia (Kan^{R})] or auxotrophic marker, for example NadC, is required. If nadC is used as a selective marker, an E. coli host strain containing deletion of the nadC gene (ΔnadC) is used for construction of the Pop-upN and production of the nanorods using an NPS as described herein comprising a pPop-up529LacYMN plasmid. Minimal media containing casamino acids (and lacking NAD) is used for auxotrophic selection using NadC marker.
   THE two-plasmid system

The second type of the BSFnano production system described herein is composed of two plasmids. This two-plasmid system is also referred to herein as a dual plasmid system. As with the single plasmid Pop-up system described herein, these plasmids are transformed into a specific E. coli host strain: a nanorod replication-assembly plasmid containing a BSFnano replication-assembly cassette or variant thereof (pBSFnano series) and a helper plasmid expressing all necessary Ff phage proteins for replication of the nanorod (+) strand circular ssDNA from the BSFnano replication-assembly cassette, and assembly of short nanorods or variants thereof (pHP series). The helper plasmid also serves as a display vector allowing functionalization of nanorods. For example, the coding sequences in the helper plasmid can also be modified to allow expression of Ff phage proteins that are functionalization-ready.

The use of two plasmids in an NPS as described herein facilitates combination of different BSFnano replication-assembly cassettes with various different functionalities encoded by the helper plasmid variants without a need to make new recombinant DNA constructs.

### Helper Plasmid (pHP Series)

The helper plasmid contains the same components as the Pop-up plasmid described above, except that the BSFnano replication-assembly cassette is absent.
Ff phage genes are organized into two operons, gII(gX)-gV-gVII-gIX-gVIII and gIII-gVI-gI-gIV, encoding all functions required for replication of the BSFnano replication-assembly cassettes and assembly of the BSF nanorods. Ff genes are functionally categorized into those encoding replication functions, gII(gX), and encoding packaging-substrate-forming function, gV; (Block ii in the Helper plasmids, FIG. 8); coat proteins, gVII, gIX, gVIII, gIII and gVI (Block iii in the Helper plasmids, FIG. 8), and assembly, gI(gXI) and gIV (Block iv in the Helper plasmids, FIG. 8). In some embodiments, a nucleic acid construct comprising these operons further comprises multiple elements whose modification serves to increase the nanorod production or introduce functional groups in an orthogonal fashion, in specific positions and copy number in the nanorods, depending on the application for which the system is engineered.
   Promoter of the gII(gX)-gV-gVII-gIX-gVIII Operon
A surprising technical advance provided by the present disclosure is the ability to regulate production of replication functions, encoded by gII(gX) and substrate-packaging-function encoded by gV, in order to induce replication of the BSF replication-assembly cassette at sufficiently high cell density. The nucleic acid constructs of the NPS as described herein (e.g., pBSFp or pBSFpn plasmids) are introduced into E. coli by transformation at a transformation efficiency of about 10⁷ transformed cells per litre of culture (in contrast to a full culture that has a total of about 3×10¹² cells per litre). Therefore, the number of generations (cell divisions) between the transformation and harvesting of the nanorods is ˜20. Based on the quantitative monitoring and derived mathematical modelling (Smeal et al., 2017a, b), phage production falls to a baseline after 7 E. coli cell division times. Applied to the culture of transformed cells (starting number 10⁷ per L), 7 generations correspond to only 10⁹ cells per litre, an equivalent to only 1 mL of full overnight culture. Given that each cell produces a finite number of nanorods, this small number of cells producing the nanorods decreases the overall yield of the nanorods that can be produced from a litre of transformed cells.

What the inventors have surprisingly determined is that, if pII expression is induced only after the transformed cell culture reaches a higher cell density but while the culture is still in the exponential growth phase (˜10¹¹ cells per L; OD₆₀₀˜0.1), the production of nanorods will peak when the culture contains the highest cell numbers (10¹¹-6×10¹² per L). In this way, a drop in the nanorod production by the time that the culture reaches higher density is avoided. To achieve delayed pII production, gII(gX)-gV-gVII-gIX-gVIII operon expression was placed under an inducible promoter by replacement of the native (constitutive) Ff promoter P_{A} with an inducible promoter (lacUV5; Block i, SEQ ID NO: 90, FIG. 49). The new family of constructs were engineered that contained lacUV5 promoter instead of the P_{A} promoter in the pPop-up or helper plasmids, resulting, respectively, in the pPop-upLac and pHP1Lac series (FIGS. 7 and 8). Analyses of the nanorod production showed that synchronization of the optimal cell density with the efficient BSF nanorod production by inducible expression of pII increased the nanorod numbers by 10-fold, from 4.6×10¹⁴ to 4.8×10¹⁵ (Table 8; FIGS. 12 and 13, Example 6).

### The gII Allele

The phage-encoded pII used in this disclosure contains a mutation IR1-B (Enea and Zinder, 1982) that allows efficient replication from the core (+) on (domain A).

### Coat Proteins

Ff phage (and the BSF nanorods) are composed of five different coat proteins. Of those, pVIII (50 aa in length) is the major coat protein forming the shaft of the nanorod, present in large number of copies. The exact copy number of pVIII per nanorod depends on the length of the packaged ssDNA (1 pVIII subunit per 2.3 nt (Newman et al., 1977)). The remaining two pairs of “minor” coat proteins are present in small, fixed numbers (5 each per virion), forming two distinct ends of the virion (pIII and pVI at the proximal end and pVII and pIX on the distal end). The nanorod itself has a fivefold axial symmetry (Newman et al., 1977).

As shown in the art of phage display technology, Ff coat proteins each represent a platform for display of functionalities of interest, guided by specific applications (O'Neil and Hoess, 1995; Petrenko, 2008; Rakonjac et al., 2011). Protein fusions are constructed between the coat proteins and heterologous protein sequences, resulting in display of heterologous sequence on the surface of the virions. Alternatively, specific mutations or additional codons are introduced into the coding sequences of the coat proteins to serve as handles for site-specific modification (by “tag and modify” strategy; (Chalker et al., 2011)).
Insertion of heterologous peptide sequences or modification of the coat proteins has to be done in such a way that they do not interfere with the nanorod assembly. Each coat protein has a specific optimal site of insertion and/or segments that can be modified. Heterologous sequences can be inserted between the signal sequence and the mature portion of pIII and pVIII, at the C-terminus of pIII or pVI, or at the N-terminus of pVII and pIX (Fuh and Sidhu, 2000; Gao et al., 1999; Haaparanta and Huse, 1995; Jespers et al., 1996). Some of these fusions do not prevent assembly into the virion if the wild-type copies are also present but cannot mediate phage assembly on their own. In the latter case co-expression of a wild-type copy with a mutated counterpart (fusion) in the same cell allows assembly of the nanorods. As described herein, an additional copy of the wild-type or fusion coat protein (i.e., the mutated counterpart) can be either inserted into the plasmid containing the rest of the phage genes, or expressed from a second plasmid in the same cell (Barbas III et al., 2001). Fusions that can be incorporated into the Ff phage but cannot drive assembly on their own include insertions at the C-termini of pIII and pVI (Fuh and Sidhu, 2000; Jespers et al., 1996) and inserts longer than 6 amino acid residues between the signal sequence of pVIII and its mature portion. The 6-residue insertion effect is sequence-specific, with some sequences tolerated better than others (Iannolo et al., 1995). In some embodiments contemplated herein, a second copy of pVIII (a pVIII fusion to long peptides or proteins) can be expressed from a BSF nano replication-assembly cassette within the pBSF plasmid as described herein (FIG. 6B;

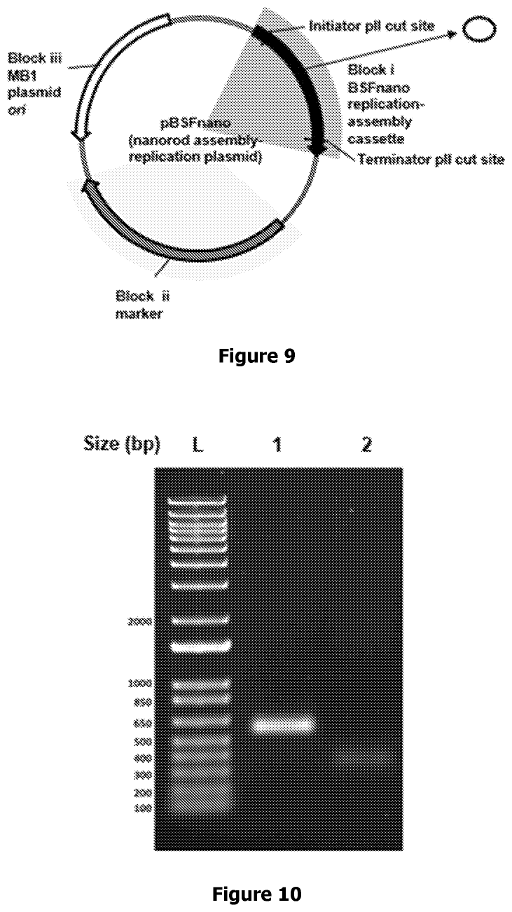

FIG. 9, Block i). In one non-limiting example, expression of a second copy of an Ff phage protein from a BSF replication-assembly cassette filler nucleic acid sequence is shown by expression of pVII and pIX from such a cassette as shown in FIG. 6B and FIG. 41 (SEQ NOs: 52-55).

Further contemplated herein, additional expression constructs, including plasmids can be used to supply secondary copies of pVIII coat proteins when the inserted heterologous sequences interfere with assembly of the nanorods in the absence of the wild-type counterpart. These additional plasmids have to have an origin of replication compatible to the both the helper plasmid (pHP series) and the nanorod replication plasmid (pBSFnano series) in the two-plasmid system, e.g., chloramphenicol resistance (cat; Cm^{R}) marker and ColD origin of replication.
Further modifications to the coat-protein-encoding genes to create functionalization handles, known as “tag and modify” strategy, are made to allow targeted chemical or enzymatic modification. For example, engineering pVIII containing extra 3 Glycines or ≥2 Alanines at the N-terminus of the mature coat protein pVIII or pIII or pVII and pIX (addition of heterologous signal sequence may be required for the two latter proteins) creates a motif that can be used for enzymatic attachment of protein or non-protein molecules conjugated to C-terminal LPXTA or LPXTG motifs, where the attachment of a molecule of interest is catalyzed by enzyme sortase A (SrtA) of Streptococcus pyogenes (SrtA Sp) or Staphylococcus aureus (SrtA Sa), respectively (Hess et al., 2012). Exchangeable blocks (FIG. 8, Block iii) have been generated for our NPS that produce nanorods with pVIII displaying, at the N-terminus, 4 Gly residues, or 2 Ala residues (FIGS. 30-32 and SEQ NOs: 19-23; 27-28) or 5 residues (FIG. 54, SEQ NOs: 97-98).

Reactive groups of amino acids, such as the amine groups of the N-terminal residues, lysines, cysteines, tyrosines, aspartic acids, and glutamic acids can be used for chemical modification (Bernard and Francis, 2014). Alternatively, other motifs that are subject to enzymatic or chemical covalent attachment to non-protein molecules, such as SNAP-tag, be directly or indirectly inserted into the nanorods, to allow attachment of a diverse array of molecules. Also described herein are exchangeable blocks that display unpaired Cys residues on pIII, to allow modifications by maleimide-conjugated proteins and small molecules or other chemistries targeting —SH groups (FIG. 8, Block iii; FIG. 36, SEQ NOs: 33-34).

Furthermore, insertion of ATG codons into the coding sequences corresponding to exposed residues of pVIII allows in vivo labelling with unnatural amino acid azidohomoalanine (structurally similar to ATG-encoded residue Met) during translation. Azide groups on the surface of the nanorod provide reactive groups for attachment of molecules using “click” chemistry (Petrie, 2015). To enable this, also described herein are exchangeable blocks (FIG. 8; Block iii) containing a pVIII variant comprising exposed Met residues (Ala9 mutated to Met) and buried Met28 residue mutated to Leu (FIG. 33; SEQ ID NOs: 23, 24). This pVIII mutant allows for in vivo incorporation of unnatural amino acid azidohomoalanine (Aha) into an surface-exposed position on pVIII during translation (Ala9 to Met) without disturbance of the virion assembly and structure that would have been caused by insertion of Aha at position 28 that was prevented by mutation of Met28 into Leu (Petrie, 2015). Aha contains azide group in its side-chain, allowing attachment into the virion of small molecules using click chemistry which targets azide groups.

In one non-limiting example, described herein is the fluorescent labelling of the BSF nanorods with amine-reactive fluorescent dye DyLight 550 (FIG. 20, Example 11). These nanorods display a binding molecule (fibronectin-binding domain of S. pyogenes protein SOF22 (Rakonjac et al., 1995) as fusion to pIII; SEQ NOs: 37, 38, FIG. 37) and have been used for lateral flow assay for detection of the analyte (fibronectin; FIG. 20C, Example 11). As will be appreciated by the skilled worker, using this approach any amine-reactive fluorescent or any other dye or other small molecule or biological or chemical polymer that is designed to be amine-reactive is expected to be suitable for attachment to nanorods. Each pVIII subunit has three acidic amino acid residues containing each a side-chain carboxyl group (Glu2, Asp4 and Asp5) exposed on the surface of the nanorod. Accordingly, the carboxyl-reactive molecules can also be chemically conjugated to the nanorods. Other reactive groups, such as the Tyr residue aromatic hydroxyl group can also been used to attach suitable reactive groups as is known in the art (Bernard and Francis, 2014). As will be appreciated, by the skilled worker using this approach any carboxyl-reactive fluorescent or any other dye or other small molecule or biological or chemical polymer that is designed to be amine-reactive is expected to be suitable for attachment to nanorods.

The molecules attached to the nanorods as described herein may be organic molecules of any kind, including but not limited to biotin, which serves to bind commercially available or in-house made fusions of biotin-binding proteins such as avidin. In this fashion, the nanorods described herein may be modified to display a broad array of avidin fusions to antibodies, dyes or other functional molecules, providing a skilled worker with multiple methods of indirectly visualizing nanorods. As will also be appreciated by the skilled person, nanorods displaying a detector molecule as described herein can bind an analyte and be visualized either indirectly via a phage-specific antibody or directly, such as by a chemically attached fluorescent molecules (FIG. 20). In some embodiments, the nanorods described herein may labelled with two or more different chemically attached detector molecules, e.g., different fluorescent molecules, allowing such multiply labelled nanorods to be used in methods of multiplex detection.

The skilled worker will appreciate that all of the known modifications applied in the Ff-based phage display and material science applications can also be applied for functionalization of nanorods as described herein. In one non-limiting example, the insertion of 4 Gly residues at the N-terminus of mature pVIII that we constructed (FIG. 33, SEQ ID NO 27, SEQ ID NO 28), results in a minor drop in the nanorod production. In contrast, insertion of Ala followed by Gly residue between Ala1 and Gly2 and deletion of Pro6 of the wild-type mature pVIII at the N-terminus of the mature pVIII that we constructed (FIG. 32, SEQ ID NO 17, SEQ ID NO 18) results in the interference of nanorod production. To overcome this latter problem, we “evolved” the gVIII sequence to increase efficiency of this functionalized pVIII variant. This was achieved by transferring the coding sequence into the backbone of an Ff phage (VCSM13). The resulting modified phage gave very small plaques and low titres, however three rounds of phage growth where the host cells were infected at a low multiplicity of infection (1 phage to 1000 E. coli cells) resulted in the appearance of “large-plaque” mutants. Sequencing of gVIII from the evolved phage identified two gVIII variants containing each different compensatory mutations (D5A and L27S as described herein above; FIGS. 32, 33; SEQ ID NO 19, SEQ ID NO 20, SEQ ID NO 21, SEQ ID NO 22). These two alleles were each transferred back into the inducible pPop-up plasmid backbone and showed to give rise to the BSF nanorods. As will be appreciated by the skilled person, the inventors believe that it is possible to evolve the coding sequences of various Ff phage proteins to allow other modifications that may interfere with the BSF nanorod assembly.

In one non-limiting example of enzymatic modification, BSF nanorods were produced that contain the evolved pVIII (SEQ NOs: 19, 20) displaying AlaAlaGlyGly motif on each pVIII copy along the nanorod. They were further enzymatically modified with LPETA-(Leu Pro Glu Thr Ala)-tagged fluorescent dye FITC or the small molecule biotin via enzymatic attachment using S. pyogenes Sortase (SrtA Sp; FIG. 21). Analysis by native virion electrophoresis showed high intensity fluorescence corresponding to the nanorod band after the LPTA-FITC enzymatic conjugation (FIG. 22). Analysis of enzymatically biotinylated nanorods by transmission electron microscopy using avidin-coated gold beads shows Sortase-dependent binding along the length of nanorods (FIG. 23). For immunodetection assays avidin-alkaline phosphatase may be attached to nanorods (FIG. 24A; 25-27). Enzymatic visualization of such avidin-alkaline phosphatase labeled nanorods was carried out by native agarose gel electrophoresis, blotted onto a membrane and detected using a chromogenic substrate (FIG. 24A).

In another non-limiting example LPETG-β-glucosidase (GUS) was enzymatically attached directly to the nanorods displaying an N-terminal 5-Gly peptide. Attachment of GUS to the nanorods was analysed by agarose gel electrophoresis followed by in-gel assay using a chromogenic substrate (FIG. 24B).

Labelled nanorods displaying analyte-specific molecules such as antibodies can also be used in immunoassays. In one non-limiting example, nanorods were produced that display pIII fusion proteins that specifically bind a SARS-CoV-2 spike-specific single-chain antibody (FIG. 38, SEQ NOs: 39, 40) or a SARS-CoV-2 nucleoprotein-specific camelid single-domain antibody VHH (FIG. 55, SEQ NOs: 99, 100). These pIII fusions were combined with pVIII displaying N-terminal Ala-Ala-Gly-Gly (AAGG) evolved to assemble nanorods efficiently (FIG. 32, SEQ NO: 18; FIG. 33, SEQ NO: 20). LPETA-biotin has been enzymatically attached to the nanorods using S. pyogenes Sortase A as described in the Methods section. Thus, modified nanorods were used in dot-blot, ELISA and lateral flow assays (FIGS. 25-27) as described in methods. Avidin-alkaline phosphatase or avidin-horseradish-peroxidase fusions were used as secondary or indirect detection reagents of the biotin-modified nanorods, to allow alkaline-phosphatase-or horseradish-peroxidase-mediated enzymatic visualization using chromogenic or chemiluminescent substrates of these two enzymes (FIGS. 25-27).
The copy number and position of displayed functionalities, be it heterologous proteins or “handles” for modifications, depends on the coat protein that is used as the platform. In one non-limiting example, use of pVIII as a platform for display allows a high-copy-number of displayed peptides along the shaft of the nanorod. The copy number of displayed peptides (or other functionalities) depends on the number of pVIII subunits per nanorod, which in turn depends on the length of the ssDNA scaffold. The copy number of functionalities (fluorescent dyes, small molecules or enzymes) depends on the length of the nanorod. For example, for a phage that is 1,000 nm in length it is expected that ˜400 copies of a fluorophore or biotin per phage particle may be attached, whether chemically or enzymatically (Hess et al., 2012; Li et al., 2010). More than one different fluorescent dye can be mixed for the purpose of labelling to allow bar-coding or other more complex methods or detection.

Use of the minor coat proteins as platforms allows display of up to 5 copies per nanorod (for each pIII, pVII and pIX; reviewed in (Rakonjac et al., 2017). Furthermore, display on both pVII and pIX allows up to 10 copies per nanorod. Using different fusions or attached molecules to different minor Ff phage coat proteins, a number of different functionalities can be displayed on a single nanorod, such as with two functionalities being displayed at one end of the nanorod (the pVII-pIX end) and one functionality being displayed at the other (the pIII end). Such modifications have been demonstrated for the full-length Ff phage as known in the phage display art.

—Overcoming Toxicity of pVIII and Amber Mutations

Importantly, the major coat protein pVIII is toxic when expressed in E. coli in the absence of phage assembly. This toxicity leads to mutations that remove the gVIII promoter in the course of cloning, or in poor growth of transformed E. coli cells expressing pVIII, even when expression is controlled by an inducible promoter. To overcome this problem, gVIII suppressible (nonsense) mutants were used to construct helper plasmids. Construction was carried out in an E. coli host that does not contain a suppressor mutation, thereby preventing translation of most of the pVIII protein. Two different amber (TAG) mutants were used, one containing a G to T mutation that converted the GAG codon 25 encoding glutamic acid at position two of the mature protein to TAG (SEQ NOs: 13-24, FIGS. 32-33), and one where TCT codon 4 for serine within the signal sequence was replaced with TAG (SEQ NOs: 25-28, FIG. 33). A suppressor D mutation (supD) of the serine tRNA was used to suppress these two amber mutations, with an E. coli a strain containing this mutation used nanorod production (Table 1).

An additional advantage of the gVIII suppressed amber mutants described herein as compared to E. coli cell expressing wild-type gVIII is seen in a decrease of pVIII produced in the cells. This decrease is due to the lower translation efficiency of the suppressor tRNA in comparison to the cognate tRNA reading the sense codons, favoring assembly of short over long nanorods by decreasing the ratio of the shaft protein pVIII vs. end-cap proteins pIII, pVI, pVII and pIX.

BSFnano Replication-Assembly Plasmid (pBsfnano Series)

Components of the BSFnano replication-assembly plasmid used in the two-plasmid system are a BSFnano replication-assembly cassette, a plasmid origin of replication and a selective marker.

BSFnano replication-assembly cassette variants are equivalent to those described in the Pop-up plasmid (e.g., BSFp and BSFpn). “Filler” nucleic acid sequence of a predetermined length can be inserted between (+) ori1 and (+) ori2 to construct nanorods of specific lengths of interest as described herein (FIGS. 5, 6, 9; 40, 42, 44, 46, 48, 57; SEQ ID NO: 46, SEQ ID NO: 47, SEQ ID NO: 49, SEQ ID NO: 56; SEQ ID NO: 60, SEQ ID NO: 66, SEQ ID NO: 67, SEQ ID NO: 75; SEQ ID NO: 82, SEQ ID NO: 85; SEQ ID NO: 86, SEQ ID NO: 104). In some embodiments no filler nucleic acid sequences are inserted.

In one non-limiting example, a BSFnano replication-assembly cassette in the pBSF plasmid series is a combination of the following units:
Initiator (+) ori1, a functional positive-strand origin of replication (+) on that allows binding of the replication protein pII (a DNA-strand-transferase) and cutting of the (+) strand to form a primer (FIGS. 4-6; 39-48; 56-57; SEQ ID NO: 45, SEQ ID NO: 65, SEQ ID NO: 81; SEQ ID NO: 103).
Packaging signal (PS) that is required for targeting of the (+) strand circular ssDNA replicated from a BSFnano replication-assembly cassette to the trans-envelope assembly machinery for assembly of the nanorods (FIGS. 4-6; 40, 42, 44, 46, 48; 57; SEQ ID NO: 48, SEQ ID NO: 68, SEQ ID NO: 76, SEQ ID NO: 83, SEQ ID NO: 84).
The (−) on allows replication of the negative strand using the short BSFnano (+) strand ssDNA as a template, to increase the copy number of the (+) stand circular ssDNAs produced from a BSFnano replication-assembly cassette (FIGS. 4-6; 40, 42, 44, 46, 57; SEQ ID NO: 50).
Terminator ((+) ori2) is a truncated (+) on mutant (Δ29) that allows cutting of the template (+) strand whose replication started at (+) ori1, and ligation of the two ends of the (+) strand to produce a (+) strand circular ssDNA that serves as a backbone for nanorod assembly as described herein (FIGS. 4-6; 40, 42, 44, 46, 48, 57; SEQ ID NO: 51, SEQ ID NO: 69, SEQ ID NO: 87).
Properties and variants of the BSFnano replication-assembly cassettes:

The initiator, (+) ori1, can be either the minimal or core domain of (+) on (A or I) only (FIGS. 4-6, 46, 48; SEQ ID NO: 74, SEQ ID NO: 81), or the complete (+) on (both A and B domains; (FIGS. 4-6, 40, 42, 44, 57; Seq ID NO: 45, SEQ ID NO: 65, SEQ ID NO: 103), with the latter being more efficient at initiation than the former, due to the presence of the complete pII binding sequence.

The lengths of produced nanorods are determined by the sizes of scaffold nucleic acid sequences comprised in the BSFnano replication-assembly cassettes as described herein. The scaffold nucleic acid sequences are positioned between a first pII nick site in (+) ori1 and a second pII nick site in (+) ori2 (GTTCTT [symbol] AATA)(SEQ ID NO: 88) in the BSFnano replication-assembly cassettes (FIGS. 4-6; 39-48; 56-57; SEQ ID NO: 41, SEQ ID NO: 43, SEQ ID NO: 52, SEQ ID NO: 54, SEQ ID NO: 61, SEQ ID NO: 63. SEQ ID NO: 70, SEQ ID NO: 72, SEQ ID NO: 77, SEQ ID NO: 79, SEQ ID NO: 101)

### BSFp Replication-Assembly Cassette

For example, a BSFnano replication-assembly cassette composed of the initiator (+) ori1 comprising only (+) on core (or domain A), packaging signal and terminator (+) ori2 corresponding to (+) on Δ29, we named here BSFp, results in production of the circular (+) ssDNA of 152 or 221 nt and assembly, respectively, nanorods of 40 or 50 nm in length (FIGS. 1, 4-6, 47-48, SEQ ID NO: 79, SEQ ID NO: 80, SEQ ID NO: 77, SEQ ID NO: 78). The 40 nm nanorods are the shortest Ff-derived nanorods produced to date.

### BSFpn Replication-Assembly Cassettes

In another example, replication-assembly cassette we named BSFpn contains a combination of initiator ((+) ori1) corresponding to the complete (+) on (domains AB), a packaging signal, a (−) on and (+) ori2 (a terminator, (+) on Δ29). In the presence of pII this replication-assembly cassette results in replication of the (+) strand ssDNA of 395, 529, 707, 711, 728, 748 nt, and nanorods that are 70, 80, 100 or 110 nm in length (FIGS. 1, 4-6, 39-44, SEQ ID NO: 41, SEQ ID NO: 43, SEQ ID NO: 52, SEQ ID NO: 54, SEQ ID NO: 61, SEQ ID NO: 63). Longer BSFnano nanorods can be produced if DNA sequence be inserted between the (+) ori1 and the PS.

Another variation of the BSFpn replication-assembly cassette is possible where the (+) ori1 would contain only the core (+) on (domain A) as the initiator and would still include the (−) on (FIGS. 45, 46; SEQ ID NO: 70, SEQ ID NO: 72; SEQ ID NO: 74). The ssDNA produced from such a BSFpn cassette would be 313 or 289 nt, resulting in nanorod of a calculated length of, respectively, 57 or 54 nm (˜50-60 nm).

### Scalability of BSF Nanorods

In both the single and dual plasmid NPSs described herein, a scaffold nucleic acid sequence is comprised in the BSFnano replication-assembly cassette between the pII cut sites ((GTTCTTT [symbol] AATA) (SEQ ID NO:88, FIG. 49) in (+) ori1 (initiator) and in (+) ori2 (terminator; FIGS. 1,4-6, 39-48). A person of skill in the art recognizes that a scaffold nucleic acid sequence of the appropriate size to produce a nanorod and/or plurality of nanorods of a desired size can be readily selected for use in an NPS as described herein based on the disclosure of the present specification and as known in the art.

As noted previously, the length of the (+) strand circular ssDNA backbone (scaffold) produced by rolling circle replication of the BSFnano replication-assembly cassettes is determined by the number of nucleotides between the pII cut sites in the (+) ori1 (initiator) and (+) ori2 (terminator). The length of the scaffold nucleic acid sequence can be decreased in order to reduce the size of the nanorods by removing the (−) on (as done in the BSFp replication-assembly cassettes, completely removing the filler sequences and by reducing the size of the (+)ori1 and (+)ori2 in BSFpn replication-assembly cassette (Table 9; e.g. FIGS. 43 and 44, SEQ ID NO: 63, SEQ ID NO: 64, SEQ ID NO: 66 vs SEQ ID NO: 67; FIGS. 45 and 46, SEQ ID NO: 70, SEQ ID NO: SEQ ID NO: 71, SEQ ID NO:72, SEQ ID NO: 73; SEQ ID NO: 74 vs SEQ ID NO: 65; FIGS. 47 and 48, SEQ ID NO: 77, SEQ ID NO: 78, SEQ ID NO: 79, SEQ ID NO: 80, SEQ ID NO: 74). Conversely, the length of the nanorods can be extended by inserting “filler” nucleic acid sequences between the initiator ((+) ori1) and the PS, and between the PS and the (−) on in BSFpn or PS and the (+) ori2 in BSFp replication-assembly cassette (FIG. 5B and C; FIG. 6; FIGS. 56-57, SEQ ID 104). Consequently, the length of the nanorod is extended to a desired length by designing filler nucleic acid sequences of a suitable length. Based on structural analyses of the Ff phage shaft, it can be calculated precisely that addition of every nucleotide to the ssDNA genome increases the length of the nanorod by 0.133 nm (Newman et al., 1977).

Protein-Encoding Genes within the Replication-Assembly Cassette

Also contemplated herein, the filler nucleic acid sequences can encode a second copy of gVIII that will be used as a platform for expression of pVIII fusion to long peptides or proteins (FIG. 6B; FIG. 9, Block i). Alternatively, a second copy of gVIII can be encoded on a compatible plasmid, supplying Ff phage protein to be incorporated into the nanorods produced as it is usually done in phage display art. An example of expression from a BSF replication-assembly cassette was given by expression of pVII and pIX from the said cassette (FIG. 6B; FIG. 41, SEQ NOs: 52-55). In addition to Ff proteins expressed in E. coli, filler sequences could be used to accommodate a eukaryotic gene expression cassette.

### Plasmid Origin of Replication and Selective Marker

Any theta-replicating plasmid origin of replication can be used in the nanorod replication-assembly plasmid, as long as it is compatible with the plasmid origin of the helper plasmid, e.g., MB1 or ColEI in the pBSFnano replication-assembly plasmid and pA15 in the pHP helper plasmid (FIG. 9, block iii).

The selective marker for maintenance of the pBSFnano replication-assembly plasmid once transformed into E. coli (FIG. 9, block ii) can be an antibiotic selective marker, as long as the marker is different from the marker in the helper plasmid (e.g., bla gene encoding for ampicillin resistance marker P lactamase). Alternatively, an auxotrophic marker (e.g., nadC) can be used to avoid the production of antibiotic-resistance-containing nanorods that have been detected at a low frequency of 1/10⁶. These rare antibiotic-resistance-encoding nanorods that contain an entire nanorod replication-assembly plasmid as described herein is a result of aborted termination at (+) ori2 or recombination between (+) ori1 and (+) ori2, resulting in the presence of a single positive origin of replication. In a specific and preferred embodiment of the invention provided herein, the selective marker on the nanorod replication plasmid is an auxotrophic marker as described herein.

### Additional Plasmids

Further contemplated herein, additional plasmids can be used to supply secondary copies of coat proteins when the inserted heterologous sequences interfere with assembly of the nanorods in the absence of the wild-type counterpart. These additional plasmids have to have an origin of replication compatible to the both the helper plasmid (pHP series) and the nanorod replication plasmid (pBSFnano series) in the two-plasmid system, e.g., chloramphenicol resistance marker (cat; Cm^{R}) and ColD origin of replication.

In a first aspect, the present invention relates to a nanorod production system (NPS) comprising a single nucleic acid expression construct, the construct comprising
a BSFnano replication-assembly cassette
at least one auxotrophic marker,
at least one inducible promoter operably linked to a nucleic acid sequence encoding at least one Ff phage protein, and
at least one plasmid origin of replication not located in the BSFnano replication-assembly cassette.

In one embodiment the nucleic acid expression construct is, or is comprised in, a vector. In one embodiment, the nucleic acid expression construct is a vector.

In one embodiment the vector is selected from the group consisting of plasmids, bacterial artificial chromosomes (BACs), P1-derived artificial chromosomes (PACs), yeast artificial chromosomes (YACs), bacteriophage, phagemids, and cosmids. In one embodiment the vector is a plasmid.

In one embodiment the nucleic acid expression construct is or is comprised in, a plasmid. In one embodiment the nucleic acid expression construct is a plasmid.

In one embodiment the BSFnano replication-assembly cassette comprises at least two (+) ori's. In one embodiment the BSFnano replication-assembly cassette comprises at least one (−) ori. In one embodiment the BSFnano replication-assembly cassette comprises two (+) ori's and one (−) ori.

In one embodiment one (+) ori is a DNA replication initiator. The (+) ori that is a DNA replication initiator is termed (+) ori1 herein. In one embodiment one (+) ori is a DNA replication terminator. The (+) ori that is a DNA replication terminator is termed (+) ori2 herein.

In one embodiment one (+) ori is a DNA replication initiator (“(+)ori1”) and one (+) ori is a DNA replication terminator (“(+) ori2”). In one embodiment the BSFnano replication-assembly cassette comprises (+) ori1, (+) ori2, and one (−) ori.

In one embodiment the BSFnano replication-assembly cassette comprises a packaging signal (PS). In one embodiment the PS is between (+) ori1 and (+) ori2. In one embodiment the PS is between (+) ori1 and the (−) ori. In one embodiment (+) ori1 and (+) ori2 comprise pII cut sites.

In one embodiment the BSFnano replication-assembly cassette comprises a scaffold nucleic acid sequence.

In one embodiment the BSFnano replication-assembly cassette comprises a scaffold nucleic acid sequence plus flanking sequences required for the (+) strand replication.

In one embodiment the flanking sequences are located upstream of the pII cut site in ori (1) and downstream of the pII cut in ori (2). In one embodiment the flanking nucleic acid sequences bind pII and/or bind modified pII.

In one embodiment the scaffold nucleic acid sequence is positioned between the (+) ori1 and (+) ori2. In one embodiment the scaffold nucleic acid sequence is positioned between pII cut sites in (+) ori1 and (+) ori2.

In one embodiment the scaffold nucleic acid sequence is positioned between sequences (GTTCTTAATA; SEQ ID NO: 88, FIG. 49) in (+) ori1 (initiator) and in (+) ori2 (terminator).

In one embodiment the scaffold nucleic acid sequence is positioned in the BSFnano replication-assembly cassette as shown in FIGS. 5 and 6.

In one embodiment replication of the scaffold nucleic acid sequence in the presence of pII produces a circular ssDNA.

In one embodiment the scaffold nucleic acid sequence does not comprise any filler nucleic acid sequence. In one embodiment the scaffold nucleic acid sequence comprises at least one filler nucleic acid sequence. In one embodiment the scaffold nucleic acid sequence comprises two filler nucleic acid sequences.

In one embodiment the scaffold nucleic acid sequence comprises a filler nucleic acid sequence comprising an additional nucleic acid sequence positioned to extend the length of a (+) strand ssDNA produced by replication of the scaffold nucleic acid sequence.

In one embodiment a filler nucleic acid sequence is positioned as shown in FIGS. 5 and 6, “Filler”.

In one embodiment the scaffold nucleic acid sequence comprises a filler nucleic acid sequence positioned between (+) ori1 and the PS. In one embodiment the scaffold nucleic acid sequence comprises a filler nucleic acid sequence positioned between the PS and (+) ori2. In one embodiment the scaffold nucleic acid sequence comprises a filler nucleic acid sequence between (+) ori1 and the PS and between the PS and (+) ori2.

In one embodiment the scaffold nucleic acid sequence comprises a filler nucleic acid sequence comprising 0 to about 6000 nt, 0 to about 5000, 0 to about 4000, 0 to about 3000, 0 to about 2000, 0 to about 1000, 0 to about 750, 0 to about 500, 0 to about 400, 0 to about 300, 0 to about 200, 0 to about 100, 0 to about 50, 0 to about 40, 0 to about 30, 0 to about 25, 0 to about 20, 0 to about 15, 0 to about 10, 0 to about 5, or 0 nt. In one embodiment the scaffold nucleic acid sequence comprises a filler nucleic acid sequence comprising 0 to 6000 nt, 0 to 5000, 0 to 4000, 0 to 3000, 0 to 2000, 0 to 1000, 0 to 750, 0 to 500, 0 to 400, 0 to 300, 0 to 200, 0 to 100, 0 to 50, 0 to 40, 0 to 30, 0 to 25, 0 to 20, 0 to 15, 0 to 10, 0 to 5, or 0 nt.

In one embodiment the scaffold nucleic acid sequence comprises a filler nucleic acid sequence comprising 0, 5, 23, 24, 31, 145, 315, 319, 336, 356, 700, 1400 or 2100 nt. In one embodiment the filler nucleic acid sequence comprises, consists essentially of, or consists of a filler nucleic acid sequence as identified in Table 9. The skilled worker appreciates that the size of the filler may be varied to accommodate the production of nanorods of various sizes depending on the lengths (i.e., number of nucleotides) of the other functional sequence elements of the scaffold nucleic acid sequence including (+) on 1, (−) on and (+) on 2.

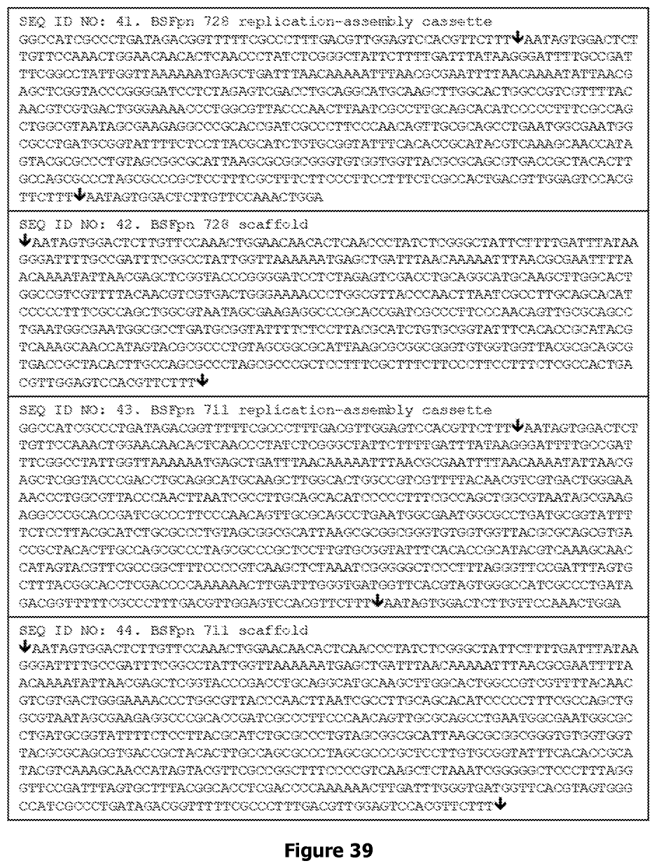

In one embodiment the single nucleic acid construct comprises SEQ ID NO: 41, SEQ ID NO: 43, SEQ ID NO: 52, SEQ ID NO: 54, SEQ ID NO: 61, SEQ ID NO: 63, SEQ ID NO: 70, SEQ ID NO: 72, SEQ ID NO: 77, SEQ ID NO: 79 (FIGS. 39-48) or SEQ NO: 101 (FIG. 56).

In one embodiment the scaffold nucleic acid sequence comprises a filler nucleic acid sequence that codes for at least one, preferably at least two Ff phage coat and/or Ff phage modified coat proteins. In one embodiment the at least one coat and/or modified coat protein is pVII or pIX. In one embodiment the at least two coat and/or modified coat proteins are pVII and pIX.

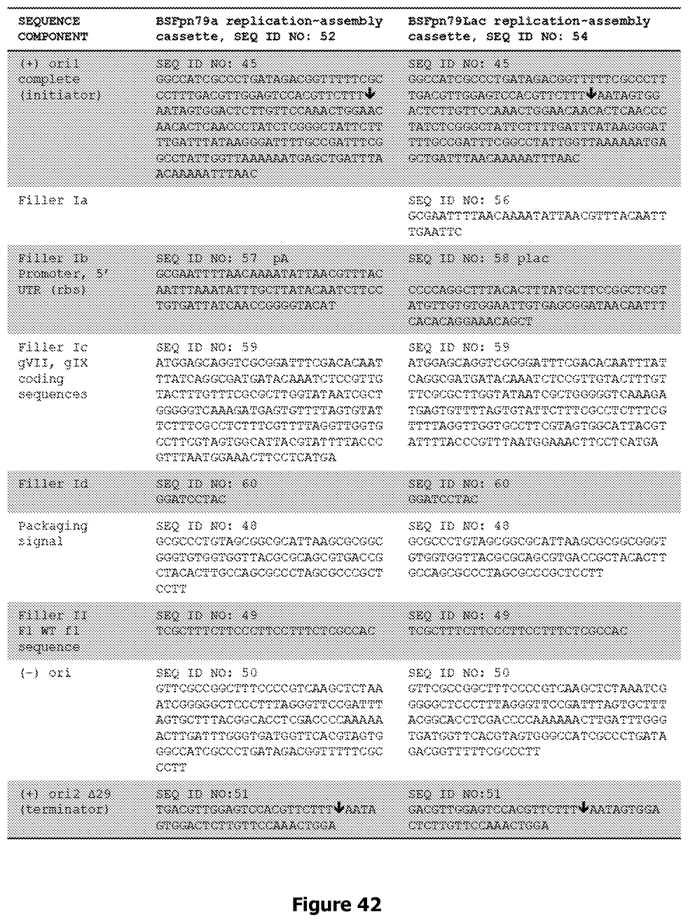

In one embodiment the at least two coat and/or modified coat proteins are operably linked to a promoter. In one embodiment the promoter is a constitutive or inducible promoter. In one embodiment the promoter is a constitutive promoter. In one embodiment the promoter is an inducible promoter. In one embodiment the constitutive promoter is a phage promoter, preferably pA. In one embodiment the inducible promoter is selected from the group consisting of lac, tac, araC, or trp promoters, preferably a lac promoter. In one embodiment the lac promoter is a lac promoter regulated by the inducer (IPTG). In one embodiment the lac promoter mutant is susceptible to repression by glucose (catabolite repression). In one embodiment the lac promoter is the lac promoter (FIG. 42, SEQ ID NO: 58).

In one embodiment the lac promoter is a lac promoter mutant regulated solely by the inducer (IPTG). In one embodiment the lac promoter mutant is not susceptible to repression by glucose (catabolite repression).

In one embodiment the lac promoter is the lacUV5 promoter (FIG. 49, SEQ ID NO: 90).

In one embodiment, enzymatic replication of the scaffold nucleic acid sequence produces a plurality of replicated (+) strand circular ssDNA molecules. In one embodiment, enzymatic replication is rolling circle replication.

In one embodiment the replicated (+) strand ssDNAs bind at least one Ff phage coat protein or Ff phage modified coat protein or both. In one embodiment the replicated (+) strand ssDNAs bind a plurality of different Ff phage coat and/or Ff phage modified coat proteins.

In one embodiment the replicated (+) strand ssDNAs are bound by at least one Ff phage coat protein, at least one modified Ff phage coat protein and/or a plurality of different Ff phage coat and/or modified coat proteins within the plurality of nanorods.

In one embodiment the replicated (+) strand ssDNA sequence comprises from 152 to 221 nucleotides (FIGS. 47-48, SEQ ID NO: 80, SEQ ID NO: 78). In one embodiment the replicated (+) strand ssDNA comprises, consists, or consists essentially of 152 nt.

In one embodiment the replicated (+) strand ssDNA comprises 289, 313, 395, 529, 707. 711, 728, 748 nt or 1400 nt (FIG. 45, SEQ ID NO: 73, SEQ ID NO: 71; FIG. 43, SEQ ID NO: 63, SEQ ID NO: 61, FIG. 41, SEQ ID NO: 55, SEQ ID NO: 53; FIG. 39, SEQ ID NO:44, SEQ ID NO: 42; FIG. 56, SEQ ID NO: 102; Table 9).

In one embodiment the at least one auxotrophic marker is selected from the group consisting of metE, glyA, infA, thyA, argE, delta-thi-1, thi1, leuB, proAB, ara, and nadC.

In one embodiment the at least one auxotrophic marker is nadC (FIG. 50, SEQ ID NO: 91, SEQ ID NO: 93).

In one embodiment the at least one inducible promoter is selected from the group consisting of lac, tac, araC, or trp promoters. In one embodiment the at least one inducible promoter is a lac promoter. In one embodiment the lac promoter is a lac promoter mutant regulated solely by the inducer (IPTG). In one embodiment the lac promoter mutant is not susceptible to repression by glucose (catabolite repression). In one embodiment the lac promoter is the lacUV5 promoter (FIG. 49, SEQ ID NO: 90).

In one embodiment the at least one inducible promoter is operably linked to a nucleic acid sequence encoding at least one Ff phage replication protein or at least one Ff phage coat protein or both.

In one embodiment the at least one inducible promoter is operably linked to a nucleic acid sequence encoding at least one Ff phage protein selected from the group consisting of pII, pV, pVII, pVIII, and pIX.

In one embodiment the at least one inducible promoter is operably linked to a nucleic acid sequence encoding the Ff phage proteins pII, pV, pVII, pVIII, and pIX.

In one embodiment the at least one Ff phage replication protein is pII.

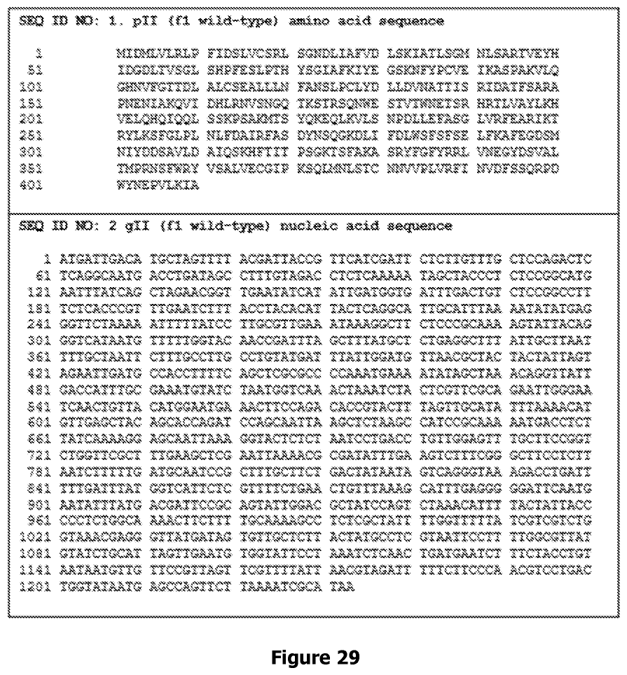

In one embodiment the amino acid sequence of pII comprises, consists, or consists essentially of SEQ ID NO: 1 (FIG. 29). In one embodiment the nucleic acid sequence encoding pII comprises at least 70%, 80%, 90%, 95% or 99% nucleic acid sequence identity with SEQ ID NO: 2 (FIG. 29). In one embodiment the nucleic acid sequence encoding pII comprises, consists, or consists essentially of SEQ ID NO: 2 (FIG. 29).

In one embodiment the at least one Ff phage coat protein is pVIII.

In one embodiment the amino acid sequence of pVIII comprises, consists, or consists essentially of SEQ NO: 11 (FIG. 32). In one embodiment the nucleic acid sequence encoding pVIII comprises at least 70%, 80%, 90%, 95% or 99% sequence identity with SEQ ID NO: 12 (FIG. 32). In one embodiment the nucleic acid sequence encoding pVIII comprises, consists, or consists essentially of SEQ ID NO: 12 (FIG. 32).

In one embodiment the inducible promoter is operably linked to a nucleic acid sequence encoding at least one modified Ff phage replication protein or at least one modified Ff phage coat protein or both.

In one embodiment the at least one modified Ff phage replication or coat protein comprises at least one amino acid addition, deletion or substitution as compared to the corresponding wild type Ff phage coat protein.

In one embodiment the inducible promoter is operably linked to a nucleic acid sequence encoding at least one modified Ff phage replication protein. In one embodiment the modified Ff phage-encoded replication protein is a modified pII protein.

In one embodiment the amino acid sequence of the modified pII protein comprises, consists, or consists essentially of SEQ ID NO: 3, wherein SEQ ID NO: 3 comprises a Thr182IIe amino acid change relative to wild type pII (FIG. 30).

In one embodiment the nucleic acid sequence encoding the modified pII protein comprises, consists, or consists essentially of SEQ ID NO: 4, wherein SEQ ID NO: 4 comprises a C545T change. The skilled worker appreciates that the C545T change is identified by counting from the ATG start codon of the nucleic acid sequence encoding the modified pII protein.

In one embodiment the inducible promoter is operably linked to a nucleic acid sequence encoding at least one modified Ff phage coat protein. In one embodiment the at least one modified Ff phage coat protein is a modified pVIII.

In one embodiment modified pVIII comprises at least one amber mutation. In one embodiment the amino acid sequence of the modified pVIII comprises, consists, or consists essentially of the amino acid sequence of SEQ ID NO: 13. In one embodiment the amino acid sequence of the modified pVIII comprises, consists, or consists essentially of SEQ ID NO: 19.

In one embodiment, the amino acid sequence of pV comprises, consists, or consists essentially of SEQ ID NO: 5 (FIG. 31). In one embodiment the nucleic acid sequence encoding pV comprises at least 70%, 80%, 90%, 95% or 99% sequence identity with SEQ ID NO: 6 (FIG. 31). In one embodiment the nucleic acid sequence encoding pV comprises, consists, or consists essentially of SEQ ID NO: 6 (FIG. 31).

In one embodiment the amino acid sequence of pVII comprises, consists, or consists essentially of SEQ ID NO: 7 (FIG. 31). In one embodiment the nucleic acid sequence encoding pVII comprises at least 70%, 80%, 90%, 95% or 99% sequence identity with SEQ ID NO: 8 (FIG. 31). In one embodiment the nucleic acid sequence encoding pVII comprises, consists, or consists essentially of SEQ ID NO: 8 (FIG. 31).

In one embodiment the amino acid sequence of pIX comprises, consists, or consists essentially of SEQ ID NO: 9. (FIG. 31). In one embodiment the nucleic acid sequence encoding pIX comprises at least 70%, 80%, 90%, 95% or 99% sequence identity with SEQ ID NO: 10 (FIG. 31). In one embodiment the nucleic acid sequence encoding pIX comprises, consists, or consists essentially of SEQ ID NO: 10 (FIG. 31).

In one embodiment, the single nucleic acid expression construct comprises a nucleic acid sequence encoding at least one additional Ff phage protein, preferably at least two additional Ff phage proteins.

In one embodiment the nucleic acid sequence encoding at least one additional Ff phage protein is operably linked to a promoter. In one embodiment the promoter is an inducible or constitutive promoter, preferably the promoter is a constitutive promoter, preferably pZ.

In one embodiment the additional Ff phage proteins are selected from the group consisting of pIII and pVI. In one embodiment the additional Ff phage proteins are pIII or pVI or both.

In one embodiment, the amino acid sequence of pIII comprises, consists, or consists essentially of SEQ ID NO: 29 (FIG. 34). In one embodiment the nucleic acid sequence encoding pIII comprises at least 70%, 80%, 90%, 95% or 99% sequence identity with SEQ ID NO: 30 (FIG. 34). In one embodiment the nucleic acid sequence encoding pIII comprises, consists, or consists essentially of SEQ ID NO: 30 (FIG. 34).

In one embodiment, the amino acid sequence of modified pIII comprises, consists, or consists essentially of SEQ ID NO: 31 (FIG. 35). In one embodiment the nucleic acid sequence encoding pIII comprises at least 70%, 80%, 90%, 95% or 99% sequence identity with SEQ ID NO: 32 (FIG. 35). In one embodiment the nucleic acid sequence encoding modified pIII comprises, consists, or consists essentially of SEQ ID NO: 32 (FIG. 35).

In one embodiment, the amino acid sequence of modified pIII comprises, consists, or consists essentially of SEQ ID NO: 33 (FIG. 36). In one embodiment the nucleic acid sequence encoding modified pIII comprises at least 70%, 80%, 90%, 95% or 99% sequence identity with SEQ ID NO: 34 (FIG. 36). In one embodiment the nucleic acid sequence encoding modified pIII comprises, consists, or consists essentially of SEQ ID NO: 34 (FIG. 36).

In one embodiment, the amino acid sequence of pVI comprises, consists, or consists essentially of SEQ ID NO: 35 (FIG. 36). In one embodiment the nucleic acid sequence encoding pVI comprises at least 70%, 80%, 90%, 95% or 99% sequence identity with SEQ ID NO: 36 (FIG. 36). In one embodiment the nucleic acid sequence encoding pVI comprises, consists, or consists essentially of SEQ ID NO: 36 (FIG. 36).

In one embodiment, the nucleic acid expression construct comprises a nucleic acid sequence encoding a fusion protein comprising at least one Ff phage protein or modified Ff phage protein or functional portion thereof fused to a binding protein or binding portion thereof. In one embodiment the Ff phage protein or modified Ff phage protein or functional portion thereof is a Ff phage coat or modified Ff phage coat protein or functional portion thereof.

In one embodiment the nucleic acid sequence encoding the fusion protein comprises a first nucleic acid coding sequence encoding the at least one Ff phage protein or at least one modified Ff phage protein.

In one embodiment the nucleic acid sequence encoding the fusion protein comprises a second nucleic acid coding sequence, wherein expression of the first and second nucleic acid sequences produces the fusion protein.

In one embodiment the second nucleic acid coding sequence encodes a protein or functional portion thereof that is displayed on the surface of the nanorod. In one embodiment the second nucleic acid sequence encodes an antibody or antigen binding portion thereof, or a binding protein or binding portion thereof.

In one embodiment the antibody or antigen binding portion thereof is selected from the group consisting of a SARS CoV-2-Spike-specific single-chain antibody, preferably C121; a SARS CoV-2 nucleocapsid-specific antigen-binding fragment of a heavy-chain-only antibody (VHH), preferably N3 (VHH N3) and a Botulinum neurotoxin-specific VHH.

In one embodiment the binding protein or binding portion thereof is selected from the group consisting of the FnB fibronectin binding domain of the S. pyogenes M-type 22 protein Sof, the botulinum toxin-binding domain of the synaptic vesicle glycoprotein 2C (SV2C) and SARS-CoV-2 Spike (S), or matrix (M) derived peptides that interact with the SARS-CoV-2 nucleocapsid protein (N).

In one embodiment the first nucleic acid sequence comprises, consists essentially of, or consists of modified gIII (SEQ ID NO: 32; FIG. 35).

In one embodiment the nucleic acid sequence encoding the fusion protein comprises, consists essentially of, or consists of a nucleic acid sequence encoding the single-chain variable domain of antibody C121 (scFvC121) fused to a nucleic acid sequence encoding the full-length pIII (SEQ ID NO: 40; FIG. 38). In one embodiment the fusion protein comprises SEQ ID NO: 40.

In one embodiment the nucleic acid sequence encoding the fusion protein comprises, consists essentially of, or consists of a nucleic acid sequence encoding the antigen-binding fragment of a heavy-chain-only antibody N3 (VHH N3) fused to a nucleic acid sequence encoding the full-length pIII (SEQ ID NO: 100; FIG. 55). In one embodiment the fusion protein comprises SEQ ID NO: 99.

In one embodiment the nucleic acid sequence encoding the fusion protein comprises, consists essentially of, or consists of the nucleic acid coding sequence for the FnB fibronectin binding domain of the S. pyogenes M-type 22 protein Sof fused to the full-length gIII coding sequence (SEQ ID NO: 38; FIG. 37). In one embodiment the fusion protein comprises SEQ ID NO: 37; FIG. 37.

The skilled person will appreciate that the amino acid sequences of any of pIII, pVI, pVII, pVIII or pIX can be modified as described herein and as known in the art for the purposes of peptide display. All such modifications are contemplated herein and are believed to be within the skill of the art when combined with the disclosure of the present specification.

In one embodiment the inducible promoter is operably linked to a first operon comprising, consisting of, or consisting essentially of Ff phage genes gII(gX), gV, gVII, gIX and gVIII.

In one embodiment Ff phage genes gII(gX), gV, gVII, gIX and gVIII comprise at least 70%, 80%, 90%, 95% or 99% nucleic acid sequence identity with SEQ ID NO: 4 (FIG. 30), SEQ ID NO: 6, SEQ ID NO: 8, SEQ ID NO: 10 (FIG. 31) and one of SEQ ID NO: 14, SEQ ID NO: 16, SEQ ID NO: 18 (FIG. 32), SEQ ID NO: 20, SEQ ID NO: 22, SEQ ID NO: 24, SEQ ID NO: 26 or SEQ ID NO: 28 (FIG. 33), SEQ ID NO: 98 (FIG. 54), respectively.

In one embodiment Ff phage genes gII(gX), gV, gVII, gIX and gVIII comprise, consist or consist essentially of SEQ ID NO: 4 (FIG. 30), SEQ ID NO: 6, SEQ ID NO: 8, SEQ ID NO: 10 (FIG. 31) and one of SEQ ID NO: 14, SEQ ID NO: 16, SEQ ID NO: 18 (FIG. 32), SEQ ID NO: 20, SEQ ID NO: 22, SEQ ID NO: 24, SEQ ID NO: 26 or SEQ ID NO: 28 (FIG. 33), SEQ ID NO: 98 (FIG. 54), respectively.

In one embodiment Ff phage genes gIII and gVIII are modified to encode modified Ff phage coat proteins pIII and pVIII, respectively.

In one embodiment modified pVIII comprises at least one amber mutation.

In one embodiment the amino acid sequence of the modified pVIII comprises, consists, or consists essentially of the amino acid sequence of SEQ ID NO: 13 (FIG. 32). In one embodiment the amino acid sequence of the modified pVIII comprises, consists, or consists essentially of the amino acid sequence of SEQ ID NO: 15 (FIG. 32). In one embodiment the amino acid sequence of the modified pVIII comprises, consists, or consists essentially of the amino acid sequence of SEQ ID NO: 17 (FIG. 32). In one embodiment the amino acid sequence of the modified pVIII comprises, consists, or consists essentially of SEQ ID NO: 19 (FIG. 32). In one embodiment the amino acid sequence of the modified pVIII comprises, consists, or consists essentially of the amino acid sequence of SEQ ID NO: 21 (FIG. 33). In one embodiment the amino acid sequence of the modified pVIII comprises, consists, or consists essentially of the amino acid sequence of SEQ ID NO: 23 (FIG. 33). In one embodiment the amino acid sequence of the modified pVIII comprises, consists, or consists essentially of the amino acid sequence of SEQ ID NO: 25 (FIG. 33). In one embodiment the amino acid sequence of the modified pVIII comprises, consists, or consists essentially of the amino acid sequence of SEQ ID NO: 27 (FIG. 33). In one embodiment the amino acid sequence of the modified pVIII comprises, consists, or consists essentially of the amino acid sequence of SEQ ID NO: 97 (FIG. 54).

In one embodiment the at least one plasmid origin of replication (p-ori) is a theta origin of plasmid replication. In one embodiment the p-ori is selected from the group consisting of ColE1, pMB1, pSC101, R6K, ColD and 15A. In one embodiment the p-ori is 15A.

In one embodiment the nucleic acid construct comprises a second operon comprising, consisting of, or consisting essentially of Ff phage genes gIII, gVI, gI(gXI) and gIV. In one embodiment the second operon is operatively linked to a constitutive or inducible promoter, preferably a constitutive promoter, preferably an inducible promoter. In one embodiment the inducible promoter is as described herein for the NPS aspects of the invention.

In one embodiment Ff phage gene gIII comprises at least 70%, 80%, 90%, 95% or 99% nucleic acid sequence identity with SEQ ID NO: 30 (FIG. 34), SEQ ID NO: 32 (FIG. 35) or SEQ ID NO: 34 (FIG. 36). In one embodiment Ff phage gene gIII comprises, consists, or consists essentially of SEQ ID NO: 30 (FIG. 34), SEQ ID NO: 32 (FIG. 35) or SEQ ID NO: 34 (FIG. 36).

In one embodiment Ff phage gene gVI comprises at least 70%, 80%, 90%, 95% or 99% nucleic acid sequence identity with SEQ ID NO: 36 (FIG. 36). In one embodiment Ff phage gene gVI comprises, consists, or consists essentially of SEQ ID NO: 36 (FIG. 36).

In a second aspect, the invention relates to a nanorod production system (NPS) comprising
i) a nucleic acid replication-assembly construct comprising
   a BSFnano replication-assembly cassette,
   at least one auxotrophic marker, and
   at least one plasmid origin of replication not located in the BSFnano replication-assembly cassette, and
ii) a helper nucleic acid expression construct comprising
   at least one selective marker, and
   at least one inducible promoter operably linked to a nucleic acid sequence encoding at least one Ff phage protein.

In one embodiment, the nucleic acid replication construct in i) is or is comprised in, a vector. In one embodiment the nucleic acid replication construct in i) is a vector. In one embodiment the vector is selected from the group consisting of plasmids, bacterial artificial chromosomes (BACs), P1-derived artificial chromosomes (PACs), yeast artificial chromosomes (YACs), bacteriophage, phagemids, and cosmids. In one embodiment the vector is a plasmid.

In one embodiment the nucleic acid expression construct in i) is or is comprised in, a plasmid. In one embodiment the nucleic acid replication construct in i) is a plasmid. In this embodiment the plasmid is termed a BSFnano replication-assembly plasmid.

In one embodiment, the helper nucleic acid expression construct in ii) is or is comprised in, a vector. In one embodiment the helper nucleic acid expression construct in ii) is a vector. In one embodiment the vector is selected from the group consisting of plasmids, bacterial artificial chromosomes (BACs), P1-derived artificial chromosomes (PACs), yeast artificial chromosomes (YACs) and cosmids. In one embodiment the vector is a plasmid.

In one embodiment the helper nucleic acid expression construct in ii) is or is comprised in, a plasmid. In one embodiment the helper nucleic acid expression construct in ii) is a plasmid. In this embodiment the plasmid is termed a helper plasmid.

In one embodiment the BSFnano replication-assembly cassette comprises at least two (+) ori's. In one embodiment the BSFnano replication-assembly cassette comprises at least one (−) ori. In one embodiment the BSFnano replication-assembly cassette comprises two (+) ori's and one (−) ori.

In one embodiment one (+) ori is a DNA replication initiator. The (+) ori that is a DNA replication initiator is termed (+) ori1 herein. In one embodiment one (+) ori is a DNA replication terminator. The (+) ori that is a DNA replication terminator is termed (+) ori2 herein.

In one embodiment one (+) ori is a DNA replication initiator (“(+)ori1”) and one (+) ori is a DNA replication terminator (“(+) ori2”). In one embodiment the BSFnano replication-assembly cassette comprises (+) ori1, (+) ori2, and one (−) ori.

In one embodiment the BSFnano replication-assembly cassette comprises a packaging signal (PS). In one embodiment the PS is between (+) ori1 and (+) ori2. In one embodiment the PS is between (+) ori1 and the (−) ori. In one embodiment (+) ori1 and (+) ori2 comprise pII cut sites.

In one embodiment the BSFnano replication-assembly cassette comprises a scaffold nucleic acid sequence. In one embodiment the BSFnano replication-assembly cassette comprises a scaffold nucleic acid sequence plus flanking sequences required for the (+) strand replication.

In one embodiment the scaffold nucleic acid sequence is positioned between the (+) ori1 and (+) ori2. In one embodiment the scaffold nucleic acid sequence is positioned between pII cut sites in (+) ori1 and (+) ori2.

In one embodiment the scaffold nucleic acid sequence is positioned between sequences [(GTTCTTAATA) (SEQ ID NO:88, FIG. 49) in (+) ori1 (initiator) and in (+) ori2 (terminator)]. In one embodiment the scaffold nucleic acid sequence is positioned in the BSFnano replication-assembly cassette as shown in FIGS. 5 and 6.

In one embodiment replication of the scaffold nucleic acid sequence in the presence of pII produces a circular ssDNA.

In one embodiment the scaffold nucleic acid sequence comprises no filler nucleic acid sequence. In one embodiment the scaffold nucleic acid sequence comprises at least one filler nucleic acid sequence. In one embodiment the scaffold nucleic acid sequence comprises two filler nucleic acid sequences.

In one embodiment the scaffold nucleic acid sequence comprises a filler nucleic acid sequence comprising an additional nucleic acid sequence positioned to extend the length of a (+) strand ssDNA produced by replication of the scaffold nucleic acid sequence. In one embodiment a filler nucleic acid sequence is positioned as shown in FIGS. 5 and 6, “Filler”.

In one embodiment the scaffold nucleic acid sequence comprises a filler nucleic acid sequence positioned between (+) ori1 and the PS. In one embodiment the scaffold nucleic acid sequence comprises a filler nucleic acid sequence positioned between the PS and (+) ori2. In one embodiment the scaffold nucleic acid sequence comprises a filler nucleic acid sequence between (+) ori1 and the PS and between the PS and (+) ori2.

In one embodiment the scaffold nucleic acid sequence comprises a filler nucleic acid sequence comprising 0 to about 6000 nt, 0 to about 5000, 0 to about 4000, 0 to about 3000, 0 to about 2000, 0 to about 1000, 0 to about 750, 0 to about 500, 0 to about 400, 0 to about 300, 0 to about 200, 0 to about 100, 0 to about 50, 0 to about 40, 0 to about 30, 0 to about 25, 0 to about 20, 0 to about 15, 0 to about 10, 0 to about 5, or 0 nt. In one embodiment the scaffold nucleic acid sequence comprises a filler nucleic acid sequence comprising 0 to 6000 nt, 0 to 5000, 0 to 4000, 0 to 3000, 0 to 2000, 0 to 1000, 0 to 750, 0 to 500, 0 to 400, 0 to 300, 0 to 200, 0 to 100, 0 to 50, 0 to 40, 0 to 30, 0 to 25, 0 to 20, 0 to 15, 0 to 10, 0 to 5, or 0 nt.

In one embodiment the scaffold nucleic acid sequence comprises a filler nucleic acid sequence comprising 0, 5, 23, 24, 31, 145, 315, 319, 336, 356, 700, 1400 or 2100 nt.

In one embodiment the filler nucleic acid sequence comprises, consists essentially of, or consists of a filler nucleic acid sequence as identified in Table 9. The skilled worker appreciates that the size of the filler may be varied to accommodate the production of nanorods of various sizes depending on the lengths (i.e., number of nucleotides) of the other functional sequence elements of the scaffold nucleic acid sequence including (+) on 1, (−) on and (+) on 2.

In one embodiment the single nucleic acid construct comprises SEQ ID NO: 41, SEQ ID NO: 43, SEQ ID NO: 52, SEQ ID NO: 54, SEQ ID NO: 61, SEQ ID NO: 63, SEQ ID NO: 70, SEQ ID NO: 72, SEQ ID NO: 77, SEQ ID NO: 79 or SEQ ID NO: 101 (FIGS. 39-48; 56-57).

In one embodiment the scaffold nucleic acid sequence comprises a filler nucleic acid sequence codes for at least one, preferably at least two Ff phage coat and/or Ff phage modified coat proteins. In one embodiment the at least one coat and/or modified coat protein is pVII or pIX. In one embodiment the at least two coat and/or modified coat proteins are pVII and pIX.

In one embodiment the at least two Ff phage coat and/or modified coat proteins are operably linked to a promoter. In one embodiment the promoter is a constitutive or inducible promoter. In one embodiment the promoter is a constitutive promoter. In one embodiment the promoter is an inducible promoter. In one embodiment the constitutive promoter is a phage promoter, preferably pA. In one embodiment the inducible promoter is selected from the group consisting of lac, tac, araC, or trp promoters.

In one embodiment the promoter is a lac promoter. In one embodiment the lac promoter is regulated by the inducer (IPTG). In one embodiment the lac promoter is susceptible to repression by glucose (catabolite repression). In one embodiment the lac promoter is the lac promoter (FIG. 42, SEQ ID NO: 58).

In one embodiment the promoter is a lac promoter. In one embodiment the lac promoter is a lac promoter mutant regulated solely by the inducer (IPTG). In one embodiment the lac promoter mutant is not susceptible to repression by glucose (catabolite repression). In one embodiment the lac promoter is the lacUV5 promoter (FIG. 49, SEQ ID NO: 90).

In one embodiment the BSFnano replication-assembly cassette comprises the scaffold nucleic acid sequence comprising flanking nucleic acid sequences within (+) ori 1 and (+) ori2. In one embodiment the flanking nucleic acid sequences bind pII and/or bind modified pII.

In one embodiment, enzymatic replication of the scaffold nucleic acid sequence produces a plurality of replicated (+) strand circular ssDNA molecules. In one embodiment, enzymatic replication is rolling circle replication.

In one embodiment the replicated (+) strand ssDNAs bind at least one Ff phage coat protein or Ff phage modified coat protein or both. In one embodiment the replicated (+) strand ssDNAs bind a plurality of different Ff phage coat and/or modified coat proteins.

In one embodiment the replicated (+) strand ssDNAs are bound by at least one Ff phage coat protein, at least one modified Ff phage coat protein and/or a plurality of different Ff phage coat and/or modified coat proteins within the plurality of nanorods.

In one embodiment the replicated (+) strand ssDNA comprises 152 to 221 nucleotides (nt) (FIG. 47, SEQ ID NO: 80, SEQ ID NO: 78). In one embodiment the replicated (+) strand ssDNA comprises, consists, or consists essentially of 152 nt.

In one embodiment the replicated+ssDNA comprises 289, 313, 395, 529, 707, 711, 728, 748 or 1400 nt (FIG. 45, SEQ ID NO: 73, SEQ ID NO: 71; FIG. 43, SEQ ID NO: 63, SEQ ID NO: 61, FIG. 41, SEQ ID NO: 55, SEQ ID NO: 53; FIG. 39, SEQ ID NO:44, SEQ ID NO: 42; FIG. 56, SEQ ID NO: 102; Table 9).

In one embodiment the auxotrophic marker is selected from the group consisting of metE, glyA, infA, thyA, argE, delta-thi-1, thi1, leuB, proAB, ara, and nadC. In one embodiment the auxotrophic marker is nadC (FIG. 50, SEQ ID NO: 91, SEQ ID NO: 92).

In one embodiment the plasmid origin of replication in i) (p-ori) is a theta origin of plasmid replication. In one embodiment the p-ori is selected from the group consisting of ColE1, pMB1, pSC101, R6K, ColD and pA15. In one embodiment the p-ori is pMB1.

In one embodiment the helper plasmid in ii) comprises a plasmid origin of replication. In one embodiment the plasmid origin of replication in ii) (p-ori) is a theta origin of plasmid replication. In one embodiment the p-ori is selected from the group consisting of ColE1, pMB1, pSC101, R6K, ColD and pA15.

In one embodiment the at least one selective marker in ii) is an antibiotic resistance or auxotrophic marker. In one embodiment the at least one selective marker is an antibiotic resistance marker. In one embodiment at least one selective marker is an auxotrophic marker.

In one embodiment the at least one inducible promoter in ii) is selected from the group consisting of lac, tac, araC, or trp promoters. In one embodiment the at least one inducible promoter is a lac promoter. In one embodiment the lac promoter is a lac promoter mutant regulated solely by the inducer (IPTG). In one embodiment the lac promoter mutant is not susceptible to repression by glucose (catabolite repression). In one embodiment the lac promoter is the lacUV5 promoter (FIG. 49, SEQ ID NO: 90).

In one embodiment the at least one inducible promoter in ii) is operably linked to a nucleic acid sequence encoding at least one Ff phage replication protein or at least one Ff phage coat protein or both.

In one embodiment the at least one inducible promoter in ii) is operably linked to a nucleic acid sequence encoding at least two Ff phage replication proteins or at least two Ff phage coat proteins or both.

In one embodiment, the at least one inducible promoter is operably linked to a nucleic acid sequence encoding at least two Ff phage coat proteins. In one embodiment the at least two Ff phage coat proteins are minor coat proteins.

In one embodiment the at least two minor coat proteins are pVII and pIX.

In one embodiment, the at least one inducible promoter is operably linked to a nucleic acid sequence encoding at least one, preferably at least two Ff phage replication proteins.

In one embodiment the inducible promoter is operably linked to a nucleic acid sequence encoding at least one Ff phage protein selected from the group consisting of pII, pV, pVII, pVIII, and pIX.

In one embodiment the inducible promoter is operably linked to a nucleic acid sequence encoding the Ff phage proteins pII, pV, pVII, pVIII, and pIX.

In one embodiment the at least one Ff phage replication protein is pII.

In one embodiment the amino acid sequence of pII comprises, consists, or consists essentially of SEQ ID NO: 1 (FIG. 29). In one embodiment the nucleic acid sequence encoding pII comprises at least 70%, 80%, 90%, 95% or 99% nucleic acid sequence identity with SEQ ID NO: 2 (FIG. 29). In one embodiment the nucleic acid sequence encoding pII comprises, consists, or consists essentially of SEQ ID NO: 2 (FIG. 29).

In one embodiment the at least one Ff phage coat protein is pVIII.

In one embodiment the amino acid sequence of pVIII comprises, consists, or consists essentially of SEQ NO: 11 (FIG. 32). In one embodiment the nucleic acid sequence encoding pVIII comprises at least 70%, 80%, 90%, 95% or 99% sequence identity with SEQ ID NO: 12 (FIG. 32). In one embodiment the nucleic acid sequence encoding pVIII comprises, consists, or consists essentially of SEQ ID NO: 12 (FIG. 32).

In one embodiment the inducible promoter is operably linked to a nucleic acid sequence encoding at least one modified Ff phage replication protein or at least one modified Ff phage coat protein or both. In one embodiment the at least one modified Ff phage replication or coat protein comprises at least one amino acid addition, deletion or substitution as compared to the corresponding wild type Ff phage coat protein.

In one embodiment the inducible promoter is operably linked to a nucleic acid sequence encoding at least one modified Ff phage replication protein. In one embodiment the modified Ff phage-encoded replication protein is a modified pII protein.

In one embodiment the amino acid sequence of the modified pII protein comprises, consists, or consists essentially of SEQ ID NO: 3, wherein SEQ ID NO: 3 comprises a Thr182IIe amino acid change relative to wild type pII (FIG. 30).

In one embodiment the nucleic acid sequence encoding the modified pII protein comprises, consists, or consists essentially of SEQ ID NO: 4, wherein SEQ ID NO: 4 comprises a C545T change. The skilled worker appreciates that the C545T change is identified by counting from the ATG start codon of the nucleic acid sequence encoding the modified pII protein.

In one embodiment the inducible promoter is operably linked to a nucleic acid sequence encoding at least one modified Ff phage coat protein. In one embodiment the at least one modified Ff phage coat protein is a modified pVIII. In one embodiment modified pVIII comprises at least one amber mutation.

In one embodiment the amino acid sequence of the modified pVIII comprises, consists, or consists essentially of the amino acid sequence of SEQ ID NO: 13. In one embodiment the amino acid sequence of the modified pVIII comprises, consists, or consists essentially of SEQ ID NO: 19.

In one embodiment, the amino acid sequence of pV comprises, consists, or consists essentially of SEQ ID NO: 5 (FIG. 31). In one embodiment the nucleic acid sequence encoding pV comprises at least 70%, 80%, 90%, 95% or 99% sequence identity with SEQ ID NO: 6 (FIG. 31). In one embodiment the nucleic acid sequence encoding pV comprises, consists, or consists essentially of SEQ ID NO: 6 (FIG. 31).

In one embodiment the amino acid sequence of pVII comprises, consists, or consists essentially of SEQ ID NO: 7 (FIG. 31). In one embodiment the nucleic acid sequence encoding pVII comprises at least 70%, 80%, 90%, 95% or 99% sequence identity with SEQ ID NO: 8 (FIG. 31). In one embodiment the nucleic acid sequence encoding pVII comprises, consists, or consists essentially of SEQ ID NO: 8 (FIG. 31).

In one embodiment the amino acid sequence of pIX comprises, consists, or consists essentially of SEQ ID NO: 9. (FIG. 31). In one embodiment the nucleic acid sequence encoding pIX comprises at least 70%, 80%, 90%, 95% or 99% sequence identity with SEQ ID NO: 10 (FIG. 31). In one embodiment the nucleic acid sequence encoding pIX comprises, consists, or consists essentially of SEQ ID NO: 10 (FIG. 31).

In one embodiment the helper plasmid in ii) comprises a nucleic acid sequence encoding at least one additional Ff phage protein, preferably at least two additional Ff phage proteins. In one embodiment the nucleic acid sequence encoding at least one additional Ff phage protein is operably linked to a promoter. In one embodiment the promoter is an inducible or constitutive promoter, preferably the promoter is a constitutive promoter, preferably pZ.

In one embodiment the additional Ff phage proteins are selected from the group consisting of pIII and pVI. In one embodiment the additional Ff phage proteins are pIII or pVI or both.

In one embodiment, the amino acid sequence of pIII comprises, consists, or consists essentially of SEQ ID NO: 29 (FIG. 34). In one embodiment the nucleic acid sequence encoding pIII comprises at least 70%, 80%, 90%, 95% or 99% sequence identity with SEQ ID NO: 30 (FIG. 34). In one embodiment the nucleic acid sequence encoding modified pIII comprises, consists, or consists essentially of SEQ ID NO: 30 (FIG. 34).

In one embodiment the at least one modified Ff phage coat protein is a modified pIII protein. In one embodiment the modified pIII comprises, consists essentially of, or consists of SEQ ID NO: 31 (FIG. 35). In one embodiment the nucleic acid sequence encoding the modified pIII comprises at least 70%, 80%, 90%, 95% or 99% sequence identity with SEQ ID NO: 32 (FIG. 35). In one embodiment the nucleic acid sequence encoding modified pIII comprises, consists, or consists essentially of SEQ ID NO: 32 (FIG. 35).

In one embodiment, the amino acid sequence of modified pIII comprises, consists, or consists essentially of SEQ ID NO: 33 (FIG. 36). In one embodiment the nucleic acid sequence encoding modified pIII comprises at least 70%, 80%, 90%, 95% or 99% sequence identity with SEQ ID NO: 34 (FIG. 36). In one embodiment the nucleic acid sequence encoding modified pIII comprises, consists, or consists essentially of SEQ ID NO: 34 (FIG. 36).

In one embodiment, the amino acid sequence of pVI comprises, consists, or consists essentially of SEQ ID NO: 35 (FIG. 36). In one embodiment the nucleic acid sequence encoding pVI comprises at least 70%, 80%, 90%, 95% or 99% sequence identity with SEQ ID NO: 36 (FIG. 36). In one embodiment the nucleic acid sequence encoding pVI comprises, consists, or consists essentially of SEQ ID NO: 36 (FIG. 36).

In one embodiment, the helper plasmid in ii) comprises a nucleic acid sequence encoding a fusion protein comprising at least one Ff phage protein or modified Ff phage protein or functional portion thereof fused to a binding protein or binding portion thereof.

In one embodiment the Ff phage protein or modified Ff phage protein or functional portion thereof is a Ff phage coat or modified Ff phage coat protein or functional portion thereof. In one embodiment the nucleic acid sequence encoding the fusion protein comprises a first nucleic acid coding sequence encoding the at least one Ff phage protein or at least one modified Ff phage protein.

In one embodiment the nucleic acid sequence encoding the fusion protein comprises a second nucleic acid coding sequence, wherein expression of the first and second nucleic acid sequences produces the fusion protein.

In one embodiment the second nucleic acid coding sequence encodes a binding protein or binding portion thereof that is displayed on the surface of the nanorod. In one embodiment the binding protein is an antibody or antigen binding portion thereof, or a binding protein or binding portion thereof.

In one embodiment the antibody or antigen binding portion thereof is selected from the group consisting of a SARS CoV-2-Spike-specific single-chain antibody, preferably C121 (scFv C121); a SARS CoV-2 nucleocapsid-specific antigen-binding fragment of a heavy-chain-only antibody (VHH), preferably N3 (VHH N3), and a Botulinum neurotoxin-specific VHH.

In one embodiment the binding protein or binding portion thereof is selected from the group consisting of the FnB fibronectin binding domain of the S. pyogenes M-type 22 protein Sof, the botulinum toxin-binding domain of the synaptic vesicle glycoprotein 2C (SV2C) and SARS-CoV-2 spike (S), or matrix (M) derived peptides that interact with the SARS-CoV-2 nucleocapsid protein (N).

In one embodiment the first nucleic acid sequence nucleic acid sequence comprises, consists essentially of, or consists of modified gIII (SEQ ID NO: 32; FIG. 35).

In one embodiment the nucleic acid sequence encoding the fusion protein comprises, consists essentially of, or consists of a nucleic acid sequence encoding the single-chain variable domain of antibody C121 (scFvC121) fused to a nucleic acid sequence encoding the full-length pIII (SEQ ID NO: 40; FIG. 38). In one embodiment the fusion protein comprises SEQ ID NO: 40.

In one embodiment the nucleic acid sequence encoding the fusion protein comprises, consists essentially of, or consists of a nucleic acid sequence encoding the antigen-binding fragment of a heavy-chain-only antibody N3 (VHH N3) fused to a nucleic acid sequence encoding the full-length pIII (SEQ ID NO: 100; FIG. 55). In one embodiment the fusion protein comprises SEQ ID NO: 99.

In one embodiment the nucleic acid sequence encoding the fusion protein comprises, consists essentially of, or consists of the nucleic acid coding sequence for the FnB fibronectin binding domain of the S. pyogenes M-type 22 protein Sof fused to the full-length gIII coding sequence (SEQ ID NO: 38; FIG. 37). In one embodiment the fusion protein comprises SEQ ID NO: 37; FIG. 37.

The skilled person will appreciate that the amino acid sequences of any of pIII, pVI, pVII, pVIII or pIX can be modified as described herein and as known in the art, such as for the purposes of peptide display. All such modifications are contemplated herein and are believed to be within the skill of the art when combined with the disclosure of the present specification.

In one embodiment the inducible promoter in ii) is operably linked to a first operon comprising, consisting of, or consisting essentially of Ff phage genes gII(gX), gV, gVII, and gVIII.

In one embodiment Ff phage genes gII(gX), gV, gVII, and gVIII comprise at least 70%, 80%, 90%, 95% or 99% nucleic acid sequence identity with SEQ ID NO: 4, (FIG. 30), SEQ ID NO: 6, SEQ ID NO: 8, SEQ ID NO: 10 (FIG. 31), SEQ ID NO: 14, SEQ ID NO: 16, SEQ ID NO: 18 (FIG. 32), SEQ ID NO: 20, SEQ ID NO: 22, SEQ ID NO: 24, SEQ ID NO: 26 and SEQ ID NO: 28 (FIG. 33), SEQ ID NO: 98 (FIG. 54), respectively.

In one embodiment Ff phage genes gII(gX), gV, gVII, gIX and gVIII comprise, consist or consist essentially of SEQ ID NO: 4, (FIG. 30), SEQ ID NO: 6, SEQ ID NO: 8, SEQ ID NO: 10 (FIG. 31), SEQ ID NO: 14, SEQ ID NO: 16, SEQ ID NO: 18 (FIG. 32), SEQ ID NO: 20, SEQ ID NO: 22, SEQ ID NO: 24, SEQ ID NO: 26 or SEQ ID NO: 28 (FIG. 33), SEQ ID NO: 98 (FIG. 54), respectively.

In one embodiment Ff phage genes gIII and gVIII encode modified Ff phage coat proteins pIII and pVIII, respectively.

In one embodiment modified pVIII comprises at least one amber mutation.

In one embodiment the amino acid sequence of the modified pVIII comprises, consists, or consists essentially of the amino acid sequence of SEQ ID NO: 13 (FIG. 32). In one embodiment the amino acid sequence of the modified pVIII comprises, consists, or consists essentially of the amino acid sequence of SEQ ID NO: 15 (FIG. 32). In one embodiment the amino acid sequence of the modified pVIII comprises, consists, or consists essentially of the amino acid sequence of SEQ ID NO: 17 (FIG. 32). In one embodiment the amino acid sequence of the modified pVIII comprises, consists, or consists essentially of SEQ ID NO: 19 (FIG. 32). In one embodiment the amino acid sequence of the modified pVIII comprises, consists, or consists essentially of the amino acid sequence of SEQ ID NO: 21 (FIG. 33). In one embodiment the amino acid sequence of the modified pVIII comprises, consists, or consists essentially of the amino acid sequence of SEQ ID NO: 23 (FIG. 33). In one embodiment the amino acid sequence of the modified pVIII comprises, consists, or consists essentially of the amino acid sequence of SEQ ID NO: 25 (FIG. 33). In one embodiment the amino acid sequence of the modified pVIII comprises, consists, or consists essentially of the amino acid sequence of SEQ ID NO: 27 (FIG. 33). In one embodiment the amino acid sequence of the modified pVIII comprises, consists, or consists essentially of the amino acid sequence of SEQ ID NO: 97 (FIG. 54).

In one embodiment the helper plasmid in ii) comprises a second operon comprising, consisting of, or consisting essentially of Ff phage genes gIII, gVI, gI (gXI) and gIV. In one embodiment the second operon is operatively linked to a constitutive or inducible promoter, preferably a constitutive promoter, preferably an inducible promoter. In one embodiment the inducible promoter is as described herein for the NPS aspects of the invention.

In one embodiment Ff phage gene gIII comprises at least 70%, 80%, 90%, 95% or 99% nucleic acid sequence identity with SEQ ID NO: 30 (FIG. 34), SEQ ID NO: 32 (FIG. 35) or SEQ ID NO: 34 (FIG. 36). In one embodiment Ff phage gene gIII comprises, consists, or consists essentially of SEQ ID NO: 30 (FIG. 34), SEQ ID NO: 32 (FIG. 35) or SEQ ID NO: 34 (FIG. 36).

In one embodiment Ff phage gene gVI comprises at least 70%, 80%, 90%, 95% or 99% nucleic acid sequence identity with SEQ ID NO: 36 (FIG. 36). In one embodiment Ff phage gene gVI comprises, consists, or consists essentially of SEQ ID NO: 36 (FIG. 36).

In another aspect the invention relates to a composition comprising a plurality or population of nanorods as described herein or produced from an NPS as described herein or made by a method of making a nanorod as described herein.

In one embodiment the composition comprises at least 1.0×10¹⁴, preferably at least 1.0×10¹⁵ nanorods/L. In one embodiment the composition comprises about 1.0×10¹⁴, preferably about 1.0×10¹⁵, preferably about 1.0×10¹⁶ nanorods/L. In one embodiment the composition comprises 1.0×10¹⁴, preferably 1.0×10¹⁵, preferably 1.0×10¹⁶ nanorods/L.

The skilled person appreciates, with relation to the length of a nanorod set forth in the following embodiments and in other embodiments throughout the specification, that the stated length value refers to the stated length value+/−5 nm.

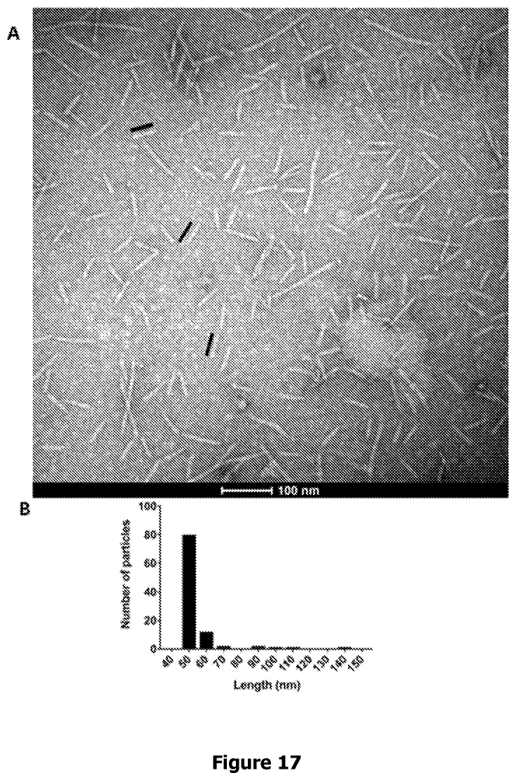

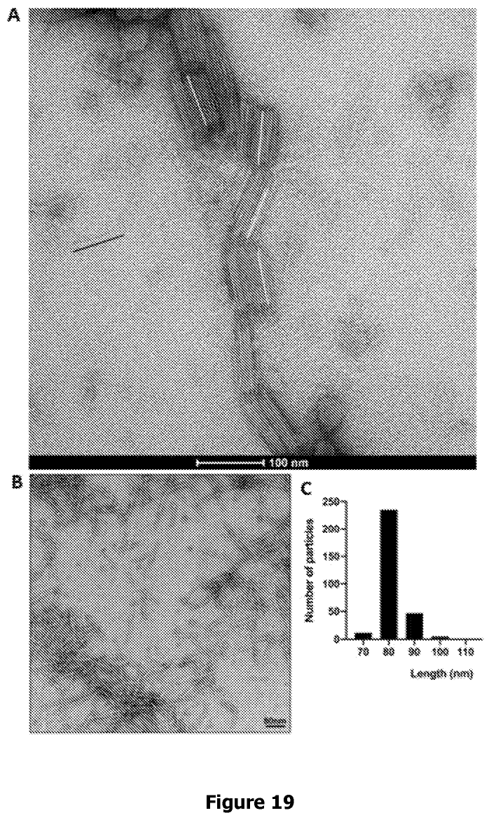

In one embodiment at least 70%, at least 75%, preferably at least 80% of the nanorods are about 40 nm to about 1000 nm in length, preferably about 40 nm to about 400 nm in length, preferably about 100 nm to 300 nm in length. In one embodiment at least 70%, at least 75%, preferably at least 80% of the nanorods are at least 40 nm, preferably at least 50 nm (FIG. 17), 60 nm, 70 nm, 80 nm (FIG. 19), 100 nm, 110 nm, 200 nm, 300 nm, 400 nm, 500 nm, 600 nm, 700 nm, 800 nm, 900 nm, 1000 nm, 2000 nm, 3000 nm, 4000 nm, 5000 nm, or at least 6000 nm in length. In one embodiment at least 70% of the nanorods are about 40 nm, preferably about 50 nm (FIG. 17), 60 nm, 70 nm, 80 nm (FIG. 19), 100 nm, 110 nm, 200 nm, 300 nm, 400 nm, 500 nm, 600 nm, 700 nm, 800 nm, 900 nm, 1000 nm, 2000 nm, 3000 nm, 4000 nm, 5000 nm, or 6000 nm in length. In one embodiment at least 70%, at least 75%, preferably at least 80% of the nanorods are 40 nm, 50 nm (FIG. 17), 60 nm, 70 nm, 80 nm (FIG. 19), 100 nm, 110 nm, 200 nm, 300 nm, 400 nm, 500 nm, 600 nm, 700 nm, 800 nm, 900 nm, 1000 nm, 2000 nm, 3000 nm, 4000 nm, 5000 nm, or 6000 nm in length. In one embodiment, at least 70%, at least 75%, preferably at least 80% of the nanorods are 40 nm in length.

In one embodiment at least 70%, at least 75%, preferably at least 80% of the nanorods are at least 40 nm, 50 nm (FIG. 17), 60 nm, 70 nm, or 80 nm (FIG. 19) in length. In one embodiment at least 70%, at least 75%, preferably at least 80% of the nanorods are about 40 nm, 50 nm (FIG. 17), 60 nm, 70 nm, or 80 nm (FIG. 19) in length. In one embodiment at least 70%, at least 75%, preferably at least 80% of the nanorods are 40 nm, 50 nm (FIG. 17), 60 nm, 70 nm, or 80 nm (FIG. 19) in length. In one embodiment at least 70%, at least 75%, preferably at least 80% of the nanorods are at least 200 nm, 300 nm, 400 nm, 500 nm, 600 nm, 700 nm, 800 nm, 900 nm or at least 1000 nm in length. In one embodiment at least 70%, at least 75%, preferably at least 80% of the nanorods are about 200 nm, 300 nm, 400 nm, 500 nm, 600 nm, 700 nm, 800 nm, 900 nm or about 1000 nm in length. In one embodiment at least 70%, at least 75%, preferably at least 80% of the nanorods are 200 nm, 300 nm, 400 nm, 500 nm, 600 nm, 700 nm, 800 nm, 900 nm, or 1000 nm in length.

In one embodiment at least 70%, at least 75%, preferably at least 80% of nanorods are about 80 nm in length (FIG. 19). In one embodiment at least 70%, at least 75%, preferably at least 80% of nanorods are 80 nm in length. In one embodiment at least 70%, at least 75%, preferably at least 80% of nanorods are about 100 nm, 110 nm, 200 nm, or 300 nm in length. In one embodiment at least 70%, at least 75%, preferably at least 80% of nanorods are 100 nm, 110 nm, 200 nm, or 300 nm in length.

In one embodiment the nanorods comprise a (+) strand ssDNA that comprises an Ff phage origin of replication. In one embodiment the nanorods comprise a (+) strand ssDNA that does not comprise a selective marker. In one embodiment the nanorods comprise a (+) strand ssDNA that does not comprise an antibiotic resistance marker.

In one embodiment the nanorods comprise a (+) strand ssDNA that encodes at least one, preferably at least two Ff phage coat proteins as described herein.

In some embodiments the nanorods comprise at least one modified Ff phage protein as described herein. In one embodiment the nanorods comprise at least one fusion protein as described herein.

Specifically contemplated as embodiments of this aspect of the invention directed to a composition comprising a plurality or population of nanorods are any and/or all of the embodiments set forth in the other aspects of the invention related to nanorod production systems (NPS), nanorods, nanorod conjugates, and methods of making nanorods as described herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, promoters, selectable markers, scaffold and filler nucleic acid sequences, replicated+strand cc ssDNAs, binding agents, detection moieties, and fusion proteins.

In another aspect, the invention relates to a nanorod production system (NPS) comprising a nucleic acid expression construct comprising a replication-assembly cassette comprising a filamentous phage (+) ori1, a packaging signal (PS) and an (+) ori2, at least one plasmid origin of replication not located in the replication-assembly cassette allowing the construct to be replicated in bacteria, at least one inducible promoter operably linked to a nucleic acid sequence encoding at least one Ff phage replication protein, wherein the expression construct expresses the Ff phage replication protein, and generates from the replication-assembly cassette, an excised and replicated DNA sequence which forms a circular single-stranded DNA encapsulated within nanorods.

In some embodiments the nucleic acid construct comprises a BSFnano replication assembly construct or variant thereof as described herein. The replication-assembly construct can express the Ff phage protein and generate an excised and replicated DNA sequence from the replication-assembly cassette, which forms a circular single-stranded DNA encapsulated within nanorods. Excision occurs by cleavage within (+) ori1 and within (+) ori2. Thus, the excised and replicated sequence from the replication assembly cassette (herein named scaffold; see FIG. 6) includes the intervening sequence between the cleaved (+) ori1 and (+) ori2 flanked by residual portions of (+) ori1 and (+) ori2. In some embodiments, the NPS can also include a (−) on between the packaging signal and (+) ori2 to increase efficiency of nanorod production. In one embodiment the expression construct is a plasmid. In some embodiments the expression construct encodes at least one Ff phage replication protein which effects cleavage of (+) ori1 and (+) ori2. In some embodiments the expression construct encodes from one to all of each of Ff phage proteins pI-pXI. In one embodiment the Ff phage replication protein is pII. In one embodiment the NPS lacks a second nucleic acid construct encoding one more filamentous phage proteins.

In some embodiments, any Ff phage proteins pI to pXI not encoded by the nucleic acid expression construct can be encoded by a second expression construct that may be referred to as a helper construct. If the nucleic acid expression construct including the replication-assembly cassette encodes all Ff phage proteins pI-pXI, a helper construct is not needed. In some embodiments, any of pIII, pVI, pVII, pVIII, and pIX, whether encoded by the expression construct including the replication-assembly cassette or other helper construct, can be fused to a heterologous polypeptide. In a preferred embodiment, the nucleic acid expression construct including the replication-assembly cassette comprises a nucleic acid sequence encoding Ff phage replication protein pII, wherein the nucleic acid sequence encoding pII is operably linked to an inducible promoter. Induction of the promoter and consequent expression of pII initiates excision, replication and packaging of the scaffold DNA from the replication-assembly cassette.

In some embodiments, the expression construct including the replication assembly cassette comprises a sequence encoding Ff phage protein pVIII that includes an amber mutation to reduce toxicity of pVIII to bacterial cells. In some embodiments the expression construct also includes a nucleic acid sequence encoding a marker to facilitate selection of cells that have taken up the construct. In some embodiments the marker is an auxotrophic marker. In some embodiments the marker is not an auxotrophic marker. In some embodiments the replication assembly cassette includes a filler nucleic acid sequence between the (+) ori1 and the PS or between the PS and the (−) on (if present) or PS and (+) ori2 (if the (−) on is absent). In some embodiments the replication assembly cassette does not include a filler nucleic acid sequence. In some embodiments, the filler nucleic acid sequence encodes at least one filamentous phage protein. In some embodiments the filler nucleic acid sequence encodes pVII and pIX, which can result in increased production of nanorods. In some embodiments the filler nucleic acid sequence encodes pVII, pVIII and/or pIX. In some embodiments, the filler nucleic acid sequences encode heterologous proteins and/or peptides fused to pVII, pVIII or pIX. In some embodiments these fusions facilitate the display of long peptides. In addition to Ff phage proteins expressed in E. coli, filler nucleic acid sequences could be used to accommodate one more eukaryotic gene expression cassettes allowing expression in eukaryotic cells. In some embodiments the filler nucleic acid sequence further encodes a prokaryotic or eukaryotic protein of interest.

Specifically contemplated as embodiments of this aspect of the invention directed to an NPS are any and/or all of the embodiments set forth in the other aspects of the invention related to nanorod production systems (NPS), nanorods, nanorod conjugates, and methods of making nanorods as described herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, promoters, selectable markers, scaffold and filler nucleic acid sequences, replicated+strand cc ssDNAs, binding agents, detection moieties, and fusion proteins.

In another aspect the invention relates to a nanorod production system (NPS) comprising i) a nucleic acid expression construct comprising a replication-assembly cassette comprising a filamentous phage (+) ori1, packaging signal (PS) and an (+) ori2, and at least one plasmid origin of replication not located in the replication-assembly cassette allowing the construct to be replicated in bacteria, and ii) a helper nucleic acid expression construct (termed a “helper construct”) comprising at least one selective marker, and at least one inducible promoter operably linked to a nucleic acid sequence encoding at least one Ff phage protein wherein the helper nucleic acid construct expresses the Ff phage replication protein and generates an excised and replicated DNA sequence from the replication-assembly cassette, which forms a circular single-stranded DNA encapsulated within nanorods. In some embodiments the helper nucleic acid construct can express the Ff phage replication protein and generate an excised and replicated scaffold DNA sequence from the replication-assembly cassette, which forms a circular single-stranded DNA encapsulated within nanorods. This NPS operates similarly to the NPS described in the previous paragraph, but the replication assembly construct does not necessarily encode any Ff phage proteins. Rather the system includes a helper construct that encodes Ff phage protein(s) needed to form nanorods encapsulating the scaffold DNA. In some embodiments, a single helper construct encodes any and/or all of each of the Ff phage proteins pI-pXI, although it is possible to use multiple helper constructs which together can be expressed to supply all of the Ff phage proteins pI-pXI needed to form nanorods encapsulating the scaffold DNA. In some embodiments, the replication assembly cassette further comprises a (−) on between the packaging signal and (+) ori2. In one embodiment, the helper construct comprises a nucleic acid sequence encoding Ff phage replication protein pII operably linked to an inducible promoter such that on induction pII is expressed and initiates excision and replication of DNA from the replication-assembly cassette. In some embodiments, the replication-assembly cassette encodes a selectable marker to facilitate selection of cells comprising the construct. In one embodiment the selectable marker is an auxotrophic marker. In one embodiment the selection marker is not an auxotrophic marker.

Specifically contemplated as embodiments of this aspect of the invention directed to an NPS are any and/or all of the embodiments set forth in the other aspects of the invention related to nanorod production systems (NPS), nanorods, nanorod conjugates, and methods of making nanorods as described herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, promoters, selectable markers, scaffold and filler nucleic acid sequences, replicated+strand cc ssDNAs, binding agents, detection moieties, and fusion proteins.

In another aspect the invention relates to an isolated host cell comprising an NPS as described herein.

In another aspect, the invention relates to a method of producing nanorods comprising culturing isolated host cells comprising an NPS as described herein and supplying the host cells with an inducer to the inducible promoter at an optimal growth phase, whereby an Ff phage replication protein is expressed in the cells, generating an excised and replicated DNA sequence that forms a circular single-stranded DNA encapsulated within the nanorods. In one embodiment the optimal growth phase is determined by the optical density (OD₆₀₀) of the host cells. In one embodiment the Ff phage replication protein is pII.

Specifically contemplated as embodiments of this aspect of the invention directed to a method of producing nanorods are any and/or all of the embodiments set forth in the other aspects of the invention related to nanorod production systems (NPS), nanorods, nanorod conjugates, and methods of making nanorods as described herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, promoters, selectable markers, scaffold and filler nucleic acid sequences, replicated +strand cc ssDNAs, binding agents, detection moieties, and fusion proteins.

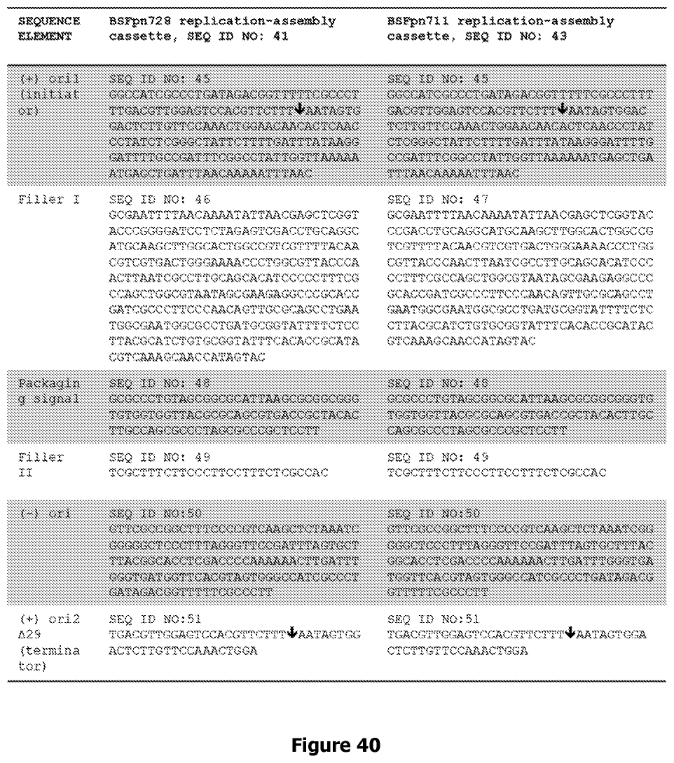

In another aspect the invention relates to a nanorod of length about 60-800 nm encapsulating a circular single stranded DNA termed scaffold, excised by pII cleavage of a replication-assembly cassette comprising a filamentous phage (+) ori1, packaging signal (PS) and a (+) ori2, and a filler nucleic acid sequence encoding at least one filamentous phage protein. In some embodiments the filler nucleic acid sequence is located between (+) ori1 and the PS (filler I; Table 9, SEQ NOs: 46, 47; FIG. 40; SEQ NOs: 56-59, FIG. 42; SEQ NOs: 66-67, FIG. 44: SEQ NO: 75, FIG. 46; SEQ NO: 82, FIG. 48; SEQ NO: 104, FIG. 57). In some embodiments the filler nucleic acid is located between the PS and (+) ori2. In some embodiments the replication assembly cassette comprises a (−) on between the PS and the (+) on 2. In some embodiments the filler nucleic acid is located between the PS and the (−) on (filler II; Table 9, SEQ NO: 49, FIGS. 40, 42, 44, 57; SEQ NO: 85, 86, FIG. 48).

These nanorods differ from previously described nanorods in that the filler DNA is used to encode at least one Ff protein. The presence of the protein-encoding genes in the filler DNA increases the minimum length of the nanorod proportionally to the number of added nucleotides, as the length of the nanorods correlates linearly to the distance between the pII cut sites in (+) ori1 and (+) ori2. Each nucleotide added to the ssDNA genome increases the length of the nanorod by 0.133 nm (Newman et al., 1977). The upper length limit can be any of the upper limits mentioned above depending on the length of the filler DNA. The length of the filler DNA depends on how many Ff proteins it encodes as well as how much, if any, other filler DNA is present. Such nanorods can be produced from a replication assembly cassette with or without a (−) ori between the PS and (+) ori2. If a (−) ori is present the filler 2 position is between PS and the (−) ori. If a (−) ori is present in the replication assembly cassette, it is also present in the excised and replicated DNA included in nanorods. In some embodiments, the filler DNA encodes Ff phage protein pVII and/or pIX, which has been found to increase production of nanorods. A preferred length of such nanorods is about 95-125 nm. In some embodiments, pVIII is encoded by a filler nucleic acid sequence. In some embodiments, the filler nucleic acid sequence encodes Ff phage proteins pVII, pVIII and/or pIX or encodes modified Ff phage proteins pVII, pVIII and/or pIX or a combination thereof. In some embodiments the nucleic acid sequence encoding the pVII, pVIII and/or pIX and/or the modified pVII, pVIII and/or pIX is fused to a nucleic acid sequence encoding a heterologous polypeptide. In some embodiments the filler nucleic acid sequence further encodes a heterologous polypeptide that may or may not be fused to a Ff phage protein or modified Ff phage protein. A preferred length of such nanorods is about 95-125 nm.

In another aspect the invention relates to a population of nanorods encapsulating a circular single stranded DNA termed scaffold excised by pII cleavage of a replication-assembly cassette comprising a filamentous phage (+) ori1, packaging signal (PS), a (−) ori and a (+) ori2, and a filler nucleic acid sequence between (+) ori1 and the PS or between the PS and (+) ori2, the filler nucleic acid sequence encoding at least one filamentous phage protein, wherein at least 70% of nanorods in the population are about 40 to about 800 nm in length. In one embodiment the replication assembly cassette further comprises a (−) ori between the packaging signal and (+) ori2, wherein at least 70% of nanorods in the population are about 60 to about 800 nm in length. In one embodiment at least 70% of the nanorods in the population are about 60 to about 400 nm in length. In one embodiment at least 70% of the nanorods in the population are about 60 to about 300 nm in length. In one embodiment at least 70% of the nanorods in the population are about 95 to about 125 nm in length.

In another aspect the invention relates to a nanorod encapsulating a circular single stranded DNA termed scaffold, excised by pII cleavage of a replication-assembly cassette comprising a filamentous phage (+) ori1, packaging signal (PS) and a (+) ori2, and lacking a (−) ori. The lack of (−) ori results in such nanorods have a smaller minimal size, e.g., less than 50 nm down to about 40 nm than previously described nanorods.

However, such nanorods can also have any of the upper size limits described above depending on the length of filler DNA included between (+1) ori1 and (+) ori2. Thus, the invention provides a population of nanorods in which at least 70% of nanorods in the population have a length of 40-800 nm. The invention also provides a population of nanorods in which at least 70% of nanorods in the population have a length of 40-50 nm.

In another aspect the invention relates to a nanorod of about 35 to about 45 nm in length encapsulating a circular single stranded DNA excised by pII cleavage of a replication-assembly cassette comprising a filamentous phage (+) ori1, packaging signal (PS) and a (+) ori2, and lacking a (−) ori.

In another aspect the invention relates to a population of nanorods comprising a plurality of nanorods of about 35 to about 45 nm in length encapsulating a circular single stranded DNA excised by pII cleavage of a replication-assembly cassette comprising a filamentous phage (+) ori1, packaging signal (PS) and a (+) ori2, and lacking a (−) ori, wherein at least 70% of nanorods in the population are about 38 to about 42 nm in length. In one embodiment at least 70% of nanorods in the population have a length of about 40 nm.

Specifically contemplated as embodiments of the aspects of the invention directed to nanorods and/or populations of nanorods are any and/or all of the embodiments set forth in the other aspects of the invention related to nanorod production systems (NPS), nanorod conjugates, and methods of producing and/or making nanorods as described herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, promoters, selectable markers, scaffold and filler nucleic acid sequences, replicated+strand cc ssDNAs, binding agents, detection moieties, and fusion proteins.

In another aspect the invention relates to a method of making a plurality of nanorods, the method comprising inducing the production of at least 1.0×10¹³ nanorods/L of host cell culture.

In one embodiment the method comprises inducing the production of at least 1.0×10¹⁴, preferably at least 1.0×10¹⁵ nanorods/L. In one embodiment the method comprises inducing the production of about 1.0×10¹⁴, preferably about 1.0×10¹⁵, preferably about 1.0×10¹⁶ nanorods/L. In one embodiment the method comprises inducing the production of 1.0×10¹⁴, preferably 1.0×10¹⁵, preferably 1.0×10¹⁶ nanorods/L.

In one embodiment the host cell culture is a eukaryotic cell culture, or a prokaryotic cell culture. In one embodiment the prokaryotic cell culture is a bacterial cell culture. In one embodiment the bacterial cell culture is a gram (−) bacterial cell culture. In one embodiment the gram (−) bacterial cell culture is an E. coli culture.

In one embodiment the E. coli culture comprises at least 1.0×10¹¹ cells/L, preferably at least 1.0×10¹² per L, at least 2.0×10¹² cells/L, at least 3.0×10¹² cells/L, at least 4.0×10¹² cells/L, preferably at least 5.0×10¹² cells/L.

In one embodiment the E. coli culture comprises about 1.0×10¹¹ cells/L, preferably about 1.0×10¹² per L, about 2.0×10¹² cells/L, about 3.0×10¹² cells/L, about 4.0×10¹² cells/L, preferably about 5.0×10¹² cells/L.

In one embodiment the E. coli cells comprise a mutation that allows the suppression of the stop codons within at least one Ff phage coat protein. Preferably the mutation is in Ff phage gene gVIII as described herein. Preferably the coat protein is pVIII.

In one embodiment the E. coli cells comprise a mutation that inhibits the background expression from an inducible promoter. In one embodiment the inducible promoter is any inducible promoter as described herein for the aspects of the invention set forth above. Preferably the inducible promoter is a lac promoter, preferably lacUV5.

In one embodiment the E. coli cells are strain K2091 (Table 1).

In one embodiment the E. coli cells are strain K2485 (Table 1).

In one embodiment the E. coli cells comprise at least one, preferably two auxotrophic mutations. In one embodiment the auxotrophic mutations are ΔnadC727 and ΔmetE774.

The ΔnadC727 mutation allows auxotrophic selection of plasmids expressing NadC in the minimal media supplemented with casamino acids (casein hydrolysate) the absence of NAD.

ΔmetE774 mutation allows auxotrophic selection of plasmids expressing MetE in the minimal media in the absence of methionine. This mutation also allows in vivo incorporation of artificial amino acid azidohomoalanine (Aha) into the proteins at the ATG codons in the minimal media containing a specific mix of Methionine and Aha.

In one embodiment induction comprises contacting the E. coli cells with an inducer. In one embodiment the inducer is an inducer of a lac promoter, preferably a mutant lac promoter, preferably lacUV5. In one embodiment the inducer is IPTG.

In one embodiment method comprises inducing nanorod production in the E. coli cells at an optimal growth phase. In one embodiment the optimal growth phase is determined by the optical density (OD600) of the E. coli cells in the culture.

In one embodiment the optimal growth phase is determined by an OD600 of at least 0.1., 0.11, 0.12, 0.13, 0.14, 0.15, 0.16, 0.17, 0.18, 0.19, 0.2, 0.21 or 0.22, preferably at least 0.1. In one embodiment the optimal growth phase is determined by an OD600 of about 0.09 to about 0.22, preferably of about 0.1 to about 0.2, preferably of 0.1 to 0.2.

In one embodiment the optimal growth phase is determined by an OD600 of about 0.1.

In one embodiment the optimal growth phase is determined by an OD600 of about 0.15.

In one embodiment the optimal growth phase is determined by an OD600 of or about 0.2. In one embodiment the optimal growth phase is determined by an OD600 of 0.1. In one embodiment the optimal growth phase is determined by an OD600 of 0.15. In one embodiment the optimal growth phase is determined by an OD600 of or 0.2.

In one embodiment induction results in replication of (+) strand circular ssDNA that comprises the nucleic acid coding sequences for at least one, preferably two Ff phage coat proteins or modified coat proteins or both. In one embodiment induction results in the expression of at least one, preferably two Ff phage coat proteins or modified coat proteins that bind to the (+) strand circular ssDNA.

In one embodiment the two Ff phage coat proteins or modified coat proteins are pVII and pIX.

Specifically contemplated as embodiments of pVII and pIX and modified pVII and pIX within this method aspect of the invention are all of the embodiments of pVII and pIX and modified pVII and pIX as set out in the previous aspects of the invention directed to NPS aspects of the invention.

In one embodiment induction results in replication of (+) strand circular ssDNA that binds at least one, preferably at least two, preferably at least three different Ff phage coat proteins and/or different modified Ff phage coat proteins. In one embodiment the at least one, two, or three different Ff phage coat proteins and/or one, two or three different modified Ff phage coat proteins are selected from the group consisting of pVIII, pIII, pVII, pIX and pVI.

In one embodiment the E. coli cells comprise a single nucleic acid construct that mediates the production of the nanorods. In one embodiment the single nucleic acid construct is a vector, preferably a plasmid, as described herein. In one embodiment the single nucleic acid is a pPop-up plasmid as described herein.

In one embodiment inducing the production comprises a single transformation of the E. coli cells only. In one embodiment the single transformation comprises transforming the E. coli cells with a single nucleic acid construct only. In one embodiment the single nucleic acid construct mediates the production of the nanorods. In one embodiment the single nucleic acid construct is a vector, preferably a plasmid, as described herein. In one embodiment the single plasmid is a pPop-up plasmid as described herein.

In one embodiment transformation of the E. coli cells with the single nucleic acid construct results in at least 10×, preferably at least 100× more transformed E. coli cells compared to transformation of the E. coli cells with dual nucleic acid constructs.

In one embodiment the single nucleic acid construct is a vector, preferably a plasmid, preferably a pPop-up plasmid as described herein.

Specifically contemplated as embodiments of the single nucleic acid expression construct are all of the embodiments of the single nucleic acid expression construct comprising the BSFnano replication-assembly cassette, the at least one auxotrophic marker, the at least one inducible promoter operably linked to a nucleic acid sequence encoding at least one Ff phage protein, and the at least one plasmid origin of replication not located in the BSFnano replication-assembly cassette that are set forth above in the first NPS aspect of the invention.

In one embodiment inducing the production comprises a dual transformation of the E. coli cells only. In one embodiment the dual transformation comprises transforming the E. coli cells with a nucleic acid replication-assembly construct and a helper nucleic acid expression construct as described herein. In one embodiment the dual nucleic acid constructs mediate the production of the nanorods. In one embodiment the dual nucleic acid constructs are vectors, preferably plasmids as described herein. In one embodiment the dual plasmids are the pBSF and pHP plasmid series as described herein.

In one embodiment the dual nucleic constructs are vectors, preferably plasmids, preferably plasmids of the pBSF and pHP series as described herein.

In one embodiment the dual nucleic acid constructs are different nucleic acid constructs.

In one embodiment dual transformation is sequential transformation with the different nucleic acid constructs wherein a first transformation is separated from a second transformation by at least 24h, preferably at least 32h, 40h, preferably at least 48h. In one embodiment dual transformation is sequential transformation with the different nucleic acid constructs wherein a first transformation is separated from a second transformation by about 24h, preferably about 32h, 40h, preferably about 48h.

In one embodiment the method comprises preparing transformation competent cells from cells that have undergone the first transformation.

In one embodiment the first transformation comprises transformation with a helper nucleic acid expression construct comprising at least one selective marker and at least one inducible promoter operably linked to a nucleic acid sequence encoding at least one Ff phage protein.

Specifically contemplated as embodiments of the helper nucleic acid expression construct are all of the embodiments relating to ii) a helper nucleic acid expression construct as set forth above in the second NPS aspect of the invention.

In one embodiment the second transformation comprises transformation with a nucleic acid replication-assembly construct comprising a BSFnano replication-assembly cassette, at least one auxotrophic marker, and at least one plasmid origin of replication not located in the BSFnano replication-assembly cassette.

Specifically contemplated as embodiments of the nucleic acid replication-assembly construct are all of the embodiments relating to i) a nucleic acid replication-assembly construct as set forth above in the second NPS aspect of the invention.

In one embodiment at least 70%, at least 75%, preferably at least 80% of the nanorods are about 40 nm to about 1000 nm in length, preferably about 40 nm to about 400 nm in length, preferably about 100 nm to 300 nm in length. In one embodiment at least 70%, at least 75%, preferably at least 80% of the nanorods are at least 40 nm, preferably at least 50 nm (FIG. 17), 60 nm, 70 nm, 80 nm (FIG. 19), 100 nm, 110 nm, 200 nm, 300 nm, 400 nm, 500 nm, 600 nm, 700 nm, 800 nm, 900 nm, 1000 nm, 2000 nm, 3000 nm, 4000 nm, 5000 nm, or at least 6000 nm in length. In one embodiment at least 70% of the nanorods are about 40 nm, preferably about 50 nm (FIG. 17), 60 nm, 70 nm, 80 nm (FIG. 19), 100 nm, 110 nm, 200 nm, 300 nm, 400 nm, 500 nm, 600 nm, 700 nm, 800 nm, 900 nm, 1000 nm, 2000 nm, 3000 nm, 4000 nm, 5000 nm, or 6000 nm in length. In one embodiment at least 70%, at least 75%, preferably at least 80% of the nanorods are 40 nm, 50 nm (FIG. 17), 60 nm, 70 nm, 80 nm (FIG. 19), 100 nm, 110 nm, 200 nm, 300 nm, 400 nm, 500 nm, 600 nm, 700 nm, 800 nm, 900 nm, 1000 nm, 2000 nm, 3000 nm, 4000 nm, 5000 nm, or 6000 nm in length. In one embodiment, at least 70%, at least 75%, preferably at least 80% of the nanorods are 40 nm in length.

In one embodiment at least 70%, at least 75%, preferably at least 80% of the nanorods are at least 40 nm, 50 nm (FIG. 17), 60 nm, 70 nm, or 80 nm (FIG. 19) in length. In one embodiment at least 70%, at least 75%, preferably at least 80% of the nanorods are about 40 nm, 50 nm (FIG. 17), 60 nm, 70 nm, or 80 nm (FIG. 19) in length. In one embodiment at least 70%, at least 75%, preferably at least 80% of the nanorods are 40 nm, 50 nm (FIG. 17), 60 nm, 70 nm, or 80 nm (FIG. 19) in length. In one embodiment at least 70%, at least 75%, preferably at least 80% of the nanorods are at least 200 nm, 300 nm, 400 nm, 500 nm, 600 nm, 700 nm, 800 nm, 900 nm or at least 1000 nm in length. In one embodiment at least 70%, at least 75%, preferably at least 80% of the nanorods are about 200 nm, 300 nm, 400 nm, 500 nm, 600 nm, 700 nm, 800 nm, 900 nm or about 1000 nm in length. In one embodiment at least 70%, at least 75%, preferably at least 80% of the nanorods are 200 nm, 300 nm, 400 nm, 500 nm, 600 nm, 700 nm, 800 nm, 900 nm, or 1000 nm in length.

In one embodiment at least 70%, at least 75%, preferably at least 80% of nanorods are about 80 nm in length (FIG. 19). In one embodiment at least 70%, at least 75%, preferably at least 80% of nanorods are 80 nm in length. In one embodiment at least 70%, at least 75%, preferably at least 80% of nanorods are about 100 nm, 110 nm, 200 nm, or 300 nm in length. In one embodiment at least 70%, at least 75%, preferably at least 80% of nanorods are 100 nm, 110 nm, 200 nm, or 300 nm in length.

In one embodiment the nanorods comprise a (+) strand circular ssDNA that comprises an Ff phage origin of replication. In one embodiment the nanorods comprise an (+) strand circular ssDNA that does not comprise a selective marker. In one embodiment the nanorods comprise an (+) strand circular ssDNA that does not comprise an antibiotic resistance marker.

In one embodiment the nanorods comprise a (+) strand circular ssDNA that encodes at least one, preferably at least two Ff phage coat proteins as described herein.

In some embodiments the nanorods comprise at least one modified Ff phage protein as described herein. In one embodiment the nanorods comprise at least one fusion protein as described herein in the above first and second NPS aspects of the invention.

In another aspect the invention relates to a method of making a plurality of nanorods comprising inducing replication of a circular ssDNA in a host cell culture from a single nucleic acid construct, the construct comprising a scaffold nucleic acid sequence encoding at least two Ff phage coat proteins or modified Ff phage coat proteins.

In one embodiment the scaffold nucleic acid sequence (FIG. 41, SEQ ID NO: 53, SEQ ID NO: 55; FIG. 42, SEQ ID NO: 59,) encodes pVII (SEQ ID NO: 8, FIG. 31) and pIX (SEQ ID NO: 10, FIG. 31).

In one embodiment the amino acid sequence of pVII comprises, consists, or consists essentially of SEQ ID NO: 7 (FIG. 31). In one embodiment the nucleic acid sequence encoding pVII comprises at least 70%, 80%, 90%, 95% or 99% sequence identity with SEQ ID NO: 8 (FIG. 31). In one embodiment the nucleic acid sequence encoding pVII comprises, consists, or consists essentially of SEQ ID NO: 8 (FIG. 31).

In one embodiment the amino acid sequence of pIX comprises, consists, or consists essentially of SEQ ID NO: 9. (FIG. 31). In one embodiment the nucleic acid sequence encoding pIX comprises at least 70%, 80%, 90%, 95% or 99% sequence identity with SEQ ID NO: 10 (FIG. 31). In one embodiment the nucleic acid sequence encoding pIX comprises, consists, or consists essentially of SEQ ID NO: 10 (FIG. 31).

Specifically contemplated as embodiments of this aspect of the invention are all of the embodiments set forth in the NPS, nanorod, composition and method aspects herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, including amino acid and nucleic acid sequences and all contemplated variations and modifications, promoters, inducible promoters and operable linkages, selective markers, auxotrophic markers, (+) strand circular ssDNAs, fusion proteins, induction of production, host cells and host cell cultures, replicated ssDNAs, single and dual transformations, lengths of nanorods produced, and (+) strand circular ssDNAs.

In another aspect the invention relates to a method of making a plurality of nanorods comprising inducing the replication of a (+) strand circular ssDNA from a single nucleic acid construct comprising
a BSFnano replication-assembly cassette,
a scaffold nucleic acid sequence,
two nucleic acid sequences comprising a pII cut site each, each cut site located in a (+) ori, and
at least one nucleic acid sequence encoding at least one modified Ff phage coat protein.

In one embodiment the nucleic acid construct comprises a nucleic acid sequence encoding at least two modified Ff phage proteins. In one embodiment at least one modified Ff phage protein is a modified coat protein as described herein. In one embodiment at least one modified Ff phage protein is a modified replication protein as described herein. In one embodiment the single nucleic acid construct comprises Ff phage protein pII operably linked to an inducible promoter.

As the skilled worker will appreciate, the scaffold nucleic acid sequence corresponds to the sequences between the vertical arrows as shown in FIGS. 39, 41, 43, 45, 47.

In one embodiment the single nucleic acid construct comprises SEQ ID NO: 41, SEQ ID NO: 43 (FIG. 39), SEQ ID NO: 52, SEQ ID NO: 54 (FIG. 41), SEQ ID NO: 61, SEQ ID NO: 63 (FIG. 43); SEQ ID NO: 70, SEQ ID NO: 72 (FIG. 45); SEQ ID NO: 77, or SEQ ID NO: 79 (FIG. 47); SEQ ID NO: 101 (FIG. 56).

In one embodiment inducing replication of the (+) strand circular ssDNA is inducing replication from a nucleic acid construct comprising SEQ ID NO: 41, SEQ ID NO: 43 (FIG. 39), SEQ ID NO: 52, SEQ ID NO: 54 (FIG. 41), SEQ ID NO: 61, SEQ ID NO: 63 (FIG. 43); SEQ ID NO: 70, SEQ ID NO: 72 (FIG. 45); SEQ ID NO: 77, SEQ ID NO: 79 (FIG. 47) or SEQ ID NO: 101 (FIG. 56).

In one embodiment the (+) strand circular ssDNA comprises, consists essentially of or consists of SEQ ID NO: 42, SEQ ID NO: 44 (FIG. 39), SEQ ID NO: 53, SEQ ID NO: 55 (FIG. 41), SEQ ID NO: 62, SEQ ID NO: 64 (FIG. 43); SEQ ID NO: 71, SEQ ID NO: 73 (FIG. 45); SEQ ID NO: 78, SEQ ID NO: 80 (FIG. 47), or SEQ ID NO: 102 (FIG. 56). Each of these (+) strand circular ssDNAs is set out in, defined by, and located between, the arrows shown in each of FIGS. 39-47 and 56, the arrows indicating pII cut sites.

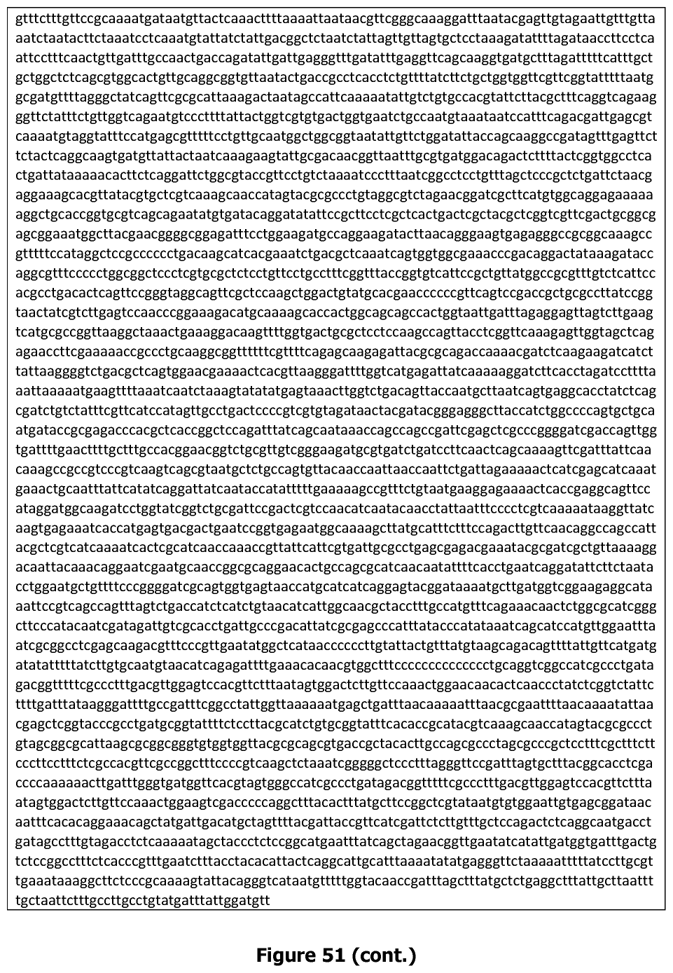

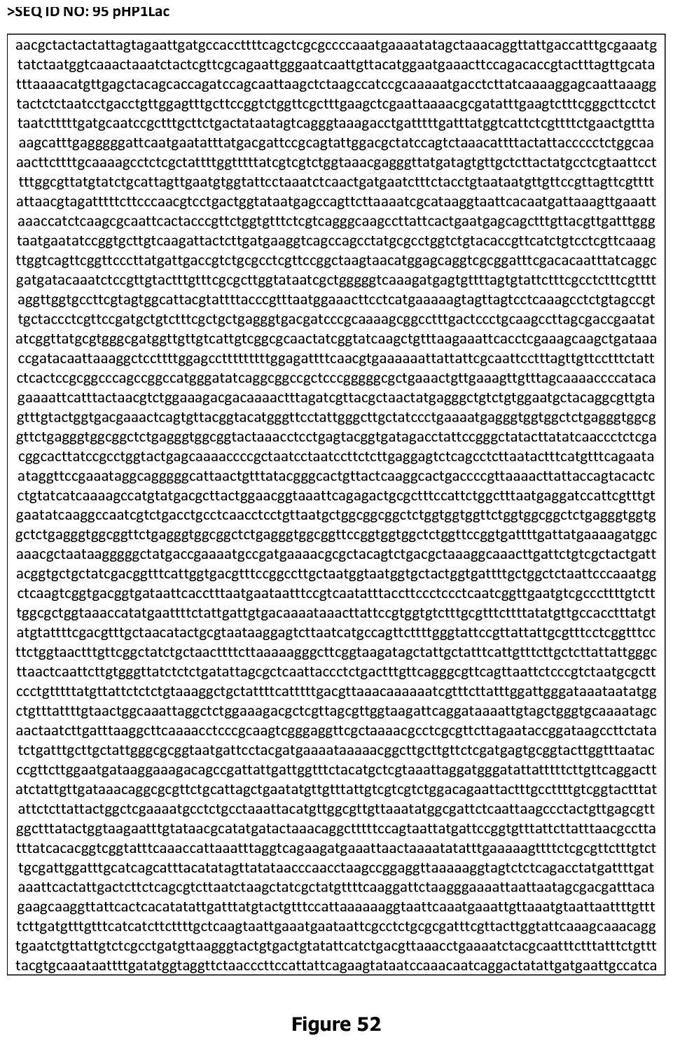

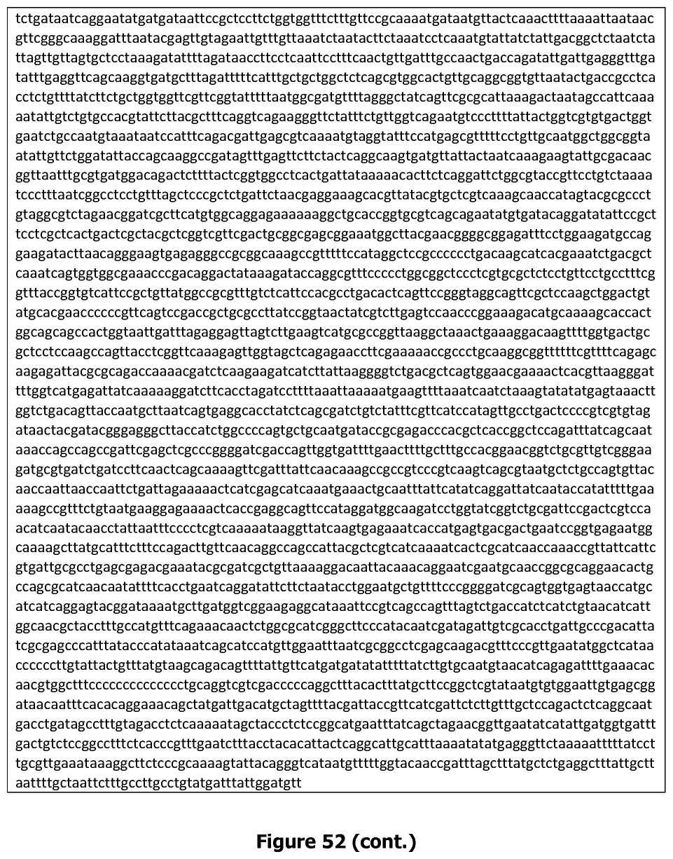

In one embodiment the single nucleic acid construct is pPop-up529LacYM (SEQ ID NO: 94, FIG. 51).

Specifically contemplated as embodiments of this method aspect of the invention are all of the embodiments set forth in the NPS, composition, nanorod and method aspects herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, including amino acid and nucleic acid sequences and all contemplated variations and modifications, promoters, inducible promoters and operable linkages, selective markers, auxotrophic markers, (+) strand circular ssDNAs, fusion proteins, induction of production, host cells and host cell cultures, replicated ssDNAs, single and dual transformations, lengths of nanorods produced, and (+) strand circular ssDNAs.

In another aspect the invention relates to a method of making a nanorod—binding agent conjugate comprising conjugating a binding agent to a nanorod as described herein or produced from an NPS as described herein.

In one embodiment conjugating comprises the formation of at least one covalent bond between an amino acid residue comprised in the nanorod and the binding agent.

In one embodiment the binding agent is selected from the group consisting of small molecules or polypeptides (e.g., biotin, antibodies, antibody-derived single-chain variable domain (scFv), nanobodies, camelid heavy-chain only antibodies or variable domain (VHH) or other types of analyte-binding polypeptides).

In one embodiment the conjugate further comprises a detection agent.

In one embodiment the detection agent is selected from the group consisting of small molecules, biotin, fluorophores, quantum dots, inorganic molecules, metal alloys, fluorescent or colored proteins, and enzymes that catalyse chromogenic reactions.

In one embodiment the nanorods comprise a (+) strand ssDNA that comprises an Ff phage origin of replication. In one embodiment the nanorods comprise an (+) strand ssDNA that does not comprise a selective marker. In one embodiment the nanorods comprise a (+) strand ssDNA that does not comprise an antibiotic resistance marker.

In one embodiment the nanorods comprise a (+) strand ssDNA that encodes at least one, preferably at least two Ff phage coat proteins as described herein. In some embodiments the nanorods comprise at least one modified Ff phage protein as described herein. In one embodiment the nanorods comprise at least one fusion protein as described herein.

In another aspect the invention relates to a method of making a nanorod—detection agent conjugate comprising conjugating a detection agent to a nanorod as described herein or produced from an NPS as described herein.

In one embodiment conjugating comprises the formation of at least one covalent bond between an amino acid residue comprised in the nanorod and the detection agent.

In one embodiment the detection agent is selected from the group consisting of small molecules, biotin, fluorophores, quantum dots, inorganic molecules, metal alloys, fluorescent or colored proteins, and enzymes that catalyse chromogenic reactions.

In one embodiment the conjugate further comprises a binding agent.

In one embodiment the binding agent is selected from the group consisting of small molecules or polypeptides (e.g., biotin, antibodies, antibody-derived single-chain variable domain (scFv), nanobodies, camelid heavy-chain only antibodies or variable domain (VHH) or other types of analyte-binding polypeptides).

In one embodiment the nanorods comprise a (+) strand ssDNA that comprises an Ff phage origin of replication. In one embodiment the nanorods comprise an (+) strand ssDNA that does not comprise a selective marker. In one embodiment the nanorods comprise a (+) strand ssDNA that does not comprise an antibiotic resistance marker.

In one embodiment the nanorods comprise a (+) strand ssDNA that encodes at least one, preferably at least two Ff phage coat proteins as described herein. In some embodiments the nanorods comprise at least one modified Ff phage protein as described herein. In one embodiment the nanorods comprise at least one fusion protein as described herein.

The following aspects relate to both nanorod-binding agent and nanorod detection agent conjugates. In one embodiment the nanorods comprise modifications to the Ff phage coat-proteins that create functionalization handles. Such modifications are known as “tag and modify” modifications, are made to allow targeted chemical or enzymatic modification of the Ff phage coat proteins. For example, engineering pVIII containing extra ≥3 Glycines or ≥2 Alanines at the N-terminus of the mature coat protein pVIII or pIII or pVII and pIX (addition of heterologous signal sequence may be required for the two latter proteins) creates a motif that can be used for enzymatic attachment of protein or non-protein molecules conjugated to C-terminal LPXTA or LPXTG motifs, where the attachment of a molecule of interest is catalyzed by the enzyme sortase A (SrtA) of Streptococcus pyogenes (SrtA Sp) or Staphylococcus aureus (SrtA Sa), respectively (Hess et al., 2012). Exchangeable blocks (FIG. 7, Block iv; FIG. 8, Block iii) have been generated for our NPS that produce nanorods with pVIII displaying, at the N-terminus, 4 Gly residues, or 2 Ala residues (FIGS. 30-32 and SEQ NOs: 19-23; 27-28) or 5 residues (FIG. 54, SEQ NOs: 97-98).

In some embodiments, the reactive groups of amino acids, such as the amine groups of the N-terminal residues, lysines, cysteines, tyrosines, aspartic acids, and glutamic acids can be used for chemical modification (Bernard and Francis, 2014). Alternatively, other motifs that are subject to enzymatic or chemical covalent attachment to non-protein molecules, such as SNAP-tag, be directly or indirectly inserted into the nanorods, to allow attachment of a diverse array of molecules. Also described herein are exchangeable blocks have been generated that display unpaired Cys residues on pIII, to allow modifications by maleimide-conjugated proteins and small molecules or other chemistries targeting —SH groups (FIG. 7, Block iv; FIG. 8, Block iii; FIG. 36, SEQ NOs: 33-34).

The skilled worker will appreciate that a pVIII variant comprising exposed Met residues (Ala9 mutated to Met) and buried Met28 residue mutated to Leu (FIG. 33; SEQ ID NOs: 23, 24) allows for in vivo incorporation of unnatural amino acid azidohomoalanine (Aha) into an surface-exposed position on pVIII without disturbance of the virion assembly and structure (Petrie, 2015). Aha contains azide group in its side-chain, allowing attachment into the virion of small molecules using click chemistry which targets azide groups. In some embodiments the nanorods described herein comprise such modifications.

The skilled worker will appreciate that all of the known modifications applied in the Ff-based phage display and material science applications can also be applied for functionalization of nanorods as described herein. All of such modifications are contemplated as embodiments herein. In one embodiment, the insertion of 4 Gly residues at the N-terminus of mature pVIII that we constructed (FIG. 33, SEQ ID NO 27, SEQ ID NO 28), results in a minor drop in the nanorod production. In contrast, insertion of Ala followed by Gly residue between Ala1 and Gly2 and deletion of Pro6 of the wild-type mature pVIII at the N-terminus of the mature pVIII that we constructed (FIG. 32, SEQ ID NO 17, SEQ ID NO 18) results in the interference of nanorod production. To overcome this latter problem, we “evolved” the gVIII sequence to increase efficiency of this functionalized pVIII variant. This was achieved by transferring the coding sequence into the backbone of an Ff phage (VCSM13). The resulting modified phage gave very small plaques and low titres, however three rounds of phage growth where the host cells were infected at a low multiplicity of infection (1 phage to 1000 E. coli cells) resulted in the appearance of “large-plaque” mutants. Sequencing of gVIII from two evolved phage identified two compensatory mutations, one in each mutant (D5A and L27S as described herein above; FIGS. 32, 33; SEQ ID NO 19, SEQ ID NO 20, SEQ ID NO 21, SEQ ID NO 22). These alleles were transferred back into the inducible pPop-up an pHP plasmid backbones and showed to give rise to the BSF nanorods. As will be appreciated by the skilled person, the inventors believe that it is possible to evolve the coding sequences of various Ff phage proteins to allow other modifications that may interfere with the BSF nanorod assembly.

In one embodiment of enzymatic modification, BSF nanorods were produced that contain the evolved pVIII (SEQ NOs: 19, 20) displaying AlaAlaGlyGly motif on each pVIII copy along the nanorod. They were further enzymatically modified with LPETA-(Leu Pro Glu Thr Ala)-tagged fluorescent dye FITC or the small molecule biotin via enzymatic attachment using S. pyogenes Sortase (SrtA Sp; FIG. 21). Analysis by native virion electrophoresis showed high intensity fluorescence corresponding to the nanorod band after the LPTA-FITC enzymatic conjugation (FIG. 22A). Analysis of enzymatically biotinylated nanorods by transmission electron microscopy using avidin-coated gold beads shows Sortase-dependent binding along the length of nanorods (FIG. 23). For immunodetection assays avidin-alkaline phosphatase may be attached to nanorods (FIG. 24A; 25-27). Enzymatic visualization of such avidin-alkaline phosphatase labeled nanorods was carried out by native agarose gel electrophoresis, blotted onto a membrane and detected using a chromogenic substrate (FIG. 24A).

In another embodiment, LPETG-β-glucosidase (GUS) was enzymatically attached directly to the nanorods displaying N-terminal 5-Gly peptide. Attachment of GUS to the nanorods was analysed by agarose gel electrophoresis followed by in-gel assay using a chromogenic substrate (FIG. 24B).
The copy number and position of displayed functionalities, be it heterologous proteins or “handles” for modifications, depends on the coat protein that is used as the platform. In some embodiments, use of pVIII as a platform for display allows high-copy-number of displayed peptide along the shaft of the nanorod. The copy number of displayed functionalities depends on the number of pVIII subunits per nanorod, which in turn depends on the length of the ssDNA scaffold. The copy number of functionalities (fluorescent dyes, small molecules, polymers and/or enzymes) depends on the length of the nanorod. For example, for a phage that is 1,000 nm in length it is expected that ˜400 copies of a fluorophore or biotin per phage nanorod may be attached, whether chemically or enzymatically. More than one different fluorescent dye can be mixed for the purpose of labelling to allow bar-coding or other more complex methods or detection.

In some embodiments, the use of the minor coat proteins as platforms allows display of up to 5 copies per nanorod (for each pIII, pVII and pIX; reviewed in (Rakonjac et al., 2017). Furthermore, display on both pVII and pIX allows up to 10 copies per nanorod. In some embodiments contemplated herein are different fusions or attached molecules to different minor Ff phage coat proteins. In this manner a number of different functionalities can be displayed on a single nanorod, such as with two functionalities being displayed at one end of the nanorod (the pVII-pIX end) and one functionality being displayed at the other (at the pIII end). Such modifications have been demonstrated in various methods of phage display using the full-length Ff phage.

In another embodiment, the toxicity of the major coat protein pVIII has been overcome by introduction of amber mutations. Major coat protein pVIII is toxic to E. coli when expressed in the absence of phage assembly. This toxicity leads to mutations that remove the gVIII promoter in the course of cloning, or in poor growth of transformed E. coli cells expressing pVIII, even when expression is controlled by an inducible promoter.

To overcome this problem, gVIII suppressible (nonsense) mutants were used to construct helper plasmids. Construction was carried out in an E. coli host that does not contain a suppressor mutation, thereby preventing translation of most of the pVIII protein. Two different amber (TAG) mutants were used, one containing a G to T mutation that converted the GAG codon 25 encoding Glutamic acid at position two of the mature protein to TAG (SEQ NOs: 13-24, FIGS. 32-33), and one where TCT codon 4 for Serine within the signal sequence was replaced with TAG (SEQ NOs: 25-28, FIG. 33). A suppressor D mutation (supD) of the serine tRNA was used to suppress these two amber mutations, with an E. coli strain containing this mutation used for nanorod production (Table 1).

An additional advantage of the gVIII suppressed amber mutants described herein as compared to E. coli cell expressing wild-type gVIII is seen in a decrease of pVIII produced in the cells due to the lower translation efficiency of suppressor in comparison to the cognate tRNA reading the sense codons, favoring assembly of short over long nanorods by decreasing the ratio of the shaft protein pVIII vs. end-cap proteins pIII, pVI, pVII and pIX.

Specifically contemplated as embodiments of this aspect of the invention are all of the embodiments set forth in the NPS, composition, nanorod and method aspects herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, including amino acid and nucleic acid sequences and all contemplated variations and modifications, promoters, inducible promoters and operable linkages, selective markers, auxotrophic markers, (+) strand circular ssDNAs, fusion proteins, induction of production, host cells and host cell cultures, replicated ssDNAs, single and dual transformations, lengths of nanorods produced, and (+) strand circular ssDNAs.

In another aspect the invention relates to a nanorod-binding agent conjugate comprising a nanorod comprising at least one modified Ff phage coat protein, wherein the nanorod is produced from an NPS as described herein, or is a nanorod as described herein or is made by a method of making a nanorod as described herein.

In one embodiment the nanorod-binding agent conjugate comprises at least one detection moiety that allows detection of the nanorod-binding agent conjugate.

In another aspect the invention relates to a composition comprising a nanorod-binding agent conjugate as described herein.

Specifically contemplated as embodiments of these aspects of the invention are all of the embodiments set forth in the NPS, composition, nanorod and method aspects herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, including amino acid and nucleic acid sequences and all contemplated variations and modifications, promoters, inducible promoters and operable linkages, selective markers, auxotrophic markers, (+) strand circular ssDNAs, fusion proteins, induction of production, host cells and host cell cultures, replicated ssDNAs, single and dual transformations, lengths of nanorods produced, nanorod-binding agent conjugates and (+) strand ssDNAs.

In another aspect the invention relates to a nanorod-detection agent conjugate comprising a nanorod comprising at least one Ff phage coat protein comprising a covalently bound detection moiety, wherein the nanorod is a nanorod as described herein, produced from an NPS as described herein or made by a method as described herein.

In one embodiment the Ff phage protein is a modified coat protein as described herein.

In one embodiment the detection moiety allows detection of the nanorod-detection agent conjugate.

In one embodiment detection is by detecting a chemical, spectral, linear-dichroic, fluorescence, visual, chemiluminescence, paramagnetic, sound, electrical, surface plasmon resonance, isotopic, radioactive or other chemical or physical signal.

In one embodiment the nanorod-detection agent conjugate comprises at least one detection moiety covalently bound to the at least one modified Ff phage coat protein.

In one embodiment the nanorod-detection agent conjugate comprises a plurality of detection moieties covalently bound to a plurality of the at least one modified Ff phage coat protein.

In one embodiment the nanorod-detection agent conjugate comprises at least two different types of modified Ff phage coat proteins.

In one embodiment the nanorod-detection agent conjugate comprises at least two different detection moieties.

In one embodiment the nanorod-detection agent conjugate comprises at least two different detection moieties, each covalently bound to a different type of modified Ff phage protein.

In one embodiment the nanorod-detection agent conjugate comprises a plurality of each of at least two different detection moieties, each covalently bound to a plurality of at least two different types of modified Ff phage proteins.

In one embodiment the nanorod-detection agent conjugate is comprised in a population of nanorod-detection agent conjugates.

In one embodiment the nanorod-detection agent conjugate is comprised in a composition comprising the population of nanorod-detection agent conjugates.

In one embodiment at least some of the nanorod-detection agent conjugates in the population or the composition comprise different detection moieties.

In one embodiment the detection moieties are selected from the group consisting of fluorophores, small molecules, peptides, proteins, polymers, nucleic acids, inorganic molecules, dyes, radioisotopes, semiconductors, and paramagnetic compounds.

In one embodiment the detection moiety is a fluorophore, chromogenic substrate, dye, chemiluminescent, paramagnetic, molecule, semiconductor, conductor, nucleic acid, polypeptide, polymer, quantum dot, or radioisotope.

In one embodiment the fluorophore or chromogenic substrate is a fluorophore or chromogenic substrate.

In one embodiment the nanorod-detection agent conjugate comprises at least three, four, five, six, seven, eight, nine or more different detection moieties.

In one embodiment the nanorod-detection agent conjugate comprises at least 10, 15, 20, 25, 30, 35, 40, 45, 50, 55, 60, 65, 70, 75, 80, 85, 90, 100, 200, 300, 400 or more detection moieties.

In one embodiment the nanorod-detection agent comprises one detection moiety per about each 7 copies of an Ff phage coat protein pVIII or modified pVIII comprised in the nanorod.

In one embodiment the nanorod-detection agent conjugate further comprises a binding agent.

In one embodiment the binding agent is covalently bound to at least one Ff phage coat protein. In one embodiment the at least one Ff phage coat protein is a modified coat protein.

In another aspect the invention relates to a composition comprising at least one nanorod-detection agent conjugate as described herein.

In one embodiment the composition comprises at least two nanorod-detection agent conjugates wherein each nanorod detection agent conjugate comprises at least one different detection moiety.

In another aspect the invention relates to a kit comprising one or more nanorod-detection agent conjugates as described herein.

Specifically contemplated as embodiments of these aspects of the invention are all of the embodiments set forth in the NPS, composition, nanorod and method aspects herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, including amino acid and nucleic acid sequences and all contemplated variations and modifications, promoters, inducible promoters and operable linkages, selective markers, auxotrophic markers, (+) strand circular ssDNAs, fusion proteins, induction of production, host cells and host cell cultures, replicated ssDNAs, single and dual transformations, lengths of nanorods produced, nanorod-binding agent conjugates and (+) strand ssDNAs.

In another aspect the invention relates to a method of detecting a target molecule in a sample comprising
a) contacting a sample containing or suspected of containing the target molecule with a nanorod-binding agent conjugate and
b) detecting the binding of the nanorod-binding agent conjugate to the target molecule, wherein the nanorod-binding agent conjugate comprises at least one modified Ff phage coat protein, wherein the nanorod in the nanorod-binding agent conjugate is produced from an NPS as described herein or is a nanorod as described herein or is comprised in a population of nanorods as described herein or is made by a method of making nanorods as described herein.

In one embodiment the nanorod-binding agent conjugate comprises at least one detection moiety covalently bound to the at least one modified Ff phage coat protein

In one embodiment the nanorod-binding agent conjugate comprises one detection moiety per about each 7 copies of an Ff phage coat protein pVIII or modified pVIII comprised in the nanorod.

In one embodiment the nanorod-binding agent conjugate comprises a plurality of detection moieties covalently bound to a plurality of the at least one modified Ff phage coat protein.

In one embodiment the nanorod-binding agent conjugate comprises at least three, four, five, six, seven, eight, nine or more detection moieties.

In one embodiment the nanorod-binding agent conjugate comprises at least two different types of modified Ff phage coat proteins.

In one embodiment the nanorod-binding agent conjugate comprises at least two different types of detection moieties.

In one embodiment the nanorod-binding agent conjugate comprises at least three, four, five, six, seven, eight, nine or more different types of detection moieties.

In one embodiment the nanorod-binding agent conjugate comprises a plurality of each of at least two different detection moieties, each covalently bound to a plurality of at least two different types of modified Ff phage coat proteins.

In one embodiment wherein the nanorod-binding agent conjugate is comprised in a population of nanorod-binding agent conjugates.

In one embodiment the binding agent is selected from the group consisting of small molecules or polypeptides.

In one embodiment the polypeptides are selected from the group consisting of antibodies, antibody-derived single-chain variable domains (scFv), camelid single-chain antibody domain VHH and other types of antibodies and analyte-binding polypeptides.

In one embodiment the target molecule is immobilized on a solid support by binding to a support-attached capture molecule.

In one embodiment the target molecule is selected from the group consisting of viral or bacterial proteins, disease markers or any other molecules (analytes) of interest in the food, environment, animals, or humans. In one embodiment the target molecule is a SARS CoV-2 molecule.

In one embodiment detecting comprises detecting a chemical, spectral, linear-dichroic, fluorescence, visual, chemiluminescence, paramagnetic, sound, electrical, surface plasmon resonance, isotopic, radioactive or other chemical or physical signal.

In one embodiment detecting comprises fluorescent signal detection or visual detection via enzymatic reaction using chromogenic or chemiluminescent substrates.

In one embodiment the method of detecting is a dot blot assay, lateral flow assay (LFA) or an enzyme linked immunosorbent assay (ELISA).

In one embodiment the method of detecting comprises flow cytometry or microfluidics.

In one embodiment the nanorod-binding agent conjugate comprises a plurality of detection moieties covalently bound to a plurality of the at least one modified Ff phage coat protein.

In one embodiment the nanorod-binding agent conjugate comprises at least two different detection moieties, each covalently bound to a different type of modified Ff phage protein.

In one embodiment the nanorod-binding agent conjugate comprises a plurality of each of at least two different detection moieties, each covalently bound to a plurality of at least two different types of modified Ff phage proteins.

In one embodiment the nanorod-binding agent conjugate is comprised in a composition comprising the population of nanorod-binding agent conjugates.

In one embodiment at least some of the nanorod binding agent conjugates in the population or the composition comprise different detection moieties.

In one embodiment the detection moiety is a moiety that produces a detectable chemical, spectral, linear-dichroic, fluorescence, visual, chemiluminescence, paramagnetic, sound, electrical, surface plasmon resonance, isotopic, radioactive or other chemical or physical signal.

In one embodiment the detection moiety is fluorophore, chromogenic substrate, dye, chemiluminescent, paramagnetic compound, small molecule, semiconductor, conductor, nucleic acid, polypeptide, polymer, quantum dot, or radioisotope.

In one embodiment the fluorophore or chromogenic substrate is a fluorophore or chromogenic substrate as described herein.

In one embodiment the detection moieties are selected from the group consisting of fluorophores, small molecules, peptides, proteins, polymers, nucleic acids, inorganic molecules, dyes, radioisotopes, semiconductors, and paramagnetic compounds.

In one embodiment the nanorod-binding agent conjugate comprises at least three, four, five, six, seven, eight, nine or more different detection moieties.

In one embodiment the nanorod-binding agent conjugate comprises at least 10, 15, 20, 25, 30, 35, 40, 45, 50, 55, 60, 65, 70, 75, 80, 85, 90, 100, 200, 300, 400 or more detection moieties.

In one embodiment the nanorod-binding agent comprises one detection moiety per about each 7 copies of the Ff phage coat pVIII comprised in the nanorod.

It will be appreciated that by using the approaches described herein any amine-reactive fluorescent or any other dye or other small molecule that is amine-reactive should be suitable for attachment to nanorods. In one non-limiting example, the inventors have demonstrated fluorescent labelling of BSF nanorods as described herein with the amine-reactive fluorescent dye, DyLight 550 (Example 11, FIG. 20C). In this example nanorods also display a binding molecule due to the fusion to pIII to FnB (Fibronectin-binding domain of S. pyogenes; SEQ NOs: 37, 38, FIG. 37) and have been used for lateral flow assay for detection of the analyte (fibronectin; Example 11, FIG. 20C). Such labeling is contemplated as an embodiment herein.

BSF nanorods in each pVIII subunit have three surface-exposed amino acid residues, Glu2, Asp4 and Asp5 that contain side-chain carboxyl groups; hence the carboxyl-reactive molecules can also be chemically conjugated to the nanorods. Other reactive groups, such as the Tyr residue aromatic hydroxyl group can also been used to attach suitable reactive groups as is known in the art (Bernard and Francis, 2014). The molecules attached could be organic molecules of any kind, including biotin, which serves to bind commercially available or in-house made fusions of biotin-binding proteins such as avidin. In this manner, a broad array of avidin fusions to antibodies, dyes or other functional molecules allows multiple ways to visualize nanorods in an indirect way. Nanorods displaying a detector molecule can bind an analyte and be visualized either indirectly via phage-specific antibody or chemically attached fluorescent dyes (FIG. 20). Such labeled nanorods are contemplated as embodiments herein.

Labelled nanorods also displaying analyte-specific molecules such as antibodies can also be used in immunoassays. In one non-limiting example nanorods were produced that display pIII fusion proteins that specifically bind a SARS-CoV-2 spike-specific single-chain antibody (FIG. 38, SEQ NOs: 39, 40) or SARS-CoV-2 nucleoprotein-specific camelid single-domain antibody VHH (FIG. 55, SEQ NOs: 99, 100). These pIII fusions were combined with pVIII displaying N-terminal Ala-Ala-Gly-Gly (AAGG) evolved to assemble nanorods efficiently (FIG. 32, SEQ NO: 18; FIG. 33, SEQ NO: 20). LPETA-biotin has been enzymatically attached to the nanorods and the avidin-alkaline phosphatase fusion was further attached to the biotin-modified nanorods to allow visualization of the nanorods via indirect labelling. Thus modified nanorods were used in dot-blot, ELISA and lateral flow assays (FIGS. 25-27) as described in methods. Such modified nanorods and methods of use are all contemplated as embodiments herein.

Specifically contemplated as embodiments of this aspect of the invention are all of the embodiments set forth in the NPS, composition, nanorod and method aspects herein including but not limited to embodiments related to nucleic acid expression constructs, scaffold and filler nucleic acid sequences, vectors, plasmids, nanorod replication-assembly plasmids, helper plasmids, BSFnano replication-assembly cassettes, origins of replication, Ff phage proteins, modified Ff phage proteins, Ff phage genes, modified Ff phage genes, including amino acid and nucleic acid sequences and all contemplated variations and modifications, promoters, inducible promoters and operable linkages, selective markers, auxotrophic markers, (+) strand circular ssDNAs, fusion proteins, induction of production, host cells and host cell cultures, replicated ssDNAs, single and dual transformations, lengths of nanorods produced, nanorod-binding agent conjugates, and (+) strand ssDNAs.

Further embodiments described below as a set of potential claims are provided in the interests of providing the reader with a better understanding of the invention and its practice and are illustrative only.

### EXEMPLARY NUMBERED EMBODIMENTS

1. A nanorod production system (NPS) comprising a single nucleic acid expression construct, the construct comprising
a BSFnano replication-assembly cassette
at least one auxotrophic marker,
at least one inducible promoter operably linked to a nucleic acid sequence encoding at least one Ff phage protein, and
at least one plasmid origin of replication not located in the BSFnano replication-assembly cassette.
2. The NPS of embodiment 1 wherein the nucleic acid expression construct is or is comprised in, a plasmid.
3. The NPS of embodiment 1 or embodiment 2 wherein the BSFnano replication-assembly cassette comprises a packaging signal (PS), a (+) ori1, a (+) ori2, and a (−) ori.
4. The NPS of any one of embodiments 1 to 3 wherein the BSFnano replication-assembly cassette comprises a scaffold nucleic acid sequence.
5. The NPS of embodiment 4 wherein the scaffold nucleic acid sequence comprises zero, one or two filler nucleic acid sequences.
6. The NPS of embodiment 4 or embodiment 5 wherein the scaffold nucleic acid sequence comprises a filler nucleic acid sequence that codes for at least one, preferably at least two Ff phage coat or Ff phage modified coat proteins, preferably that codes for pVII and pIX or modified pVII and pIX.
7. The NPS of any one of embodiments 1 to 6 wherein the at least one auxotrophic marker is selected from the group consisting of metE, glyA, infA, thyA, argE, delta-thi-1, thi1, leuB, proAB, ara, and nadC, preferably nadC.
8. The NPS of any one of embodiments 1 to 7 wherein the at least one inducible promoter is selected from the group consisting of lac, tac, araC, or trp promoters, preferably a lac promoter.
9. The NPS of any one of embodiment claims 1 to 8 wherein the at least one inducible promoter is operably linked to a nucleic acid sequence encoding at least one Ff phage replication protein or at least one Ff phage coat protein or both.
10. The NPS of embodiment 9 wherein the at least one Ff phage replication protein is pII or modified pII.
11. The NPS of embodiment 9 wherein the at least one Ff phage coat protein is pVIII or modified pVIII.
12. The NPS of embodiment 11 wherein the modified pVIII comprises at least one amber mutation.
13. The NPS of any one of embodiments 1 to 12 wherein the single nucleic acid expression construct comprises a nucleic acid sequence encoding at least one additional Ff phage protein, preferably at least two additional Ff phage proteins.
14. The NPS of embodiment 13 wherein the additional Ff phage proteins are selected from the group consisting of pIII and pVI. In one embodiment the additional Ff phage proteins are pIII or pVI or both.
15. The NPS of any one of embodiments 1 to 14 wherein the nucleic acid expression construct comprises a nucleic acid sequence encoding a fusion protein comprising at least one Ff phage protein or modified Ff phage protein or functional portion thereof fused to a binding protein or binding portion thereof.
16. The NPS of any one of embodiments 1 to 15 wherein the at least one plasmid origin of replication (p-ori) is a theta origin of plasmid replication, preferably wherein the p-ori is selected from the group consisting of ColE1, pMB1, pSC101, R6K, ColD and 15A, preferably wherein the p-ori is 15A.
17. A nanorod production system (NPS) comprising
i) a nucleic acid nanorod replication-assembly construct comprising
a BSFnano replication-assembly cassette,
at least one auxotrophic marker, and
at least one plasmid origin of replication not located in the BSFnano replication-assembly cassette, and
ii) a helper nucleic acid expression construct comprising at least one selective marker, and
at least one inducible promoter operably linked to a nucleic acid sequence encoding at least one Ff phage protein.
18. A method of making a plurality of nanorods, the method comprising inducing the production of at least 1.0×10¹³ nanorods/L of host cell culture.
19. The method of embodiment 18 comprising transforming E. coli cells with a single nucleic acid construct only.
20. The method of embodiment 19 wherein the single nucleic acid construct comprises a BSFnano replication-assembly cassette, at least one auxotrophic marker, at least one inducible promoter operably linked to a nucleic acid sequence encoding at least one Ff phage protein, and at least one plasmid origin of replication not located in the BSFnano replication assembly cassette.
21. A method of making a plurality of nanorods comprising inducing the replication of a (+) strand circular ssDNA from a single nucleic acid construct comprising a BSFnano replication-assembly cassette, a scaffold nucleic acid sequence, two nucleic acid sequences comprising a pII cut site each, each cut site located in a (+) ori, and at least one nucleic acid sequence encoding at least one modified Ff phage coat protein, preferably at least two, preferably two Ff phage coat proteins.

### EXAMPLES

### Methods and Experimental Procedures

### Bacteria, Bacteriophage, and Plasmids

All bacterial strains used in this disclosure are derived from E. coli non-pathogenic laboratory strain K12 (Table 1), containing one, two or three compatible plasmids (Tables 2, 3 and 4). Bacteriophage used in various aspects of nanorod production are derived from Ff (f1 and M13; Table 5).

### Media and Growth Conditions

The liquid medium 2×YT was used at a concentration of 1× (16 g/L Tryptone, 10 g/L Yeast Extract, 5.0 g/L NaCl, pH 7.4-7.6). 2×YT is a standard microbial growth medium used for the cultivation of E. coli and Ff bacteriophage. This nutrient-rich microbial broth contains peptides, amino acids, and water-soluble vitamins in a low-salt formulation. When required as a solid medium, 2×YT was used at a concentration of 1× (16 g/L Tryptone, 10 g/L Yeast Extract, 5.0 g/L NaCl, 1-2% Agar, pH 7.4-7.6). Agar (BD Difco) was used as a solidifying agent.

Minimal M9 media contained 1×M9 salts (final concentrations 15 g/L KH₂PO₄, 64 g/L Na₂HPO₄, 2.5 g/L NaCl, 5 g/L NH₄Cl, pH 7.2.), 2 g/L MgSO₄, and 0.1 g/L CaCl₂. It was supplemented with 2 g/L Glucose and 2 g/L Casamino Acids. Casamino acids is a mixture of amino acids and oligopeptides obtained from casein by acid hydrolysis; typically used in microbial growth media. It has all the essential amino acids except tryptophan, which is degraded during casein hydrolysis. Casamino acids do not contain NA (nicotinic acid), hence they allow for use of nadC as a selective auxotrophic marker.

Bacteria were cultured in Difco™ 2×YT (Becton-Dickinson, BD) or the M9 minimal containing Glucose and Casamino acids and supplemented, as required, with Nicotinic acid (NA). Liquid cultures were incubated at 37° C. with continuous shaking (200 rpm) unless otherwise stated. To make plates, media described above were solidified by adding Bacto-agar, BD (1%). Antibiotics were supplemented when required at the following concentrations: ampicillin (Amp) at 100 μg/mL; kanamycin (Kan) at 50 μg/mL; chloramphenicol (Cm) at 25 μg/mL.

### Recombinant DNA Technology Methods

General molecular biology and recombinant DNA techniques such as PCR, restriction digests of DNA, ligation, DNA sequencing, DNA agarose gel electrophoresis, preparation of competent cells, transformation and purification of plasmid DNA were carried out as previously described. (Sambrook and Russell, 2001). DNA fragments for construction of recombinant plasmids and phage were either custom-synthesised or PCR-amplified. Any specific modifications are indicated in the protocols described below.

### Titration of Infectious Ff Phage or Phage-Like Particles

Ff phage or phage-like particles containing antibiotic resistance were quantified by titration using an overlay plating method. The 2×YT plates were used for titration of phage; these were supplemented with appropriate antibiotic for titration of phage-like particles containing antibiotic resistance markers. For titration of particles containing Kan^{R} marker, a middle (9 mL) layer of 2×YT without antibiotic was poured immediately preceding titration, to allow growth of bacteria for a few hours (prior to diffusion of antibiotic), which is required for successful transfection. Once this layer has solidified, overnight culture of appropriate indicator strain (100 μL) was mixed with 2.5 mL of molten (50° C.) 2×YT soft agar (0.5% agar); the mix was poured on the surface of the solidified intermediate layer. Once the overlay was solidified, 5 μL of 100-fold serial dilutions of phage or phage-derived particles were spotted onto the surface. The plates were incubated at 37° C. overnight, and the following day the phage titres were calculated based on the plaque counts, whereas the number of infectious marker-containing particles was determined from the number of antibiotic resistant transductants. The titres were expressed as plaque forming units (pfu) or transducing particles (tdp) per mL.

Evolving the pVIII AlaGly ΔP6 mutant to restore filamentous phage assembly

Bacteriophage R786 encoding engineered pVIII for enzymatic attachment of LPXTA-tagged proteins or small molecules contains AlaGly insertion between mature positions 1 and 2 and deletion of Pro at position 6. This phage (R786) gave titres around 10¹⁰, about 100-fold lower in comparison to a control phage, R785 (which gave titres of around 10¹², typical for the Ff phage). Difference in titres was therefore attributed to the inserted AlaGly between position 1 and 2 of the mature pVIII and/or deletion of Pro at the position 6. In order to “evolve” R786 to give titre matching that of R785, the original R786 stock was passaged through the host strain K2091 by three rounds of growth in a liquid culture, where the stock was mass-transferred from one round to the next, without plaque purification. Each round was seeded with phage at a low m.o.i. (1 phage to 1000 bacteria). The phage stock after the third round of growth was diluted and plated on a K2091 lawn to obtain 100-300 plaques per plate. Large plaques, similar to those of R785, were detected on these plates. Phage from three large well-separated plaques were clonally purified. The stocks were grown from the clonally purified in a standard manner and analysed by titration. Three evolved mutants that demonstrated an increased titre matching that of R785 were shown to have acquired point mutations in pVIII. Mutated phage were tested as helpers for a standard phagemid vector pUC118 and titrated. The phage giving highest titres contained mutation L27S. This phage was named R788. Sequence encompassing pVIII was amplified and inserted into the pHP backbone to obtain pHP1Aev and pHP1AevIIICM.

### Agarose Gel Electrophoresis of Native Ff Phage and Ff-Derived Nanorods

Agarose gels electrophoresis was used for rapid detection and characterisation of the native Ff phage and phage-derived nanorods (Nelson et al., 1981). The running buffers were 1×TAE (40 mM Tris, 2 mM EDTA, 20 mM Acetic Acid), pH 9.0 or 8.3.

The pH 9.0 buffer was used for the nanorod variants containing Ser instead of Glu at position 2 in the mature pVIII (gVIII^{am25}) in the presence of supD tRNA from the host (Table 3). Samples were mixed with the native loading buffer (final concentration 1×TAE, 5% glycerol and 0.05% BPB; pH 9.0 or pH 8.3) before loading the gel. Electrophoreses were run for 15 h at 20 V (1.5 V/cm) and stained in ethidium bromide (10 μg/mL EtBr, 1×TAE, pH 8.3) for 20 min to visualise free DNA and RNA in the sample. The native, intact nanorods should not be visible at this stage since their DNA is inside the intact nanorod. To visualise the nanorods, coat proteins were removed, and ssDNA exposed by soaking the gel in 0.2 M NaOH for 45 min. After rinsing in MiliQ water for 10 min, the gel was neutralised by soaking in 0.45 mM Tris (pH 7.1) and stained again in EtBr for another 20 min, followed by de-staining in water and imaging using a CCD camera. Fluorescently labelled nanorods were visualized directly, without staining.

### Agarose Gel Electrophoresis of SDS-Disassembled Ff Phage and Phage-Derived Nanorods

Gels contained 0.8% to 1.2% (w/v) agarose (depending on the size of analysed ssDNA) in 1×TAE buffer, pH 8.3. or 9.0. Particles were disassembled by mixing with SDS buffer (1% SDS, 1×TAE, 5% glycerol, 0.05% BPB) and heating at 99° C. for 10 to 15 min. After equilibration to room temperature the samples were loaded onto an agarose gel. Electrophoresis was run for 150 min at 3.7 V/cm; the gel was stained in EtBr for 20 min, followed by destaining, and visualised with the GelDoc XR.

### Nanorod Production

For nanorod production, high-efficiency electrocompetent cells of the appropriate strain were transformed, in the case of the single-plasmid production system, with the pPop-up single nanorod-producing plasmid. In the two-plasmid nanorod production system, cells already containing a helper plasmid were transformed with the pBSFnano template plasmid. After transformation cells were recovered for 1 h in the SOC medium. For antibiotic selection, in 2×YT medium, transformed cells were suspended in 10 mL of liquid media containing appropriate concentration(s) of antibiotic(s) as required. For auxotrophic selection, after the recovery in the SOC medium cells were washed twice in 0.5% NaCl to remove nutrients and resuspended in 10 mL M9 Glucose Cas medium containing appropriate concentration(s) of antibiotic(s). Resuspended cells (5 mL) were added to 500 mL of the pre-warmed medium containing the same ingredients, in a 2 L flask, and incubated overnight at 37° C. with aeration. For the pPop-up or helper plasmids containing gII driven by the lacUV5 promoter, IPTG was added to the culture at OD₆₀₀=0.1. After a 16-h incubation, the cells were removed from the culture by centrifugation (8000×g at 4° C.) and the nanorods from the supernatant were concentrated by PEG precipitation (2×YT cultures) or ultrafiltration (M9 Cas Glucose cultures).

### Concentration of Nanorods by PEG Precipitation

The culture supernatant was poured into sterile centrifuge bottles and the PEG8000 powder was added to 5% for nanorods 100 nm in length and up to 15% for nanorods of ≤100 nm in length. After the PEG was dissolved, NaCl powder was added to 0.5 M, dissolved, and the suspension was incubated on ice for 2 hours. Mixture was then pelleted by centrifugation at 8000×g for 30 minutes at 4° C. Supernatant was decanted and the “empty” centrifuge bottles were centrifuged again under the same conditions for 5 minutes to collapse the nanorod pellet to the bottom of the bottle. This is required because the filamentous phage PEG pellet precipitates during centrifugation as a sticky film along the wall of the bottle. Pellet obtained after PEG precipitation was re-suspended in 5 ml of 1× TBS (pH 7.6) and the remaining insoluble debris was pelleted by centrifugation at 8000×g for 30 minutes at 4° C. DNAse- and RNAse-containing buffer (final concentration 12 μg/mL DNase, 40 μg/mL RNase, 5 mM MgCl₂, 10 mM TRIS pH 8.0) was then added to the supernatant and incubated at room temperature for 1 hour. DNAse and RNAse were then inactivated by the addition of EDTA at a final concentration of 20 mM. Particles were re-purified by precipitation in 5% to 15% PEG, 0.5 M NaCl solution as described above. The nanorod pellet was re-suspended in 0.5 mL 1× TBS (pH 7.6) and centrifuged again at 4000× g for 10 minutes at room temperature to remove the insoluble debris.

### Concentration of Nanorods by Ultrafiltration

The culture supernatant was filtered through a bottle-top filter (0.22 μm) to remove the remaining cells and cell debris. Nanorods from the filtered supernatant were concentrated by ultrafiltration, using an Amicon Stirred Cell 400 mL pressure system as per the method outlined in (Rakonjac and Model, 1998) with additional washing steps (three washes, each with 100 ml of TBS pH 7.3). Retentate was collected into a test tube and free DNA and RNA were removed from the nanorod suspension by adding DNAse and RNAse as described in the previous section. Nanorods were precipitated with PEG as described in the paragraph above.

### Purification of Nanorods by CsCl Gradient Ultracentrifugation

Caesium chloride gradient centrifugation was used to separate the concentrated nanorods from the fine cellular debris and bacterial proteins. About 1 mL of 1000-fold concentrated nanorods in were mixed with 2 mL of the same buffer as the one in which the nanorods are resuspended containing 1.5 g solid CsCl, vortexed briefly, and the volume was adjusted to 4 mL with the buffer, to obtain a final concentration of 0.375 g/mL CsCl. Ultracentrifugation at 100,000×g at 18° C. for 16 h resulted in the formation of density gradient and separation of the nanorods from cellular debris and remaining DNA and RNA (Sattar et al., 2015). Depending on the amount of nanorods, they were either visible as a grey band or were not visually detectable. In both cases the nanorods were collected using a hypodermic needle. When a visible band was observed the tube was punctured just underneath the band. When a band was not visible, the centrifuge tubes were punctured at the bottom, and the 100 μL (4 drops) fractions were collected.

The fractions were analysed by agarose gel electrophoresis of SDS-disassembled nanorods to detect the fractions that contained nanorods and were devoid of cell-derived DNA or RNA. The fractions that contained the strongest nanorod ssDNA band, and no residual RNA and DNA, were combined and dialysed against 3,000 volumes of 1× PBS or TBS buffer or 50 mM Tris-HCl pH 8 at 4° C., using 50 kDa cut-off Slide-a-Lyzer™ dialysis cassettes. Alternatively, they were concentrated and desalted by the spin-ultrafiltration as described below.

### Purification of Nanorods by Anion Exchange Chromatography

If the removal of residual proteins that fractionated with nanorods in the CsCl gradient centrifugation was required, the samples were subjected to another step of purification, by anion exchange chromatography. For this purpose, a strong anion Q, −N+(CH₃)₃ column, SepFast™ (BioToolomics), was used. The column was equilibrated with 10 column volumes (CV) of binding buffer (buffer A: 50 mM Tris-HCl pH 8). The sample containing the nanorods was then passed through the column, followed by a washing step with buffer A. Subsequently, the bound nanorods were eluted from the column by a gradient of NaCl from 0 to 1.5 M (in the 50 mM Tris buffer, pH 8). Column fractions corresponding to absorption peaks at the 280 nm wavelength were collected and analysed by SDS-PAGE to identify those containing pure nanorods based on the known Ff protein pattern.

### Concentration of Purified Nanorods by Spin-Ultrafiltration

When required, purified nanorods were concentrated and desalted by filtration through a 50 kDa-cut-off filter using centrifugal force in the Vivaspin system (GE Healthcare) according to manufacturer's instructions. If the buffer exchange or desalting was required, up to 6 washes with the desired buffer were performed. Nanorods were detached from the filters by storing the filter units overnight at 4° C., making sure that the filters were covered with the buffer. The following day, the buffer was gently pipetted up and down over the filter, followed by collection into suitable sterile vials or tubes.

### Quantification of Nanorods

Nanorods do not carry any markers, hence they were quantified by densitometry of ssDNA from SDS-disassembled after separation by agarose gel electrophoresis (Rakonjac and Model, 1998). Each quantification gel was loaded with a series of known amounts of purified ssDNA extracted from the nanorods of similar size, to obtain a standard curve for densitometry. Images of EtBr-stained gels were analysed using ImageJ software and Microsoft Excel.

Alternatively, highly purified nanorods (after the CsCl gradient centrifugation or Ion exchange chromatography) were quantified by spectrophotometry using the ε=3.84 ml/(mg*cm) value at the wavelength of 269 nm (Day, 1969).

### Staining and Transmission Electron Microscopy of Nanorods

All transmission electron microscopy images (micrographs) were collected at the Manawatu Microscopy and Imaging Centre (MMIC), School of Fundamental Sciences, Massey University, Manawatu Campus. Purified phages or nanorod samples were diluted in MiliQ water to a final concentration of 10¹⁰ nanorods/mL. An 80 μL drop of the sample was placed in a glass petri dish lined with Parafilm™ (Bemis Company Inc., USA). A formvar/carbon-coated 200 mesh copper grid (Agar Scientific, coated in the lab) was placed facing the film side down, onto the sample droplet and left for 4 minutes to allow adsorption of phages onto the grid. The grid was carefully lifted and placed on the side of Whatman No1 filter paper to remove excess liquid.

The film with the adsorbed phage nanorods was placed on a drop of 2% Uranyl Acetate in MilliQ and incubated for 4 min at room temperature to stain. Excess fluid was drained again, and the film was placed onto Whatman No1 paper to dry. Images were collected in TEM at 100 kV (FEI Tecnai G2 Spirit BioTWIN, Czech Republic).

### Fibronectin Lateral Flow Assays

Previously prepared dipsticks (containing printed collagen and pVIII-specific mouse monoclonal antibody on the T and C lines, respectively), were stored in the zip-lock bags, protected from light. Before use, dipsticks were blocked overnight at 4° C. in Odyssey® blocking buffer supplemented with 1:1,500 monoclonal anti-Fn antibody to minimise the unspecific binding of FnB-displaying nanorods to potentially Fn-contaminated collagen on the T line. The 96-well microtiter plate that was used for the reaction mixtures was blocked with the same buffer without the Fn-specific antibody, under the same conditions. After blocking, the dipsticks were rinsed twice with PBST buffer and dried for 2 hours at 37° C. A total of 10¹¹ nanorods per assay were mixed with serial dilutions of analyte in 1× PBS, in a total volume of 50 μL, in a 96-well plate and incubated at room temperature for 30 min.

Dried blocked dipsticks were dipped into the wells containing the reaction mixtures for 15 min at room temperature, then taken out of the wells and placed on the filter paper to dry at 37° C. for 1 h. Unlabelled nanorods bound to the dipstick were visualised using rabbit M13-specific antibodies, followed by secondary AP-conjugated antibodies. Fluorescently labelled nanorods (DyLight® 550) were directly visualised using the Azure c600 fluoroimager.

### High-Density Sortase-Mediated Labelling of the BSFnanorods

His-tagged Sortase A of Streptococcus pyogenes, SrtA Sp, was expressed from plasmid pET28a-SpySrtA (Table 4) and affinity purified using Ni-NTA agarose. Sortase reactions were performed in a volume of 500 μL in a microfuge tube. For biotin labelling, the reaction mixture contained 50 μM of SrtA Sp, 200 μM of K(biotin)-LPETAA (GenScript), and 5 nM of nanorods displaying Spike-specific antibody C121 (BSFnano728Aev1C121; ˜3×10¹² nanorods/mL) in the Sortase buffer (50 mM Tris pH 7.5, 150 mM NaCl). The mixture was incubated at 37° C. with continual shaking for 3 h. After incubation, 1 mL of the Sortase buffer was added into the microfuge tube containing the reaction mixture to dilute the substrate and enzyme. The mixture was then transferred into a pre-equilibrated VivaSpin tube (GE HealthCare, 100 kDa cut-off, capacity 2 mL) and centrifuged at 4000×g at 4° C. for 10 min or more until the remaining volume was ˜150 μL. The flowthrough was discarded, and the solution in the concentrator was refilled with TBS (25 mM Tris pH 7.6, NaCl 150 mM) to a volume of 1.5 mL. The centrifugation, removal of flowthrough and the volume refilling steps were performed for two more times. After that, the Vivaspin tube was centrifuged at 4000×g 4° C. for 10 min or more until the desirable concentration (˜150 μM). The concentrate, that now contained biotin labelled nanorods, was transferred to a microfuge tube and stored at 4° C. until further characterization and uses.

The sortase-mediated labelling of the BSFnano nanorods with fluorescein isothiocyanate (FITC) was implemented, as described for biotin labelling, with some modifications. The reaction mixture contained 50 μM of SrtA Sp, 200 μM of FITC(Ahx)-LPETAA (Mimotopes, Australia), and 5 nM of the BSF nanorods(˜3×10¹² nanorods/mL) in the Sortase buffer (50 mM Tris pH 7.5, 150 mM NaCl). The mixture was then incubated in the dark.

Dot Blot Assay for Detection of SARS-CoV-2 Spike Ectodomain (ECD) Using Nanorods Displaying Spike-Specific scFvs

On a nitrocellulose membrane strip (2×9 cm, 0.2 μm pore size, Advantec), 2 μL of each sample were gently pipetted onto the pre-defined areas in the following order: SARS-CoV-2 Spike ectodomain (ECD, SinoBiological Cat: 40589-V08B1) at 50 ng/μL, 5 ng/μL and 0.5 ng/μL, the biotinylated BSFnano728Aev1C121 (10¹¹ nanorods/mL) as a positive control and 2 μL of TBS (20 mM Tris pH 7.5, 150 mM NaCl) as negative control. The membrane was left to dry at room temperature (RT) for 30 min and then transferred to a 15 mL Falcon tube. All the following steps, unless stated, were completed with the membrane inside the same tube. 10 mL of the blocking buffer (3% bovine serum albumin, 20 mM Tris pH 7.5, 150 mM NaCl, 0.05% Tween20) were added into the tube and incubated with continual rotation at RT for 2 h. The blocking buffer was then discarded. The biotinylated BSFnano728Aev1C121 nanorods, 2 mL at 10¹¹ nanorods/mL diluted from the stock in TBST (20 mM Tris pH 7.5, 150 mM NaCl, 0.05% Tween20), was pipetted into the tube and incubated at room temperature with continual rotation for 1 h. The membrane was then washed three times with 5 mL of TBST, 5 min each, before being labelled with 5 mL of the Streptavidin-Alkaline phosphatase conjugate at 1:5000 dilution (Sigma) for 1 h at RT. The membrane was then washed five times with 5 mL of TBST, 5 min each. The visualization was done by incubating with 2 mL of the SIGMAFAST™ BCIP®/NBT working solution for 15 min at RT.

### Enzyme-Linked Immunosorbent Assay (ELISA) Using Unlabelled BSFnano Nanorods

ELISAs were performed on 96-well microplate (F96 Maxisorp Nunc-Immuno, ThermoFisher Scientific). The plate was first coated with 100 μL of the CR3022 antibody (Abcam) per well at 1 mg/mL in PBS (pH 7.4) overnight at 4° C., then washed one time with 200 μL of the TBST buffer per well (20 mM Tris pH 7.5, 150 mM NaCl, 0.05% Tween-20) and incubated with 200 μL of the blocking buffer per well (5% low-fat milk powder in TBST) for 2 h at RT. The blocking buffer was discarded, and the plate was washed one time with TBST (200 μL/well). Next, the SARS-CoV-2 ECD solution, 100 μL per well, was added into pre-defined wells at 10-fold-diluted concentrations from 10 ng/μL to 0.001 ng/μL, prepared in the blocking buffer, and incubated for 1 h at RT. Each ECD concentration treatment was performed in triplicates. The wells incubated with 100 μL of the blocking buffer were included as negative control. The plate was then washed five times with TBST (200 μL/well). The nanorod solution, BSFnano728Aev1C121 at 10¹⁰ nanorods/mL prepared in the blocking buffer, was added with a volume of 100 μL per well and incubated at RT for 1 h. The plate was then washed five times with TBST (200 μL/well). The M13-specific rabbit polyclonal antibody solution at 1:1000 dilution, prepared in the blocking buffer (Invitrogen P_{A}1-26758), was added at 100 μL/well and incubated at RT for 1 h. The plate was then washed five times with TBST (200 μL/well). The HRP-conjugated anti-rabbit monoclonal antibody (NA934vs, Cytiva) at the 1:5000 dilution in the blocking buffer was added at 100 μL/well, incubated at RT for 30 min, and washed five times with TBST (200 μL/well). The signal was developed by addition of 100 μL of 1-Step™ Ultra TMB-ELISA Substrate Solution (ThermoFisher Scientific) and incubation at RT for 30 min; the reaction was stopped by addition of 100 μL of H₂SO₄ 2 M. The absorbance was measured at 450 nm.

### Elisa Assay Using Biotinylated BSFnano Nanorods

ELISAs were performed on 96-well microplate F96 Maxisorp Nunc-Immuno and NUNC immobilizer amino plates were used, respectively, for assays where antibodies and aptamers were immobilised as capture molecules, both from ThermoFisher Scientific). For the SARS-CoV-2 Spike protein ELISA, the plate was first coated with 100 μL of the SARS-CoV spike-specific capture antibody CR3022 (Abcam) per well at 1 mg/mL in PBS (pH 7.4) or for the SARS-CoV-2 nucleocapsid protein (NC) ELISA 100 μL of the custom-synthetised aminated cognate aptamer (Cho et al., 2011) was added at 50 ng/mL. The plates were incubated overnight at 4° C. or room temperature, respectively, then washed one time with 200 μL of the PBST wash buffer per well (PBS pH 7.4, 0.05% Tween-20) and incubated with 200 μL of the Odyssey blocking buffer (Licor) for 2 h at RT. The blocking buffer was discarded, and the plate was washed one time with PBST (200 μL/well). Next, antigen solutions prepared in PBST were added into pre-defined wells (100 μL/well). Spike ECD was added as 10-fold-serial dilutions giving concentrations from 10 to 0.001 ng/μL, whereas recombinant NC, was added at dilutions from 10 to 0.0000001 ng/μL and incubated for 1 h at room temperature. Each antigen concentration was assayed in triplicates. The wells incubated with 100 μL of the PBST buffer were included as negative control. The plate was then washed five times with PBST (200 μL/well). The biotinylated BSFnano728Aev1C121 (Spike ECD ELISA) or BSFnano728AevN3 (NC ELISA) nanorod solution at 109 nanorods/mL prepared in PBST, were added at a volume of 100 μL per well and incubated at RT for 1 h. The plate was then washed five times with PBST (200 μL/well). The HRP-Streptavidin conjugate (BD Pharmingen) at the 1:5000 dilution in the PBST buffer was added at 100 μL/well, incubated at RT for 30 min, and washed five times with TBST (200 μL/well). The signal was developed by addition of 100 μL of 1-Step™ Ultra TMB-ELISA Substrate Solution (ThermoFisher Scientific) and incubation at RT for 30 min; the reaction was stopped by addition of 100 μL of H₂SO₄ 2 M. The absorbance was measured at 450 nm.

### Lateral Flow Assay for Detection of SARS-CoV-2 Spike Protein Extracellular Domain (ECD)

Lateral flow strips were pre-printed at the test line with pan-SARS-CoV Spike-specific antibody CR3022 (Abcam ab273073, 0.5 mg/mL) and at the control line with M13-specific rabbit antibody (0.5 mg/mL, Invitrogen P_{A}1-26758). For the NP detection lateral flow assays the test and control reagents were spotted onto the pre-cut strips. The 1 μL test spot contained 100 μmoles of the NC-specific aptamer and the control spot 200 ng of M13 polyclonal antibody.

Each assay mixture was prepared in the binding buffer (20 mM Tris pH 7.5, 150 mM NaCl, 0.05% Tween20, 0.2% Tropix I-Block reagent) in a total volume of 50 μL, containing 10¹¹ or 10¹⁰ nanorods/mL of the biotinylated nanorod BSFnano728Aev1C121 or BSFnano728AevN3, respectively, and antigen, SARS-CoV-2 Spike ECD at 1 ng/μL, or NC at 50 ng/μL. The assay mixtures without the antigen were used in each assay as negative controls. The assay mixtures were pipetted into pre-defined wells of a non-treated polystyrene 96-well plate (Jet Biofil) and incubated at room temperature for 30 min with 180-rpm shaking.

The strips were vertically dipped into the reaction mixture for 10 min and then transferred into another well containing 100 μL of the running buffer to allow finish the fluid migration for 20 min. The absorbent pad was then trimmed from the strip and the remaining membrane strip was further incubated in 1 mL of the running buffer for 30 min in a 2 mL microtube. The strip was then incubated with 0.5 mL of Streptavidin-Alkaline phosphatase conjugate at 200 mU/mL (Sigma Aldrich, 11089161001 Roche) at room temperature for 30 min, before being washed five times each with 1 mL of TBST (20 mM Tris pH 7.5, 150 mM NaCl, 0.05% Tween20) for 5 min at room temperature. After washing, the strip was transferred to another 2-mL microtube containing 500 μL SIGMAFAST™ BCIP®/NBT solution and incubated for 5 min for signal development.

### Example 1. Single-Plasmid BSFnano Production System

One type of the BSF nanorods production platform that is described in this invention is the single-plasmid system named Pop-up (FIG. 2B). It is composed of a single plasmid and E. coli cells containing this plasmid (named pPop-up; Table 2). E. coli strains used for production of nanorods, and their genotypes are listed in Table 1 and specification of their use is described below.

Due to the toxicity of pVIII to E. coli in the absence of assembly, gene VIII in the Pop-up plasmids contains engineered amber mutation in codon 4 of the CDS (Signal sequence, residue −20 relative to the N-terminus of mature pVIII). This mutation is suppressible in supD tRNA mutant strains that read UAG codon as Ser, or in strains expressing supD tRNA from a plasmid. Construction of plasmids was carried out in non-suppressor strains (e.g., K2245; Table 1), to prevent production of pVIII and thereby avoid toxicity that could result in selection for mutations that could eliminate pVIII production (e.g., mutations in the promoter or coding sequence). The non-suppressor strain K2245 was also used for purifying plasmid DNA that was then used to transform a nanorod-production strain.

A supD strain was transformed with the purified DNA of a pPop-up plasmid in order to produce nanorods. Aside from supD tRNA (expressed from the chromosome or plasmid) other mutations or plasmids in the strain can be used, depending on the properties of a particular pPop-up plasmid (e.g., inducible promoter used for controlled expression, or marker used for selection of transformants).

The single plasmid in this system is represented by a series of individual plasmids with a generic prefix pPop-up (standing for plasmid Pop-up). The novelty of the single plasmid system is in that in the absence of a helper phage or other plasmids, it produces Ff-derived nanorods that cannot replicate on their own, carry no markers and no coding sequences. pPop-up contains all components required for replication and assembly of nanorods, and the plasmid's own replication and maintenance in E. coli (FIG. 7):
A) The BSF replication-assembly cassette;
B) Ff phage genes;
C) Plasmid origin of replication and selection marker(s).

Each of these components is assembled from smaller exchangeable units or blocks. The map of the pPop-up series is given in FIG. 7, and details of components and their functions in Tables 6 and 7). These can be combined to attribute specific properties to the BSF nanorods.

Examples of Pop-up plasmids (listed and described in Table 2) are:
pPop-up529YM
pPop-up529LacYM
pPop-up529LacAev
pPop-up529LacYMN
pPop-up395LacYM
pPop-up221YM
pPop-up221LacYM
pPop-up152LacYM

The names of plasmids include the following components:

pPop-up, helper and template plasmid in the single-plasmid nanorod production system.

Number (529, 395, 313, 289, 221 or 152) denotes the size of circular ssDNA replicated from the pPop-up plasmid and packaged into the nanorods.

YM denotes the plasmid containing: (i) the gVIII variant: gVIII-20am pVIII Y21M (FIG. 32; SEQ ID NO: 13, SEQ ID NO: 14) and (ii) Wild-type gIII (FIG. 34, SEQ ID NO: 29, SEQ ID NO: 30).

Lac denotes the plasmid where Ff promoter P_{A} (FIG. 49, SEQ ID NO: 89) was replaced by placUV5 (FIG. 49, SEQ ID NO: 90).

Aev denotes a gVIII variant: gVIII-20am pVIII nAAGG, ΔP6, S17L, L27S (FIG. 32, SEQ ID NO: 19; FIG. 33, SEQ ID NO: 20).

N denotes nadC auxotrophic marker instead of the kanamycin resistance (Kan^{R}) marker (FIG. 50, SEQ ID NO: 91, SEQ ID NO: 93). Kan^{R} marker is not specified in the name of the plasmid.

Specifications of the pPop-up plasmids (examples listed above):

pPop-up529YM (Table 2; FIG. 10). Basic Pop-up plasmid that expresses pII-pVIII from constitutive phage promoter P_{A}; produces 80-nm nanorods (e.g., FIG. 1D) in SupD E. coli strains (e.g., K1030 or K2091; Table 1).

pPop-up529LacYM (Table 2, FIG. 12); Pop-up plasmid that expresses pII-pVIII from inducible promoter lacUV5 for increased nanorod production (see Example 6, inducible production of nanorods; FIG. 12). To construct this plasmid, the lacUV5 promoter sequence (FIG. 49, SEQ ID NO: 90) together with the 5′ moiety of gII (SalI-HpaI fragment) was custom-synthetised, cut with SaIlI and HpaI, and ligated to the large fragment of the pPop-up529YM plasmid cut with these two restriction enzymes. This plasmid produces 80-nm nanorods in SupD lac^{Iq} E. coli strains (e.g., K2091; Table 1). Mutation lac^{Iq} in the host strain is required for tight regulation of the lacUV5 promoter.

pPop-up529LacAev (Table 2, FIG. 13); Pop-up plasmid that expresses pII-pVIII from inducible promoter lacUV5. SupD lac^{Iq} E. coli strains are required for production of nanorods (e.g., K2091; Table 1). This plasmid contains a variant of gene VIII gVIII-20am, encoding mature pVIII containing the following changes: nAAGG, ΔP6, S17L, L27S displaying AlaAlaGlyGly at the N-terminus of pVIII within the nanorod due to insertion of AlaGly-encoding sequence between residues 2 and 3 of mature pVIII (or between codons 25 and 26 of gVIII) and a deletion of proline residue at the position 6 of mature pVIII (wild-type mature numbering; FIG. 32, SEQ ID NO: 19; FIG. 33, SEQ ID NO: 20). Mutation S17L has been isolated during construction of R777 helper phage possibly as an adaptation to MCS::gIII (Sattar et al., 2015). N-terminal AlaAla motif allows enzymatic attachment of LPXTA-tagged molecules using enzyme Sortase A (SrtA) of Streptococcus pyogenes (SrtA Sp). This plasmid was constructed by ligating the small SnaBI-BamHI fragment from phage R788 to the large SnaBI-BamHI fragment of the plasmid pPop-up529LacYM.

pVIIInAAGG ΔP6 mutations prevent nanorod assembly in the plasmid system. When introduced into the Ff phage genome, these mutations caused reduction of the plaques to pin-point size and decrease the phage titres by ˜100-fold. To overcome the interference with assembly, a laboratory evolution experiment of the f1 phage encoding pVIIInAAGG ΔP6 mutant was carried out (as described in Example 8) to obtain an evolved phage that produced large plaques in the presence of N-terminal AAGG and deletion of Pro at position 6 (Example 8). The large-plaque mutant contained additional mutation, L27S, and the phage titres were restored to the level of the parent containing wild-type pVIII mature sequence (˜10¹²). This phage was named R788. When L27S mutation was transferred back into the pPop-up529Lac backbone to generate pPop-up529LacAev, the production of 80-nm nanorods was regained in SupD lac^{q} strains. Other gVIII mutations were able to recover nanorod production of pVIIInAAGG ΔP6 mutant, e.g., Asp to Ala at position 5 (D5A; mature wild type pVIII numbering; FIG. 33; SEQ ID NO: 21, SEQ ID NO: 22) instead of L27S. The L27S pVIII was chosen based on the highest efficiency of nanorod production among the evolved mutants, the second best being D5A.

pPop-up529LacYMN (Table 2); has identical sequence to pPop-up529LacYM in all phage genes and replication-assembly cassette, however its selective marker is NadC (Dong et al., 2010). This auxotrophic marker allows use of antibiotic-free medium for production of nanorods. The E. coli strain K2487 used for production of nanorods using this plasmid, in addition to the SupD lac^{q} has the ΔnadC mutation, in order to allow auxotrophic selection based on the NadC marker expressed by the plasmid. The nadC gene including its promoter was amplified using E. coli chromosomal DNA as template and appropriate primers. Restriction sites AhdI and XhoI were introduced via primers. The cut PCR product (FIG. 50, SEQ ID NO: 93) was ligated to the large fragment of the AhdI-XhoI-cut pPop-up529LacYM.

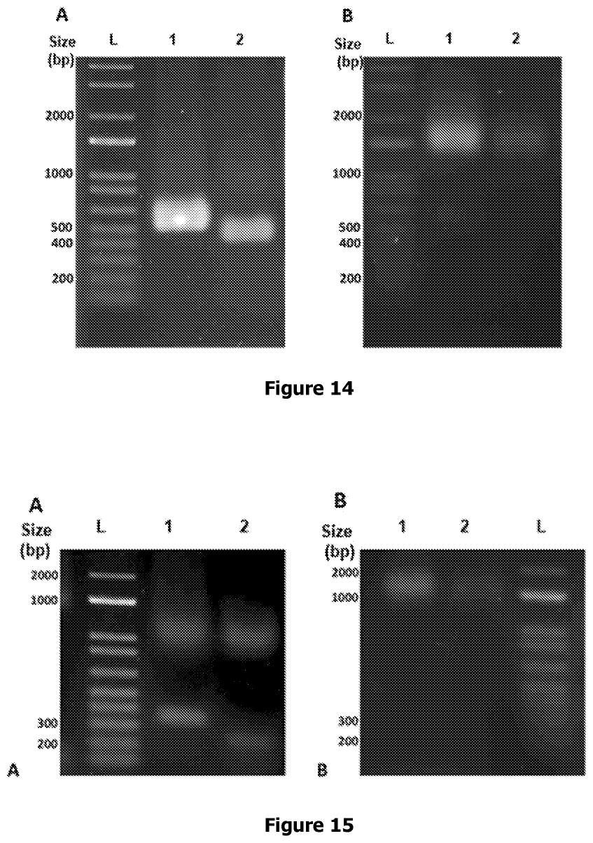

pPop-up395LacYM (Table 2, FIG. 14) is a modified pPop-up529LacYM, where the replication-assembly cassette BSFpn529 (Block i) is replaced by custom-synthetised replication-assembly cassette BSFpn395 (FIG. 43, SEQ ID NO: 63). Briefly, PstI-SalI-cut synthetic fragment corresponding to the BSFpn395 replication-assembly cassette was ligated to the large PstI-SalI fragment of pPop-up529LacYM. The BSFpn395 cassette results in production and assembly of circular ssDNA of 395 nucleotides using both (+) and (−) f1 ori. It is the shortest feasible f1 replicon that still contains the complete (+) and (−) ori. The length of nanorods produced from pPop-up395LacYM is −70 nm (FIG. 1C).

pPop-up221YM (Table 2, FIG. 10) is a modified pPop-up529YM, where the replication-assembly cassette BSFpn529 is replaced by BSFp221 replication-assembly cassette PCR-amplified using pBSF221 plasmid as template and primers that introduced PstI and SaIlI recognition sites (FIG. 47; SEQ ID NO: 77). PstI-SalI-cut BSFp221 replication-assembly cassette was ligated to the large PstI-SalI fragment of pPop-up529YM. Nanorods produced from the pPop-up221YM are 50 nm in length, shorter than the shortest nanorods produced from the BSFpn cassettes, due to the shorter size of the replicated circular ssDNA. The amount of produced nanorods, however, is lower, due to the absence of (−)-ori-dependent negative strand replication from the replication-assembly cassette (FIGS. 4, 5C, 6C and 10).

pPop-up221LacYM (Table 2). This is the pPop-up221YM plasmid in which Ff promoter P_{A} controlling expression of pII-pVIII from was replaced by inducible promoter lacUV5 (FIG. 49, SEQ ID NO: 90) for increased nanorod production of 50-nm nanorods in SupD lac^{Iq} E. coli strains (e.g., K2091; Table 1). BSFp221 replication-assembly cassette (FIG. 47; SEQ ID NO: 77) was PCR-amplified using pBSF221 plasmid as template and primers that introduced PstI and SaIlI recognition sites. PstI-SalI-cut BSFp221 replication-assembly cassette was ligated to the large PstI-SalI fragment of pPop-up529LacYM. Mutation lac^{Iq} in the host strain is required for tight regulation of the lacUV5 promoter. This plasmid demonstrates higher production of nanorods in comparison to pPop-up221YM containing the P_{A} promoter (Table 8).

pPop-up152LacYM (Table 2, FIG. 15). This plasmid contains a trimmed-down BSFp replication-assembly cassette, to produce a shorter circular ssDNA of only 152 nt (FIG. 47; SEQ ID NO: 79). BSFp221 replication-assembly cassette was custom-synthesised.

The synthetic fragment contained PstI and SaIlI recognition sites. PstI-SalI-cut BSFp152 replication-assembly cassette was ligated to the large PstI-SalI fragment of pPop-up529LacYM. The produced nanorods are only 40 nm in length, the shortest ever Ff-derived nanorods made (FIG. 1A). The replication-assembly cassette in this plasmid was designed by removing all sequences between the (+) ori1, PS and (+) ori2 that are potentially not utilised in replication or assembly, to trim the ssDNA for packaging to a minimum and assembling these novel, shortest-ever Ff-derived nanorods (40 nm in length), shorter by 10 nm (20%) than the 50-nm nanorods.

### Example 2. Specifications of the Two-Plasmid BSFnano Nanorod Production System (NPS)

A second type of the BSF nanorods production system described in this invention is a two-plasmid helper-template system pHP-pBSFnano (FIG. 2A; Table 3). It is composed of two plasmids: (i) a helper plasmid variant from a plasmid series named in this disclosure pHP, encoding all Ff proteins required for replication and assembly of nanorods (FIG. 8), (ii) a nanorod replication-assembly plasmid variant from a plasmid series named in this disclosure pBSF, containing a BSFnano replication-assembly cassette (FIG. 9), and E. coli cells containing these two plasmids. E. coli strains used for production of nanorods, and their genotypes are listed in Table 1 and specification of their use is described below.

The novelty of the two-plasmid system is in that in the absence of a helper phage, it produces Ff-derived nanorods that cannot replicate on their own, carry no markers and no coding sequences. While use of two plasmids requires two sequential transformations, and therefore more time in contrast to one transformation in case of the single-plasmid pPop-up system, an advantage of two-plasmid over the single-plasmid system is that it provides greater flexibility in gauging the lengths of nanorods determined by the different pBSFnano template plasmid variants and combining them with different display options determined by the pHP helper plasmid variant, without a need for additional cloning that would be required to combine various lengths of the nanorods with various display options in the single pPop-up plasmid system.

### Specifications of pHP Helper Plasmid Series

Helper plasmids for nanorod production (FIG. 8) express all phage proteins required for replication of the BSFnano replication-assembly cassette and assembly of the resulting circular ssDNA into a nanorod (FIG. 2A). This plasmid is modular; it is composed of exchangeable cassettes corresponding to functional blocks delimited by unique restriction sites and containing a multiple cloning site in gIII (MCS; FIG. 8).

Not only short circular ssDNA derived from the BSF replication-assembly cassette, but also any other replicon containing Ff on and packaging signal will also assemble into an Ff-derived particle, including phagemid particles derived from phagemid vectors. The host cells that express supD tRNA that allows translation of stop codons in pVIII into Ser are required for the nanorod production. Helper plasmids lacking one or more coat-encoding genes are conceivable, if it is desired to manipulate assembly or supply coat protein variants in trans, from a third plasmid. All helper plasmids contain a pA15 origin of replication and a Kan^{R} marker; albeit it is envisaged that these can be replaced by any origin and selective marker compatible with that of pBSF template plasmid or any additional plasmids containing compatible plasmid origin of replication and marker in the same cell.

Due to the toxicity of pVIII to E. coli in the absence of nanorod assembly, gene VIII in the Helper plasmids (pHP series) contain engineered amber stop codon which prevents pVIII production during culturing required for construction of the plasmid and purification of plasmid DNA. There are two groups of pHP helper plasmids, based on the position of the amber codon within gene VIII CDS. In the pHP1 series codon 4 (TCT) encoding Ser in the wild-type gene VIII CDS is changed into TAG (amber) stop codon (FIG. 32-33, SEQ ID NOs: 13-24). Ser4 is located in the pVIII signal sequence, residue −20 relative to the N-terminus of mature protein and is not included in the mature pVIII that is packaged into the nanorod. In the pHP2 series, codon 25 (GAG encoding Glu) in gVIII CDS is converted to TAG (amber stop codon). Codon 25 corresponds to position 2 in the mature pVIII (FIG. 33, SEQ ID NOs: 25-28).

Amber codons are suppressible in supD tRNA mutant strains that read UAG codon as Ser, or in strains expressing supD tRNA from a separate compatible plasmid as described above. In cells transformed with the pHP1, pVIII protein sequence is unchanged when mRNA is translated in a supD strain (given that suppressor D tRNA translates the amber codon into a serine). In SupD cells transformed with the pHP2 helper plasmid series, Ser is incorporated by supD tRNA at the 2^{nd} residue of mature pVIII. In the wild-type pVIII there is Glu at position 2, hence the produced protein is mutated. As a consequence of Glu to Ser change, the isoelectric point of pVIII changes from 6.8 to 8.3, and the overall isoelectric point of the nanorod, which contains hundreds of pVIII copies. This affects migration of the native nanorods in the agarose gels (whose pH is 8.3), resulting in a smear instead of a band. Increase of pH of the agarose gel and running buffer to pH 9 results in focusing of the smears into the bands. Agarose gel and buffer for analysis of the native nanorods containing gVIII^{amber25} produced in a supD strain were therefore all set at pH 9.0 Strains containing supE tRNA mutation insert Gln in the amber codon position during translation, however this suppressor was not used as it results in a much-lower-efficiency suppression in comparison to supD, based on titration of gVIII^{amber25} phage R777 (Sattar et al., 2015).

Further changes of pVIII involving charged residues could be engineered in order to gauge the isoelectric point of the nanorods, positive or negative, at specific pH values of the solvent. Gauging the nanorod charge in turn is of interest for specific applications that require specific charge of nanorods.

Components of the two-plasmid BSFnano production system are:
B) Helper plasmid series (FIG. 8; Table 3), containing:
   b. Ff phage genes (listed in FIG. 3).
   c. Plasmid origin of replication and selection marker(s).
   B) BSFnano replication-assembly plasmid series (FIG. 9; Table 3), containing:
   d. The BSF replication-assembly cassette.
   e. Plasmid origin of replication and selective marker(s) for maintenance of the plasmid in E. coli cells.

Each of the plasmids is composed of smaller exchangeable units (blocks; FIG. 8, Table 6 and 7) that can be combined to attribute specific properties to the BSF nanorods.

### Helper Plasmid Series pHP

Helper phage pHP plasmid variants (Table 3) have been designed for different options of display and functionalisation. Variants were constructed in a modular fashion, with functional segments of the helper plasmids arranged into exchangeable functional blocks (FIG. 8, 30-38, 49, 52; SEQ ID NOs: 3-36, 89-90, 95). The generic map of the pHP series is given in FIG. 8; the pHP series variants are listed below:
pHP1
pHP1Lac (SEQ ID NO: 95; FIG. 52)
pHP1YM
pHP1YMLac
pHP1Az
pHP1A
pHP1Aev
pHP1LacAz
pHP1LacAev
pHP2
pHP2G8
pHP1C
pHP1AevC
pHP1LacAevC121
pHP1LacAevN3
pHP1LacAevG8
pHP2FnB
pHP2G8FnB

The names of pHP plasmids include the following components:
pHP1, standing for Helper Plasmid variant 1, helper plasmid containing gVIII-20am (FIG. 32-33, SEQ ID NOs: 13-24). Isoelectric point of pVIII assembled with assistance of the pHP1 series is ˜pH6.8 in the absence of additional pVIII modifications.
pHP2, standing for Helper Plasmid variant 2, helper plasmid containing gVIII2am (FIG. 33, SEQ ID NOs: 25-28). Isoelectric point of pVIII assembled with assistance of this series of helpers is ˜pH8.3 in supD host due to replacement of Glu residue at position 2 of the mature pVIII by a Ser residue, unless additional changes in the number of charged amino acid residues are introduced into pVIII.

YM denotes helper plasmid containing: (i) the gVIII variant: gVIII-20am pVIII Y21M and (ii) Wild-type uninterrupted gIII (FIG. 32, SEQ ID NOs: 13-14; FIG. 34; SEQ ID NO: 29, SEQ ID NO: 30).

Absence of YM from a pHP plasmid name denotes gIII::MCS containing multiple cloning site that places inserted peptide-coding sequences between the signal sequence and the rest of pIII, thereby allowing construction of display fusions with pIII as the platform; and pVIII containing wild-type residue at position 21 (Tyr) (FIG. 32, SEQ ID NOs: 13-14; FIG. 34; SEQ ID NO: 29, SEQ ID NO: 30).

Lac denotes the plasmid containing placUV5a Ff promoter driving expression of gII(gX)-gV-gVII-gIX-gVIII operon instead of the intrinsic Ff phage promoter P_{A} (FIG. 12-13, 49; SEQ ID 90).

Aev denotes a gVIII variant: gVIII-20am pVIII nAAGG, ΔP6, S17L, L27S (wild-type mature numbering; FIG. 32, SEQ ID NO: 19, FIG. 33, SEQ ID NO: 20).

G8 denotes insertion of 4 Gly residues in gVIII at codon 23, i.e., immediately upstream of the position 1 in mature pVIII (FIG. 33, SEQ ID NO: 27, SEQ ID NO: 28). Four glycines are therefore displayed on the surface of each pVIII subunit of the nanorod shaft. Alternatively, it denotes insertion of 4 Gly residues in gVIII at codon 23, and an additional Gly residue replacing the N-terminal Ala (A1G) in the mature wild-type pVIII.

This variant of pVIII also contains an amber mutation replacing the Ser codon at position 4 in the open reading frame (or −20 counting towards the C-terminus from the codon 1 of the mature protein; gVIII-20^{am} nGGGG A1G) FnB denotes insertion of sequence encoding fibronectin-binding repeats (FnB) from Streptococcus pyogenes fibronectin-binding protein Sof22 into the MCS of pIII so that it forms a fusion. BSFnanorods containing this fusion display FnB repeats on pIII as a platform (FIG. 37, SEQ ID NO: 37, SEQ ID NO: 38).

C121 denotes insertion of sequence encoding single-chain variable domain (scFv) specific for the SARS-CoV-2 Spike protein receptor-binding domain (derived from a complete antibody sequence of the same name (Robbiani et al., 2020) (FIG. 38, SEQ ID NO: 39, SEQ ID NO: 40).

N3 denotes insertion of sequence encoding the antigen-binding domain of a heavy-chain-only (VHH) antibody specific for the SARS-CoV-2 nucleocapsid (NC) protein (Sherwood and Hayhurst, 2021) (FIG. 55, SEQ ID NO: 99, SEQ ID NO: 100).

### Specification of Individual pHP Plasmids:

pHP1 is a helper plasmid containing gVIII-20^{am} S17L (FIG. 33, SEQ ID NO: 15, SEQ ID NO: 16) and gIII::MCS (FIG. 35, SEQ ID NO: 31, SEQ ID NO: 32). This plasmid was constructed by ligating a custom-synthesized SnaBI-NcoI fragment to the large SnaBI-NcoI fragment of pHP2.

pHP1Lac (SEQ ID NO: 95, FIG. 52) is a helper plasmid containing gVIII-20^{am} S17L (FIG. 33, SEQ ID NO: 15, SEQ ID NO: 16) and gIII::MCS (FIG. 35, SEQ ID NO: 31, SEQ ID NO: 32). This plasmid was constructed by ligating a custom-synthetised PstI-HpaI fragment containing the lacUV5 promoter and the 5′ moiety of the gII CDS to the large PstI-HpaI-fragment of pHP1.

pHP1YM helper plasmid combines gVIII-20am, Y21M (FIG. 32, SEQ ID NO: 13, SEQ ID NO: 14) with wild-type pIII (FIG. 34 SEQ ID NO: 29, SEQ ID NO: 30). Expression of the gII(gX)-gV-gVII-gIX-gVIII operon is under the control of the native phage promoter P_{A} (FIG. 49, SEQ ID NO: 89). It was constructed by ligating a custom-synthesised SnaBI-BamHI fragment containing gVIII-20am, Y21M (FIG. 32, SEQ ID NO: 13, SEQ ID NO: 14) and 5′ moiety of the wild-type pIII (FIG. 34, SEQ ID NO: 29, SEQ ID NO: 30) to the large SnaBI-BamHI fragment of pHP2. Y21M mutation confers uniform conformation to all pVIII subunits and is used in structural analyses of the virion shaft (Marvin et al., 2006).

pHP1YMLac helper plasmid is identical pHP1YM, except that the P_{A} promoter is replaced by a lacUV5 promoter (FIG. 49, SEQ ID NO: 90). This plasmid was constructed by ligating a custom-synthetised PstI-HpaI fragment containing the lacUV5 promoter and the 5′ moiety of the gII CDS to the large PstI-HpaI-fragment of pHP1YM. Expression of gII encoding the replication protein and downstream genes in the operon gII(gX)-gV-gVII-gIX-gVIII is inducible by IPTG.

pHP1Az is a helper plasmid containing gVIII-20am, A9M, S17L, M28L and gIII::MCS (FIG. 33, SEQ ID NO: 23, SEQ ID NO: 24). It was constructed by ligating a synthetic SnaBI-NcoI fragment containing the gVIII-20am, A9M, S17L, M28L and gIII::MCS (FIG. 35, SEQ ID NO: 31, SEQ ID NO: 32) to the large SnaBI-NcoI fragment of pHP1. Modifications of gVIII are designed to produce nanorods containing in-vivo-incorporated azides in a medium containing artificial amino acid azido homoalanine (Aha) for chemical functionalization of pVIII via click chemistry (Petrie, 2015).

pHP1Aev is a helper plasmid containing gVIII-20am nAAGG, ΔP6, S17L, A27S (FIG. 32, SEQ ID 19, FIG. 33, SEQ ID NO: 20) and gIII::MCS (FIG. 35, SEQ ID NO: 31, SEQ ID NO: 32). Modified gVIII allows enzymatic attachment of proteins or peptides containing C-terminal tag LPXTA, or small molecules conjugated to this motif, catalyzed by sortase A (SrtA Sp) of Streptococcus pyogenes. The parent helper plasmid pHP1A was constructed by ligation of a synthetic SnaBI-NcoI fragment containing gVIII-20am nAAGG, ΔP6, S17L (FIG. 32, SEQ ID NO: 17, SEQ ID NO: 18) and gIII::MCS (FIG. 35, SEQ ID NO: 31, SEQ ID NO: 32) to the large SnaBI-NcoI fragment of plasmid pHP2. This helper plasmid was a very poor producer of nanorods, therefore in vitro evolution in the context of a full-length phage was used to obtain secondary mutations with improved ability to produce nanorods. Briefly, the SnaBI-NcoI fragment containing the gVIII −20am nAAGG, ΔP6, S17L and gIII::MCS was ligated to the SnaBI-NcoI fragment of phage R784 (Table 5) to obtain phage R785. This phage formed very small plaques and gave ˜100-fold lower titres than the parent helper phage VCSM13. R785 was subjected to three rounds of evolution in the lab to select for high-titre mutants as described in the Material and Methods section and Example 1 (pPop-up529LacAev). A27S is mutation that increased the titre of phage containing N-terminal AAGG tag in gVIII by ˜100-fold. The gVIII from evolved phage (SnaBI-NcoI fragment) was cloned back into the pHP1A backbone to replace original gVIII with the evolved gVIII sequence (FIG. 32, SEQ ID NO: 19, FIG. 33, SEQ ID NO: 20).

pHP1LacAz is identical to pHP1Az, except that the P_{A} promoter (FIG. 49, SEQ ID NO: 89) is replaced by a lacUV5 promoter (FIG. 49, SEQ ID NO: 90). This plasmid was constructed by ligating a custom-synthetised PstI-HpaI fragment containing the lacUV5 promoter and the 5′ moiety of gII to the large PstI-HpaI-fragment of pHP1Az. Expression of gII encoding the replication protein and downstream genes is inducible by IPTG.

pHP1LacAev is identical to pHPAev, except that the P_{A} promoter (FIG. 49, SEQ ID NO: 89) was replaced by a lacUV5 promoter (FIG. 49, SEQ ID NO: 90). This plasmid was constructed by ligating a custom-synthetised PstI-HpaI fragment containing the lacUV5 promoter and the 5′ moiety of gII to the large PstI-HpaI-fragment of pHP1Aev. Expression of gII encoding the replication protein and downstream genes is inducible by IPTG. This helper plasmid produces nanorods at ˜10-fold higher amount in comparison to its parent pHP1Aev (FIG. 13).

pHP1LacAevG8 is identical to pHP1LacAev, except that 5 Gly residues are displayed at the N-terminus of mature pVIII protein (gVIII −20am nGGGG A1G, FIG. 54, SEQ ID NO: 97, SEQ ID NO: 98). This plasmid was constructed by ligating a custom-synthetised SnaBI-NcoI fragment containing gVIII-20am nGGGG A1G, FIG. 54, SEQ ID NO: 97, SEQ ID NO: 98) and the pIII::MCS sequence (FIG. 35, SEQ ID NO: 31, SEQ ID NO: 32) to the large SnaBI-NcoI fragment of pHP1Lac. The N terminal GlyGlyGlyGlyGly motif allows enzymatic attachment of proteins and peptides containing C-terminal LPXTG motif, or small molecules conjugated chemically to an LPXTG motif using enzyme Sortase (SrtA Sa) of Staphylococcus aureus.

pHP2 is a helper plasmid containing gVIII 2am S17L (FIG. 33, SEQ ID 25, SEQ ID NO: 26) and gIII::MCS (FIG. 35, SEQ ID NO: 31, SEQ ID NO: 32). It was constructed by ligating two PCR-amplified fragments. One PCR-amplified fragment contained all phage genes apart from the f1 origin of replication using phage R779 (Sattar et al., 2015) as a template. The primers introduced SaIlI and XbaI sites into the product. The second fragment amplified Kn^{R} marker and pA15 origin of replication using VCSM13 phage as a template. Primers introduced SaIlI and XbaI restriction sites. Both fragments were cut with SaIlI and XbaI and ligated to each other to form pHP2.

pHP2G8 helper plasmid is similar to pHP2, except that pVIII displays GGGG at the N-terminus of mature protein (gVIII 2am nGGGG; S17L, FIG. 33, SEQ ID NO: 27, SEQ ID NO: 28). This plasmid was constructed by ligating a custom-synthetised SnaBI-NcoI fragment containing gVIII 2am nGGGG; S17L, FIG. 33, SEQ ID NO: 27, SEQ ID NO: 28) and the pIII::MCS sequence (FIG. 35, SEQ ID NO: 31, SEQ ID NO: 32) to the large SnaBI-NcoI fragment of pHP2. The N terminal GlyGlyGlyGly motif allows enzymatic attachment of proteins and peptides containing C-terminal LPXTG motif, or small molecules conjugated chemically to an LPXTG motif using enzyme Sortase (SrtA Sa) of Staphylococcus aureus.

pHP1C helper plasmid is similar to pHP1 except for gene III, which is a truncated mutant gIIIC::MCS (FIG. 36, SEQ ID NO: 33, SEQ ID NO: 34). This plasmid was constructed by ligating the SnaBI-BamHI fragment containing the gVIII-20am nAAGG, ΔP6, S17L and gIII::MCS to the large SnaBI-BamHI fragment of pHP2 plasmid. The synthetic fragment contains a pIII::MCS that includes a BamHI site. When ligated to a large SnaB-BamHI fragment of a pHP helper, it introduces deletion of the 5′ moiety of gIII encompassing the coding sequences for entire N1 domain and most of the N2 domain. Moreover, this pIII fragment was designed to contain 3 unpaired Cys residues at the N-terminus for functionalization by chemical conjugation of molecules via SH groups. Similarly to pHP1C, this plasmid was constructed by ligating the SnaBI-BamHI fragment containing the gVIII-20am nAAGG, ΔP6, S17L and gIII::MCS to the large SnaBI-BamHI fragment of pHP2 plasmid.

pHP1AevC helper plasmid is similar to pHP1Aev except for gene III, which is a truncated mutant gIIIC::MCS (FIG. 36, SEQ ID NO: 33, SEQ ID NO: 34). Similarly to pHP1C, this plasmid was constructed by ligating the SnaBI-BamHI fragment containing the gVIII-20am nAAGG, ΔP6, S17L, L27S (FIG. 32, SEQ ID NO: 19, FIG. 33, SEQ ID NO: 20) and gIII::MCS (FIG. 36, SEQ ID NO: 33, SEQ ID NO: 34) to the large SnaBI-BamHI fragment of pHP2 plasmid. This C-terminal domain of pIII displaying three unpaired cysteine residues at the N-terminus of the mature protein, suitable for chemical conjugation of molecules via SH groups (Zhang et al., 2020).

pHP1LacAevC121 helper plasmid is functionalised plasmids derived from pHP1LacAev encoding a fusion of a single-chain variable domain derived from two human antibodies (C121, that interacts with the receptor-binding domain (RBD) of the SARS-CoV-2 Spike protein (Robbiani et al., 2020); (FIG. 38, SEQ ID NO: 39, SEQ ID NO: 40). This plasmid was constructed by inserting coding sequences of a single-chain variable domain derived from a heavy chain and a light chain of antibody C121 that is specific to the SARS-CoV-2 Spike (S) protein. The single chain variable fragment (scFv) for this antibody was designed by bridging the Heavy (H) chain and the Light (L) chain through the [GGGGS]3 linker. The nucleotide sequence for each scFv was optimized for expression in Escherichia coli. Restriction sites NcoI and NotI designed to flank the scFv sequence were used for cloning. The NcoI-NotI-cut synthetic fragment was ligated to the large NcoI-NotI fragment of the helper plasmid pHD1LacAev. The recombinant helper plasmid thereby encoded a C121scFv-pIII fusion.

The AlaAla N-terminal functional group on the pVIII subunits of this helper plasmid was used to enzymatically attach LPETA-tagged biotin or Alkaline Phosphatase, used for visualisation of the BSFnano in the immunoassays detecting the SARS-CoV-2 Spike protein.

pHP1LacAevN3 helper plasmid is functionalised plasmids derived from pHP1LacAev encoding a fusion of an antigen-binding domain of a heavy-chain-only antibody N3 derived from a heavy-chain-only camelid antibody (Sherwood and Hayhurst, 2021) (FIG. 55, SEQ ID NO: 99, SEQ ID NO: 100). This plasmid was constructed by inserting custom-synthetised coding sequence of the antigen-binding domain of a heavy-chain-only antibody N3 between the signal sequence and the mature portion of pIII. Restriction sites NcoI and NotI designed to flank the VHH sequence were used for cloning. The NcoI-NotI-cut synthetic fragment was ligated to the large NcoI-NotI fragment of the helper plasmid pHD1LacAev. The recombinant helper plasmid thereby encoded a VHH N3-pIII fusion.

The AlaAla N-terminal functional group on the pVIII subunits of this helper plasmid was used to enzymatically attach LPETA-tagged biotin or Alkaline Phosphatase, used for visualisation of the BSFnano in the immunoassays detecting the SARS-CoV-2 NC protein.

pHP2FnB helper plasmid is a functionalised derivative of pHP2, encoding FnB-gIII fusion (FIG. 37, SEQ ID NO: 37, SEQ ID NO: 38). Nanorods produced using this helper display, at one of two ends, fibronectin-binding (FnB) domain of Sof22, Streptococcus pyogenes fibronectin binding protein II (SfbII). This domain binds human (and mammalian) fibronectin with high affinity (Rakonjac et al., 1995). Nanorods containing this fusion producing using a helper phage (Rnano3FnB) were shown to be superior to the long phage in a lateral flow dipstick assay (Sattar et al., 2015). To construct this plasmid, fibronectin-binding repeats were amplified using phage R780 (Sattar et al. 2015) as a template and primers that introduced restriction sites SfiI (forward) and NotI (reverse). The SfiI-NotI-cut PCR product was ligated to the large SfiI-NotI fragment of pHP2 helper plasmid.

pHP2G8FnB helper plasmid is a functionalised derivative of pHP2G8 encoding the pIII-displayed FnB identical to the one in pHP2FnB (FIG. 37, SEQ ID NO: 37, SEQ ID NO: 38). Nanorods produced using this helper plasmid will, in addition to FnB displayed at the pIII end, also contain GGGG-displaying pVIII that can be functionalised enzymatically using SrtA Sa. This plasmid was constructed in the same manner as pHP2FnB, except that pHP2G8 vector cut by SfiI-NotI was used instead of pHP2.

Specifications pBSFnano Replication-Assembly Plasmids:

Several pBSFnano plasmids (Table 3) have been constructed to have two types of BSF replication-assembly cassettes, “p” (FIGS. 4A, 5C, 6C), containing only positive truncated origin of replication and “pn” (FIGS. 4B 5B, 6A and B), containing both positive and negative origins of replication.

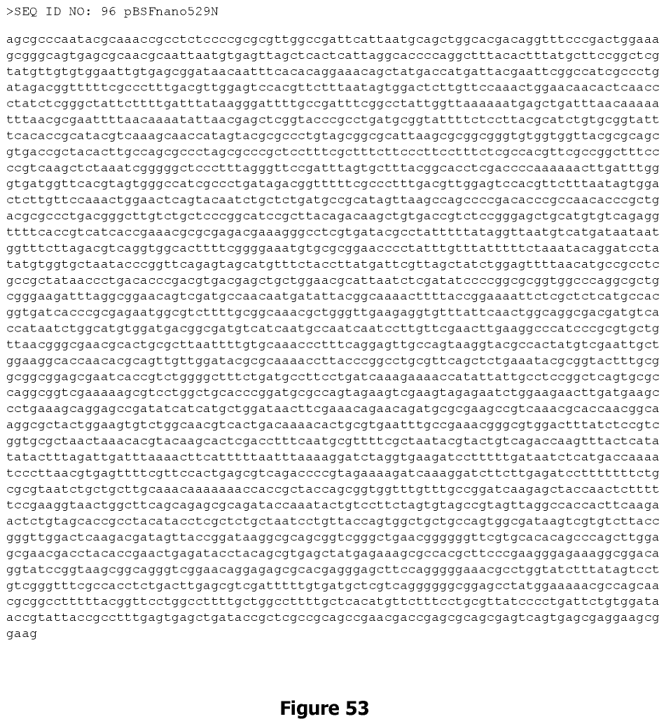

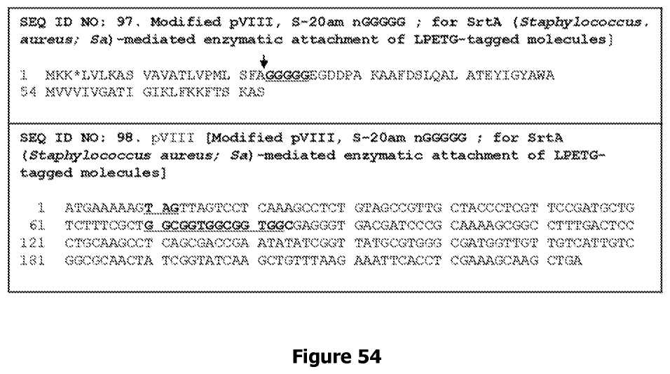

The BSFpn cassette variants constructed to date producing circular ssDNA of up to 748 nt in length (Table 6 and 7). Some applications, however, e.g., in diagnostic devices using linear dichroism, require long filaments; even longer than the wild-type Ff phages, to increase the signal and sensitivity of detection. The length extension is not possible in the Ff replicon (that has single-stranded DNA as replication intermediate) due to an increased tendency to be selected for loss of inserted DNA relative to double-stranded theta-replicating plasmids. The pBSF plasmids can be engineered to take up large inserts (fillers) between the segments required for replication and assembly, e.g., complete positive origin of replication (+) ori1 and packaging signal (PS), or downstream of PS (FIGS. 5B, 6A and B; segments labelled “filler”) and will replicate via the single-stranded and RF intermediates in the presence of pII produced from a helper plasmid. A generic map of the pBSFnano plasmid series is depicted in FIG. 9 and the elements and variants tabulated in Table 6 and 7. The pBSFnano variants are listed below:
pBSFnano-pn529
pBSFnano-pn529N (FIG. 9; SEQ ID NO: 96, FIG. 53)
pBSFnano-pn711;
pBSFnano-pn728;
pBSFnano-pn1400;
pBSFnano-pn79a;
pBSFnano-pn79Lac;
pBSFnano-pn313
pBSFnano-pn289
pBSFnano-p221.

The names of pBSF plasmids (pBSFnano-pnNumber or pBSFnano-pNumber) include the following components pBSFnano, standing for Biological Scalable Ff-derived nanorod (template plasmid).
pnNumber (-pn1400, -pn728, -pn711, -pn529, -pn395, -pn313, -pn289) denoting a replication-assembly cassette containing both the positive and negative f1 origin of replication (-pn; FIG. 5B, 6A) and a number indicating the size of circular ssDNA packaged into the BSF nanorods.
pNumber (-p221, -152) denoting a replication-assembly cassette containing the positive origin of replication and packaging signal (FIG. 5C, 6C; FIG. 47, SEQ ID NO: 77, SEQ ID NO: 79).
pn79a, denoting a BSFnano replication-assembly cassette that encodes minor coat proteins pVII and pIX expressed from phage promoter P_{A} (FIG. 6B; FIG. 41, SEQ ID NO: 52; FIG. 42, SEQ ID NO: 57, SEQ ID NO: 59).
pn79Lac denoting a BSFnano replication-assembly cassette that encodes minor coat proteins pVII and pIX expressed from promoter plac (FIG. 6B; FIG. 41, SEQ ID NO: 54, FIG. 42, SEQ ID NO: 59).

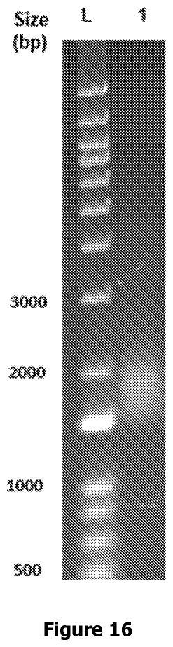

N denotes nadC auxotrophic marker (FIG. 50; SEQ ID NOs: 91, 92) instead of the ampicillin resistance (Amp^{R}) marker. Amp^{R} marker is not specified in the names of the pBSFnano plasmids (FIG. 16).

Specifications of Individual pBSFnano Replication-Assembly Plasmids

pBSFnano-pn728 is a BSFnano replication-assembly plasmid containing a BSFpn 728-nt replication-assembly cassette (FIG. 5C, 6A) comprising positive and negative origins of replication and packaging signal (FIG. 39, SEQ ID NO: 41, FIG. 40, SEQ ID NO: 45, SEQ ID NO: 51, SEQ ID NO: 50, SEQ ID NO: 48). Circular 728-nt ssDNA (FIG. 39, SEQ ID NO: 42) is produced and packaged into a BSFnano biological nanorods −100 nm in length in the presence of a helper plasmid from pHP series.

pBSFnano-pn728 was constructed in two steps. Firstly, phagemid pUC118 was amplified by inverse PCR. The resulting product contained the intact packaging signal and the (−) ori, however (+) ori was truncated, creating a Δ29 mutant that can only serve as terminator of pII-dependent (+) strand replication. This product was re-circularised, forming plasmid pUC118 Δ29. In the next step the complete (+) ori was amplified by PCR using f1 phage as a template, and primers that introduced EcoRI (forward) and SacI (reverse). The EcoRI-SacI-cut insert was inserted upstream of the f1 ori (into EcoRI-SacI of the MCS of pUC118 Δ29), to serve as the replication initiator within the BSFnano replication-assembly cassette.

pBSFnano-pn1400 is a BSFnano replication-assembly plasmid containing a BSFpn1400-nt replication-assembly cassette (FIG. 5C, 6A; FIG. 56, SEQ ID NO: 101) com composed of the complete (+) ori followed by a 986-nt filler I composed of a random DNA sequence that did not match any sequences in the NCBI database, PS, (−) ori and (+) ori Δ29 (FIG. 57 SEQ ID NO: 103, SEQ ID NO: 104; SEQ ID NO: 76, SEQ ID NO: 49, SEQ ID NO: 50; SEQ ID NO: 51). Circular 1400-nt ssDNA (FIG. 56, SEQ ID NO: 102) is produced and packaged into a BSFnano biological nanorods −200 nm in length (FIG. 1F) in the presence of a helper plasmid from pHP series.

DNA fragment corresponding to BSFnano-pn1400 replication-assembly cassette (FIG. 56, SEQ ID NO: 102) flanked by an EcoRI and an AatII site at the 5′ and 3′, respectively was custom-synthesized. The EcoRI-AatII-cut fragment was ligated to the large EcoRI-AatII fragment of plasmid pBSFnano-pn728.

pBSFnano-pn711 is a BSFnano replication-assembly plasmid containing a BSFpn 711-nt replication-assembly cassette (FIG. 5B, 6A) comprising positive and negative origins of replication and packaging signal (FIG. 39, SEQ ID NO: 43). Closed circular 711-nt ssDNA (FIG. 39, SEQ ID NO: 44) is produced and packaged into a BS nanorods of 100 nm in length in the presence of a helper plasmid of pHP series. The pBSFnano-pn711 plasmid was constructed by taking out 17 nucleotides from the sequence between the (+) ori1 and PS using blunt-cutting enzymes SmaI and HincII, followed by the self-ligation.

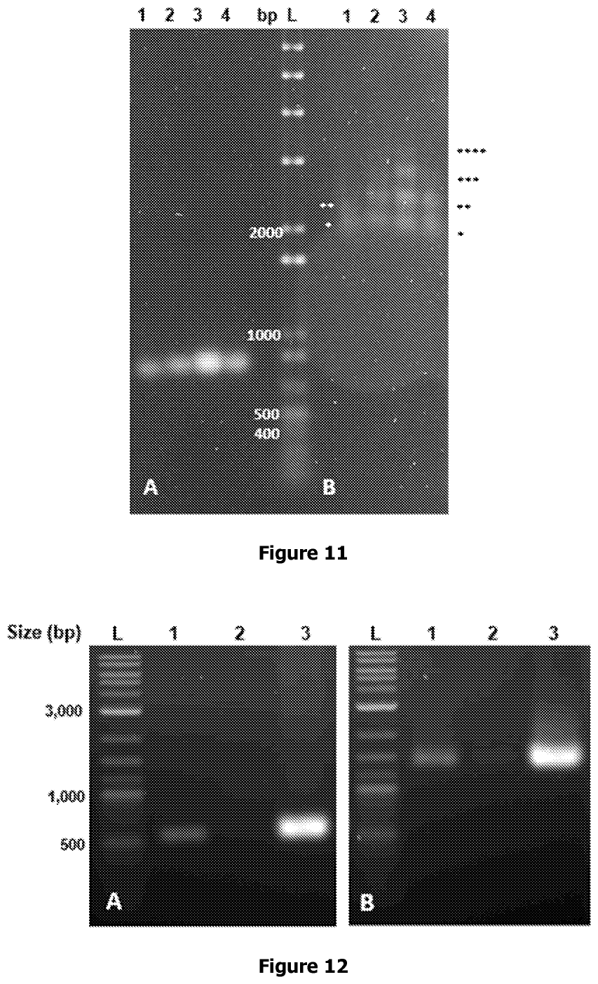

pBSFnano-pn79a is a BSFnano replication-assembly plasmid containing BSFpn cassette expressing pVII and pIX from the Ff P_{A} promoter. The promoter and gVII and gIX coding sequences were inserted between the (+) ori1 and PS (FIG. 42, SEQ ID NO: 57). Circular 707-nt ssDNA is produced and packaged in the presence of a helper plasmid from the pHP series (FIG. 42, SEQ ID NO: 58). Cis-expression of pVII and pIX from the 707-residue replicon that contains packaging signal increases the production of nanorods, by about two-fold in comparison to pBSFnano-pn711, due to the increased rate of nanorod initiation of assembly (FIG. 11; Table 8). This plasmid was constructed by inserting a custom-synthetized DNA fragment containing complete (+) ori1 followed by the phage P_{A} promoter driving expression of gVII and gIX. The P_{A} promoter was directly fused to the ribosome binding site (RBS) upstream of gII coding sequence and did not include the 5′ untranslated segment of gII mRNA which contains a site for binding of pV, which inhibits translation. The removal of the pV binding site prevents negative regulation of gVII gIX bi-cistronic mRNA translation. The RBS and sequence down to ATG codon was in turn fused to the gVIIgIX bicistronic coding sequence (FIG. 42, SEQ ID NO: 59). This fragment was designed to contain EcoRI and BamHI restriction sites at the 5′ and 3′ end, respectively, and was ligated to the large EcoRI-BamHI fragment of pBSFnano-pn728 between the (+) ori1 and PS.

pBSFnano-pn79Lac is a BSFnano replication-assembly plasmid containing BSFpn cassette expressing pVII and pIX from the lac promoter (FIG. 41, SEQ ID NO: 54). The promoter and gVII and gIX coding sequences were inserted between the (+) ori1 and PS (FIG. 42, SEQ ID NO: 58, SEQ ID NO: 59). Circular 748-nt ssDNA is produced and packaged in the presence of a helper plasmid from the pHP series. Inducible promoter allows gauging of pVII and pIX expression. Cis-expression of pVII and pIX from the 748-residue replicon that contains packaging signal increases the rate of nanorod initiation of assembly in the presence of IPTG by about five-fold relative to the pBSFnano-pn711 (FIG. 11, Table 8). This plasmid was constructed in the same manner as pBSFnano-pn79a, apart from the difference in the synthetic DNA fragment, in which the P_{A} promoter was replaced with the lac promoter, to allow gauging of the gVII and gIX production by the lac promoter inducer IPTG.

pBSFnano-pn529 is a BSFnano replication-assembly plasmid containing a BSFpn 529-nt replication-assembly cassette comprising positive and negative origins of replication and packaging signal (FIG. 43, SEQ ID NO: 61). Closed circular 529-nt ssDNA is produced and packaged into a BSF nanorods in the presence of a helper plasmid from the pHP series. This plasmid was constructed by removing 207 nucleotides between (+) ori1 and PS from pBSFnano-pn728. This was achieved by cutting pBSFnano-pn728 with SmaI (CCCGGG) and SfoI (GGCGCC; both blunt cutters) and self-ligation of the resulting large fragment.

pBSFnano-pn529N has the same BSFnano replication-assembly cassette as pBSFnano-pn529. Closed circular 529-nt ssDNA is produced and packaged into BSF nanorods in the presence of a helper plasmid from the pHP series. Selective marker in this plasmid is auxotrophic marker NadC (FIG. 59, SEQ ID NO: 91, SEQ ID NO: 92). The complete plasmid sequence is presented in FIG. 53 (SEQ ID NO: 96). This plasmid was constructed by ligation of two PCR-amplified fragments, one containing amplified nadC gene including its intrinsic promoter, and another encompassing the pBSFnano-pn529, apart from its bla (Amp^{R}) gene. The nadC gene was amplified using purified chromosomal DNA of E. coli K12 as template and two primers containing engineered restriction sites BamHI (forward) and SnaBI (reverse). pBSFnano-pn529 portion (without the bla gene encoding for Amp^{R} marker) was amplified using two primers that contained SnaBI (forward) and BamHI (reverse) restriction sites. The two amplified fragments were cut with BamHI and SnaBI and ligated to each other. The ligation mixture was subsequently transformed into a ΔnadC727 strain (K2486; Table 1). Transformants containing the new plasmid (pBSFnano-pn529N) were selected on the M9 minimal medium containing casamino acids (but lacking NAD).

pBSFnano-pn313 (Table 2) is a modified plasmid pUC57 containing a BSFnano-pn313 replication-assembly cassette (FIG. 45, SEQ ID NO: 70). Briefly, synthetic fragment corresponding to the BSFpn313 replication-assembly cassette (FIG. 45, SEQ ID NO: 70) flanked by a PstI and a SaIlI sites was custom-synthetised and inserted as a blunt dsDNA insert into the multiple cloning site of pUC57 by the manufacturer of the custom DNA fragment. The BSFpn313 cassette results in production and assembly of circular ssDNA of 313 nucleotides (FIG. 45, SEQ ID NO: 71), It is composed of (+) ori1, PS, (−) on and (+) ori2 (FIG. 46, SEQ ID NOs: 74, 76, 50 and 69). The (+) ori1 in this cassette is shortened by using truncated (+) ori1 that corresponds to only A portion of the f1 (+) origin (FIG. 46, SEQ ID NO: 74). This cassette has filler 1 of 24 nucleotides (FIG. 46, SEQ ID NO:75), and no filler II. The length of nanorods produced from pBSFnano-pn313 is −60 nm.

pBSFnano-pn289 (Table 2) is a modified plasmid pUC57 containing a BSFnano-pn289 replication-assembly cassette (FIG. 45, SEQ ID NO: 72). Briefly, synthetic fragment corresponding to the BSFpn313 replication-assembly cassette (FIG. 45, SEQ ID NO: 72) flanked by a PstI and a SaIlI sites was custom-synthetised and inserted as a blunt dsDNA insert into the multiple cloning site of pUC57 by the manufacturer of the custom DNA fragment. The BSFpn289 cassette results in production and assembly of circular ssDNA of 289 nucleotides (FIG. 45, SEQ ID NO: 73), It is composed of (+) ori1, PS, (−)ori and (+) ori2 (FIG. 46, SEQ ID NOs: 74, 76, 50 and 69). The (+) ori1 in this cassette is shortened by using truncated (+) ori1 that corresponds to only A portion of the f1 (+) origin (FIG. 46, SEQ ID NO: 74). This replication-assembly cassette contains no fillers (FIG. 46). The length of nanorods produced from pBSFnano-pn289 is −60 nm.

pBSFnano-p221 is a template BSFnano replication-assembly cassette containing a BSFp-221-nt replication-assembly cassette comprising positive origins of replication and packaging signal is derived from pNJB7. Circular 221-nt ssDNA is produced and packaged into nanorods 50 nm in length in the presence of a helper plasmid of pHP series. This plasmid was constructed by deletion of Km^{R} gene from pNJB7 (Sattar et al. 2015). The remaining selective marker is Amp^{R}.

### Specifications of the Host Strains Used in Two-Plasmid BSFnano Production System

Construction of plasmids was carried out in non-suppressor strains (e.g., K2245; Table 1), to prevent production of pVIII encoded by gVIII^{amber} mutants of the helper plasmids, thereby avoiding the toxicity of pVIII that results in selection for mutations that eliminate pVIII production (e.g., mutations in promoter or coding sequence; personal observation). These strains also contained lac^{Iq} mutation that minimizes expression from the lac-operator-controlled promoters (e.g., lac, lacUV5, tac). These non-suppressor strains were also used for purifying the pHP helper plasmid series DNA for later use to transform a nanorod-production strain.

For production of nanorods in the two-plasmid system, double-transformed supD host strain (e.g., K2091) was used. The host strain was first transformed with purified DNA of a pHP plasmid. Electrocompetent pHP-containing cell aliquots were prepared and stored at −80° C. Cells were transformed with a pBSFnano replication-assembly plasmid using electroporation. Nanorod production was performed from the pool of transformed cells as described in Material and Methods.

Aside from supD tRNA (expressed from a chromosome or plasmid) other mutations or plasmids in the BSFnano-producer strain can be used, depending on the properties of a particular combination of pHP and pBSF helper plasmid (e.g., depending on inducible promoter used for controlled expression, or marker used for selection of transformants).

Additional mutations in E. coli cells were required for helper plasmid variants that were engineered for in vivo azide incorporation into nanorods via insertion positions into pVIII at specific positions (pHP1Az, pHP1lacAz, pHP1AzCM, pHP2AzCM) as described in detailed specifications of the four pHP1 series plasmids listed above. In vivo azide incorporation into pVIII and nanorods occurs by insertion of unnatural amino acid azidohomoalanine (Aha) during translation of ATG (Met) codons instead of Methionine.

To be able to control incorporation of Met and Aha into proteins at Met codons the host cells have to be methionine auxotrophs (Kiick et al., 2002). Met auxotrophy is achieved in E. coli strains containing loss-of-function metE allele (Hondorp and Matthews, 2006). A metE::Kan^{R} replacement (null) allele (Baba et al., 2006) was introduced by generalized P1 transduction [Methods; (Sternberg and Maurer, 1991)] into the production strain K2091 (that also has supD and lac^{Iq} mutations) and K1030 (supD). Kan^{R} cassette (flanked by frt sites) was removed from the transductants' genomes using transiently expressed frt-specific recombinase FLP [(Baba et al., 2006; Cherepanov and Wackernagel, 1995); Methods] expressed from plasmid pCP20 (Table 4).

Host strains may contain accessory plasmids for mosaic or trans-expression of pVIII, pVII, pIX, pVI or pIII. Expression of coat proteins from accessory plasmids is required in case of constructing fusions that prevent assembly of Ff (and by extension the BSFnano nanorods) unless they are combined with copies that do not display any peptides, or if an application dictates smaller number of displayed peptide copies than the number of copies of the given protein in the nanorod. In case of display on pVIII, it is known that many peptides longer than 6 residues prevent assembly of the Ff phage (Iannolo et al., 1995) and have to be combined with pVIII copies that do not display any peptide. This is termed “mosaic” display. In the case of the nanorod assembly system, the non-display copy of pVIII is expressed from the pPop-up or the helper plasmid pHP, whereas the pVIII fusion containing displayed peptide is expressed from a compatible accessory plasmid. The non-display copies of pVIII and the copies displaying a fusion are made to be identical over the portion of pVIII that mediates subunit-subunit interactions that form the nanorod in order to assemble into “mosaic” nanorods. For this reason, if a helper-phage-encoded pVIII contains changes in the portion that mediates subunit-subunit interactions in the virion, the pVIII expressed by accessory plasmids contain equivalent changes.

The accessory plasmids must have a plasmid origin of replication and a selective marker that is compatible with the Pop-up or pHP and pBSFnano plasmids, for example oriD origin of replication and Cm^{R} marker.

### Example 3. BSF Nanorod Nomenclature and Production

Given the plethora of plasmids in the single-plasmid and two-plasmid nanorod production systems, pPop-up and pHP+pBSFnano, respectively, result in a series of different nanorods in the terms of length and displayed functional groups or peptides, it is helpful to have a clear nomenclature of the BSF nanorods produced by this system according to the scheme detailed in the text below.

The nanorod names follow a scheme: BSFnano[number][pVIII modification][1 or 2][pIII modification], where:

BSFnano refers to the biological scalable Ff-derived nanorods.
[number] refers to the length (in nucleotides) of the circular ssDNA packaged into the nanorods
[pVIII modification] refers to motifs allowing enzymatic modification (e.g., G8 or Aev)
[1 or 2] refers to positions of the amber mutations in gVIII (codons 4 and 25, respectively) and in pVIII (Ser-20amber and Glu2amber)
[pIII modification] refers to peptide or protein displayed on pIII, e.g., FnB, scFvC121 or VHH N3.

Other annotations are: YM, referring to pVIII variant containing Y21M and wild-type pIII without MCS.

For example, BSFnano529YM1 corresponds to BSF nanorod containing amber codon in position 4 (or −20 relative to the mature pVIII), pVIIIY21M and wild-type pIII, containing a 529-nucleotide circular ssDNA.

Nanorod production and purification is carried out as described in Material and Methods.

### Example 4. Comparison of the Nanorod Production by pPop-Up Plasmids Containing BSFpn Vs. BSFp Replication-Assembly Cassette

In this disclosure a novel replication-assembly cassette, BSFpn, containing the Ff (−) ori, was designed to achieve a higher production of nanorods relative to the existing replication cassette that contains only the (+) ori, and then only the A (I) portion, and no negative origin [BSFp or “miniphage” cassette; (Specthrie et al., 1992)].

To produce nanorods, electrocompetent cells of nanorod production strain K2091 were transformed with the pPop-up529YM or pPop-up221YM plasmid and incubated overnight in a medium containing kanamycin to select for the plasmid. Bacteria were removed by centrifugation and nanorods concentrated by PEG precipitation followed by purification using CsCl gradient and anion exchange chromatography (as described in Materials and Methods section). Purified nanorods were analysed by disassembled-nanorod agarose gel electrophoresis (FIG. 10) and quantified using densitometry as described in Materials and Methods section. The amount of nanorods produced by pPop-up529YM per litre of culture was 20-fold higher than by pPop-up221YM (4.8×10¹⁴ vs. 2.4×10¹³; respectively; Table 8). Therefore, including the (−) ori within the nanorod replication-assembly cassette BSFpn has increased nanorod production approximately twenty-fold relative to the BSFp cassette containing only the A domain of the (+) strand origin in (+) ori1 and no (−) ori, when replication is driven by an IR1-B mutant of pII. There is, however, a trade-off in that the more-efficiently-replicating BSFpn cassette cannot produce nanorods as short as BSFp cassette (70 nm vs. 40 nm; FIG. 1A vs. C). Shorter BSFpn cassettes, BSFpn 313 and BSFpn 289, that contain a truncated (+) strand ori (A or I portion) as (+) ori1 and (−) ori have been designed that are predicted to produce very short nanorods (˜60 nm and ˜55 nm in length), respectively, at higher amounts in comparison to the BSFp nanorod replication-assembly cassettes production of very short nanorods (FIG. 45, SEQ ID NO: 70, SEQ ID NO: 71, SEQ ID NO: 72 SEQ ID NO: 73).

### Example 5. Increase of the BSF Nanorod Production in the Two-Plasmid System by Expression of Proteins pVII and pIX from the BSFnano Replication-Assembly Cassette

In order to increase the rate of initiation (that requires assembly-initiating minor coat proteins pVII and pIX) and thereby increase the number of assembled nanorods, genes encoding these two small proteins (33 and 32 residues in length, respectively) were within the “filler I” sequence between the (+) ori1 and packaging signal (FIG. 5B, 6B) under the control of constitutive Ff phage P_{A} promoter (plasmid pBSFnano79a; Table 2) or inducible lac promoter (plasmid pBSFnano79Lac; Table 2). In the presence of protein pII, these two plasmids contain the nanorod replication-assembly cassettes for replication of circular ssDNA 707 nt and 748 nt in length, respectively. Due to the presence of a (−) ori, short circular double-stranded RF form is produced by replication of the (−) strand. These short RFs are templates for the (+) strand replication and expression of pVII and pIX. Nanorods were produced from these pVII- and pIX-expressing replication-assembly cassettes in the presence of the helper plasmid pHP2, giving rise to nanorods (BSFnano79a.2 and BSFnano79Lac.2). The amount of produced nanorods was compared to control nanorods BSFnano711.2 generated from control plasmid pBSFnano-pn711, a template plasmid that does not express any proteins from the replicated BSF cassette (FIG. 11A). The final concentration of BSFnano711.2 nanorods (quantified based on the amount of encapsulated ssDNA as described in Material and Methods section) was 2.6×10¹⁴ per L of cell culture, while the final concentration of BSFnano79a.2 was 4.3×10¹⁴ per L. The concentration of BSFnano79Lac.2 nanorods produced by the uninduced batch was 6.8×10¹⁴ per L, whereas the IPTG induced BSFnano79Lac.2 nanorod production was 1.5×10¹⁵ per L (Table 8). Highest production was therefore observed using pBSFnano79Lac template plasmid, under the conditions where expression of pVII and pIX was induced (in the presence of 0.1 mM IPTG). Increased production of nanorods, however, resulted in increase of double- triple- and quadruple-length nanorods (FIG. 11B; bands labelled with two, three or four asterisks in lanes 1-4). The reason for this could have been the increased ratio of assembly initiation proteins pVII and pIX relative to assembly termination proteins pVI and pIII, possibly causing relatively higher efficiency of initiation relative to termination and release (Rakonjac, 1998).

### Example 6. Increase of the Nanorod Production by Introduction of Inducible Expression of the Replication Protein pII

Based on published work (Lerner and Model, 1981; Smeal et al., 2017a, b), Ff phage production falls to a low level after about 10 E. coli division times after infection. Given that the in the nanorod production system plasmids are introduced into a host cell by transformation, the initial number of transformed cells is rather low (˜107 per transformation, diluted into 1 L of media). Ten cell divisions would bring the transformed cell number up to 10¹⁰. Given that the nanorods are produced in a large-volume cultures (e.g., 1 L), the production of nanorods is predicted to cease at a low titre (10⁷ cells/mL). Given the nanorod production tapers and essentially ceases after 7 cell divisions, there is a limited number of the nanorods each cell can produce, hence the low starting cell numbers result in an overall low number of produced nanorods.

In order to overcome the nanorod replication and assembly plateau at a low cell titre, this invention included a solution to the problem whereby nanorod replication and assembly was delayed until the transformed E. coli cells increased in density. This was achieved by introducing inducible expression of replication protein pII. To achieve inducible expression, constitutive Ff promoter P_{A} upstream of gII was replaced by inducible lacUV5 promoter (e.g., pPop-up529LacYM). In this way expression of gII could be delayed until the density of the transformed cell culture reached 0.1 (corresponding to a cell titre of ˜5×10⁷ per mL), by adding IPTG to the transformed cell culture. High-efficiency nanorod replication and assembly occurs over 4 h post-induction, at high titres of cells containing the pPop-up plasmid. The nanorod numbers produced by pPop-up529LacYM under these conditions were about 10-fold higher in comparison to the nanorods produced by pPop-up529YM plasmid expressing pII under the constitutive phage promoter P_{A} (5.0×10¹⁵ vs. 4.8×10¹⁴; FIG. 12; Table 8). A ten-fold improvement is very important, given that it decreases the required volume of the culture by 10-fold, thereby minimising the labour and reagents that go into production and purification of nanorods. In contrast to the system expressing pVII and pIX described in Example 6 (FIG. 11B), nanorods produced under inducible pII expression produced were unit-length and no double- or triple-length nanorods were detectable by agarose gel electrophoresis of native purified nanorods (FIG. 12B, 19).

Not only inducible-replication single-plasmid, but also two-plasmid nanorod production system was constructed and tested, also resulting in increased production of the nanorods producing exclusively unit-length nanorods (e.g., FIG. 13).

### Example 7. Construction of Minimal-Length BSFpn and BSFp Cassettes Producing Minimal-Length BSF Nanorods

Shortened BSFpn and BSFp cassettes were further designed and constructed in a pPop-up single-plasmid system to minimise the length of the produced nanorods (pPop-up395LacYM and pPop-up152LacYM). BSFpn and BSFp cassettes in these plasmids were reduced in length by removing the sequences between the secondary motifs corresponding to the (+) and (−) on and the packaging signal (Table 9). The shortened nanorod replication-assembly cassettes were custom-synthetised and inserted into the pPop-up backbone. Minimal length of the BSFpn cassette that contains a complete (+) on as (+) ori1 resulted in a circular ssDNA 395 nt in length (FIG. 14A, Lane 2; FIG. 43, SEQ ID NO: 63, SEQ ID NO: 64) assembling into nanorods 70 nm in length (FIG. 1C). Further reduction of the BSFpn cassette is achievable by removal of the B (or II) portion from the (+) strand origin (Table 9), to obtain the ssDNA 313 nt or 289 nt in length, with predicted length of ˜60 or ˜55 nm. A minimal BSFp cassette gave a circular ssDNA product of 152 nt (FIG. 15A, Lane 2; FIG. 47, SEQ ID NO: 79, SEQ ID NO: 80), producing nanorods that are 40 nm in length, the shortest Ff-derived nanorods produced to date (FIG. 1A). Production of the shortened nanorods from pPop-up395LacYM and pPop-up152LacYM was somewhat lower in comparison to pPop-up529YM and pPop-up221YM, but within the order of magnitude (Table 8).

### Example 8. Introduction of Auxotrophic Marker into a pBSFnano and pPop-Up Plasmids

When the BSFnano nanorods are being assembled in a two-plasmid or one-plasmid system, one particle in a million or one in a billion, respectively, packages the entire plasmid due to rare recombination events that remove the terminator copy of the (+) on (ori2) (data not shown). Given that these template plasmids include antibiotic resistance genes, the produced longer particles carry these genes and could provide opportunity to spread antibiotic resistance genes by transducing E. coli strains in the gut or environment. One way to resolve this issue is to construct new vectors that have auxotrophic selective markers, which do not have any negative effect on environment or the living organisms. Selection of plasmids containing these markers is based on complementation of auxotrophic mutation in a host strain that is unable to synthesize a metabolite required for bacterial growth (amino acid, vitamin, nucleotide, cofactor etc.). Auxotrophy for cofactor NAD was chosen, given that it does not require preparation of complex mixtures of amino acids for optimal growth, in contrast to auxotrophic markers involved in amino acid synthesis. A host strain constructed for production of the BSFnano nanorods, K2487 (Table 1) is a deletion mutant lacking one of the enzymes of the NAD biosynthetic pathway, ΔnadC, encoding the quinolinate phosphoribosyl transferase (Bhatia and Calvo, 1996; Dong et al., 2010). Plasmids pBSFnano529 and pPop-up529Lac were modified by replacing the Amp^{R} or Kan^{R} genes, respectively, with the nadC gene (FIG. 50, SEQ ID NO: 91, SEQ ID NO: 92, SEQ ID NO: 93), and the new plasmids were named pBSFnano529N and pPop-up529LacAevN. The selection for ΔnadC strain K2487 transformed with pBSFnano529N or pPop-up529LacAevN was done in minimal medium supplemented with casaminoacids (casein hydrolysate), a nutrient which does not contain NAD. Nanorods produced in the minimal media were concentrated by ultrafiltration rather than PEG precipitation. PEG at high concentration (15%) necessary to precipitate nanorods caused crystallization of salts from the minimal media and could not be used for concentration of nanorods. The nanorods concentrated by ultrafiltration, were resuspended in a suitable buffer, treated by DNAase and RNAase and further concentrated by PEG precipitation and purified CsCl gradient centrifugation as described in Materials and Methods. BSFnano nanorods were detected by native nanorod electrophoresis (FIG. 16).

### Example 10. Spontaneous Liquid Crystal Formation by the 50 nm and 80 Nm BSFnano

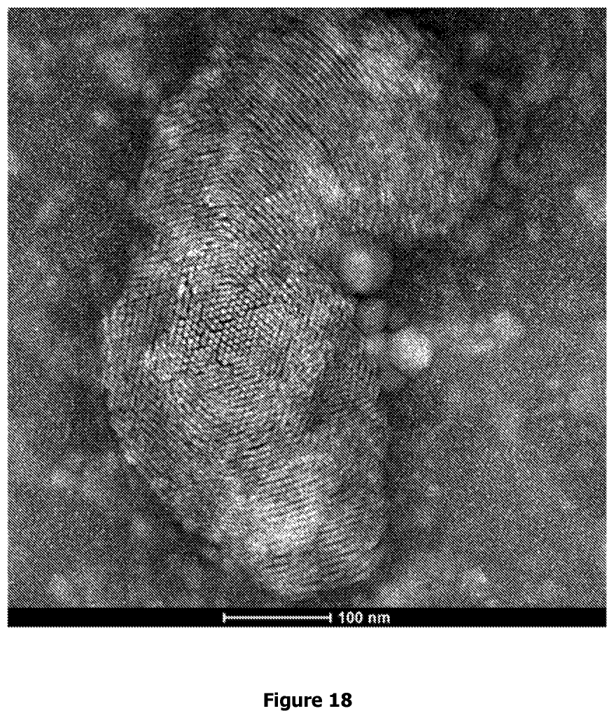

Property of the Ff filament to form liquid crystals can be used in development of sensors and new materials (Cao et al., 2016; Chen et al., 2013; Chung et al., 2014; Chung et al., 2011; Lee et al., 2013). Whereas long Ff filaments require exposure to dextrose or PEG to form liquid crystals, we observed that highly concentrated purified nanorods formed liquid crystals spontaneously in buffers such as phosphate-buffer saline, as observed by TEM. BSFnano221.2 nanorods (FIG. 17) formed three-dimensional one-layer disc-like liquid crystals (FIG. 18), whereas BSFnano529.2 have spontaneously formed two-dimensional flat-ribbon-like structures (FIG. 19). Spontaneous formation of these structures by short nanorods is of interest, as it indicates relative ease of the liquid crystal formation in comparison to full-length Ff filaments.

### Example 11. Lateral Flow Assay for Detection of Human Fibronectin Using the BSFnano Nanorods

An example of the BSF nano use in diagnostics is a dipstick assay for detection of human fibronectin using BSFnano728.FnB2 nanorods (110 nm in length) displaying FnB, a high-affinity fibronectin-binding domain from a S. pyogenes surface protein (Rakonjac et al., 1995). Nanorods 50 nm in length were demonstrated previously to be superior to long phage in detection of fibronectin (Sattar et al., 2015). Those nanorods were made using the phage system that required a very lengthy procedure to separate short nanorods from the long phage. FnB-displaying nanorods in this current invention have been constructed in the phage-free two-plasmid system, using a combination of the helper plasmid pHP2G8FnB, that encodes FnB-pIII fusion (FIG. 37, SEQ ID NO: 37, SEQ ID NO: 38) and the nanorod replication-assembly plasmid pBSFpn728 (FIG. 20). In the nanorods assembled by the plasmid system display of FnB repeats was much more efficient than in the published phage-based system (2 vs. 0.5 copies of FnB domain in the plasmid vs. phage system, respectively; FIG. 20A). This is likely due to a much shorter procedure for purification of nanorods, resulting in much less degradation of the FnB portion of the FnB-pIII fusion. Assay sensitivity i.e., limit of detection (LOD) of the nanorods produced in the plasmid system was 0.04 ng/mL (FIG. 20B) vs. 6.4 ng/mL in the phage system (Sattar et al., 2015). The 160-fold improvement is due to increased avidity of the nanorods produced in the plasmid system. A higher avidity due to a more rapid purification and decreased proteolytic degradation of displayed proteins makes the lateral flow assays using the plasmid system superior to those using the phage system. The increased avidity is, however, accompanied by a background signal in the absence of analyte in the assay where nanorods were detected by enzymatic visualization, using Alkaline Phosphatase (FIG. 20B). In contrast, nanorods that were covalently fluorescently labelled with DyLight 550 via primary amines (FIG. 20C) gave no signal in the absence of the analyte, however, the LOD using fluorescent visualization was very high (data not shown), indicating a much poorer assay sensitivity in comparison to the enzymatic visualization using Alkaline Phosphatase. Overall, this example shows that combination of enzymatic visualization with increased avidity of the nanorods is suitable for high-sensitivity lateral flow assays, such as rapid antigen tests for detection of various pathogens in food or patient samples.

### Example 12. Enzymatic Attachment of Functional Groups to Nanorods with Sortase A

Chemical functionalization of nanorods is costly and therefore not suitable for upscaling. For this reason, the nanorod production system was modified to allow enzymatic attachments of ligands, by displaying specific sequence motifs on the N-terminus of pVIII (helper plasmids pHP1LacAev, and pHP1LacAevG8 or pHP2G8). These three helper plasmids were designed to display AlaAla and GlyGlyGly motifs, respectively, that are substrates of transpeptidases Sortase A from Streptococcus pyogenes (SrtA-Sp) and Staphylococcus aureus (SrtA-Sa), respectively, which catalyse a reaction with C-terminal tags LPETA (LeuProGluThrAla) and LPETG (LeuProGluThrGly), respectively.

Here we will discuss the example allowing enzymatic attachment of ligands using SrtA-Sp. The original plasmid (pHP1LacA) was designed by adding AlaGly between the N-terminal Ala residue and Glu at position 2 and removing Pro at position 6 of the mature pVIII (SEQ ID NO: 17; AAGGEGDDAKAAFDSLQALATEYIGYAWSMVVVIVGATIGIKLFKKFTSKAS). This helper plasmid, in combination with the pBSF template plasmids, did not produce any nanorods, suggesting the AA-pVIII fusion was poorly functional in phage assembly. To overcome this impediment, sequence encoding this pVIII variant and pIII:MCS was cloned into VCSM13 phage to obtain phage R786 and checked for ability to form plaques. The phage formed pinpoint turbid plaques and the stock titers were around 10¹⁰ per mL, about two orders of magnitude lower than the titer of VCSM13. In order to improve pVIII function, R786 was “evolved” through three rounds of growth at low m.o.i. (1:1000) without plaque purification, as described in Methods and experimental procedures. After the third round the phage were plated and several large plaques were clonally purified, and the resulting stocks were titrated. The titres matched that of the parent R783 phage (a control producing wild-type gVIII). Sequence analyses detected new mutations in pVIII in the evolved phage. Two mutated gVIII sequences from the evolved phage (FIG. 32, SEQ ID NO: 19, FIG. 33, SEQ ID NO: 20, SEQ ID NO: 21, SEQ ID NO: 22) were cloned back into the pHP1Lac backbone to obtain pHP1LacAev and pHP1LacAev5 helper plasmids, containing L27S and D5A variants, respectively (FIG. 32, SEQ ID NO: 19, FIG. 33, SEQ ID NO: 20, SEQ ID NO: 21, SEQ ID NO: 22). The helper function was tested based on the ability to replicate and assemble a standard phagemid (pUC118) which can be easily titrated by transduction of Amp^{R} into an indicator strain (e.g., K561 or TG1, Table 1). Clone pHP1LacAev that had L27S mutation in pVIII (FIG. 32, SEQ ID NO: 19; FIG. 33, SEQ ID NO: 20) gave better phagemid particle titres and was used in further work. The two evolved pVIII variants differ in charge, which may be of interest to applications that involve specific charge of the nanorod (FIG. 32, SEQ ID NO: 19, FIG. 33, SEQ ID NO: 20, SEQ ID NO: 21, SEQ ID NO: 22).

Expression of the replication protein pII from the helper plasmid pHP1LacAev was controlled from a lacUV5 promoter, allowing induction of the ssDNA replication and nanorod production at the most suitable densities of transformed cells. Enzymatic attachment of LPETA-labelled functional groups, peptides, or proteins to the nanorods displaying AA motif on each copy of pVIII can be performed as presented schematically in FIG. 21.

### Example 13. Nanorods-Based Immunodetection of SARS-CoV-2

As two examples, LPETA-tagged FITC (FIG. 22) or biotin (FIGS. 23,24A) or LPETG-tagged enzyme β-glucuronidase of E. coli (UidA or GUS; FIG. 24B) were used to modify BSFnano nanorods that were used instead of cell-culture-produced antibodies to develop immunoassays (dot-blot, ELISA, and lateral-flow) for detection of the SARS-CoV-2 antigens, Spike and nucleocapsid (NC) proteins (FIGS. 25-27).

To develop nanorods for detection of the SARS-CoV-2 Spike protein, variable domains of the gene encoding a high-affinity antibody against the Spike protein isolated from a convalescent patient's B cells, C121 (Robbiani et al., 2020) were amplified and combined into a single-chain variable domain scFvC121. The scFvC121 amplicon was inserted into the helper phage pHP1LacAev to form an scFvC121-pIII fusion (FIG. 38, SEQ ID NO: 39, SEQ ID NO: 40). The resulting helper plasmid, pHP1LacAevscFvC121 was used in conjunction with the nanorod replication-assembly plasmid pBSFnano728, to produce nanorods displaying variable domains of C121 antibody (BSFnano728Aev1C121). These nanorods display AlaAla motif on every copy of the major coat protein pVIII and therefore allow for enzymatic attachment of LPETA-tagged molecules. To test the enzymatic attachment, FITC-LPETA conjugate was mixed with these nanorods in the presence of SrtA Sp as described in the Methods section. Labelled nanorods were separated from unreacted FITC-LPETA and SrtA-Sp enzyme by PEG precipitation, resuspended and analysed by the native nanorod agarose gel electrophoresis (FIG. 22). Band of the FITC-labelled nanorods showed bright fluorescence in the absence of staining (FIG. 22A, lane 1), whereas unlabelled control nanorods were not visible (FIG. 22A, lanes 2 and 3). Bands of control nanorods were visualized after in-gel nanorod disassembly by soaking in NaOH solution, neutralization and staining the exposed DNA in EtBr (FIG. 22B). biotin-LPETA attachment to nanorods was performed to allow a large array of Avidin-based tags to be displayed on the nanorods. The attachment of biotin-LPETA was attempted first, as it can be directly visualised using Streptavidin-gold nanoparticles and TEM. Purified nanorods produced using the pHP1LacAev helper plasmid were modified by attachment of biotin-LPETA by SrtA Sp. Labelled nanorods were separated from soluble

biotin-LPETA and SrtA-Sp by microfiltration using a spin-column. Purified biotin-functionalised nanorods were labelled on-grid with Streptavidin-gold nanoparticles and analysed by TEM imaging. TEM analysis indicated that there was specific labelling along the length of the nanorod by Streptavidin-gold (FIG. 23A-F), while in the control reaction without Sortase A, no labelling of the nanorods occurred (FIG. 23G-I). In a subsequent labelling experiment, high-density labeling of the nanorods was performed as described above, except that higher amounts of substrate and enzyme were used. Labelled nanorods were analysed by native agarose gel electrophoresis and western blotting, using Avidin-Alkaline phosphatase conjugate. The western blot showed very intense signal in the lane containing biotinylated nanorods (FIG. 24A, lane 1), and no signal in the lane containing unlabelled control (FIG. 24A, lane 2).

Nanorods were also produced using the helper plasmid displaying 5 Gly residues at the termini of every pVIII (BSFnano728G8). These nanorods were directly modified by enzyme β-glucuronidase (GUS) of E. coli (Feldhaus et al., 1991) expressed with the LPETG tag at the C-terminus. Agarose gel electrophoresis of the nanorods followed by in-gel detection of GUS using a chromogenic substrate (100 mM NaPO4 pH 7.0, 1 mM X-GLUC:Na, 200 μM NBT) demonstrated successful attachment of this enzyme to the nanorods. (FIG. 24B).

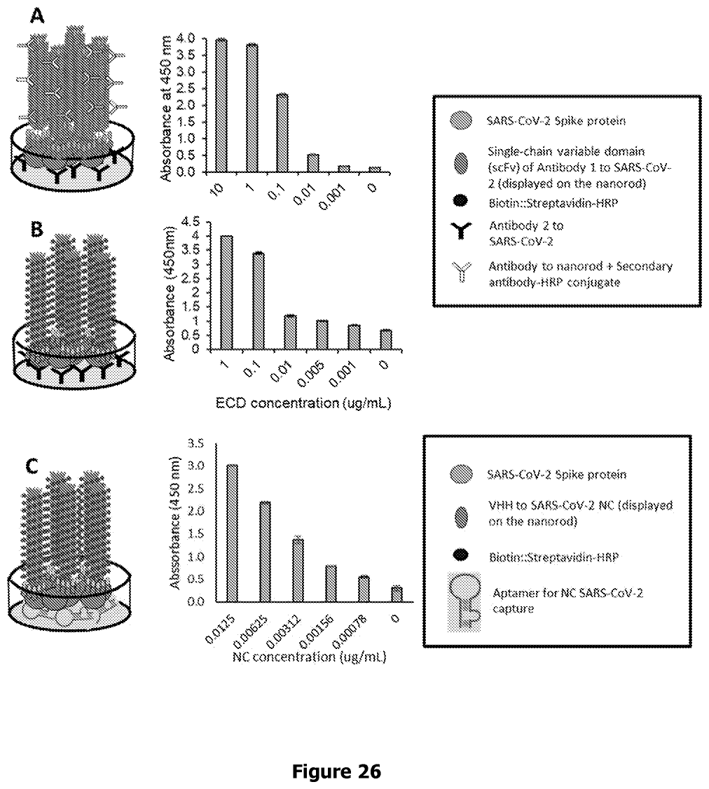

biotin-labelled and unlabelled nanorods were used for detection of the of the SARS-CoV-2 Spike protein extracellular domain (ECD) using dot-blot and ELISA assays (FIGS. 25 and 26).

In the dot-blot assay (FIG. 25) the Spike protein ECD was immobilized by binding to a membrane. After blocking, the membrane was exposed to the scFvC121-displaying biotinylated nanorods BSFnano728Aev1C121, followed by Avidin-Alkaline Phosphatase.

Membrane-bound nanorods were visualized using chromogenic substrate NBT/BCIP which form a dark-purple insoluble product in the presence of Alkaline Phosphatase. In this setup 10¹¹ he BSFnano728Aev1C121 detected as little as 1 ng of ECD.

When used in a sandwich ELISA assay, immobilised anti-Spike antibody CR3022 was used as a capture molecule for ECD, whereas either unmodified (FIG. 26A) or biotinylated (FIG. 26B) scFvC121-displaying nanorods were used for detection of ECD. In the former, nanorod-specific antibodies and secondary HRP-conjugated antibodies were used for detection of ECD, whereas in the latter, biotinylated nanorods and Avidin-HRP conjugate were used. Assay with unlabelled nanorods (FIG. 26A) showed much lower background signal and detected ECD at 10 ng/μL. Assay using the biotinylated nanorods showed very high background signal and lower sensitivity in comparison to unlabelled nanorods.

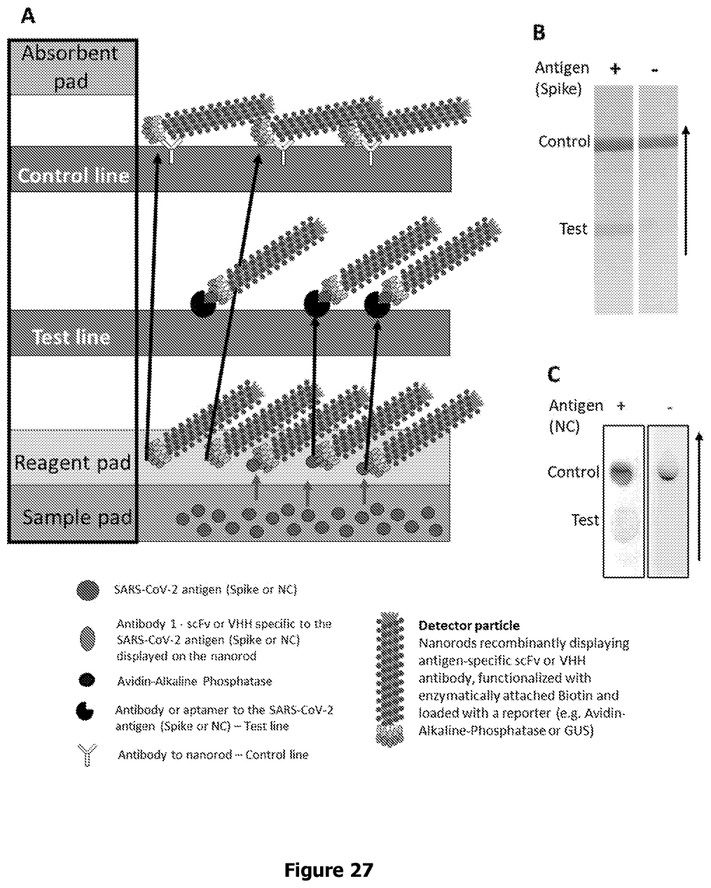

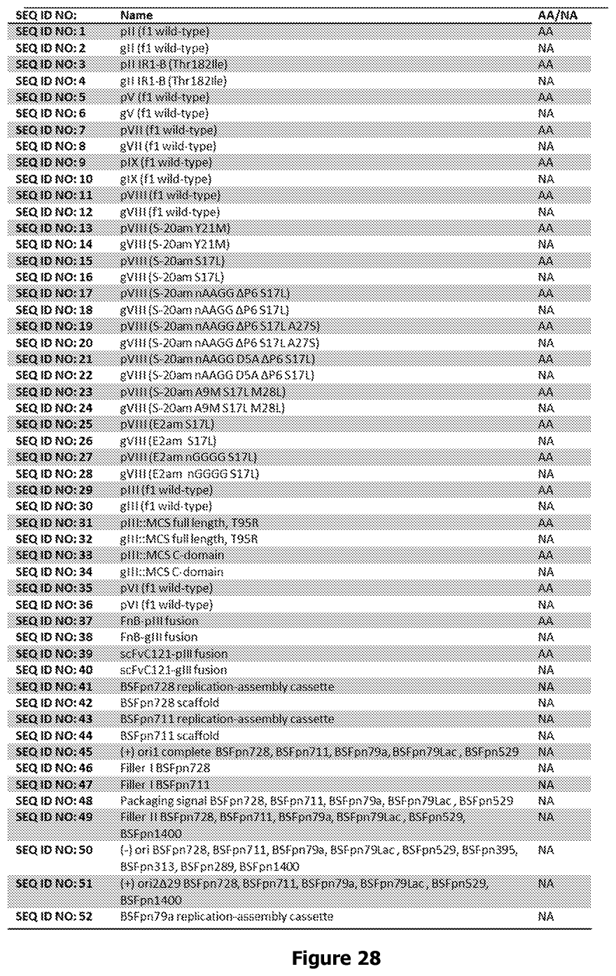

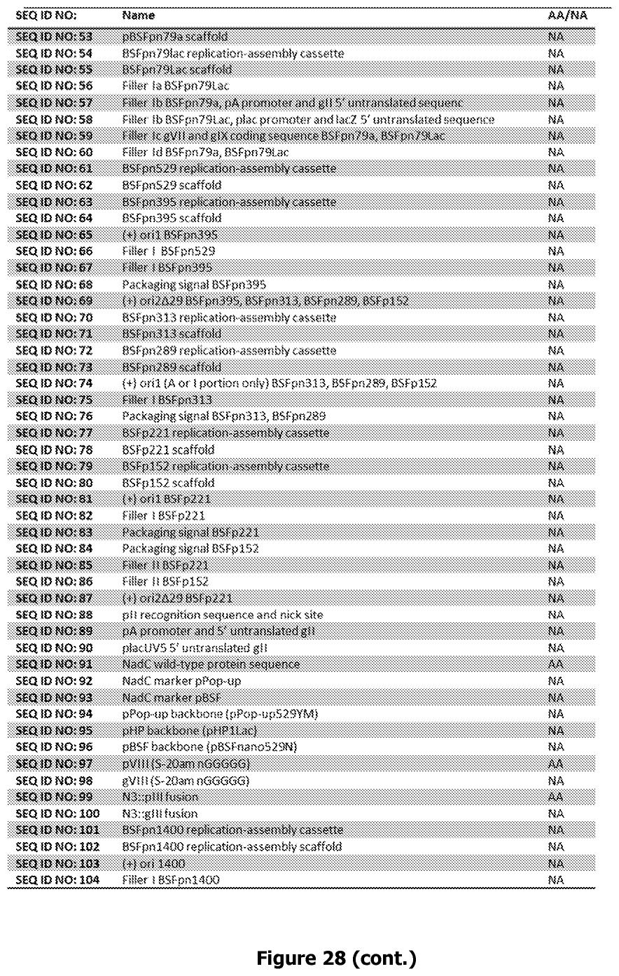

The scFvC121-displaying nanorods BSFnano728Aev1C121 were next used to develop a lateral flow Spike ECD detection assay. biotinylated BSFnano728AevpIIIC121 in a SARS-CoV-2 Spike ECD detection lateral flow assay format, using CR3022 monoclonal antibody and M13-specific polyclonal antibody in the test line and control line, respectively. The signal appeared in a dose-dependent manner as the Spike ECD is present in the testing sample at 2 μg/mL (FIG. 27B). The lateral flow assay was “clean” with no background; however, a faint signal was observed in the test line the negative control containing no ECD, indicating non-specific interaction between CR3022 and biotinylated BSFnano728Aev1C121. Note that some antibodies react with broad range of antigens, hence this signal could be a consequence of such properties of either CR3022 or scFvC121.

To develop nanorods for detection of the SARS-CoV-2 nucleocapsid (NC) protein, antigen-binding domain of the heavy-chain-only (VHH) antibody N3 was displayed as a fusion to -pIII (FIG. 55, SEQ ID NO: 99, SEQ ID NO: 100). The resulting helper plasmid, pHP1LacAevN3 was used in conjunction with the nanorod replication-assembly plasmid pBSFnano728, to produce nanorods displaying the VHH of N3 antibody (BSFnano728Aev1N3). These nanorods display AlaAla motif on every copy of the major coat protein pVIII and therefore allow for enzymatic attachment of LPETA-tagged molecules.

biotinylated BSFnano728Aev1N3 nanorods were used for detection of the SARS-CoV-2 nucleocapsid (NC) protein in an ELISA sandwich assay, using an NC-specific aptamer (synthetic ssDNA) as a capture molecule (FIG. 26C). The same nanorods were used in a lateral flow assay where the capture aptamer and the M13-specific polyclonal antibodies served a test and control spot, respectively. This assay showed no background in the absence of antigen (FIG. 27C).

### INDUSTRIAL APPLICABILITY

The virus-free nanorod production system (NPS) and method of producing nanorods as disclosed herein have industrial applicability when used as nanorods for various nanoscale applications in material science and biomedicine, including but not limited to incorporation into novel nanomaterials and use as diagnostics or for drug targeting.

### Tables

TABLE 1
E. coli strains

| Strain | Genotype | Reference |
|---|---|---|
| JW0105 | Δ(araD-araB)567, ΔlacZ4787(::rrnB-3), λ-, rph-1, | (Baba et al., 2006) |
| | Δ(rhaD-rhaB)568, hsdR514 ΔnadC727::kan | |
| JW0157 | F-, Δ(araD-araB)567, ΔdegP775::kan, | (Baba et al., 2006) |
| | ΔlacZ4787(::rrnB-3), λ-, rph-1, Δ(rhaD-rhaB)568, | |
| | hsdR514 | |
| K91 | HfrC, λ-, S26 RIE, fadL701 phoM510 mcrB rrnB ton | The Rockefeller |
| | A22, gar B10, ompF, relAl, pit 10, spoT1, T2^{R} | University collection |
| K561 | HfrC, S26 RIE, fadL701 phoM510 mcrB rrnB | The Rockefeller |
| | tonA22, garB10, ompF, relAl, pit10, spoT1, T2^{R}, | University collection |
| | lacI^{q} | |
| K1030 | K91, supD, zed508::Tn10 | The Rockefeller |
| | | University collection |
| K1466 | MC1061 redC tet^{R} (P1cl100Cm^{R} dam?1 rev6) | The Rockefeller |
| | | University collection |
| K2091 | K561 supD, zed508::Tn10 | (Sattar et al., 2015) |
| K2092 | TG1 supD, zed508::Tn1 | This disclosure |
| K2245 | Δ(araD-araB)567, ΔlacZ4787(::rrnB-3), λ-, | This disclosure |
| | ΔrecO737, rph-1, Δ(rhaD-rhaB)568, hsdR514; | |
| | F′[::Tn10 proAB + lacIq Δ(lacZ)M15] | |
| K2379 | Δ(araD-araB)567, ΔlacZ4787(::rrnB-3), λ-, | This disclosure |
| | ΔrecO737, rph-1, Δ(rhaD-rhaB)568, hsdR514; | |
| | F′[::Tn10 proAB + lacIq Δ(lacZ)M15]/pTinselPurple | |
| K2448 | K1030, ΔmetE774 | This disclosure |
| K2449 | K2091, ΔmetE774 | This disclosure |
| K2484 | K2448 ΔnadC727::kan | This disclosure |
| K2485 | K2449 ΔnadC727::kan | This disclosure |
| K2486 | K2484 ΔnadC727 | This disclosure |
| K2487 | K2485 ΔnadC727 | This disclosure |
| K2515 | JW0105 Δkan | This disclosure |
| TG1 | K-12 supE44 Δ(hsdM-mcrB)5 (rk⁻ mk⁻ McrB⁻)thi | (Sambrook and |
| | Δ(lac-proAB) F′ [traΔ36 lacI^{q} Δ(lacZ)M15 proAB] | Russell, 2001) |

TABLE 2
Pop-up plasmids of the single-plasmid nanorod production system.

| | | | BSF | | |
|---|---|---|---|---|---|
| | | | nanorod | Plasmid | Source |
| Name | Description | Markers | cassette | ori | (reference) |
| pPop- | Expresses all Ff phage | Kan | BSFpn | pA15 | This |
| up529YM | proteins; replicates BSFnano | | 529 nt | | disclosure |
| | pn 529 nt ssDNA; gII IR1- | | | | |
| | B(C143T); gVIII-20am pVIII | | | | |
| | Y21M | | | | |
| pPop- | Expresses all Ff phage | Kan | BSFpn | pA15 | This |
| up529LacYM | proteins; replicates BSFnano | | 529 nt | | disclosure |
| | pn 529 nt ssDNA; Ff | | | | |
| | promoter P_{A} replaced by | | | | |
| | placUV5; gII IR1-B(C143T); | | | | |
| | f1 gVIII-20am pVIII Y21M. | | | | |
| pPop- | Expresses all Ff phage | Kan | BSFpn | pA15 | This |
| up529LacAev | proteins; replicates BSFnano | | 529 nt | | disclosure |
| | pn 529 nt ssDNA; Ff | | | | |
| | promoter P_{A} replaced by | | | | |
| | placUV5; gII IR1-B(C143T); | | | | |
| | gVIII-20am pVIII nAAGG, | | | | |
| | ΔP6, S17L, L27S; | | | | |
| | gIII::MCS; pIII full length | | | | |
| pPop- | Expresses all Ff phage | NadC | BSFpn | pA15 | This |
| up529LacYMN | proteins; replicates BSFnano | | 529 nt | | disclosure |
| | pn 529 nt ssDNA; Ff | | | | |
| | promoter P_{A} replaced by | | | | |
| | placUV5; gII IR1-B(C143T); | | | | |
| | gVIII-20am pVIII Y21M, | | | | |
| pPop- | Expresses all Ff phage | Kan | BSFpn | pA15 | This |
| up395LacYM | proteins; replicates BSFnano | | 395 nt | | disclosure |
| | pn 395 nt ssDNA; Ff | | | | |
| | promoter P_{A} replaced by | | | | |
| | placUV5; gII IR1-B(C143T); | | | | |
| | f1 gVIII-20am pVIII Y21M. | | | | |
| pPop- | Expresses all Ff phage | Kan | BSFp | pA15 | This |
| up221YM | proteins; replicates BSFnano | | 221 nt | | disclosure |
| | p 221 nt ssDNA; gII IR1- | | | | |
| | B(C143T); gVIII-20am pVIII | | | | |
| | Y21M | | | | |
| pPop- | Expresses all Ff phage | Kan | BSFp | pA15 | This |
| up221LacYM | proteins; replicates BSFnano | | 221 nt | | disclosure |
| | p 221 ssDNA; Ff promoter | | | | |
| | P_{A} replaced by placUV5; gII | | | | |
| | IR1-B(C143T); gVIII-20am | | | | |
| | pVIII Y21M | | | | |
| pPop- | Expresses all Ff phage | Kan | BSFp | pA15 | This |
| up152LacYM | proteins; replicates BSFnano | | 152 nt | | disclosure |
| | p152 ssDNA; Ff promoter P_{A} | | | | |
| | replaced by placUV5; gII | | | | |
| | IR1-B(C143T); gVIII-20am | | | | |
| | pVIII Y21M | | | | |

TABLE 3
Helper and template plasmids of the two-plasmid nanorod production system

| | | | BSFnano | | |
|---|---|---|---|---|---|
| Name in the | | | replication- | | |
| patent | | | assembly | Plasmid | Source |
| application | Description | Markers | cassette | ori | (reference) |
| pHP1 | Helper plasmid; | Kan | | pA15 | This |
| | expresses all Ff | | | | disclosure |
| | phage proteins; gII | | | | |
| | IR1-B C143T; gVIII - | | | | |
| | 20am; S17L; | | | | |
| | gIII::MCS; pIII full | | | | |
| | length | | | | |
| pHP1Lac | Helper plasmid; | Kan | | pA15 | This |
| | expresses all Ff | | | | disclosure |
| | phage proteins; Ff | | | | |
| | promoter P_{A} replaced | | | | |
| | by placUV5; gII IR1-B | | | | |
| | C143T; gVIII -20am; | | | | |
| | S17L; gIII::MCS; pIII | | | | |
| | full length | | | | |
| pHP1YM | Helper plasmid; | Kan | | pA15 | This |
| | expresses all Ff | | | | disclosure |
| | phage proteins; gII | | | | |
| | IR1-B(C143T); gVIII- | | | | |
| | 20am, pVIII Y21M | | | | |
| pHP1YMLac | Helper plasmid; | Kan | | pA15 | This |
| | expresses all Ff | | | | disclosure |
| | phage proteins; Ff | | | | |
| | promoter P_{A} replaced | | | | |
| | by placUV5; gII IR1- | | | | |
| | B(C143T); gVIII- | | | | |
| | 20am, pVIII Y21M | | | | |
| pHP2 | Helper plasmid; | Kan | | pA15 | This |
| | expresses all Ff | | | | disclosure |
| | phage proteins; gII | | | | |
| | IR1-B(C143T); | | | | |
| | gVIII2am, pVIII | | | | |
| | S17L; gIII::MCS; pIII | | | | |
| | full length | | | | |
| pHP1Az | Helper plasmid; | Kan | | pA15 | This |
| | expresses all Ff | | | | disclosure |
| | phage proteins; gII | | | | |
| | IR1-B(C143T); gVIII- | | | | |
| | 20am, pVIII A9M, | | | | |
| | S17L, M28L; | | | | |
| | gIII::MCS; pIII full | | | | |
| | length | | | | |
| pHP1A | Helper plasmid; | Kan | | pA15 | This |
| | expresses all Ff | | | | disclosure |
| | phage proteins; gII | | | | |
| | IR1-B(C143T); gVIII- | | | | |
| | 20am AAG-pVIII, | | | | |
| | ΔP6; S17L; | | | | |
| | gIII::MCS; pIII full | | | | |
| | length | | | | |
| pHP1Aev | Helper plasmid; | Kan | | pA15 | This |
| | expresses all Ff | | | | disclosure |
| | phage proteins; gII | | | | |
| | IR1-B(C143T); gVIII- | | | | |
| | 20am AAG-pVIII, | | | | |
| | ΔP6; S17L, L27S; | | | | |
| | gIII::MCS; pIII full | | | | |
| | length | | | | |
| pHP1LacAz | Helper plasmid; | Kan | | pA15 | This |
| | expresses all Ff | | | | disclosure |
| | phage proteins; | | | | |
| | gVIII-20am, Ff | | | | |
| | promoter P_{A} replaced | | | | |
| | by placUV5; gII IR1-B | | | | |
| | C143T; pVIII 9AM, | | | | |
| | S17L, M28L; | | | | |
| | gIII::MCS; pIII full | | | | |
| | length | | | | |
| pHP1LacAev | Helper plasmid; | Kan | | pA15 | This |
| | expresses all Ff | | | | disclosure |
| | phage proteins; Ff | | | | |
| | promoter P_{A} replaced | | | | |
| | by placUV5; gII IR1-B | | | | |
| | C143T; gVIII -20am | | | | |
| | AAG-pVIII, ΔP6; | | | | |
| | S17L, L27S; | | | | |
| | gIII::MCS; pIII full | | | | |
| | length | | | | |
| pHP1LacAev5 | Helper plasmid; | Kan | | pA15 | This |
| | expresses all Ff | | | | disclosure |
| | phage proteins; Ff | | | | |
| | promoter P_{A} replaced | | | | |
| | by placUV5; gII IR1-B | | | | |
| | C143T; gVIII -20am | | | | |
| | AAG-pVIII, D5A, ΔP6; | | | | |
| | S17L; gIII::MCS; pIII | | | | |
| | full length | | | | |
| pHP1LacAevG8 | Helper plasmid; | Kan | | pA15 | This |
| | expresses all Ff | | | | disclosure |
| | phage proteins; Ff | | | | |
| | promoter P_{A} replaced | | | | |
| | by placUV5; gII IR1-B | | | | |
| | C143T; gVIII -20am | | | | |
| | nGGGG A1G-pVIII (or | | | | |
| | nGGGGG-pVIII); | | | | |
| | gIII::MCS; pIII full | | | | |
| | length | | | | |
| pHP2G8 | Helper plasmid; | Kan | | pA15 | This |
| | expresses all Ff | | | | disclosure |
| | phage proteins; gII | | | | |
| | IR1-B C143T; gVIII | | | | |
| | 2am, nGGGG-pVIII | | | | |
| | S17L; gIII::MCS; pIII | | | | |
| | full length | | | | |
| pHP1AevC | Helper plasmid, | Kan | | pA15 | This |
| | pHP1Aev gIIIC::MCS | | | | disclosure |
| pHP1C | Helper plasmid, gII | Kan | | pA15 | This |
| | IR1-B C143T; gVIII- | | | | disclosure |
| | 20am, pVIIIS17L, | | | | |
| | gIIIC::MCS | | | | |
| pHP2FnB | Helper plasmid pHP2 | Kan | | pA15 | This |
| | displaying a fusion of | | | | disclosure |
| | fibronectin-binding | | | | |
| | repeats to pIII (FnB- | | | | |
| | pIII) | | | | |
| pHP2G8FnB | Helper plasmid | Kan | | pA15 | This |
| | pHP2G8 displaying a | | | | disclosure |
| | fusion of fibronectin- | | | | |
| | binding repeats to | | | | |
| | pIII (FnB-pIII) | | | | |
| pHP1LacAevC121 | Helper plasmid | Kan | | pA15 | This |
| | PHP1LacAev | | | | disclosure: |
| | displaying scFv of | | | | antibody |
| | antibody C121against | | | | sequence is |
| | the SARS-CoV-2 | | | | from |
| | Spike protein | | | | (Robbiani et |
| | | | | | al., 2020) |
| pHP1LacAevN3 | Helper plasmid | Kan | | pA15 | This |
| | pHP1LacAev | | | | disclosure: |
| | displaying VHH of a | | | | VHH |
| | heavy-chain-only | | | | sequence is |
| | antibody N3 against | | | | from |
| | the SARS-CoV-2 | | | | (Sherwood |
| | nucleocapisd (NC) | | | | and |
| | protein | | | | Hayhurst, |
| | | | | | 2021) |
| pBSFnano-pn529 | Template plasmid: | Amp | BSFpn | pUC | This |
| | BSFpn 529 nt ssDNA | | 529 nt | | disclosure |
| | produced and | | | | |
| | packaged in the | | | | |
| | presence of helper | | | | |
| | plasmid | | | | |
| pBSFnano- | Template plasmid: | NadC | BSFpn | pUC | This |
| pn529N | BSFpn 529 nt ssDNA | (aux) | 529 nt | | disclosure |
| | produced and | | | | |
| | packaged in the | | | | |
| | presence of helper | | | | |
| | plasmid | | | | |
| pBSFnano-pn711 | Template plasmid: | Amp | BSFpn | pUC | This |
| | BSFpn 711 nt ssDNA | | 711 nt | | disclosure |
| | produced and | | | | |
| | packaged in the | | | | |
| | presence of helper | | | | |
| | plasmid | | | | |
| pBSFnano-pn728 | Template plasmid: | Amp | BSFpn | pUC | This |
| | BSFpn 728 nt ssDNA | | 728 nt | | disclosure |
| | produced and | | | | |
| | packaged in the | | | | |
| | presence of helper | | | | |
| | plasmid | | | | |
| pBSFnano-pn79a | Template plasmid; | Amp | BSFpn79a | pUC | This |
| | BSFpn cassette | | 707 nt | | disclosure |
| | expressing pVII and | | | | |
| | pIX from the Ff P_{A} | | | | |
| | (upstream of gII). | | | | |
| | 707 nt ssDNA | | | | |
| | produced and | | | | |
| | packaged in the | | | | |
| | presence of helper | | | | |
| | plasmid | | | | |
| pBSFnano- | Template plasmid; | Amp | BSFpn | pUC | This |
| pn79Lac | BSFpn cassette | | 79Lac | | disclosure |
| | expressing pVII and | | 748 nt | | |
| | pIX from the lac | | | | |
| | promoter. 748 nt | | | | |
| | ssDNA produced and | | | | |
| | packaged in the | | | | |
| | presence of helper | | | | |
| | plasmid | | | | |
| pBSFnano-pn313 | Template plasmid: | Amp | BSFpn | pUC | This |
| | BSFpn 313 nt ssDNA | | 313 nt | | disclosure |
| | produced and | | | | |
| | packaged in the | | | | |
| | presence of helper | | | | |
| | plasmid | | | | |
| pBSFnano-pn289 | Template plasmid: | Amp | BSFpn | pUC | This |
| | BSFpn 289 nt ssDNA | | 289 nt | | disclosure |
| | produced and | | | | |
| | packaged in the | | | | |
| | presence of helper | | | | |
| | plasmid | | | | |
| pBSFnano-p221 | Template plasmid: | Amp | BSFp | pCR | (Sattar, |
| | BSFp 221 nt ssDNA | | 221 nt | Blunt | 2013) |
| | produced and | | | | |
| | packaged in the | | | | |
| | presence of helper | | | | |
| | plasmid | | | | |

TABLE 4
Other plasmids

| | | | | | |
|---|---|---|---|---|---|
| Name in | | | BSF | | |
| the patent | | | nanorod | Plasmid | Source |
| application | Description | Markers | cassette | ori | (reference) |

| | | | | |
|---|---|---|---|---|
| pCP20 | Expresses FLP | Amp | Rep101^{ts} | (Cherepanov |
| | recombinase | | | and |
| | | | | Wackernagel, |
| | | | | 1995) |
| pET28a- | Expresses N-terminal | Kan | pMB1 | Addgene |
| SpySrtA | His₆ tagged | | | |
| | Streptococcus | | | |
| | pyogenes Sortase A, | | | |
| | 1_80del | | | |

TABLE 5
Bacteriophage strains

| Name in the | | | | |
|---|---|---|---|---|
| patent | | | | Source |
| application | Description/genotype | Markers | ori | (reference) |
| R783 | VCSM13, f1 (gVIII-20am, pVIII | Kan | pA15, | This |
| | S17L, gIII::MCS) | | f1 | disclosure |
| R784 | VCSM13, f1 (pVIII S17L, | Kan | pA15, | This |
| | gIII::MCS) | | f1 | disclosure |
| R786 | VCSM13, f1 (gVIII-20am, pVIII | Kan | pA15, | This |
| | nAAGG, ΔP6, S17L, gIII::MCS) | | f1 | disclosure |
| R788 | VCSM13, f1 (gVIII-20am, pVIII | Kan | pA15, | This |
| | nAAGG, ΔP6, S17L, L27S; | | f1 | disclosure |
| | gIII::MCS) | | | |
| R786a | VCSM13, f1 (gVIII-20am, pVIII | Kan | pA15, | This |
| | nAAGG, D5A, ΔP6, S17L, | | f1 | disclosure |
| | gIII::MCS) | | | |
| Rnano3 | R408 gVIII2am, pVIII S17L, | — | f1 | Sattar et al. |
| | gIII::MCS | | | (2015) |
| Rnano3FnB | R408 gVIII2am, pVIII S17L, FnB- | — | f1 | Sattar et al. |
| | gIII | | | (2015) |

TABLE 6
Exchangeable functional blocks in the BSFnano production system

| Element | Property |
|---|---|
| Selective marker | Selective markers for maintenance of |
| | plasmids in Escherichia coli |
| BSFnano replication-assembly | F1 origin and packaging signal combination |
| cassette | that results in short circular single-stranded |
| | DNA packaged into very short nanorods |
| | (40 nm, 50 nm, 70 nm, 80 nm or more in |
| | length). |
| Promoter upstream of gII, | Sequence that regulates expression of genes |
| controlling the expression of | II, X, V, VII, IX and VIII |
| gII(gX)-gV-gVII-gIX-gVIII | |
| operon | |
| CDS's (orf's) gII(gX), gV | Encoding replication protein pII (pX) and |
| | ssDNA-binding protein pV |
| CDS's (orf's) gVII, gIX, gVIII, | Encoding coat proteins pVII, pIX, pVIII, pIII, |
| gIII, gVI | pVI |
| CDS's (orf's) gI (gXI), gIV | encoding assembly proteins pI (pXI), pIV |

TABLE 7
Variants of exchangeable functional blocks used in the system

| Element | Variants constructed in this disclosure |
|---|---|
| Marker | Amp^{R}, Kan^{R}, NadC, Cm^{R} |
| BSFnano | BSFpn, replication-assembly cassette containing complete ori |
| replication-assembly | (+) or it's a segment followed by f1 intergenic sequence |
| cassette | containing a truncated (+) ori (terminator, ori(+)429 or (+) |
| | ori2 at the 3′ end (subvariants: 289, 313, 395, 529; 711; 728; |
| | 79a; 79Lac; generating circular dsDNA and ssDNA of 289, 313, |
| | 395, 529, 711, 728, 707, 748, 1400 nt. |
| | BSFp, containing A segment of (+) ori, PS; (+)ori Δ29: |
| | subvariants: 221 or 152 nt; generating circular ssDNA 221 nt or |
| | 152 nt, respectively. |
| Promoter upstream | P_{A} (wild-type phage); placUV5 (variant of lac promoter not |
| of gII, controlling | susceptible to catabolic repression) |
| the expression of | |
| gII(gX)-gV-gVII- | |
| gIX-gVIII operon | |

| Coat protein | Variants |
|---|---|
| pIII | WTpIII (pIII wild-type); |
| | gIII::MCS, pIII full length (Full-length pIII containing a cloning |
| | site for inserting peptides for display using pIII as a platform); |
| | gIIIMC3, gIII::MCS, pIIIC, pIII C-terminal domain displaying |
| | unpaired Cys residue and containing two accessible ATG codons |
| | (2 N-terminal). |
| | pIII::FnB, fusion of fibronectin binding repeats from the sof22 |
| | gene of Streptococcus pyogenes serotype 22 to full-length pIII |
| | pIII::scFvC121, fusion of the single-chain variable domains of |
| | antibody against the SARS-CoV-2 Spike protein receptor |
| | binding domain. |
| pVIII | gVIII-20am pVIII, Y21M |
| | gVIII-20am, pVIII, S17L |
| | gVIII2am, pVIII, S17L |
| | gVIII-20am, pVIII, A9M, S17L, M28L |
| | gVIII-20am, AAGG-pVIII, ΔP6, S17L |
| | gVIII-20am, AAGG-pVIII, ΔP6, S17L, L27S |
| | gVIII-20am, AAGG-pVIII, D5A, ΔP6, S17L, |
| | gVIII2am, GGGG-pVIII, S17L |
| | gVIII-20am, GGGGG-pVIII |
| pVI; pVII; pIX | Wild-type or peptide (or protein) fusions for display |

TABLE 8
Production of nanorods

| | | | | | Concentration |
|---|---|---|---|---|---|
| | | | Expression | | (nanorods |
| | | Replication- | of gII(gX)- | pVIIpIX | per |
| | Number of | assembly | gV-gVII-gIX- | from | litre of |
| Plasmids | plasmids | cassette | gVIII | pBSF | culture)^{a} |
| pPop-up221YM | 1 | (+) ori1, PS, | Constitutive | | 2.4 × 10¹³ |
| | | (+) ori2 | | | |
| pPop-up529YM | 1 | (+) ori1, PS, | Constitutive | | 4.8 × 10¹⁴ |
| | | (−) ori, (+) ori2 | | | |
| pHP1Aev + pBSF529 | 2 | (+) ori1, PS, | Constitutive | | 2.6 × 10¹⁴ |
| | | (−) ori, (+) ori2 | | | |
| PHP1Aev + pBSF529N | 2 | (+) ori1, PS, | Constitutive | | 2.1 × 10¹⁴ |
| | | (−) ori, (+) ori2 | | | |
| pHP2 + pBSF711 | 2 | (+) ori1, PS, | Constitutive | | 2.6 × 10¹⁴ |
| | | (−) ori, (+) ori2 | | | |
| pHP2 + pBSF79a | 2 | (+) ori1, PS, | Constitutive | constitutive | 4.3 × 10¹⁴ |
| | | (−) ori, (+) ori2 | | gVIIgIX | |
| pHP2 + pBSF79Lac | 2 | (+) ori1, PS, | Constitutive | plac- | 6.8 × 10¹⁴ |
| (−IPTG) | | (−) ori, (+) ori2 | | gVIIgIX | |
| | | | | not | |
| | | | | induced | |
| pHP2 + pBSF79Lac | 2 | (+) ori1, PS, | Constitutive | plac- | 1.5 × 10¹⁵ |
| (+IPTG) | | (−) ori, (+) ori2 | | gVIIgIX | |
| | | | | induced | |
| pPop-Up529LacYM | 1 | (+) ori1, PS, | placUV5, not | | 5.2 × 10¹³ |
| (−IPTG) | | (−) ori, (+) ori2 | induced | | |
| pPop-up529LacYM | 1 | (+) ori1, PS, | placUV5, | | 4.8 × 10¹⁵ |
| (+IPTG) | | (−) ori, (+) ori2 | induced | | |
| pPop-up395LacYM | 1 | (+) ori1, PS, | placUV5, | | 1.7 × 10¹⁵ |
| (+IPTG) | | (−) ori, (+) ori2 | induced | | |
| pPop-up221LacYM | 1 | (+) ori1, PS, | placUV5, | | 1.9 × 10¹⁴ |
| (+IPTG) | | (+) ori2 - | induced | | |
| pPop-up152LacYM | 1 | (+) ori1, PS, | placUV5, | | 1.8 × 10¹⁴ |
| (+IPTG) | | (+) ori2 | induced | | |

^{a}Pure nanorods after the CsCl gradient centrifugation.

TABLE 9
Length and components of the scaffold (ssDNA) generated by the pBSF
replication-assembly cassettes

| | BSFp- | BSFp- | | BSFp- | BSFp- | | | | BSFp- | | |
|---|---|---|---|---|---|---|---|---|---|---|---|
| | n1400 | n728 | BSFp- | n79a | n79Lac | BSFp- | BSFp- | BSFp | n289 | BSFp- | BSFp |
| | SEQ | SEQ | n711 | (707nt) | (748nt) | n529 | n395 | n313 | SEQ | n221 | n140 |
| | ID | ID | SEQ ID | SEQ | SEQ ID | SEQ ID | SEQ ID | SEQ ID | ID | SEQ ID | SEQ ID |
| | Nos: | Nos: | Nos: | ID Nos: | Nos: | Nos: | Nos: | Nos: | Nos: | Nos: | Nos: |
| | 101, 102 | 41, 42 | 43, 44 | 52, 53 | 54, 55 | 61, 62 | 64, 65 | 70, 71 | 72, 73 | 77, 78 | 101, 102 |
| | FIG. | FIG. | FIG. | FIG. | FIG. | FIG. | FIG. | FIG. | FIG. | FIG. | FIG. |
| Sequence | 39, 40 | 39, 40 | 39, 40 | 41, 42 | 41, 42. | 43, 44. | 43, 44. | 45, 46 | 45, 46 | 47, 48 | 47. 48 |
| (+) ori1 from | 129ª | 129 | 129 | 129 | 129 | 129 | 129 | 47 | 47 | 51 | 47 |
| pII cut | | | | | | | | | | | |
| pA promoter | | | | 81 | 40 | | | | | | |
| pBSFpn79a | | | | | | | | | | | |
| and | | | | | | | | | | | |
| extended(+) | | | | | | | | | | | |
| ori sequence | | | | | | | | | | | |
| in | | | | | | | | | | | |
| pBSFpn79Lac | | | | | | | | | | | |
| Lac promoter | | | | | 82 | | | | | | |
| gVIIgIX | | | | 197 | 197 | | | | | | |
| Filler I | 986 | 308 | 291 | 9 | 9 | 109 | 23 | 24 | | 16 | |
| PS | 85 | 91 | 91 | 91 | 91 | 91 | 86 | 85 | 85 | 83 | 84 |
| Filler II | 28 | 28 | 28 | 28 | 28 | 36 | | | | | |
| (−) ori | 149 | 149 | 149 | 149 | 149 | 141 | 141 | 141 | 141 | | |
| (+) ori 2 up to | 23 | 23 | 23 | 23 | 23 | 23 | 16 | 16 | 16 | 56 | 16 |
| pII cute | | | | | | | | | | | |
| Circular | 1400 | 728 | 711 | 707 | 748 | 529 | 395 | 313 | 289 | 221 | 152 |
| ssDNA | | | | | | | | | | | |
| (scaffold) | | | | | | | | | | | |
| total length | | | | | | | | | | | |
| Replication- | 386 | 392 | 392 | 392 | 392 | 384 | 372 | 289 | 289 | 190 | 147 |
| assembly | | | | | | | | | | | |
| sequences | | | | | | | | | | | |
| Accessory | | | 278 | 279 | | | | | | | |
| function | | | | | | | | | | | |
| (promoter- | | | | | | | | | | | |
| gVIIgIX fusion) | | | | | | | | | | | |
| Filler | 1,014 | 336 | 319 | 37 | 77 | 145 | 23 | 24 | 0 | 31 | 5 |
| sequences | | | | | | | | | | | |

ªThe lengths of sequences (in nucleotides) were derived from FIGS. 40, 42, 44, 46, 48 and 57 (SEQ ID NOs: 41-87; 101-104)

### REFERENCES

Baba, T., Ara, T., Hasegawa, M., Takai, Y., Okumura, Y., Baba, M., Datsenko, K. A., Tomita, M., Wanner, B. L., and Mori, H. (2006). Construction of Escherichia coli K-12 in-frame, single-gene knockout mutants: the Keio collection. Mol Syst Biol 2, 2006 0008.
Barbas, C. F., 3rd, Kang, A. S., Lerner, R. A., and Benkovic, S. J. (1991). Assembly of combinatorial antibody libraries on phage surfaces: the gene III site. Proc Natl Acad Sci USA 88, 7978-7982.
Barbas III, C. F., Burton, D. R., Scott, J. K., and Silverman, G. J. (2001). Phage display: a laboratory manual (Cold Spring Harbor, New York: Cold Spring Harbor Laboratory Press).
Bernard, J. M., and Francis, M. B. (2014). Chemical strategies for the covalent modification of filamentous phage. Front Microbiol 5, 734.
Bhatia, R., and Calvo, K. C. (1996). The sequencing expression, purification, and steady-state kinetic analysis of quinolinate phosphoribosyl transferase from Escherichia coli. Arch Biochem Biophys 325, 270-278.
Bowie, J. U., Reidhaar-Olson, J. F., Lim, W. A., and Sauer, R. T. (1990). Deciphering the message in protein sequences: tolerance to amino acid substitutions. Science 247, 1306-1310.
Burg, M. A., Jensen-Pergakes, K., Gonzalez, A. M., Ravey, P., Baird, A., and Larocca, D. (2002). Enhanced phagemid particle gene transfer in camptothecin-treated carcinoma cells. Cancer Res 62, 977-981.
Burtis, C. A., Bruns, D. E., Sawyer, B. G., and Tietz, N. W. (2015). Tietz fundamentals of clinical chemistry and molecular diagnostics, Seventh edition. edn (St. Louis, Missouri: Elsevier/Saunders).
Cao, B. R., Yang, M. Y., and Mao, C. B. (2016). Phage as a genetically modifiable supramacromolecule in chemistry, materials and medicine. Acc Chem Res 49, 1111-1120.
Chalker, J. M., Bernardes, G. J., and Davis, B. G. (2011). A “tag-and-modify” approach to site-selective protein modification. Acc Chem Res 44, 730-741.
Chen, P. Y., Dang, X., Klug, M. T., Qi, J., Dorval Courchesne, N. M., Burpo, F. J., Fang, N., Hammond, P. T., and Belcher, A. M. (2013). Versatile three-dimensional virus-based template for dye-sensitized solar cells with improved electron transport and light harvesting. Acs Nano 7, 6563-6574.
Cherepanov, P. P., and Wackernagel, W. (1995). Gene disruption in Escherichia coli: T_{C}^{R} and Km^{R} cassettes with the option of Flp-catalyzed excision of the antibiotic-resistance determinant. Gene 158, 9-14.
Cho, S. J., Woo, H. M., Kim, K. S., Oh, J. W., and Jeong, Y. J. (2011). Novel system for detecting SARS coronavirus nucleocapsid protein using an ssDNA aptamer. J Biosci Bioeng 112, 535-540.
Chung, W. J., Lee, D. Y., and Yoo, S. Y. (2014). Chemical modulation of M13 bacteriophage and its functional opportunities for nanomedicine. International journal of nanomedicine 9, 5825-5836.
Chung, W. J., Oh, J. W., Kwak, K., Lee, B. Y., Meyer, J., Wang, E., Hexemer, A., and Lee, S. W. (2011). Biomimetic self-templating supramolecular structures. Nature 478, 364-368.
Corrigan, N., Ciftci, M., Jung, K. W., and Boyer, C. (2021). Mediating Reaction Orthogonality in Polymer and Materials Science. Angew Chem Int Edit 60, 1748-1781.
Day, L. A. (1969). Conformations of single-stranded DNA and coat protein in fd bacteriophage as revealed by ultraviolet absorption spectroscopy. J Mol Biol 39, 265-277.
Dong, W. R., Xiang, L. X., and Shao, J. Z. (2010). Novel antibiotic-free plasmid selection system based on complementation of host auxotrophy in the NAD de novo synthesis pathway. Appi Environ Microbiol 76, 2295-2303.
Dotto, G. P., Horiuchi, K., and Zinder, N. D. (1982). Initiation and termination of phage f1 plus-strand synthesis. Proc Nat/Acad Sci USA 79, 7122-7126.
Dotto, G. P., Horiuchi, K., and Zinder, N. D. (1984a). The functional origin of bacteriophage f1 DNA replication. Its signals and domains. J Mol Biol 172, 507-521.
Dotto, G. P., Horiuchi, K., and Zinder, N. D. (1984b). The origin of DNA replication of bacteriophage f1 and its interaction with the phage gene II protein. Adv Exp Med Biol 179, 185-191.
Enea, V., Horiuchi, K., Turgeon, B. G., and Zinder, N. D. (1977). Physical map of defective interfering particles of bacteriophage f1. J Mol Biol 111, 395-414.
Enea, V., and Zinder, N. D. (1982). Interference resistant mutants of phage f1. Virology 122, 222-226.
Feldhaus, M. J., Hwa, V., Cheng, Q., and Salyers, A. A. (1991). Use of an Escherichia coli beta-glucuronidase gene as a reporter gene for investigation of Bacteroides promoters. J Bacteriol 173, 4540-4543.
Fuh, G., and Sidhu, S. S. (2000). Efficient phage display of polypeptides fused to the carboxy-terminus of the M13 gene-3 minor coat protein. FEBS Lett 480, 231-234.
Gao, C., Mao, S., Lo, C. H., Wirsching, P., Lerner, R. A., and Janda, K. D. (1999). Making artificial antibodies: a format for phage display of combinatorial heterodimeric arrays. Proc Natl Acad Sci USA 96, 6025-6030.
Haaparanta, T., and Huse, W. D. (1995). A combinatorial method for constructing libraries of long peptides displayed by filamentous phage. Mol Divers 1, 39-52.
Hess, G. T., Cragnolini, J. J., Popp, M. W., Allen, M. A., Dougan, S. K., Spooner, E., Ploegh, H. L., Belcher, A. M., and Guimaraes, C. P. (2012). M13 bacteriophage display framework that allows sortase-mediated modification of surface-accessible phage proteins. Bioconjug Chem 23, 1478-1487.
Hondorp, E. R., and Matthews, R. G. (2006). Methionine. EcoSal Plus 2.
Iannolo, G., Minenkova, O., Petruzzelli, R., and Cesareni, G. (1995). Modifying filamentous phage capsid: limits in the size of the major capsid protein. J Mol Biol 248, 835-844.
Jespers, L. S., De Keyser, A., and Stanssens, P. E. (1996). LambdaZLG6: a phage lambda vector for high-efficiency cloning and surface expression of cDNA libraries on filamentous phage. Gene 173, 179-181.
Kiick, K. L., Saxon, E., Tirrell, D. A., and Bertozzi, C. R. (2002). Incorporation of azides into recombinant proteins for chemoselective modification by the Staudinger ligation. Proc Natl Acad Sci USA 99, 19-24.
Larocca, D., and Baird, A. (2001). Receptor-mediated gene transfer by phage-display vectors: applications in functional genomics and gene therapy. Drug Discov Today 6, 793-801.
Lederberg, J. (2000). Encyclopedia of microbiology, 2nd edn (San Diego: Academic Press).
Lee, J. W., Song, J., Hwang, M. P., and Lee, K. H. (2013). Nanoscale bacteriophage biosensors beyond phage display. Internationaljournal ofnanomedicine 8, 3917-3925.
Lerner, T. J., and Model, P. (1981). The “steady state” of coliphage f1: DNA synthesis late in infection. Virology 115, 282-294.
Lewin, B., Krebs, J. E., Kilpatrick, S. T., Goldstein, E. S., and Lewin, B. (2011). Lewin's genes X, 10th edn (Sudbury, Mass.: Jones and Bartlett).
Li, K., Chen, Y., Li, S. Q., Huong, G. N., Niu, Z. W., You, S. J., Mello, C. M., Lu, X. B., and Wang, Q. A. (2010). Chemical modification of M13 bacteriophage and Its application in cancer cell imaging. Bioconj Chemistry 21, 1369-1377.
Madigan, M. T., Madigan, M. T., and Brock, T. D. (2009). Brock biology of microorganisms, 12th edn (San Francisco, CA: Pearson/Benjamin Cummings).
Marvin, D. A., Welsh, L. C., Symmons, M. F., Scott, W. R., and Straus, S. K. (2006). Molecular structure of fd (f1, M13) filamentous bacteriophage refined with respect to X-ray fibre diffraction and solid-state NMR data supports specific models of phage assembly at the bacterial membrane. J Mol Biol 355, 294-309.
Merriam, V. (1977). Stability of the carrier state in bacteriophage M13-infected cells. J Virol 21, 880-888.
Meyers, R. A. (1995). Molecular biology and biotechnology: a comprehensive desk reference (New York: VCH). Model, P., and Russel, M. (1988). Filamentous Bacteriophage. In The Bacteriophages, R. Calendar, ed. (New York: Plenum Publishing), pp. 375-456.
Nelson, F. K., Friedman, S. M., and Smith, G. P. (1981). Filamentous phage DNA cloning vectors: a noninfective mutant with a nonpolar deletion in gene III. Virology 108, 338-350.
Newman, J., Swinney, H. L., and Day, L. A. (1977). Hydrodynamic properties and structure of fd virus. J Mol Biol 116, 593-603.
O'Neil, K., and Hoess, R. H. (1995). Phage display: protein engineering by directed evolution. Current Opinion in Structural Biology 5, 443-449.
Petrenko, V. (2008). Evolution of phage display: from bioactive peptides to bioselective nanomaterials. Expert Opin Drug Deliv 5, 825-836.
Petrie, A. (2015). Expanding the bionanotechnology toolbox using an engineered bacteriophage M13 platform. In Department of Chemistry (Waterloo, Ontario, Canada: University of Waterloo).
Rakonjac, J. (1998). The roles of pIII in filamentous phage assembly (New York, NY, USA: The Rockefeller University).
Rakonjac, J., Bennett, N.J., Spagnuolo, J., Gagic, D., and Russel, M. (2011). Filamentous bacteriophage: biology, phage display and nanotechnology applications. Curr Issues Mol Biol 13, 51-76.
Rakonjac, J., and Model, P. (1998). Roles of pIII in filamentous phage assembly. J Mol Biol 282, 25-41.
Rakonjac, J., Russel, M., Khanum, S., Brooke, S. J., and Rajic, M. (2017). Filamentous Phage: Structure and Biology. Adv Exp Med Biol 1053, 1-20.
Rakonjac, J. V., Robbins, J. C., and Fischetti, V. A. (1995). DNA sequence of the serum opacity factor of group A streptococci: identification of a fibronectin-binding repeat domain. Infect Immun 63, 622-631.
Ravetch, J. V., Horiuchi, K., and Zinder, N. D. (1979). DNA sequence analysis of the defective interfering particles of bacteriophage f1. J Mol Biol 128, 305-318.
Reddy, C. A. (2007). Methods for general and molecular microbiology, 3rd edn (Washington, D.C.: ASM Press).
Robbiani, D. F., Gaebler, C., Muecksch, F., Lorenzi, J. C. C., Wang, Z., Cho, A., Agudelo, M., Barnes, C. O., Gazumyan, A., Finkin, S., Hagglof, T., Oliveira, T. Y., Viant, C., Hurley, A., Hoffmann, H. H., Millard, K. G., Kost, R. G., Cipolla, M., Gordon, K., Bianchini, F., Chen, S. T., Ramos, V., Patel, R., Dizon, J., Shimeliovich, I., Mendoza, P., Hartweger, H., Nogueira, L., Pack, M., Horowitz, J., Schmidt, F., Weisblum, Y., Michailidis, E., Ashbrook, A. W., Waltari, E., Pak, J. E., Huey-Tubman, K. E., Koranda, N., Hoffman, P. R., West, A. P., Jr., Rice, C. M., Hatziioannou, T., Bjorkman, P. J., Bieniasz, P. D., Caskey, M., and Nussenzweig, M. C. (2020). Convergent antibody responses to SARS-CoV-2 in convalescent individuals. Nature 584, 437-442.
Russel, M., Whirlow, H., Sun, T. P., and Webster, R. E. (1988). Low-frequency infection of F⁻ bacteria by transducing particles of filamentous bacteriophages. J Bacteriol 170, 5312-5316.
Sambrook, J., and Russell, D. W. (2001). Molecular cloning: a laboratory manual (3rd. Edn.), 3rd edn (Cold Spring Harbor: Cold Spring Harbor).
Sarikaya, M., Tamerler, C., Jen, A., Schulten, K., and Baneyx, F. (2003). Molecular biomimetics: nanotechnology through biology. NAT MATER 2, 577-585.
Sattar, S. (2013). Filamentous phage-derived nano-rods for applications in diagnostics and vaccines. In Institute of Fundamental Sciences (Palmerston North, New Zealand: Massey University).
Sattar, S., Bennett, N.J., Wen, W. X., Guthrie, J. M., Blackwell, L. F., Conway, J. F., and Rakonjac, J. (2015). Ff-nano, short functionalized nanorods derived from Ff (f1, fd, or M13) filamentous bacteriophage. Front Microbiol 6, 316.
Sherwood, L. J., and Hayhurst, A. (2021). Toolkit for Quickly Generating and Characterizing Molecular Probes Specific for SARS-CoV-2 Nucleocapsid as a Primer for Future Coronavirus Pandemic Preparedness. ACS Synth Biol 10, 379-390.
Singleton, P., and Sainsbury, D. (2006). Dictionary of microbiology and molecular biology, 3rd edn (Chichester, West Sussex; Hoboken, NJ: Wiley).
Smeal, S. W., Schmitt, M. A., Pereira, R. R., Prasad, A., and Fisk, J. D. (2017a). Simulation of the M13 life cycle I: Assembly of a genetically-structured deterministic chemical kinetic simulation. Virology 500, 259-274.
Smeal, S. W., Schmitt, M. A., Pereira, R. R., Prasad, A., and Fisk, J. D. (2017b). Simulation of the M13 life cycle II: Investigation of the control mechanisms of M13 infection and establishment of the carrier state. Virology 500, 275-284.
Specthrie, L., Bullitt, E., Horiuchi, K., Model, P., Russel, M., and Makowski, L. (1992). Construction of a microphage variant of filamentous bacteriophage. J Mol Biol 228, 720-724.
Sternberg, N. L., and Maurer, R. (1991). Bacteriophage-mediated generalized transduction in Escherichia coli and Salmonella typhimurium. Methods Enzymol 204, 18-43.
Wang, Z., and Tang, M. (2020). The cytotoxicity of core-shell or non-shell structure quantum dots and reflection on environmental friendly: A review. Environ Res 194, 110593.
Whitby, L. G., and Whitby, L. G. (1993). Lecture notes on clinical biochemistry, 5th edn (Oxford; Boston: Blackwell Scientific Publications).
Zhang, Y., Zang, C., An, G., Shang, M., Cui, Z., Chen, G., Xi, Z., and Zhou, C. (2020). Cysteine-specific protein multi-functionalization and disulfide bridging using 3-bromo-5-methylene pyrrolones. Nat Commun 11, 1015.

1. A nanorod production system (NPS) comprising a nucleic acid expression construct comprising
a replication-assembly cassette comprising a filamentous phage (+) ori1, a packaging signal (PS) and an (+) ori2,
at least one plasmid origin of replication not located in the replication-assembly cassette allowing the construct to be replicated in bacteria,
at least one inducible promoter operably linked to a nucleic acid sequence encoding at least one Ff phage replication protein,
wherein the expression construct expresses the Ff phage replication protein, and generates from the replication-assembly cassette, an excised and replicated DNA sequence which forms a circular single-stranded DNA encapsulated within nanorods.

2. The NPS of claim 1, wherein the at least one Ff phage replication protein is pII.

3. The NPS of claim 1, wherein the replication assembly cassette further comprises a (−) ori between the PS and the (+) ori2.

4. The NPS of claim 1, wherein the nucleic acid expression construct is a plasmid.

5. The NPS of claim 1, wherein the NPS lacks a second nucleic acid construct encoding one or more filamentous phage proteins.

6. The NPS of claim 1, wherein the nucleic acid expression construct comprises nucleic acid sequences encoding each of Ff phage pI-pXI proteins, and wherein any or all of the nucleic acid sequences encoding each of Ff phage pI-pXI proteins encodes a modified Ff phage protein.

7. (canceled)

8. The NPS of claim 1, wherein the nucleic acid construct comprises a nucleic acid sequence that encodes a modified Ff phage protein comprising a mutation that allows chemical or enzymatic conjugation of small molecules, synthetic or biological polymers to the protein, optionally wherein the modified Ff phage protein is pIII and/or pVIII.

9. The NPS of claim 1, wherein the nucleic acid construct comprises a nucleic acid sequence encoding a modified Ff phage protein pVIII that includes an amber mutation.

10. The NPS of claim 1, wherein the nucleic acid construct comprises a nucleic acid sequence encoding at least one of pIII, pVI, pVII, pVIII, and pIX that is fused to a nucleic acid sequence encoding a heterologous polypeptide.

11. The NPS of claim 1, wherein the nucleic acid expression construct further comprises a nucleic acid sequence encoding an auxotrophic marker.

12. The NPS of claim 1 wherein the nucleic acid sequence between the (+) ori1 and the PS or between the PS and the (+) ori2 is a filler nucleic acid sequence encoding at least one Ff phage protein.

13. The NPS of claim 12, wherein the filler nucleic acid sequence encodes pVII, pVIII and/or pIX or encodes a modified pVII, pVIII and/or pIX or a combination thereof.

14. The NPS of claim 12 wherein the filler nucleic acid sequence encodes pVII, pVIII and/or pIX or encodes modified pVII, pVIII and/or pIX or a combination thereof and further encodes a prokaryotic or eukaryotic protein of interest.

15. The NPS of claim 1, wherein the bacteria are E. coli.

16. (canceled)

17. A method of producing nanorods comprising culturing isolated host cells comprising an NPS claim 1 and supplying the host cells with an inducer to the inducible promoter at an optimal growth phase, whereby an Ff phage replication protein is expressed in the cells, generating an excised and replicated DNA sequence that forms a circular single-stranded DNA encapsulated within the nanorods.

18. (canceled)

19. The method of claim 17 wherein the Ff phage replication protein is pII.

20.-33. (canceled)

34. A nanorod production system (NPS) comprising
i) a nucleic acid nanorod replication-assembly construct comprising a replication-assembly cassette comprising a filamentous phage (+) ori1, packaging signal (PS) and an (+) ori2, and
at least one plasmid origin of replication not located in the replication-assembly cassette allowing the construct to be replicated in bacteria, and
ii) a helper nucleic acid expression construct comprising at least one selective marker, and
at least one inducible promoter operably linked to a nucleic acid sequence encoding at least one Ff phage replication protein,
wherein the helper nucleic acid construct expresses the Ff phage replication protein and generates an excised and replicated DNA sequence from the replication-assembly cassette, which forms a circular single-stranded DNA encapsulated within nanorods.

35. The NPS of claim 34, wherein the replication assembly cassette further comprises a (−) on between the PS and (+) ori2.

36. The NPS of claim 34, wherein the Ff phage replication protein is pII.

37.-55. (canceled)

56. The NPS of claim 34 wherein the nucleic acid sequence between the (+) ori1 and the PS or between the PS and the (+) ori2 is a filler nucleic acid sequence encoding at least one Ff phage protein, wherein the at least one phage protein is pVII, pVIII and/or pIX or a modified pVII, pVIII and/or pIX or a combination thereof, wherein the filler nucleic acid sequence further encodes a prokaryotic or eukaryotic protein of interest.


SEQUENCE LISTING (104 sequences)

SEQ ID NO: 1 - protein, 410 aa, Escherichia coli virus F1
MIDMLVLRLPFIDSLVCSRLSGNDLIAFVDLSKIATLSGMNLSARTVEYHIDGDLTVSGLSHPFESLPTHYSGIAFKIYEGSKNFYPCVEIKASPAKVLQGHNVFGTTDLALCSEALLLNFANSLPCLYDLLDVNATTISRIDATFSARAPNENIAKQVIDHLRNVSNGQTKSTRSQNWESTVTWNETSRHRTLVAYLKHVELQHQIQQLSSKPSAKMTSYQKEQLKVLSNPDLLEFASGLVRFEARIKTRYLKSFGLPLNLFDAIRFASDYNSQGKDLIFDLWSFSFSELFKAFEGDSMNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKIA

SEQ ID NO: 2 - DNA, 1233 bp, Escherichia coli virus F1
  source 1..1233: mol_type = other DNA
atgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagatccagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataa

SEQ ID NO: 3 - protein, 410 aa, synthetic construct
MIDMLVLRLPFIDSLVCSRLSGNDLIAFVDLSKIATLSGMNLSARTVEYHIDGDLTVSGLSHPFESLPTHYSGIAFKIYEGSKNFYPCVEIKASPAKVLQGHNVFGTTDLALCSEALLLNFANSLPCLYDLLDVNATTISRIDATFSARAPNENIAKQVIDHLRNVSNGQTKSTRSQNWESIVTWNETSRHRTLVAYLKHVELQHQIQQLSSKPSAKMTSYQKEQLKVLSNPDLLEFASGLVRFEARIKTRYLKSFGLPLNLFDAIRFASDYNSQGKDLIFDLWSFSFSELFKAFEGDSMNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKIA

SEQ ID NO: 4 - DNA, 1233 bp, Escherichia coli virus F1
  source 1..1233: mol_type = other DNA
atgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaacgctactactattagtagaattgatgccaccttttcagctcgcgccccaaatgaaaatatagctaaacaggttattgaccatttgcgaaatgtatctaatggtcaaactaaatctactcgttcgcagaattgggaatcaattgttacatggaatgaaacttccagacaccgtactttagttgcatatttaaaacatgttgagctacagcaccagatccagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataa

SEQ ID NO: 5 - protein, 87 aa, Escherichia coli virus F1
MIKVEIKPSQAQFTTRSGVSRQGKPYSLNEQLCYVDLGNEYPVLVKITLDEGQPAYAPGLYTVHLSSFKVGQFGSLMIDRLRLVPAK

SEQ ID NO: 6 - DNA, 264 bp, Escherichia coli virus F1
  source 1..264: mol_type = other DNA
atgattaaagttgaaattaaaccatctcaagcgcaattcactacccgttctggtgtttctcgtcagggcaagccttattcactgaatgagcagctttgttacgttgatttgggtaatgaatatccggtgcttgtcaagattactcttgatgaaggtcagccagcctatgcgcctggtctgtacaccgttcatctgtcctcgttcaaagttggtcagttcggttcccttatgattgaccgtctgcgcctcgttccggctaagtaa

SEQ ID NO: 7 - protein, 33 aa, Escherichia coli virus F1
MEQVADFDTIYQAMIQISVVLCFALGIIAGGQR

SEQ ID NO: 8 - DNA, 102 bp, Escherichia coli virus F1
  source 1..102: mol_type = other DNA
atggagcaggtcgcggatttcgacacaatttatcaggcgatgatacaaatctccgttgtactttgtttcgcgcttggtataatcgctgggggtcaaagatga

SEQ ID NO: 9 - protein, 32 aa, Escherichia coli virus F1
MSVLVYSFASFVLGWCLRSGITYFTRLMETSS

SEQ ID NO: 10 - DNA, 99 bp, Escherichia coli virus F1
  source 1..99: mol_type = other DNA
atgagtgttttagtgtattctttcgcctctttcgttttaggttggtgccttcgtagtggcattacgtattttacccgtttaatggaaacttcctcatga

SEQ ID NO: 11 - protein, 73 aa, Escherichia coli virus F1
MKKSLVLKASVAVATLVPMLSFAAEGDDPAKAAFDSLQASATEYIGYAWAMVVVIVGATIGIKLFKKFTSKAS

SEQ ID NO: 12 - DNA, 222 bp, Escherichia coli virus F1
  source 1..222: mol_type = other DNA
atgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 13 - protein, 73 aa, synthetic construct
MKKXLVLKASVAVATLVPMLSFAAEGDDPAKAAFDSLQASATEMIGYAWAMVVVIVGATIGIKLFKKFTSKAS

SEQ ID NO: 14 - DNA, 222 bp, synthetic construct
  source 1..222: mol_type = other DNA
atgaaaaagtagttagtcctcaaagcctctgtagccgttgctaccctcgttccgatgctgtctttcgctgctgagggtgacgatcccgcaaaagcggcctttgactccctgcaagcctcagcgaccgaaatgatcggttatgcgtgggcgatggttgttgtcattgtcggcgcaactatcggtatcaagctgtttaagaaattcacctcgaaagcaagctga

SEQ ID NO: 15 - protein, 73 aa, synthetic construct
MKXSLVLKASVAVATLVPMLSFAAEGDDPAKAAFDSLQALATEYIGYAWAMVVVIVGATIGIKLFKKFTSKAS

SEQ ID NO: 16 - DNA, 222 bp, synthetic construct
  source 1..222: mol_type = other DNA
atgaaaaagtagttagtcctcaaagcctctgtagccgttgctaccctcgttccgatgctgtctttcgctgctgagggtgacgatcccgcaaaagcggcctttgactccctgcaagccttagcgaccgaatatatcggttatgcgtgggcgatggttgttgtcattgtcggcgcaactatcggtatcaagctgtttaagaaattcacctcgaaagcaagctga

SEQ ID NO: 17 - protein, 75 aa, synthetic construct
MKKXLVLKASVAVATLVPMLSFAAAGGEGDDAKAAFDSLQALATEYIGYAWAMVVVIVGATIGIKLFKKFTSKAS

SEQ ID NO: 18 - DNA, 228 bp, synthetic construct
  source 1..228: mol_type = other DNA
atgaaaaagtagttagtcctcaaagcctctgtagccgttgctaccctcgttccgatgctgtctttcgctgccgctggtggcgagggtgacgatgcaaaagcggcctttgactccctgcaagccttagcgaccgaatatatcggttatgcgtgggcgatggttgttgtcattgtcggcgcaactatcggtatcaagctgtttaagaaattcacctcgaaagcaagctga

SEQ ID NO: 19 - protein, 75 aa, synthetic construct
MKKXLVLKASVAVATLVPMLSFAAAGGEGDDAKAAFDSLQALATEYIGYAWSMVVVIVGATIGIKLFKKFTSKAS

SEQ ID NO: 20 - DNA, 228 bp, synthetic construct
  source 1..228: mol_type = other DNA
atgaaaaagtagttagtcctcaaagcctctgtagccgttgctaccctcgttccgatgctgtctttcgctgccgctggtggcgagggtgacgatgcaaaagcggcctttgactccctgcaagccttagcgaccgaatatatcggttatgcgtggtcgatggttgttgtcattgtcggcgcaactatcggtatcaagctgtttaagaaattcacctcgaaagcaagctga

SEQ ID NO: 21 - protein, 75 aa, synthetic construct
MKKXLVLKASVAVATLVPMLSFAAAGGEGDAAKAAFDSLQALATEYIGYAWAMVVVIVGATIGIKLFKKFTSKAS

SEQ ID NO: 22 - DNA, 228 bp, synthetic construct
  source 1..228: mol_type = other DNA
atgaaaaagtagttagtcctcaaagcctctgtagccgttgctaccctcgttccgatgctgtctttcgctgccgctggtggcgagggtgacgctgcaaaagcggcctttgactccctgcaagccttagcgaccgaatatatcggttatgcgtgggcgatggttgttgtcattgtcggcgcaactatcggtatcaagctgtttaagaaattcacctcgaaagcaagctga

SEQ ID NO: 23 - protein, 74 aa, synthetic construct
MKKXSLVLKASVAVATLVPMLSFAAEGDDPAKMAFDSLQALATEYIGYAWALVVVIVGATIGIKLFKKFTSKAS

SEQ ID NO: 24 - DNA, 222 bp, synthetic construct
  source 1..222: mol_type = other DNA
atgaaaaagtagttagtcctcaaagcctctgtagccgttgctaccctcgttccgatgctgtctttcgctgctgagggtgacgatcccgcaaaaatggcctttgactccctgcaagccttagcgaccgaatatatcggttatgcgtgggcgctggttgttgtcattgtcggcgcaactatcggtatcaagctgtttaagaaattcacctcgaaagcaagctga

SEQ ID NO: 25 - protein, 73 aa, synthetic construct
MKKSLVLKASVAVATLVPMLSFAAGGDDPAKAAFDSLQALATEYIGYAWAMVVVIVGATIGIKLFKKFTSKAS

SEQ ID NO: 26 - DNA, 222 bp, synthetic construct
  source 1..222: mol_type = other DNA
atgaaaaagtctttagtcctcaaagcctctgtagccgttgctaccctcgttccgatgctgtctttcgctgcttagggtgacgatcccgcaaaagcggcctttgactccctgcaagccttagcgaccgaatatatcggttatgcgtgggcgatggttgttgtcattgtcggcgcaactatcggtatcaagctgtttaagaaattcacctcgaaagcaagctga

SEQ ID NO: 27 - protein, 77 aa, synthetic construct
MKKKLVLKASVAVATLVPMLSFAGGGGAXGDDPAKAAFDSLQALATEYIGYAWAMVVVIVGATIGIKLFKKFTSKAS

SEQ ID NO: 28 - DNA, 234 bp, synthetic construct
  source 1..234: mol_type = other DNA
atgaaaaagtctttagtcctcaaagcctctgtagccgttgctaccctcgttccgatgctgtctttcgctggtggcggaggggcttagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 29 - protein, 424 aa, Escherichia coli virus F1
VKKLLFAIPLVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 30 - DNA, 1275 bp, Escherichia coli virus F1
  source 1..1275: mol_type = other DNA
gtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggctctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaa

SEQ ID NO: 31 - protein, 439 aa, synthetic construct
VKKLLFAIPLVVPFYSHSAAQPAMGYQAAAPGGAETVESCLAKPHTENSFTNVWKDDKTLDRYANYEGCLWNATGVVVCTGDETQCYGTWVPIGLAIPENEGGGSEGGGSEGGGSEGGGTKPPEYGDRPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 32 - DNA, 1320 bp, synthetic construct
  source 1..1320: mol_type = other DNA
gtgaaaaaattattattcgcaattcctttagttgttcctttctattctcactccgcggcccagccggccatgggatatcaggcggccgctcccgggggcgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggctctgagggtggcggtactaaacctcctgagtacggtgatagacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaa

SEQ ID NO: 33 - protein, 234 aa, synthetic construct
VKKLLFAIPLVVPFYSHSAEMAMEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 34 - DNA, 705 bp, synthetic construct
  source 1..705: mol_type = other DNA
gtgaaaaaattattattcgcaattcctttagttgttcctttctattctcactcggccgaaatggccatggaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaa

SEQ ID NO: 35 - protein, 112 aa, Escherichia coli virus F1
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDK

SEQ ID NO: 36 - DNA, 339 bp, Escherichia coli virus F1
  source 1..339: mol_type = other DNA
atgccagttcttttgggtattccgttattattgcgtttcctcggtttccttctggtaactttgttcggctatctgctaacttttcttaaaaagggcttcggtaagatagctattgctatttcattgtttcttgctcttattattgggcttaactcaattcttgtgggttatctctctgatattagcgctcaattaccctctgactttgttcagggcgttcagttaattctcccgtctaatgcgcttccctgtttttatgttattctctctgtaaaggctgctattttcatttttgacgttaaacaaaaaatcgtttcttatttggattgggataaataa

SEQ ID NO: 37 - protein, 583 aa, synthetic construct
VKKLLFAIPLVVPFYSHSAAQPAGGHGPIVDIVEDTGTGVEGGAQNGVVSTQENKDPIVDITEDTQPGMSGSNDATVVEEDTTPQRPDVLVGGQSDPIDITEDTQPGMSGSNDATVVEEDTVPKRPDILVGGQSDPIDITEDTQPGMSGSNDATVIEEDTKPKRFFHFDNEAAAPGGAETVESCLAKPHTENSFTNVWKDDKTLDRYANYEGCLWNATGVVVCTGDETQCYGTWVPIGLAIPENEGGGSEGGGSEGGGSEGGGTKPPEYGDRPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 38 - DNA, 1752 bp, synthetic construct
  source 1..1752: mol_type = other DNA
gtgaaaaaattattattcgcaattcctttagttgttcctttctattctcactccgcggcccagccggccggaggtcatggaccgattgtcgatatcgtcgaagatactggtacaggtgttgagggcggcgctcaaaacggcgtggtttcaactcaggaaaataaagatccaatcgttgacatcaccgaagatacccaaccaggtatgtcaggctcaaatgacgcgacagttgtcgaggaagacacaacacctcaacgcccagatgtccttgtaggtggtcaaagtgatccaatcgatatcactgaagatacccaaccaggcatgtcaggctcaaatgacgcgacagttgtcgaggaagacacagtacctaaacgtccagatatccttgttggcggtcaaagtgatccaatcgatatcaccgaagatacccaaccaggtatgtcaggctcaaatgacgctactgttatcgaagaagatacgaaaccaaaacgcttcttccactttgataacgaggcggccgctcccgggggcgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggctctgagggtggcggtactaaacctcctgagtacggtgatagacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaa

SEQ ID NO: 39 - protein, 691 aa, synthetic construct
VKKLLFAIPLVVPFYSHSAAQPAMGQVQLVQSGAEVKKPGASVKVSCKASGYTFTGYYMHWVRQAPGQGLEWMGWISPVSGGTNYAQKFQGRVTMTRDTSISTAYMELSRLRSDDTAVYYCARAPLFPTGVLAGDYYYYGMDVWGQGTTVTVSSGGGGSGGGGSGGGGSQSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTLVFGGGTKLTVLAAAPGGAETVESCLAKPHTENSFTNVWKDDKTLDRYANYEGCLWNATGVVVCTGDETQCYGTWVPIGLAIPENEGGGSEGGGSEGGGSEGGGTKPPEYGDRPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 40 - DNA, 2076 bp, synthetic construct
  source 1..2076: mol_type = other DNA
gtgaaaaaattattattcgcaattcctttagttgttcctttctattctcactccgcggcccagccggccatgggacaagttcagctggttcagagcggtgcggaagtgaaaaaaccgggcgccagcgtgaaagtgagctgcaaagccagcggctacaccttcaccggttactacatgcactgggtgcgccaagccccgggtcaaggcctcgaatggatgggctggatcagcccagtgagcggcggtacgaattacgcccagaaatttcaaggccgcgttaccatgacccgcgacaccagcatcagcacggcgtacatggaactgagtcgtctgcgcagcgatgacaccgccgtttactattgcgcgcgcgccccactgtttccaaccggtgttctggcgggcgactactactactacggcatggacgtttggggtcaaggcaccaccgttaccgttagcagtggtggcggtggtagcggtggtggtggtagcggcggtggtggcagccaaagcgcgctgacccagccagccagtgttagcggtagcccgggtcagagcattaccatcagctgcaccggcaccagcagcgatgttggcagctacaatctggttagctggtaccaacagcacccgggtaaagcgccgaagctgatgatctatgagggcagcaaacgcccaagcggcgtgagcaatcgcttcagcggcagcaaaagcggcaataccgcgagtctgaccatcagcggtctgcaagccgaagatgaagcggactactactgctgcagctacgcgggcagcagtacgctggttttcggcggcggcaccaaactgaccgttctggcggccgctcccgggggcgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggctctgagggtggcggtactaaacctcctgagtacggtgatagacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaa

SEQ ID NO: 41 - DNA, 2076 bp, synthetic construct; identical to SEQ ID NO: 40
  source 1..2076: mol_type = other DNA

SEQ ID NO: 42 - DNA, 579 bp, synthetic construct
  source 1..579: mol_type = other DNA
aatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccactgacgttggagtccacgttcttt

SEQ ID NO: 43 - DNA, 766 bp, synthetic construct
  source 1..766: mol_type = other DNA
ggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgagctcggtacccgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgcgccctgtagcggcgcattaagcgcggcgggtgtggtggttacgcgcagcgtgaccgctacacttgccagcgccctagcgcccgctccttgtgcggtatttcacaccgcatacgtcaaagcaaccatagtacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactgga

SEQ ID NO: 44 - DNA, 683 bp, synthetic construct
  source 1..683: mol_type = other DNA
aatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgagctcggtacccgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgcgccctgtagcggcgcattaagcgcggcgggtgtggtggttacgcgcagcgtgaccgctacacttgccagcgccctagcgcccgctccttgtgcggtatttcacaccgcatacgtcaaagcaaccatagtacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttcttt

SEQ ID NO: 45 - DNA, 185 bp, synthetic construct
  source 1..185: mol_type = other DNA
ggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaac

SEQ ID NO: 46 - DNA, 308 bp, synthetic construct
  source 1..308: mol_type = other DNA
gcgaattttaacaaaatattaacgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtac

SEQ ID NO: 47 - DNA, 291 bp, synthetic construct
  source 1..291: mol_type = other DNA
gcgaattttaacaaaatattaacgagctcggtacccgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtac

SEQ ID NO: 48 - DNA, 91 bp, synthetic construct
  source 1..91: mol_type = other DNA
gcgccctgtagcggcgcattaagcgcggcgggtgtggtggttacgcgcagcgtgaccgctacacttgccagcgccctagcgcccgctcctt

SEQ ID NO: 49 - DNA, 28 bp, synthetic construct
  source 1..28: mol_type = other DNA
tcgctttcttcccttcctttctcgccac

SEQ ID NO: 50 - DNA, 149 bp, synthetic construct
  source 1..149: mol_type = other DNA
gttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctt

SEQ ID NO: 51 - DNA, 50 bp, synthetic construct
  source 1..50: mol_type = other DNA
tgacgttggagtccacgttctttaatagtggactcttgttccaaactgga

SEQ ID NO: 52 - DNA, 789 bp, synthetic construct
  source 1..789: mol_type = other DNA
ggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtgattatcaaccggggtacatatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaggatcctacgcgccctgtagcggcgcattaagcgcggcgggtgtggtggttacgcgcagcgtgaccgctacacttgccagcgccctagcgcccgctcctttcgctttcttcccttcctttctcgccagttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactgga

SEQ ID NO: 53 - DNA, 706 bp, synthetic construct
  source 1..706: mol_type = other DNA
aatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtgattatcaaccggggtacatatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaggatcctacgcgccctgtagcggcgcattaagcgcggcgggtgtggtggttacgcgcagcgtgaccgctacacttgccagcgccctagcgcccgctcctttcgctttcttcccttcctttctcgccagttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttcttt

SEQ ID NO: 54 - DNA, 831 bp, synthetic construct
  source 1..831: mol_type = other DNA
ggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttgaattcccccaggctttacactttatgcttccggctcgtatgttgtgtggaattgtgagcggataacaatttcacacaggaaacagctatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaggatcctacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactgga

SEQ ID NO: 55 - DNA, 748 bp, synthetic construct
  source 1..748: mol_type = other DNA
aatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttgaattcccccaggctttacactttatgcttccggctcgtatgttgtgtggaattgtgagcggataacaatttcacacaggaaacagctatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaggatcctacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttcttt

SEQ ID NO: 56 - DNA, 40 bp, synthetic construct
  source 1..40: mol_type = other DNA
gcgaattttaacaaaatattaacgtttacaatttgaattc

SEQ ID NO: 57 - DNA, 81 bp, synthetic construct
  source 1..81: mol_type = other DNA
gcgaattttaacaaaatattaacgtttacaatttaaatatttgcttatacaatcttcctgtgattatcaaccggggtacat

SEQ ID NO: 58 - DNA, 82 bp, synthetic construct
  source 1..82: mol_type = other DNA
ccccaggctttacactttatgcttccggctcgtatgttgtgtggaattgtgagcggataacaatttcacacaggaaacagct

SEQ ID NO: 59 - DNA, 197 bp, synthetic construct
  source 1..197: mol_type = other DNA
atggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatga

SEQ ID NO: 60 - skipped

SEQ ID NO: 61 - DNA, 613 bp, synthetic construct
  source 1..613: mol_type = other DNA
ggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgagctcggtacccgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaa

SEQ ID NO: 62 - DNA, 529 bp, synthetic construct
  source 1..529: mol_type = other DNA
aatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgagctcggtacccgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttcttt

SEQ ID NO: 63 - DNA, 459 bp, synthetic construct
  source 1..459: mol_type = other DNA
ttggagtccacggttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgcgccctgtagcggcgcattaagcgcggcgggtgtggtggttacgcgcagcgtgaccgctacacttgccagcgccctagcgcccgctccttgctttccccgtcaagctctaaatcgggggctccctttagggttccgatttagtgctttacggcacctcgaccccaaaaaacttgatttgggtgatggttcacgtagtgggccatcgccctgatagacggtttttcgcccttggagtccacgttctttaatagtggactcttgttccaaactggaa

SEQ ID NO: 64 - DNA, 400 bp, synthetic construct
  source 1..400: mol_type = other DNA
aatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgcgccctgtagcggcgcattaagcgcggcgggtgtggtggttacgcgcagcgtgaccgctacacttgccagcgccctagcgcccgctccttgctttccccgtcaagctctaaatcgggggctccctttagggttccgatttagtgctttacggcacctcgaccccaaaaaacttgatttgggtgatggttcacgtagtgggccatcgccctgatagacggtttttcgcccttggagtccacgttcttt

SEQ ID NO: 65 - DNA, 160 bp, synthetic construct
  source 1..160: mol_type = other DNA
ttggagtccacggttggagtccacgttctttaatagtggactcttgttccaaactggaacaacactcaaccctatctcgggctattcttttgatttataagggattttgccgatttcggcctattggttaaaaaatgagctgatttaacaaaaatttaac

SEQ ID NO: 66 - DNA, 109 bp, synthetic construct
  source 1..109: mol_type = other DNA
gcgaattttaacaaaatattaacgagctcggtacccgcctgatgcggtattttctccttacgcatctgtgcggtatttcacaccgcatacgtcaaagcaaccatagtac

SEQ ID NO: 67 - DNA, 23 bp, synthetic construct
  source 1..23: mol_type = other DNA
gcgaattttaacaaaatattaac

SEQ ID NO: 68 - DNA, 86 bp, synthetic construct
  source 1..86: mol_type = other DNA
gtgtagcggcgcattaagcgcggcgggtgtggtggttacgcgcagcgtgaccgctacacttgccagcgccctagcgcccgctcctt

SEQ ID NO: 69 - DNA, 44 bp, synthetic construct
  source 1..44: mol_type = other DNA
ggagtccacgttctttaatagtggactcttgttccaaactggaa

SEQ ID NO: 70 - DNA, 359 bp, synthetic construct
  source 1..359: mol_type = other DNA
ttggagtccacgttctttaatagtggactcttgttccaaactggaacaacactcaaccctatctctcgaattttaacaaaatattaacgtgtagcggcgcattaagcgcggcgggtgtggtggttacgcgcagcgtgaccgctacacttgccagcgccctagcgcccgctccttgctttccccgtcaagctctaaatcgggggctccctttagggttccgatttagtgctttacggcacctcgaccccaaaaaacttgatttgggtgatggttcacgtagtgggccatcgccctgatagacggtttttcgcccttggagtccacgttctttaatagtggactcttgttccaaactggaa

SEQ ID NO: 71 - DNA, 313 bp, synthetic construct
  source 1..313: mol_type = other DNA
aatagtggactcttgttccaaactggaacaacactcaaccctatctctcgaattttaacaaaatattaacgtgtagcggcgcattaagcgcggcgggtgtggtggttacgcgcagcgtgaccgctacacttgccagcgccctagcgcccgctccttgctttccccgtcaagctctaaatcgggggctccctttagggttccgatttagtgctttacggcacctcgaccccaaaaaacttgatttgggtgatggttcacgtagtgggccatcgccctgatagacggtttttcgcccttggagtccacgttcttt

SEQ ID NO: 72 - DNA, 335 bp, synthetic construct
  source 1..335: mol_type = other DNA
ttggagtccacgttctttaatagtggactcttgttccaaactggaacaacactcaaccctatctctgtagcggcgcattaagcgcggcgggtgtggtggttacgcgcagcgtgaccgctacacttgccagcgccctagcgcccgctccttgctttccccgtcaagctctaaatcgggggctccctttagggttccgatttagtgctttacggcacctcgaccccaaaaaacttgatttgggtgatggttcacgtagtgggccatcgccctgatagacggtttttcgcccttggagtccacgttctttaatagtggactcttgttccaaactggaa

SEQ ID NO: 73 - DNA, 289 bp, synthetic construct
  source 1..289: mol_type = other DNA
aatagtggactcttgttccaaactggaacaacactcaaccctatctctgtagcggcgcattaagcgcggcgggtgtggtggttacgcgcagcgtgaccgctacacttgccagcgccctagcgcccgctccttgctttccccgtcaagctctaaatcgggggctccctttagggttccgatttagtgctttacggcacctcgaccccaaaaaacttgatttgggtgatggttcacgtagtgggccatcgccctgatagacggtttttcgcccttggagtccacgttcttt

SEQ ID NO: 74 - DNA, 65 bp, synthetic construct
  source 1..65: mol_type = other DNA
ttggagtccacgttctttaatagtggactcttgttccaaactggaacaacactcaaccctatctc

SEQ ID NO: 75 - DNA, 24 bp, synthetic construct
  source 1..24: mol_type = other DNA
tcgaattttaacaaaatattaacg

SEQ ID NO: 76 - DNA, 85 bp, synthetic construct
  source 1..85: mol_type = other DNA
tgtagcggcgcattaagcgcggcgggtgtggtggttacgcgcagcgtgaccgctacacttgccagcgccctagcgcccgctcctt

SEQ ID NO: 77 - DNA, 305 bp, synthetic construct
  source 1..305: mol_type = other DNA
ggccatcgccctgatagacggtttttcgccctttgacgttggagtccacgttctttaatagtggactcttgttccaaactggaacaacactcaaccctatctcgggcaagcttggacgcgccctgtagcggcgcattaagcgcggcgggtgtggtggttacgcgcagcgtgaccgctacacttgccagcgccctagcgcccgctcccgggatcggaattccggccatcgccctgatagacggtttttcgccctttgacgttggagtccacgttctttaatagtggactcttgttccaaactggaa

SEQ ID NO: 78 - DNA, 221 bp, synthetic construct
  source 1..221: mol_type = other DNA
aatagtggactcttgttccaaactggaacaacactcaaccctatctcgggcaagcttggacgcgccctgtagcggcgcattaagcgcggcgggtgtggtggttacgcgcagcgtgaccgctacacttgccagcgccctagcgcccgctcccgggatcggaattccggccatcgccctgatagacggtttttcgccctttgacgttggagtccacgttcttt

SEQ ID NO: 79 - DNA, 198 bp, synthetic construct
  source 1..198: mol_type = other DNA
ttggagtccacgttctttaatagtggactcttgttccaaactggaacaacactcaaccctatctctgtagcggcgcattaagcgcggcgggtgtggtggttacgcgcagcgtgaccgctacacttgccagcgccctagcgcccgctcctggatcggagtccacgttctttaatagtggactcttgttccaaactggaa

SEQ ID NO: 80 - DNA, 152 bp, synthetic construct
  source 1..152: mol_type = other DNA
aatagtggactcttgttccaaactggaacaacactcaaccctatctctgtagcggcgcattaagcgcggcgggtgtggtggttacgcgcagcgtgaccgctacacttgccagcgccctagcgcccgctcctggatcggagtccacgttcttt

SEQ ID NO: 81 - DNA, 107 bp, synthetic construct
  source 1..107: mol_type = other DNA
ggccatcgccctgatagacggtttttcgccctttgacgttggagtccacgttctttaatagtggactcttgttccaaactggaacaacactcaaccctatctcgggc

SEQ ID NO: 82 - DNA, 16 bp, synthetic construct
  source 1..16: mol_type = other DNA
aagcttggacgcgccc

SEQ ID NO: 83 - DNA, 83 bp, synthetic construct
  source 1..83: mol_type = other DNA
tgtagcggcgcattaagcgcggcgggtgtggtggttacgcgcagcgtgaccgctacacttgccagcgccctagcgcccgctcc

SEQ ID NO: 84 - DNA, 84 bp, synthetic construct
  source 1..84: mol_type = other DNA
tgtagcggcgcattaagcgcggcgggtgtggtggttacgcgcagcgtgaccgctacacttgccagcgccctagcgcccgctcct

SEQ ID NO: 85 - DNA, 15 bp, synthetic construct
  source 1..15: mol_type = other DNA
cgggatcggaattcc

SEQ ID NO: 86 - skipped

SEQ ID NO: 87 - DNA, 84 bp, synthetic construct
  source 1..84: mol_type = other DNA
ggccatcgccctgatagacggtttttcgccctttgacgttggagtccacgttctttaatagtggactcttgttccaaactggaa

SEQ ID NO: 88 - DNA, 11 bp, synthetic construct
  source 1..11: mol_type = other DNA
gttctttaata

SEQ ID NO: 89 - DNA, 83 bp, synthetic construct
  source 1..83: mol_type = other DNA
gtcgaccgtttacaatttaaatatttgcttatacaatcttcctgtttttggggcttttctgattatcaaccggggtacatatg

SEQ ID NO: 90 - DNA, 91 bp, synthetic construct
  source 1..91: mol_type = other DNA
gtcgacccccaggctttacactttatgcttccggctcgtataatgtgtggaattgtgagcggataacaatttcacacaggaaacagctatg

SEQ ID NO: 91 - protein, 297 aa, synthetic construct
MPPRRYNPDTRRDELLERINLDIPGAVAQALREDLGGTVDANNDITAKLLPENSRSHATVITRENGVFCGKRWVEEVFIQLAGDDVTIIWHVDDGDVINANQSLFELEGPSRVLLTGERTALNFVQTLSGVASKVRHYVELLEGTNTQLLDTRKTLPGLRSALKYAVLCGGGANHRLGLSDAFLIKENHIIASGSVRQAVEKASWLHPDAPVEVEVENLEELDEALKAGADIIMLDNFETEQMREAVKRTNGKALLEVSGNVTDKTLREFAETGVDFISVGALTKHVQALDLSMRFR

SEQ ID NO: 92 - DNA, 981 bp, synthetic construct
  source 1..981: mol_type = other DNA
ggatcctatatgtggtgctaatacccggttcagagtagcatgtttctaccttatgattcgttagctatctggagttttaacatgccgcctcgccgctataaccctgacacccgacgtgacgagctgctggaacgcattaatctcgatatccccggcgcggtggcccaggcgctgcgggaagatttaggcggaacagtcgatgccaacaatgatattacggcaaaacttttaccggaaaattctcgctctcatgccacggtgatcacccgcgagaatggcgtcttttgcggcaaacgctgggttgaagaggtgtttattcaactggcaggcgacgatgtcaccataatctggcatgtggatgacggcgatgtcatcaatgccaatcaatccttgttcgaacttgaaggcccatcccgcgtgctgttaacgggcgaacgcactgcgcttaattttgtgcaaaccctttcaggagttgccagtaaggtacgccactatgtcgaattgctggaaggcaccaacacgcagttgttggatacgcgcaaaaccttacccggcctgcgttcagctctgaaatacgcggtactttgcggcggcggagcgaatcaccgtctggggctttctgatgccttcctgatcaaagaaaaccatattattgcctccggctcagtgcgccaggcggtcgaaaaagcgtcctggctgcacccggatgcgccagtagaagtcgaagtagagaatctggaagaacttgatgaagccctgaaagcaggagccgatatcatcatgctggataacttcgaaacagaacagatgcgcgaagccgtcaaacgcaccaacggcaaggcgctactggaagtgtctggcaacgtcactgacaaaacactgcgtgaatttgccgaaacgggcgtggactttatctccgtcggtgcgctaactaaacacgtacaagcactcgacctttcaatgcgttttcgctaatacgta

SEQ ID NO: 93 - DNA, 988 bp, synthetic construct
  source 1..988: mol_type = other DNA
gactccccgtcgtatatgtggtgctaatacccggttcagagtagcatgtttctaccttatgattcgttagctatctggagttttaacatgccgcctcgccgctataaccctgacacccgacgtgacgagctgctggaacgcattaatctcgatatccccggcgcggtggcccaggcgctgcgggaagatttaggcggaacagtcgatgccaacaatgatattacggcaaaacttttaccggaaaattctcgctctcatgccacggtgatcacccgcgagaatggcgtcttttgcggcaaacgctgggttgaagaggtgtttattcaactggcaggcgacgatgtcaccataatctggcatgtggatgacggcgatgtcatcaatgccaatcaatccttgttcgaacttgaaggcccatcccgcgtgctgttaacgggcgaacgcactgcgcttaattttgtgcaaaccctttcaggagttgccagtaaggtacgccactatgtcgaattgctggaaggcaccaacacgcagttgttggatacgcgcaaaaccttacccggcctgcgttcagctctgaaatacgcggtactttgcggcggcggagcgaatcaccgtctggggctttctgatgccttcctgatcaaagaaaaccatattattgcctccggctcagtgcgccaggcggtcgaaaaagcgtcctggctgcacccggatgcgccagtagaagtcgaagtagagaatctggaagaacttgatgaagccctgaaagcaggagccgatatcatcatgctggataacttcgaaacagaacagatgcgcgaagccgtcaaacgcaccaacggcaaggcgctactggaagtgtctggcaacgtcactgacaaaacactgcgtgaatttgccgaaacgggcgtggactttatctccgtcggtgcgctaactaaacacgtacaagcactcgacctttcaatgcgttttcgctaacctcgag

SEQ ID NO: 94 - DNA, 8869 bp, synthetic construct
  source 1..8869: mol_type = other DNA
aacgctactactattagtagaattgatgccaccttttcagctcgcgccccaaatgaaaatatagctaaacaggttattgaccatttgcgaaatgtatctaatggtcaaactaaatctactcgttcgcagaattgggaatcaattgttacatggaatgaaacttccagacaccgtactttagttgcatatttaaaacatgttgagctacagcaccagatccagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcgcaattcactacccgttctggtgtttctcgtcagggcaagccttattcactgaatgagcagctttgttacgttgatttgggtaatgaatatccggtgcttgtcaagattactcttgatgaaggtcagccagcctatgcgcctggtctgtacaccgttcatctgtcctcgttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtagttagtcctcaaagcctctgtagccgttgctaccctcgttccgatgctgtctttcgctgctgagggtgacgatcccgcaaaagcggcctttgactccctgcaagcctcagcgaccgaaatgatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggctctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaatcatgccagttcttttgggtattccgttattattgcgtttcctcggtttccttctggtaactttgttcggctatctgctaacttttcttaaaaagggcttcggtaagatagctattgctatttcattgtttcttgctcttattattgggcttaactcaattcttgtgggttatctctctgatattagcgctcaattaccctctgactttgttcagggcgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaatatgttgtttattgtcgtcgtctggacagaattactttgccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttccagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaagtaattgaaatgaataattcgcctctgcgcgatttcgttacttggtattcaaagcaaacaggtgaatctgttattgtctcgcctgatgttaagggtactgtgactgtatattcatctgacgttaaacctgaaaatctacgcaatttctttatttctgttttacgtgcaaataattttgatatggtaggttctaacccttccattattcagaagtataatccaaacaatcaggactatattgatgaattgccatcatctgataatcaggaatatgatgataattccgctccttctggtggtttctttgttccgcaaaatgataatgttactcaaacttttaaaattaataacgttcgggcaaaggatttaatacgagttgtagaattgtttgttaaatctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatttctgttggtcagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtaggcgtctagaacggatcgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgattcgagctcgcccggggatcgaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataaccccccttgtattactgtttatgtaagcagacagttttattgttcatgatgatatatttttatcttgtgcaatgtaacatcagagattttgaaacacaacgtggctttcccccccccccccctgcaggtcggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgagctcggtacccgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaagtcgacccccaggctttacactttatgcttccggctcgtataatgtgtggaattgtgagcggataacaatttcacacaggaaacagctatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 95 - DNA, 8301 bp, synthetic construct
  source 1..8301: mol_type = other DNA
aacgctactactattagtagaattgatgccaccttttcagctcgcgccccaaatgaaaatatagctaaacaggttattgaccatttgcgaaatgtatctaatggtcaaactaaatctactcgttcgcagaattgggaatcaattgttacatggaatgaaacttccagacaccgtactttagttgcatatttaaaacatgttgagctacagcaccagatccagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcgcaattcactacccgttctggtgtttctcgtcagggcaagccttattcactgaatgagcagctttgttacgttgatttgggtaatgaatatccggtgcttgtcaagattactcttgatgaaggtcagccagcctatgcgcctggtctgtacaccgttcatctgtcctcgttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtagttagtcctcaaagcctctgtagccgttgctaccctcgttccgatgctgtctttcgctgctgagggtgacgatcccgcaaaagcggcctttgactccctgcaagccttagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgcggcccagccggccatgggatatcaggcggccgctcccgggggcgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggctctgagggtggcggtactaaacctcctgagtacggtgatagacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaatcatgccagttcttttgggtattccgttattattgcgtttcctcggtttccttctggtaactttgttcggctatctgctaacttttcttaaaaagggcttcggtaagatagctattgctatttcattgtttcttgctcttattattgggcttaactcaattcttgtgggttatctctctgatattagcgctcaattaccctctgactttgttcagggcgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaatatgttgtttattgtcgtcgtctggacagaattactttgccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttccagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaagtaattgaaatgaataattcgcctctgcgcgatttcgttacttggtattcaaagcaaacaggtgaatctgttattgtctcgcctgatgttaagggtactgtgactgtatattcatctgacgttaaacctgaaaatctacgcaatttctttatttctgttttacgtgcaaataattttgatatggtaggttctaacccttccattattcagaagtataatccaaacaatcaggactatattgatgaattgccatcatctgataatcaggaatatgatgataattccgctccttctggtggtttctttgttccgcaaaatgataatgttactcaaacttttaaaattaataacgttcgggcaaaggatttaatacgagttgtagaattgtttgttaaatctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatttctgttggtcagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtaggcgtctagaacggatcgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgattcgagctcgcccggggatcgaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataaccccccttgtattactgtttatgtaagcagacagttttattgttcatgatgatatatttttatcttgtgcaatgtaacatcagagattttgaaacacaacgtggctttcccccccccccccctgcaggtcgtcgacccccaggctttacactttatgcttccggctcgtataatgtgtggaattgtgagcggataacaatttcacacaggaaacagctatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 96 - DNA, 3075 bp, synthetic construct
  source 1..3075: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgagctcggtacccgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaactcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacaggatcctatatgtggtgctaatacccggttcagagtagcatgtttctaccttatgattcgttagctatctggagttttaacatgccgcctcgccgctataaccctgacacccgacgtgacgagctgctggaacgcattaatctcgatatccccggcgcggtggcccaggcgctgcgggaagatttaggcggaacagtcgatgccaacaatgatattacggcaaaacttttaccggaaaattctcgctctcatgccacggtgatcacccgcgagaatggcgtcttttgcggcaaacgctgggttgaagaggtgtttattcaactggcaggcgacgatgtcaccataatctggcatgtggatgacggcgatgtcatcaatgccaatcaatccttgttcgaacttgaaggcccatcccgcgtgctgttaacgggcgaacgcactgcgcttaattttgtgcaaaccctttcaggagttgccagtaaggtacgccactatgtcgaattgctggaaggcaccaacacgcagttgttggatacgcgcaaaaccttacccggcctgcgttcagctctgaaatacgcggtactttgcggcggcggagcgaatcaccgtctggggctttctgatgccttcctgatcaaagaaaaccatattattgcctccggctcagtgcgccaggcggtcgaaaaagcgtcctggctgcacccggatgcgccagtagaagtcgaagtagagaatctggaagaacttgatgaagccctgaaagcaggagccgatatcatcatgctggataacttcgaaacagaacagatgcgcgaagccgtcaaacgcaccaacggcaaggcgctactggaagtgtctggcaacgtcactgacaaaacactgcgtgaatttgccgaaacgggcgtggactttatctccgtcggtgcgctaactaaacacgtacaagcactcgacctttcaatgcgttttcgctaatacgtactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 97 - protein, 77 aa, synthetic construct
MKKXLVLKASVAVATLVPMLSFAGGGGGEGDDPAKAAFDSLQALATEYIGYAWAMVVVIVGATIGIKLFKKFTSKAS

SEQ ID NO: 98 - DNA, 234 bp, synthetic construct
  source 1..234: mol_type = other DNA
atgaaaaagtagttagtcctcaaagcctctgtagccgttgctaccctcgttccgatgctgtctttcgctggcggtggcggtggcgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 99 - protein, 556 aa, synthetic construct
VKKLLFAIPLVVPFYSHSAAQPAMGKVKLQQSGGGLVQTGGSLRLSCAPSGRTFSVNAMGWYRQAPGKQREPVALISTGGSTNYADSVKGRFTISRDNVKNTVYLQMNTLKPEDTAVYYCYTERWKPRGIERDWGQGTQVTVSSAAAPGGAETVESCLAKPHTENSFTNVWKDDKTLDRYANYEGCLWNATGVVVCTGDETQCYGTWVPIGLAIPENEGGGSEGGGSEGGGSEGGGTKPPEYGDRPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 100 - DNA, 1671 bp, synthetic construct
  source 1..1671: mol_type = other DNA
gtgaaaaaattattattcgcaattcctttagttgttcctttctattctcactccgcggcccagccggccatgggaaaagtgaagttgcagcagagcgggggtggtctggtacaaacgggcggttcacttcggctgagctgcgcgccatcgggccgcacctttagcgtgaacgcgatgggctggtatcgccaagcgccgggcaaacagcgcgaaccggtggcgctgattagcacgggcggcagcaccaactatgcggatagcgtgaaaggccgctttaccattagccgcgataacgtgaaaaacaccgtgtatctgcagatgaacaccctgaaaccggaagataccgcggtgtattattgctataccgaacgctggaaaccgcgcggcattgaacgcgattggggccaaggcacccaagtgaccgtgagcagcgcggccgctcccgggggcgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggctctgagggtggcggtactaaacctcctgagtacggtgatagacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgttaatgctggcggcggctctggtggtggttctggtggcggctctgagggtggtggctctgagggtggcggttctgagggtggcggctctgagggtggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgacggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaa

SEQ ID NO: 101 - DNA, 1445 bp, synthetic construct
  source 1..1445: mol_type = other DNA
ttggagtccacgttctttaatagtggactcttgttccaaactggaacaacactcaaccctatctcgggctattcttttgatttataagggattttgccgatttcggcctattggttaaaaaatgagctgatttaacaaaaatttaacggggatcgcatgtcccacgtgaaacattgctaaaccctcaggcctctgagcgacaaaagctttaaagggaaattcgcgcccataacttggtccgaatacgggttctagcatcgttcgtctgagtttgttctatataaaacgggcgcaatgtctgctttgatcaacctccaatacctcgtatgattgtgcacccgccggtgaccactcaatgatgtggggtccccgttgcaactacgaggatttattgagaccgacctacgttcggcattgtgggcagagtgaagtattggcaaacgttaagtgccgaactagatctgacctaacggtaagagagtttcataatacgtccagccgcatgcgcagggtacatttggacagtattgaatggactctgatcaaccttcacaccgatctagaaacgagtgcgtagatcagccaggtgcaaaccaaaaattctaggttactagaagttttgcgacgttctaagagttggacgaaatgtttcgcgacctaggatgaggtcgccctagaaaatagatttctgctactctcctcataagcagtccggtgtatcgaaagtacaagactagccttgctagcaaccgcgggctgggagcctaaggcatcactcaagatacaggctcggtaacgtacgctctagccatctaactatcccctatgtcttatagggacctacgttatctgcctgtcgaaccataggattcgcatcagcgcgcaggcttgggtcgagataaaatctccagtgcccaagaccacgggcgctcggcgtcttggctaatccccgtacatgttgttataaataatcagtagaaactctgtgttagagggtggagtgaccttaaatcaaggacgatattaatcggaaggagtattcaacgtgatgaagtcgcagggttgacgtgggaatggtgcttctgtccaaacaggttagggtataacgccggaaccgtcccccaagcgtacagtgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactgga

SEQ ID NO: 102 - DNA, 1400 bp, synthetic construct
  source 1..1400: mol_type = other DNA
aatagtggactcttgttccaaactggaacaacactcaaccctatctcgggctattcttttgatttataagggattttgccgatttcggcctattggttaaaaaatgagctgatttaacaaaaatttaacggggatcgcatgtcccacgtgaaacattgctaaaccctcaggcctctgagcgacaaaagctttaaagggaaattcgcgcccataacttggtccgaatacgggttctagcatcgttcgtctgagtttgttctatataaaacgggcgcaatgtctgctttgatcaacctccaatacctcgtatgattgtgcacccgccggtgaccactcaatgatgtggggtccccgttgcaactacgaggatttattgagaccgacctacgttcggcattgtgggcagagtgaagtattggcaaacgttaagtgccgaactagatctgacctaacggtaagagagtttcataatacgtccagccgcatgcgcagggtacatttggacagtattgaatggactctgatcaaccttcacaccgatctagaaacgagtgcgtagatcagccaggtgcaaaccaaaaattctaggttactagaagttttgcgacgttctaagagttggacgaaatgtttcgcgacctaggatgaggtcgccctagaaaatagatttctgctactctcctcataagcagtccggtgtatcgaaagtacaagactagccttgctagcaaccgcgggctgggagcctaaggcatcactcaagatacaggctcggtaacgtacgctctagccatctaactatcccctatgtcttatagggacctacgttatctgcctgtcgaaccataggattcgcatcagcgcgcaggcttgggtcgagataaaatctccagtgcccaagaccacgggcgctcggcgtcttggctaatccccgtacatgttgttataaataatcagtagaaactctgtgttagagggtggagtgaccttaaatcaaggacgatattaatcggaaggagtattcaacgtgatgaagtcgcagggttgacgtgggaatggtgcttctgtccaaacaggttagggtataacgccggaaccgtcccccaagcgtacagtgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttcttt

SEQ ID NO: 103 - DNA, 147 bp, synthetic construct
  source 1..147: mol_type = other DNA
ttggagtccacgttctttaatagtggactcttgttccaaactggaacaacactcaaccctatctcgggctattcttttgatttataagggattttgccgatttcggcctattggttaaaaaatgagctgatttaacaaaaatttaac

SEQ ID NO: 104 - DNA, 986 bp, synthetic construct
  source 1..986: mol_type = other DNA
ggggatcgcatgtcccacgtgaaacattgctaaaccctcaggcctctgagcgacaaaagctttaaagggaaattcgcgcccataacttggtccgaatacgggttctagcatcgttcgtctgagtttgttctatataaaacgggcgcaatgtctgctttgatcaacctccaatacctcgtatgattgtgcacccgccggtgaccactcaatgatgtggggtccccgttgcaactacgaggatttattgagaccgacctacgttcggcattgtgggcagagtgaagtattggcaaacgttaagtgccgaactagatctgacctaacggtaagagagtttcataatacgtccagccgcatgcgcagggtacatttggacagtattgaatggactctgatcaaccttcacaccgatctagaaacgagtgcgtagatcagccaggtgcaaaccaaaaattctaggttactagaagttttgcgacgttctaagagttggacgaaatgtttcgcgacctaggatgaggtcgccctagaaaatagatttctgctactctcctcataagcagtccggtgtatcgaaagtacaagactagccttgctagcaaccgcgggctgggagcctaaggcatcactcaagatacaggctcggtaacgtacgctctagccatctaactatcccctatgtcttatagggacctacgttatctgcctgtcgaaccataggattcgcatcagcgcgcaggcttgggtcgagataaaatctccagtgcccaagaccacgggcgctcggcgtcttggctaatccccgtacatgttgttataaataatcagtagaaactctgtgttagagggtggagtgaccttaaatcaaggacgatattaatcggaaggagtattcaacgtgatgaagtcgcagggttgacgtgggaatggtgcttctgtccaaacaggttagggtataacgccggaaccgtcccccaagcgtacag